(19) United States
(12) Patent Application Publication
(10) Pub. No.: US 2025/0143180 A1
(43) Pub. Date: May 1, 2025

(54) NOVEL COMPOUND AND ORGANIC LIGHT EMITTING DEVICE COMPRISING THE SAME

(71) Applicant: LG CHEM, LTD., Seoul (KR)

(72) Inventors: Jeonghoe HEO, Daejeon (KR); Min Woo JUNG, Daejeon (KR); Hoon Jun KIM, Daejeon (KR); Miyeon HAN, Daejeon (KR); Joongsuk OH, Daejeon (KR); Hye Min CHO, Daejeon (KR); Hojung LEE, Daejeon (KR); Hyungjin PARK, Daejeon (KR)

(21) Appl. No.: 18/890,325

(22) Filed: Sep. 19, 2024

(30) Foreign Application Priority Data
Oct. 5, 2023 (KR) 10-2023-0132806

(51) Int. Cl. H10K 85/60; C07D 405/14; C07D 409/14; H10K 50/12

(52) U.S. Cl. CPC H10K 85/654; C07D 405/14; C07D 409/14; H10K 85/622; H10K 85/6572; H10K 85/6574; H10K 85/6576; H10K 50/12

(57) ABSTRACT

A compound of Chemical Formula 1:

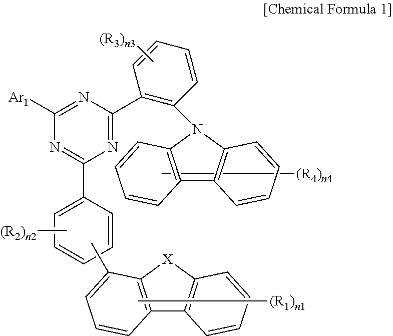

wherein: Ar₁ is a substituted or unsubstituted C₆₋₆₀ aryl or a substituted or unsubstituted C₂₋₆₀ heteroaryl containing at least one of N, O, or S as a heteroatom; X is O or S; R₁ is hydrogen, deuterium, a substituted or unsubstituted C₆₋₆₀ aryl, or a substituted or unsubstituted C₂₋₆₀ heteroaryl containing at least one heteroatom selected from the group consisting of N, O and S; each R₂ is independently hydrogen, deuterium, or a substituted or unsubstituted C₆₋₆₀ aryl; each R₃ is independently hydrogen, deuterium, or a substituted or unsubstituted C₆₋₆₀ aryl; each R₄ is independently hydrogen, deuterium, or a substituted or unsubstituted C₆₋₆₀ aryl; and the other substituents are as defined in the specification, wherein the compound of Chemical Formula 1 is substituted with at least one deuterium; and an organic light emitting device comprising the same.

### CROSS-REFERENCE TO RELATED APPLICATIONS

This application claims the benefit of and priority to Korean Patent Application No. 10-2023-0132806, filed on Oct. 5, 2023, the entire contents of which are incorporated herein by reference.

### TECHNICAL FIELD

The present disclosure relates to a novel compound and an organic light emitting device comprising the same.

### BACKGROUND ART

In general, an organic light emitting phenomenon refers to a phenomenon where electric energy is converted into light energy by using an organic material. The organic light emitting device using the organic light emitting phenomenon has characteristics such as a wide viewing angle, an excellent contrast, a fast response time, an excellent luminance, driving voltage and response speed, and thus many studies have proceeded.

The organic light emitting device generally has a structure which comprises an anode, a cathode, and an organic material layer interposed between the anode and the cathode. The organic material layer frequently has a multilayered structure that comprises different materials in order to enhance efficiency and stability of the organic light emitting device, and for example, the organic material layer may be formed of a hole injection layer, a hole transport layer, a light emitting layer, an electron transport layer, an electron injection layer and the like. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continued need to develop a new material for the organic material used in the organic light emitting device as described above.

### PRIOR ART LITERATURE

### Patent Literature

(Patent Literature 1) Korean Unexamined Patent Publication No. 10-2000-0051826

### DETAILED DESCRIPTION OF THE INVENTION

### Technical Problem

It is an object of the present disclosure to provide a novel organic light emitting material and an organic light emitting device comprising the same.

### Technical Solution

According to the present disclosure, provided is a compound represented by the following Chemical Formula 1:

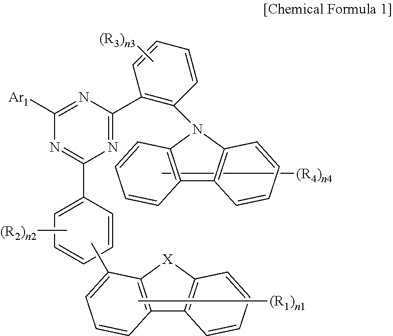

wherein in Chemical Formula 1,
Ar₁ is a substituted or unsubstituted C₆₋₆₀ aryl; or a substituted or unsubstituted C₂₋₆₀ heteroaryl containing at least one selected from the group consisting of N, O and S,
X is O or S,
R₁ is hydrogen; deuterium; a substituted or unsubstituted C₆₋₆₀ aryl; or a substituted or unsubstituted C₂₋₆₀ heteroaryl containing at least one selected from the group consisting of N, O and S,
each R₂ is independently hydrogen; deuterium; or a substituted or unsubstituted C₆₋₆₀ aryl,
each R₃ is independently hydrogen; deuterium; or a substituted or unsubstituted C₆₋₆₀ aryl,
each R₄ is independently hydrogen; deuterium; or a substituted or unsubstituted C₆₋₆₀ aryl,
n1 is an integer of 1 to 7,
n2 and n3 are each independently an integer of 1 to 4, and
n4 is an integer of 1 to 8,
wherein the compound represented by Chemical Formula 1 is substituted with at least one deuterium.

In addition, according to the present disclosure, provided is an organic light emitting device comprising: a first electrode; a second electrode that is provided opposite to the first electrode; and one or more organic material layers that are provided between the first electrode and the second electrode, wherein one or more layers of the organic material layers comprises the compound represented by Chemical Formula 1.

### Advantageous Effects

The above-mentioned compound represented by Chemical Formula 1 can be used as a material of an organic material layer in an organic light emitting device, and can improve the efficiency, achieve low driving voltage and/or improve lifetime characteristics in the organic light emitting device. In particular, the compound represented by Chemical Formula 1 can be used as a hole injection material, hole transport material, light emitting material, electron transport material, and/or electron injection material.

### BRIEF DESCRIPTION OF THE DRAWINGS

FIG. 1 shows an example of an organic light emitting device comprising a substrate 1, an anode 2, a light emitting layer 3, and a cathode 4.

FIG. 2 shows an example of an organic light emitting device comprising a substrate 1, an anode 2, a hole injection layer 5, a hole transport layer 6, an electron blocking layer 7, a light emitting layer 3, a hole blocking layer 8, an electron transport and injection layer 9, and a cathode 4.

### DETAILED DESCRIPTION OF THE EMBODIMENTS

Hereinafter, embodiments of the present disclosure will be described in more detail to help understanding of the invention.

The present disclosure provides the compound represented by Chemical Formula 1.

In the present disclosure, the notation [symbol] or [symbol] means a bond linked to another substituent group.

In the present disclosure, the term “substituted or unsubstituted” means being unsubstituted or substituted with one or more substituents selected from the group consisting of deuterium; a halogen group; a nitrile group; a nitro group; a hydroxy group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; and a heteroaryl group containing at least one of N, O and S atoms, or being unsubstituted or substituted with a substituent group to which two or more substituent groups of the above-exemplified substituent groups are linked. For example, “a substituent in which two or more substituents are linked” may be a biphenyl group. Namely, a biphenyl group may be an aryl group, or it may be interpreted as a substituent formed by linking two phenyl groups. In one example, the term “substituted or unsubstituted” may be understood as meaning “being unsubstituted or substituted with one or more substituents, for example, 1 to 5 substituents, selected from the group consisting of deuterium, halogen, a C₁₋₁₀ alkyl, a C₁₋₁₀ alkoxy, a C₆₋₂₀ aryl and a C₂₋₂₀ heteroaryl group containing at least heteroatoms of N, O and S. Alternatively, the term “substituted with one or more substituents” as used herein may be understood as meaning “being substituted with 1 to 5 substituents”, or “being substituted with one or two substituents”.

In the present disclosure, the carbon number of a carbonyl group is not particularly limited, but is preferably 1 to 40. Specifically, the carbonyl group may be a substituent having the following structural formulas, but is not limited thereto.

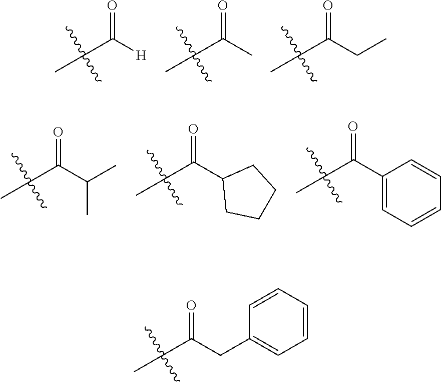

In the present disclosure, an ester group may have a structure in which oxygen of the ester group may be substituted by a straight-chain, branched-chain, or cyclic alkyl group having 1 to 25 carbon atoms, or an aryl group having 6 to 25 carbon atoms. Specifically, the ester group may be a substituent having the following structural formulas, but is not limited thereto.

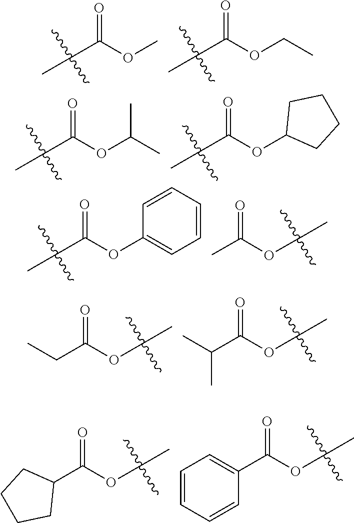

In the present disclosure, the carbon number of an imide group is not particularly limited, but is preferably 1 to 25. Specifically, the imide group may be a substituent group having the following structural formulas, but is not limited thereto.

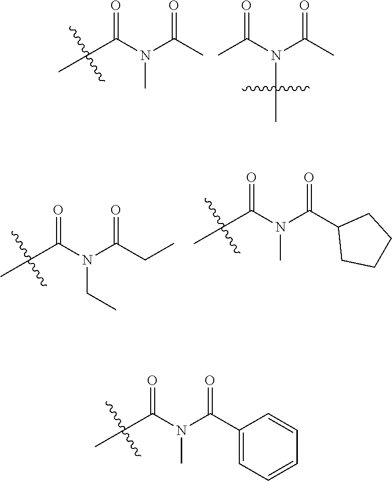

In the present disclosure, a silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group and the like, but are not limited thereto.

In the present disclosure, a boron group specifically includes a trimethylboron group, a triethylboron group, a t-butyldimethylboron group, a triphenylboron group, and a phenylboron group, but is not limited thereto.

In the present disclosure, examples of a halogen group include fluoro, chloro, bromo, or iodo.

In the present disclosure, the alkyl group may be straight-chain or branched-chain, and the carbon number thereof is not particularly limited, but is preferably 1 to 40. According to one embodiment, the carbon number of the alkyl group is 1 to 20. According to another embodiment, the carbon number of the alkyl group is 1 to 10. According to another embodiment, the carbon number of the alkyl group is 1 to 6. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 4-methylhexyl, 5-methylhexyl, and the like, but are not limited thereto.

In the present disclosure, the alkenyl group may be straight-chain or branched-chain, and the carbon number thereof is not particularly limited, but is preferably 2 to 40. According to one embodiment, the carbon number of the alkenyl group is 2 to 20. According to another embodiment, the carbon number of the alkenyl group is 2 to 10. According to still another embodiment, the carbon number of the alkenyl group is 2 to 6. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present disclosure, the cycloalkyl group is not particularly limited, but the carbon number thereof is preferably 3 to 60. According to another embodiment, the carbon number of the cycloalkyl group is 3 to 30. According to another embodiment, the carbon number of the cycloalkyl group is 3 to 20. According to another embodiment, the carbon number of the cycloalkyl group is 3 to 6. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present disclosure, an aryl group is not particularly limited, but the carbon number thereof is preferably 6 to 60, and it may be a monocyclic aryl group or a polycyclic aryl group. According to one embodiment, the carbon number of the aryl group is 6 to 30. According to one embodiment, the carbon number of the aryl group is 6 to 20. The aryl group may be a phenyl group, a biphenyl group, a terphenyl group or the like as the monocyclic aryl group, but is not limited thereto. The polycyclic aryl group includes a naphthyl group, an anthracenyl group, a phenanthrenyl group, a pyrenyl group, a perylenyl group, a chrysenyl group, a fluorenyl group, or the like, but is not limited thereto.

In the present disclosure, the fluorenyl group may be substituted, and two substituent groups may be linked with each other to form a spiro structure. In the case where the fluorenyl group is substituted,

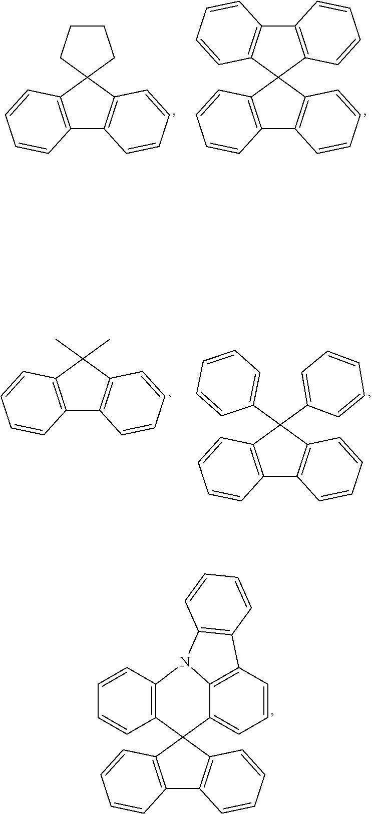

and the like can be formed. However, the structure is not limited thereto.

In the present disclosure, a heteroaryl group is a heteroaryl group containing one or more of O, N, Si and S as a heteroatom, and the carbon number thereof is not particularly limited, but is preferably 2 to 60. According to one embodiment, the carbon number of the heteroaryl group is 6 to 30. According to one embodiment, the carbon number of the heteroaryl group is 6 to 20. Examples of the heteroaryl group include a thiophene group, a furan group, a pyrrole group, an imidazole group, a thiazole group, an oxazol group, an oxadiazol group, a triazol group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazol group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, and the like, but are not limited thereto.

In the present disclosure, the aryl group in the aralkyl group, the aralkenyl group, the alkylaryl group and the arylamine group is the same as the examples of the aryl group as defined above. In the present disclosure, the alkyl group in the aralkyl group, the alkylaryl group and the alkylamine group is the same as the examples of the alkyl group as defined above. In the present disclosure, the heteroaryl in the heteroarylamine can be applied to the description of the heteroaryl as defined above. In the present disclosure, the alkenyl group in the aralkenyl group is the same as the examples of the alkenyl group as defined above. In the present disclosure, the description of the aryl group as defined above may be applied except that the arylene is a divalent group. In the present disclosure, the description of the heteroaryl as defined above can be applied except that the heteroarylene is a divalent group. In the present disclosure, the description of the aryl group or cycloalkyl group as defined above can be applied except that the hydrocarbon ring is not a monovalent group but formed by combining two substituent groups. In the present disclosure, the description of the heteroaryl group as defined above can be applied, except that the heteroaryl is not a monovalent group but formed by combining two substituent groups.

According to the present disclosure, provided is the compound represented by Chemical Formula 1. The compound represented by Chemical Formula 1 has a structure in which carbazole having hole transport characteristics and triazine having electron transport characteristics are adjacent to each other at the ortho position, and thus intra charge transfer easily occur, which provides high molecular stability and is advantageous for both hole and electron transport. Furthermore, when the carbon of 4-position of dibenzofuran or dibenzothiophene is bonded to phenyl, the efficiency is improved, particularly, when dibenzofuran/dibenzothiophene is adjacent to triazine in a meta-position, it exhibits high efficiency characteristics. In addition, by introducing various substituents to Ar₁ of Chemical Formula 1, the electron transport characteristics can be adjusted in various ways, which is advantageous for adjusting the charge balance due to changes in the common layer.

Preferably, the compound represented by Chemical Formula 1 may be represented by the following Chemical Formulas 1-1 to 1-3:

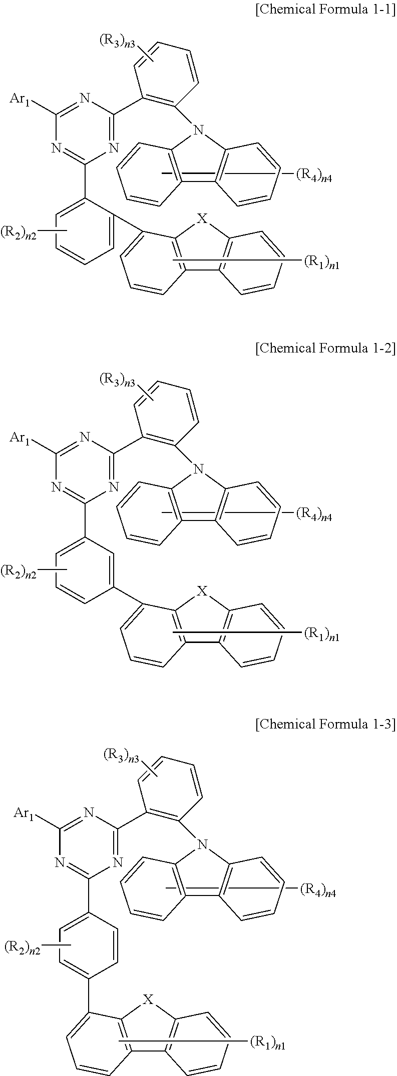

wherein in Chemical Formulas 1-1 to 1-3,
Ar₁, X, R₁ to R₄ and n1 to n4 are as defined in Chemical Formula 1.

Preferably, Ar₁ may be a substituted or unsubstituted C₆₋₂₀ aryl; or a substituted or unsubstituted C₂₋₂₀ heteroaryl containing at least one selected from the group consisting of N, O and S.

More preferably, Ar₁ may be phenyl, biphenylyl, terphenylyl, quaterphenylyl, naphthyl, phenanthrenyl, triphenylenyl, dibenzofuranyl, dibenzothiophenyl, benzothiophenyl, carbazolyl, pyridinyl, pyrimidinyl, quinolinyl, isoquinolinyl, quinoxalinyl, naphthyl phenyl, terphenylyl phenyl, phenyl substituted with one or two pyridinyls, phenyl substituted with two pyrimidinyls, carbazolyl phenyl, carbazolyl carbazolyl, or phenyl naphthyl, wherein the Ar₁ may be substituted or unsubstituted.

More preferably, Ar₁ may be phenyl, biphenylyl, terphenylyl, quaterphenylyl, naphthyl, phenanthrenyl, triphenylenyl, dibenzofuranyl, dibenzothiophenyl, benzothiophenyl, carbazolyl, pyridinyl, pyrimidinyl, quinolinyl, isoquinolinyl, quinoxalinyl, naphthyl phenyl, terphenylyl phenyl, phenyl substituted with one or two pyridinyls, phenyl substituted with two pyrimidinyls, carbazolyl phenyl, carbazolyl carbazolyl, or phenyl naphthyl, wherein the Ar₁ may be substituted or unsubstituted with at least one deuterium.

More preferably, Ar₁ may be any one selected from the group consisting of the following:

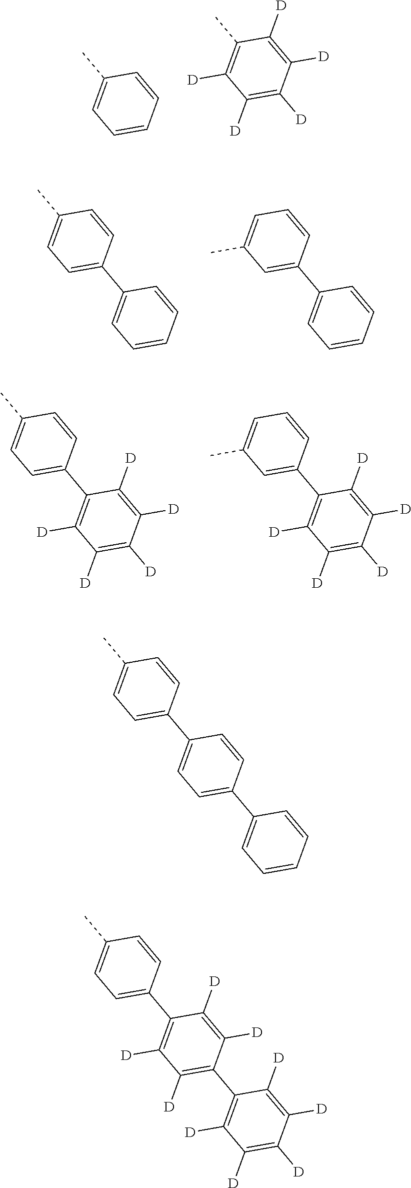

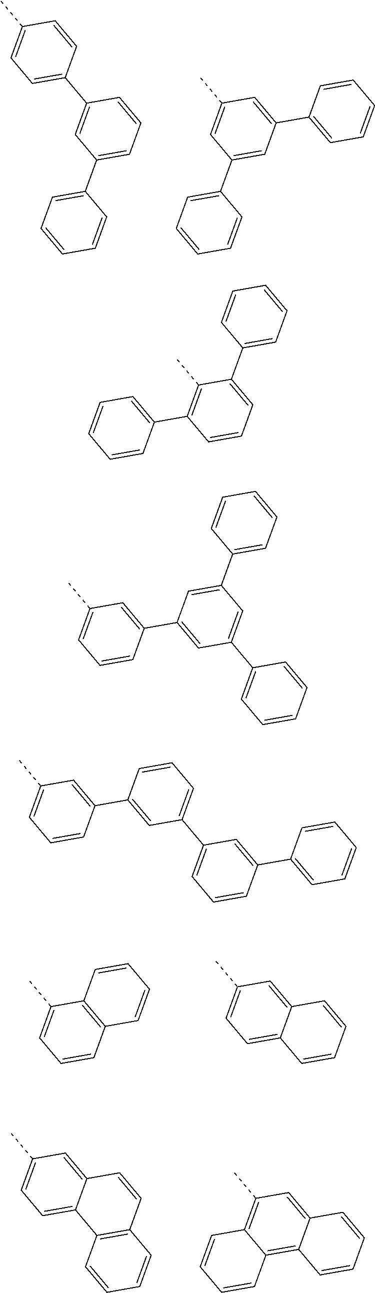

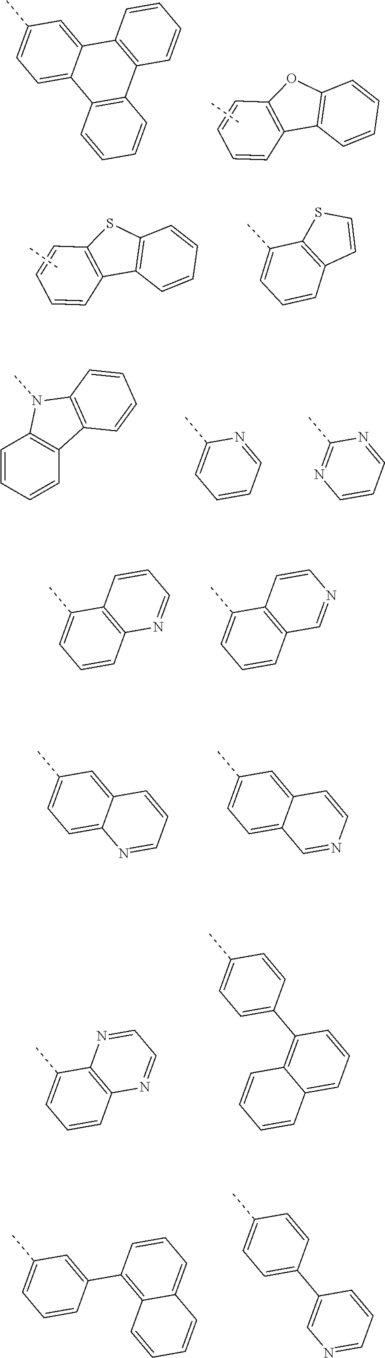

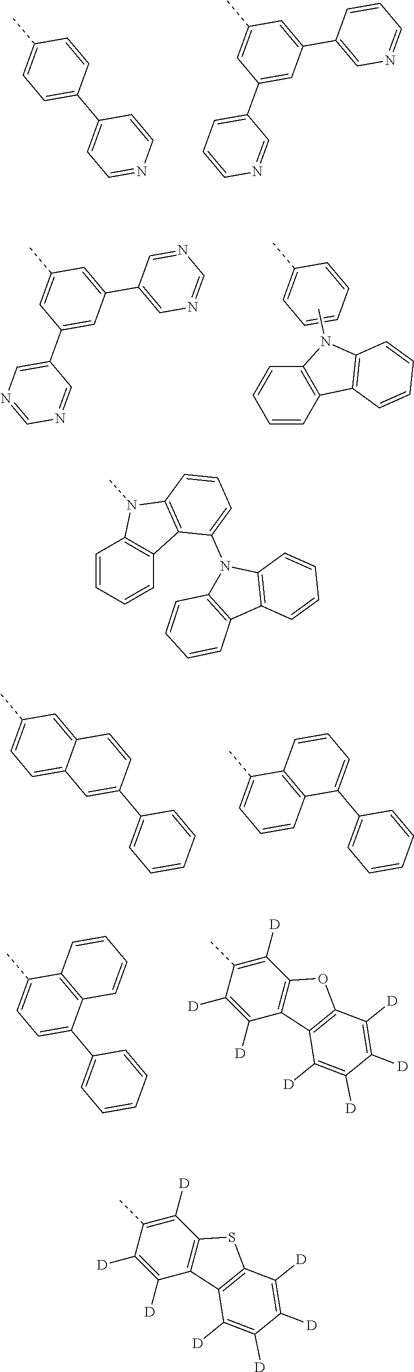

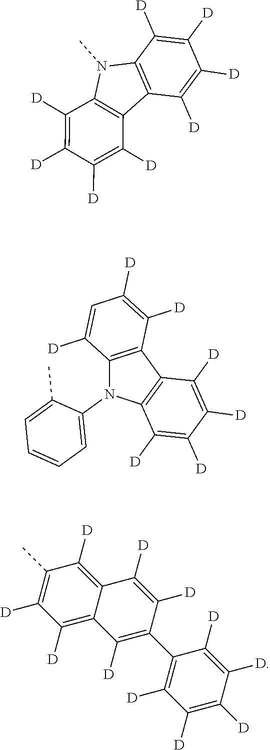

Preferably, each R₁ may be independently hydrogen; deuterium; a substituted or unsubstituted C₆₋₂₀ aryl.

More preferably, each R₁ may be independently hydrogen, deuterium, a substituted or unsubstituted phenyl, a substituted or unsubstituted dibenzofuranyl, or a substituted or unsubstituted dibenzothiophenyl.

More preferably, each R₁ may be independently hydrogen, deuterium, phenyl unsubstituted or substituted with deuterium, dibenzofuranyl unsubstituted or substituted with deuterium, or dibenzothiophenyl unsubstituted or substituted with deuterium.

Most preferably, each R₁ may be independently hydrogen, deuterium, phenyl, phenyl substituted with 3 deuteriums, phenyl substituted with 5 deuteriums, dibenzofuranyl, or dibenzothiophenyl.

Preferably, R₂ to R₄ may be each independently hydrogen; deuterium; or a substituted or unsubstituted C₆₋₂₀ aryl.

More preferably, each R₂ may be independently hydrogen, deuterium, or a substituted or unsubstituted phenyl, each R₃ may be independently hydrogen, deuterium, or a substituted or unsubstituted phenyl, and each R₄ may be independently hydrogen, deuterium, or a substituted or unsubstituted phenyl.

More preferably, each R₂ may be independently hydrogen, deuterium, or phenyl unsubstituted or substituted with deuterium, each R₃ may be each independently hydrogen, deuterium, or phenyl unsubstituted or substituted with deuterium, and R₄ may be each independently hydrogen, deuterium, or phenyl unsubstituted or substituted with deuterium.

Most preferably, each R₂ may be independently hydrogen, deuterium, phenyl, phenyl substituted with 3 deuterium, or phenyl substituted with 5 deuterium, each R₃ may be independently hydrogen, deuterium, phenyl, phenyl substituted with 3 deuterium, or phenyl substituted with 5 deuterium, and each R₄ may be independently hydrogen, deuterium, phenyl, phenyl substituted with 3 deuterium, or phenyl substituted with 5 deuterium.

Representative examples of the compound represented by Chemical Formula 1 are as follows:

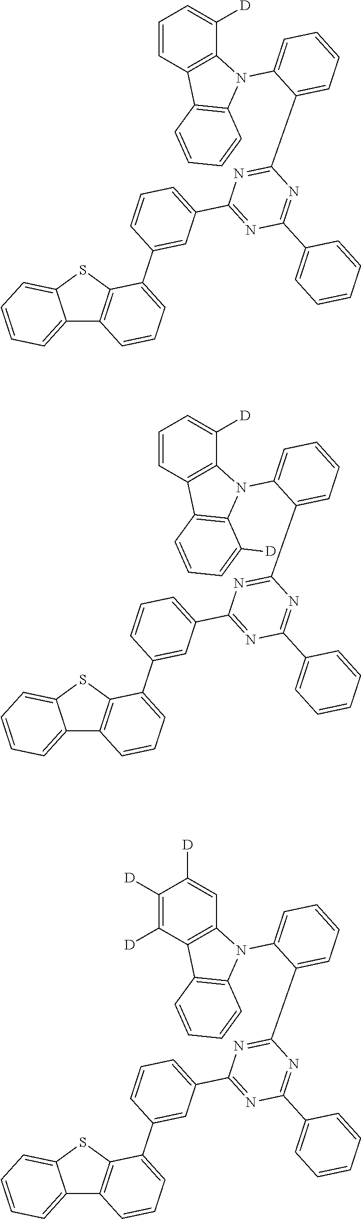 [SMILES: [2H]c1cccc2c3ccccc3n(-c3ccccc3-c3nc(-c4ccccc4)nc(-c4cccc(-c5cccc6c5sc5ccccc56)c4)n3)c12 ; [2H]c1cccc2c3cccc([2H])c3n(-c3ccccc3-c3nc(-c4ccccc4)nc(-c4cccc(-c5cccc6c5sc5ccccc56)c4)n3)c12 ; [2H]c1cc2c(c([2H])c1[2H])c1ccccc1n2-c1ccccc1-c1nc(-c2ccccc2)nc(-c2cccc(-c3cccc4c3sc3ccccc34)c2)n1]

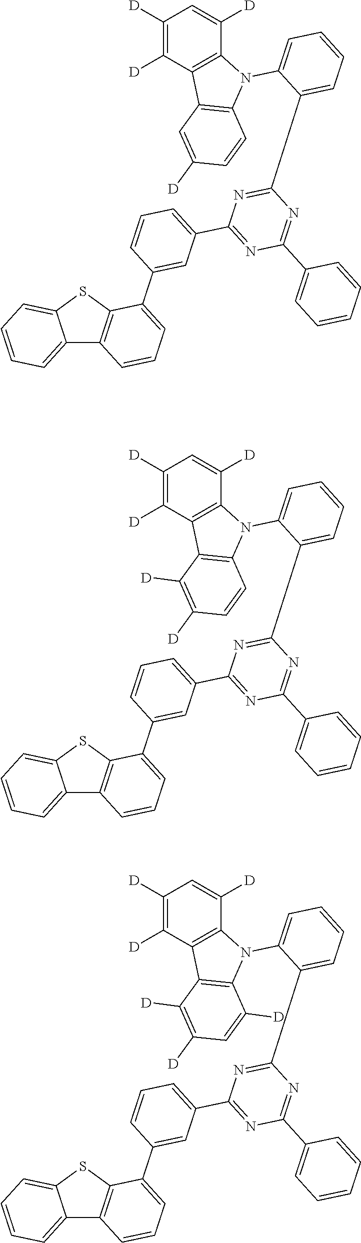 [SMILES: [2H]c1ccc2c(c1)c1c([2H])c([2H])cc([2H])c1n2-c1ccccc1-c1nc(-c2ccccc2)nc(-c2cccc(-c3cccc4c3sc3ccccc34)c2)n1 ; [2H]c1ccc2c(c1[2H])c1c([2H])c([2H])cc([2H])c1n2-c1ccccc1-c1nc(-c2ccccc2)nc(-c2cccc(-c3cccc4c3sc3ccccc34)c2)n1 ; [2H]c1cc([2H])c2c(c1[2H])c1c([2H])c([2H])cc([2H])c1n2-c1ccccc1-c1nc(-c2ccccc2)nc(-c2cccc(-c3cccc4c3sc3ccccc34)c2)n1]

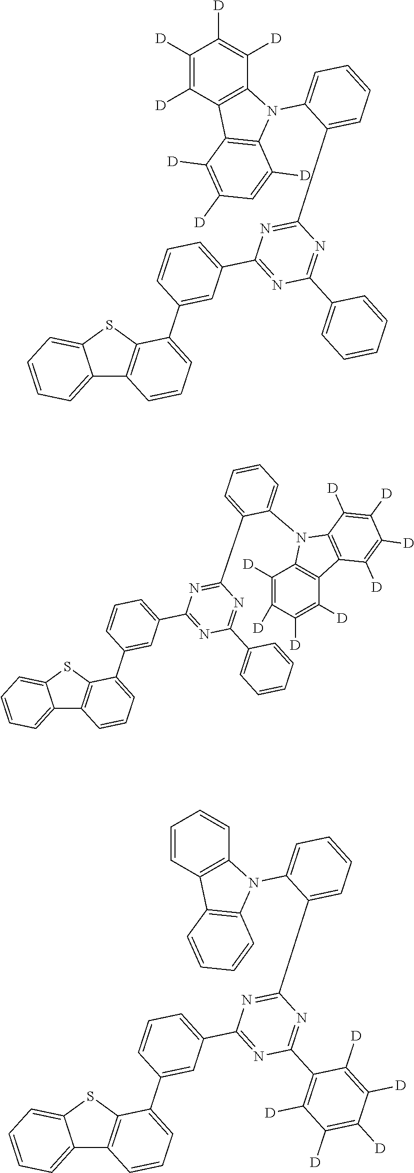 [SMILES: [2H]c1cc([2H])c2c(c1[2H])c1c([2H])c([2H])c([2H])c([2H])c1n2-c1ccccc1-c1nc(-c2ccccc2)nc(-c2cccc(-c3cccc4c3sc3ccccc34)c2)n1 ; [2H]c1c([2H])c([2H])c2c(c1[2H])c1c([2H])c([2H])c([2H])c([2H])c1n2-c1ccccc1-c1nc(-c2ccccc2)nc(-c2cccc(-c3cccc4c3sc3ccccc34)c2)n1 ; [2H]c1c([2H])c([2H])c(-c2nc(-c3cccc(-c4cccc5c4sc4ccccc45)c3)nc(-c3ccccc3-n3c4ccccc4c4ccccc43)n2)c([2H])c1[2H]]

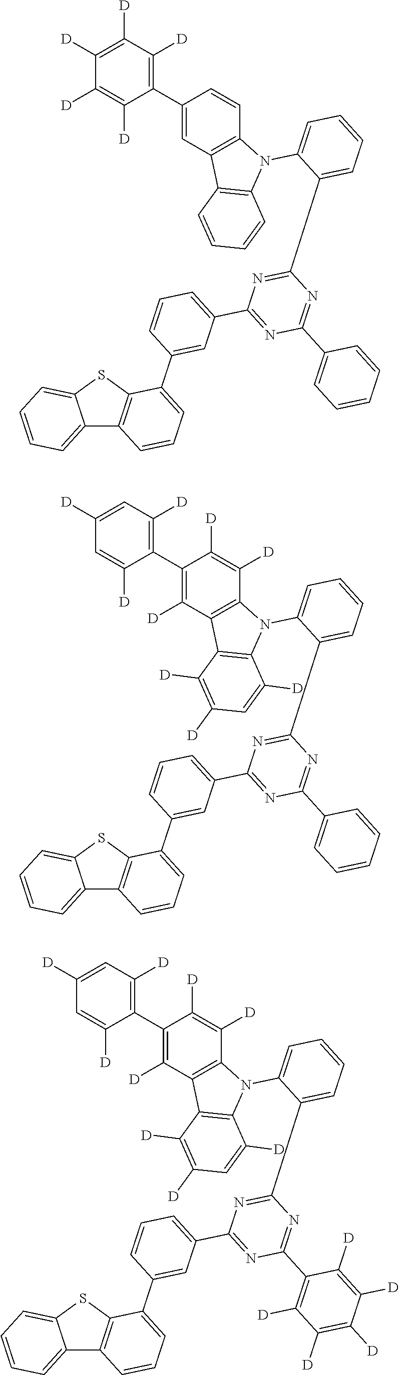 [SMILES: [2H]c1c([2H])c([2H])c(-c2ccc3c(c2)c2ccccc2n3-c2ccccc2-c2nc(-c3ccccc3)nc(-c3cccc(-c4cccc5c4sc4ccccc45)c3)n2)c([2H])c1[2H] ; [2H]c1cc([2H])c(-c2c([2H])c([2H])c3c(c2[2H])c2c([2H])c([2H])cc([2H])c2n3-c2ccccc2-c2nc(-c3ccccc3)nc(-c3cccc(-c4cccc5c4sc4ccccc45)c3)n2)c([2H])c1 ; [2H]c1cc([2H])c(-c2c([2H])c([2H])c3c(c2[2H])c2c([2H])c([2H])cc([2H])c2n3-c2ccccc2-c2nc(-c3cccc(-c4cccc5c4sc4ccccc45)c3)nc(-c3c([2H])c([2H])c([2H])c([2H])c3[2H])n2)c([2H])c1]

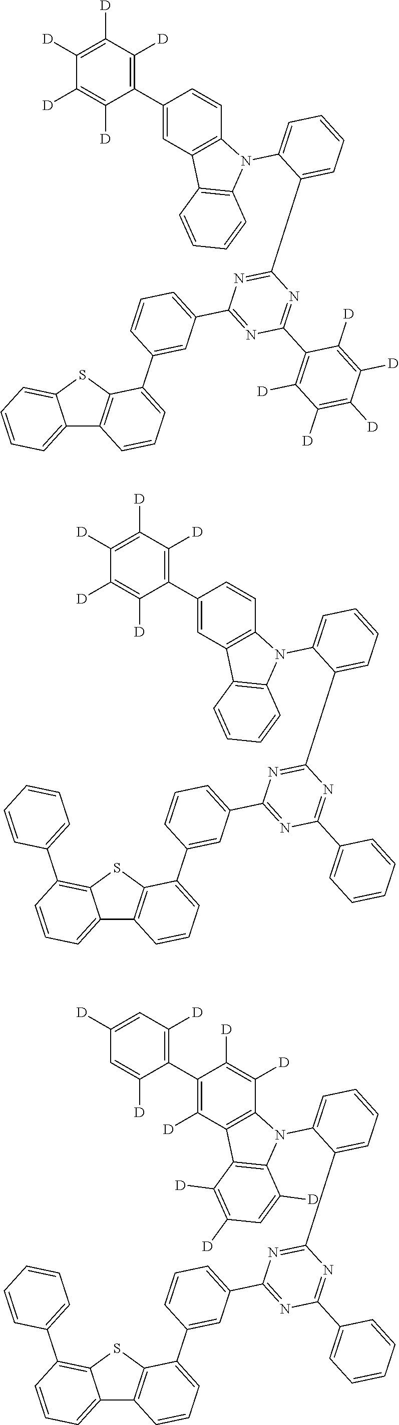 [SMILES: [2H]c1c([2H])c([2H])c(-c2ccc3c(c2)c2ccccc2n3-c2ccccc2-c2nc(-c3cccc(-c4cccc5c4sc4ccccc45)c3)nc(-c3c([2H])c([2H])c([2H])c([2H])c3[2H])n2)c([2H])c1[2H] ; [2H]c1c([2H])c([2H])c(-c2ccc3c(c2)c2ccccc2n3-c2ccccc2-c2nc(-c3ccccc3)nc(-c3cccc(-c4cccc5c4sc4c(-c6ccccc6)cccc45)c3)n2)c([2H])c1[2H] ; [2H]c1cc([2H])c(-c2c([2H])c([2H])c3c(c2[2H])c2c([2H])c([2H])cc([2H])c2n3-c2ccccc2-c2nc(-c3ccccc3)nc(-c3cccc(-c4cccc5c4sc4c(-c6ccccc6)cccc45)c3)n2)c([2H])c1]

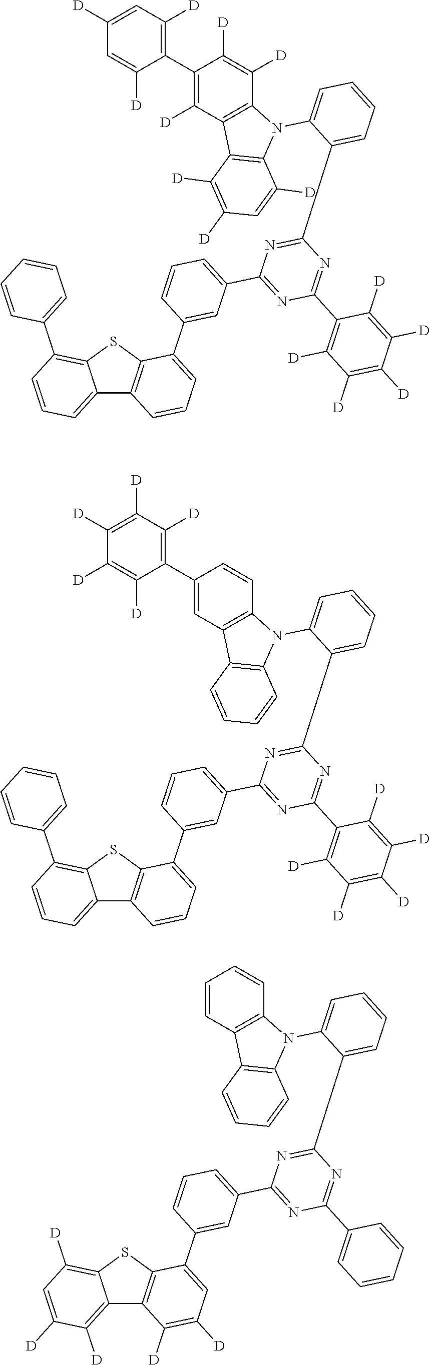 [SMILES: [2H]c1cc([2H])c(-c2c([2H])c([2H])c3c(c2[2H])c2c([2H])c([2H])cc([2H])c2n3-c2ccccc2-c2nc(-c3cccc(-c4cccc5c4sc4c(-c6ccccc6)cccc45)c3)nc(-c3c([2H])c([2H])c([2H])c([2H])c3[2H])n2)c([2H])c1 ; [2H]c1c([2H])c([2H])c(-c2ccc3c(c2)c2ccccc2n3-c2ccccc2-c2nc(-c3cccc(-c4cccc5c4sc4c(-c6ccccc6)cccc45)c3)nc(-c3c([2H])c([2H])c([2H])c([2H])c3[2H])n2)c([2H])c1[2H] ; [2H]c1cc([2H])c2sc3c(-c4cccc(-c5nc(-c6ccccc6)nc(-c6ccccc6-n6c7ccccc7c7ccccc76)n5)c4)cc([2H])c([2H])c3c2c1[2H]]

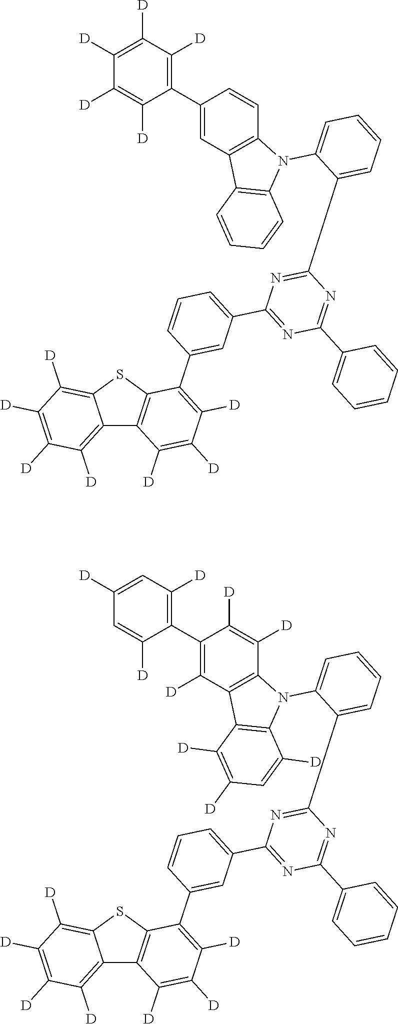 [SMILES: [2H]c1c([2H])c([2H])c(-c2ccc3c(c2)c2ccccc2n3-c2ccccc2-c2nc(-c3ccccc3)nc(-c3cccc(-c4c([2H])c([2H])c([2H])c5c4sc4c([2H])c([2H])c([2H])c([2H])c45)c3)n2)c([2H])c1[2H] ; [2H]c1cc([2H])c(-c2c([2H])c([2H])c3c(c2[2H])c2c([2H])c([2H])cc([2H])c2n3-c2ccccc2-c2nc(-c3ccccc3)nc(-c3cccc(-c4c([2H])c([2H])c([2H])c5c4sc4c([2H])c([2H])c([2H])c([2H])c45)c3)n2)c([2H])c1]

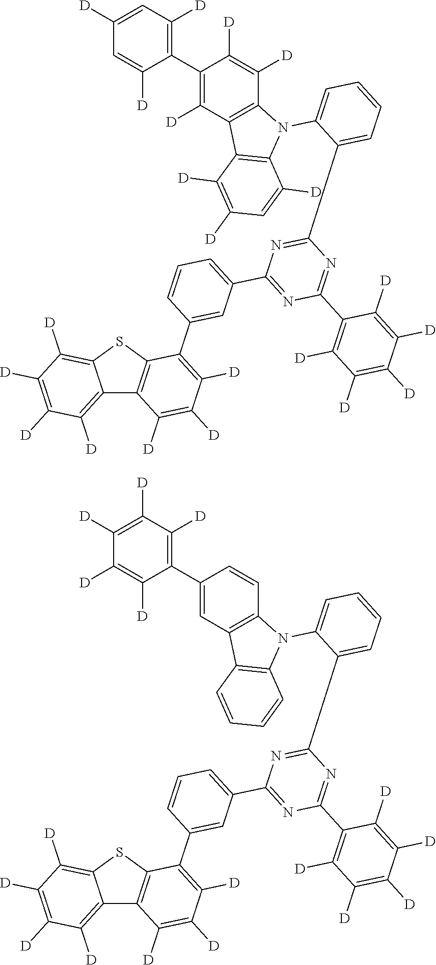

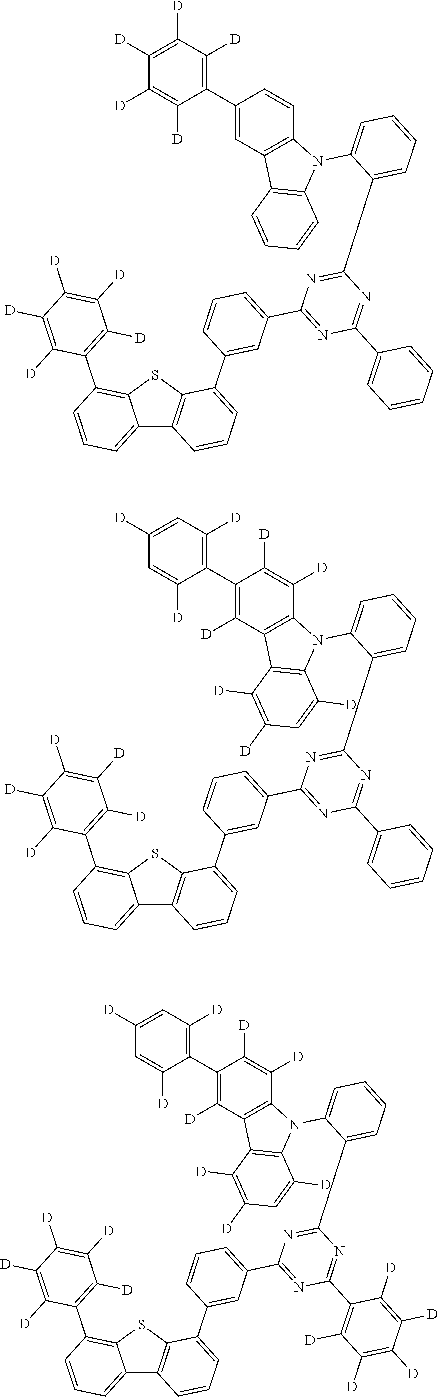

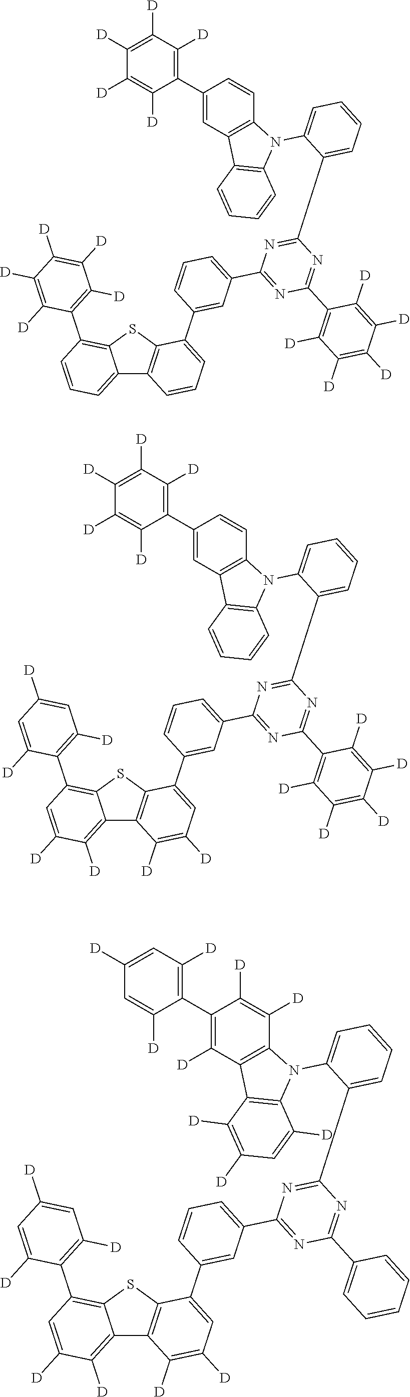

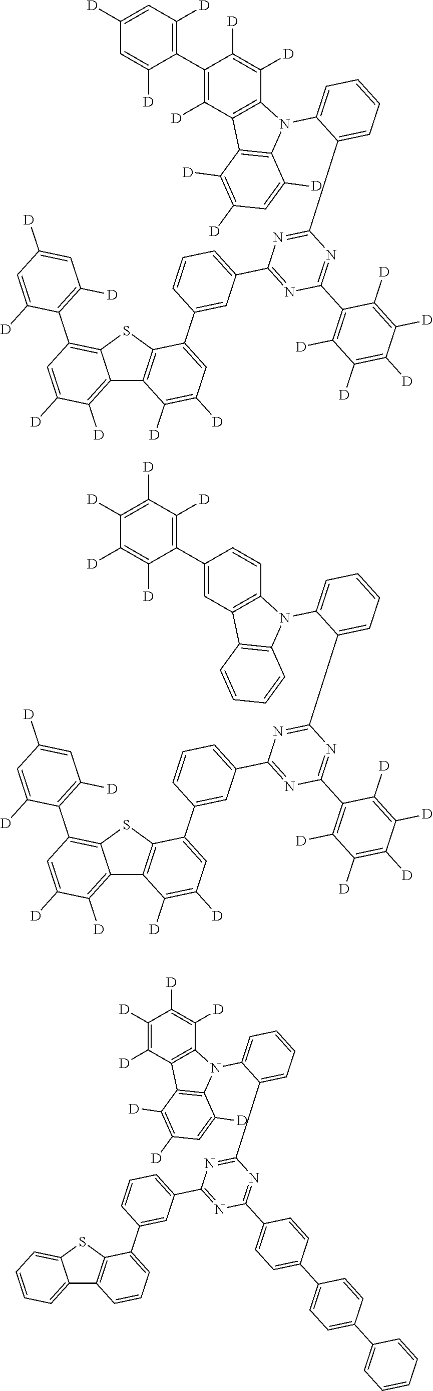

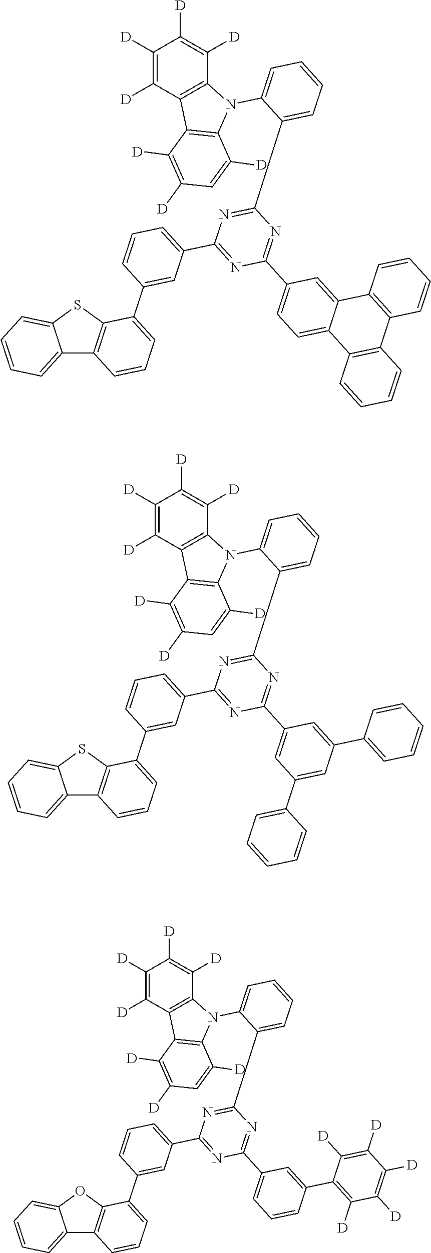 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccccc34)c2)nc(-c2ccc3c4ccccc4c4ccccc4c3c2)n1 ; [2H]c1cc([2H])c2c(c1[2H])c1c([2H])c([2H])c([2H])c([2H])c1n2-c1ccccc1-c1nc(-c2cc(-c3ccccc3)cc(-c3ccccc3)c2)nc(-c2cccc(-c3cccc4c3sc3ccccc34)c2)n1 ; [2H]c1cc([2H])c2c(c1[2H])c1c([2H])c([2H])c([2H])c([2H])c1n2-c1ccccc1-c1nc(-c2cccc(-c3c([2H])c([2H])c([2H])c([2H])c3[2H])c2)nc(-c2cccc(-c3cccc4c3oc3ccccc34)c2)n1]

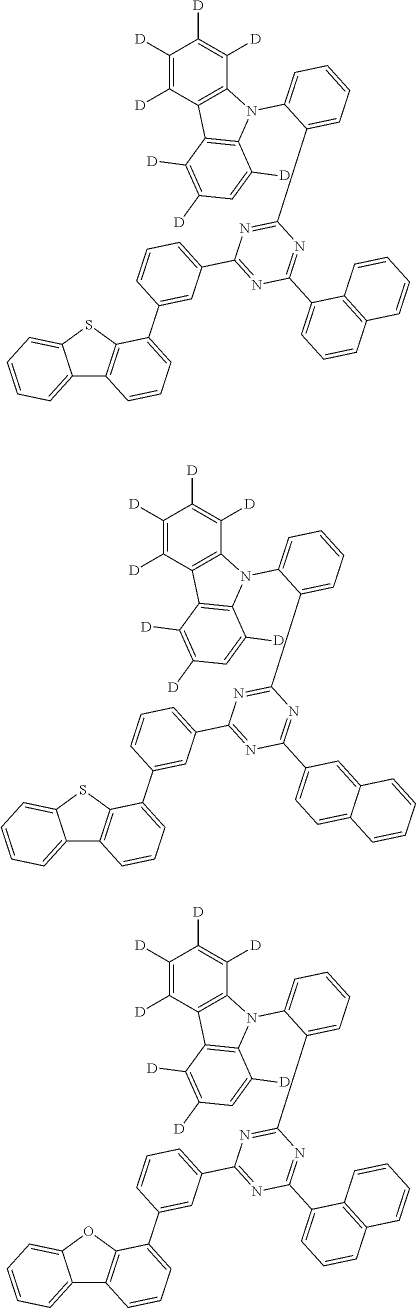 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccccc34)c2)nc(-c2cccc3ccccc23)n1 ; [2H]c1cc([2H])c2c(c1[2H])c1c([2H])c([2H])c([2H])c([2H])c1n2-c1ccccc1-c1nc(-c2cccc(-c3cccc4c3sc3ccccc34)c2)nc(-c2ccc3ccccc3c2)n1 ; [2H]c1cc([2H])c2c(c1[2H])c1c([2H])c([2H])c([2H])c([2H])c1n2-c1ccccc1-c1nc(-c2cccc(-c3cccc4c3oc3ccccc34)c2)nc(-c2cccc3ccccc23)n1]

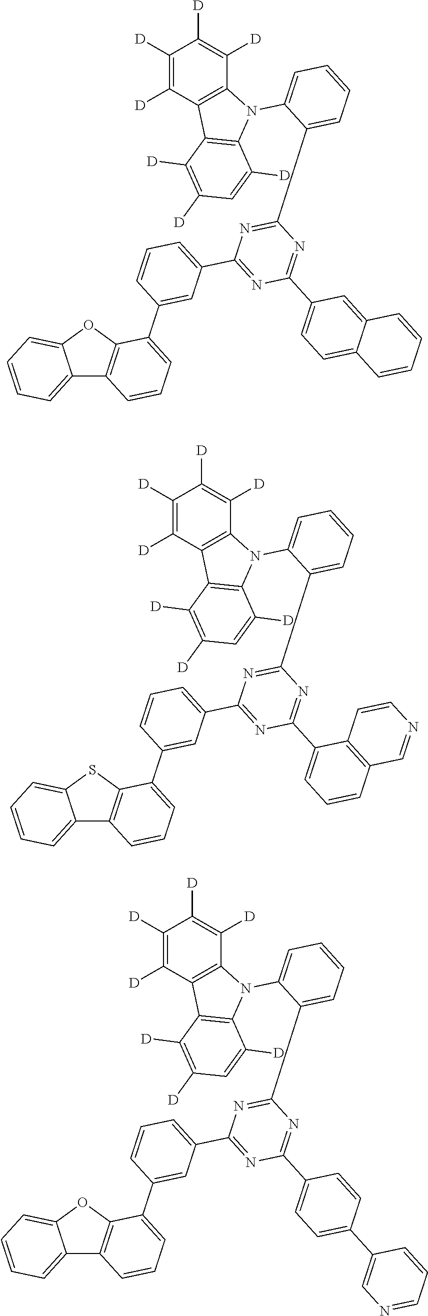 [SMILES: [2H]c1cc([2H])c2c(c1[2H])c1c([2H])c([2H])c([2H])c([2H])c1n2-c1ccccc1-c1nc(-c2cccc(-c3cccc4c3oc3ccccc34)c2)nc(-c2ccc3ccccc3c2)n1 ; [2H]c1cc([2H])c2c(c1[2H])c1c([2H])c([2H])c([2H])c([2H])c1n2-c1ccccc1-c1nc(-c2cccc(-c3cccc4c3sc3ccccc34)c2)nc(-c2cccc3cnccc23)n1 ; [2H]c1cc([2H])c2c(c1[2H])c1c([2H])c([2H])c([2H])c([2H])c1n2-c1ccccc1-c1nc(-c2ccc(-c3cccnc3)cc2)nc(-c2cccc(-c3cccc4c3oc3ccccc34)c2)n1]

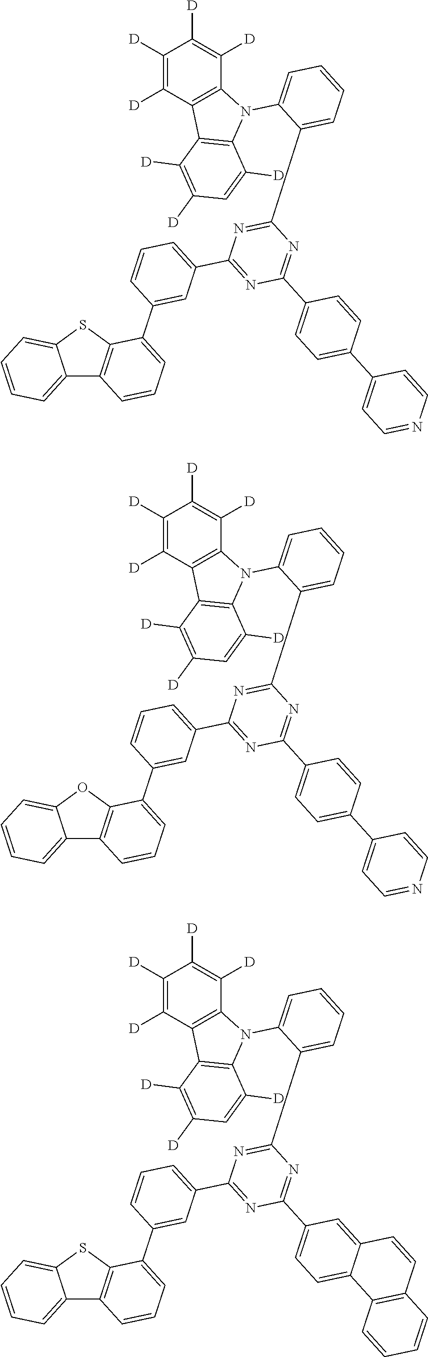 [SMILES: [2H]c1cc([2H])c2c(c1[2H])c1c([2H])c([2H])c([2H])c([2H])c1n2-c1ccccc1-c1nc(-c2ccc(-c3ccncc3)cc2)nc(-c2cccc(-c3cccc4c3sc3ccccc34)c2)n1 ; [2H]c1cc([2H])c2c(c1[2H])c1c([2H])c([2H])c([2H])c([2H])c1n2-c1ccccc1-c1nc(-c2ccc(-c3ccncc3)cc2)nc(-c2cccc(-c3cccc4c3oc3ccccc34)c2)n1 ; [2H]c1cc([2H])c2c(c1[2H])c1c([2H])c([2H])c([2H])c([2H])c1n2-c1ccccc1-c1nc(-c2cccc(-c3cccc4c3sc3ccccc34)c2)nc(-c2ccc3c(ccc4ccccc43)c2)n1]

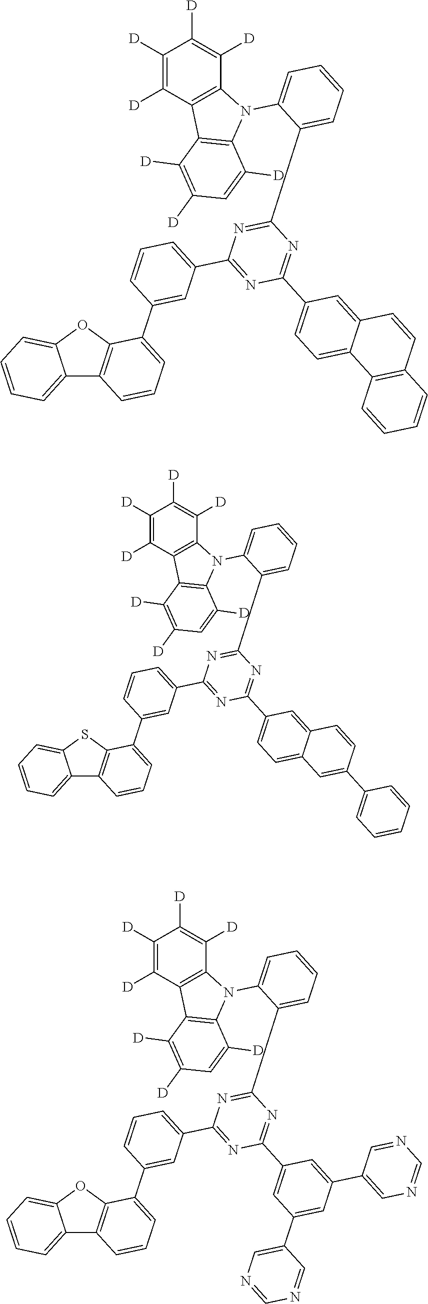 [SMILES: [2H]c1cc([2H])c2c(c1[2H])c1c([2H])c([2H])c([2H])c([2H])c1n2-c1ccccc1-c1nc(-c2cccc(-c3cccc4c3oc3ccccc34)c2)nc(-c2ccc3c(ccc4ccccc43)c2)n1 ; [2H]c1cc([2H])c2c(c1[2H])c1c([2H])c([2H])c([2H])c([2H])c1n2-c1ccccc1-c1nc(-c2cccc(-c3cccc4c3sc3ccccc34)c2)nc(-c2ccc3cc(-c4ccccc4)ccc3c2)n1 ; [2H]c1cc([2H])c2c(c1[2H])c1c([2H])c([2H])c([2H])c([2H])c1n2-c1ccccc1-c1nc(-c2cc(-c3cncnc3)cc(-c3cncnc3)c2)nc(-c2cccc(-c3cccc4c3oc3ccccc34)c2)n1]

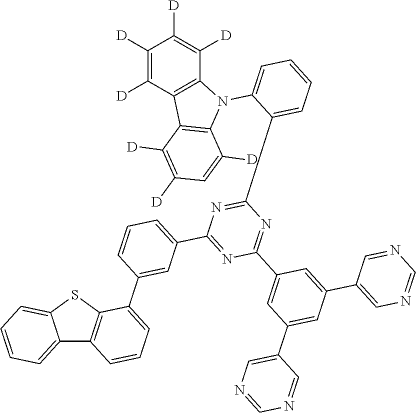 [SMILES: [2H]c1cc([2H])c2c(c1[2H])c1c([2H])c([2H])c([2H])c([2H])c1n2-c1ccccc1-c1nc(-c2cc(-c3cncnc3)cc(-c3cncnc3)c2)nc(-c2cccc(-c3cccc4c3sc3ccccc34)c2)n1]

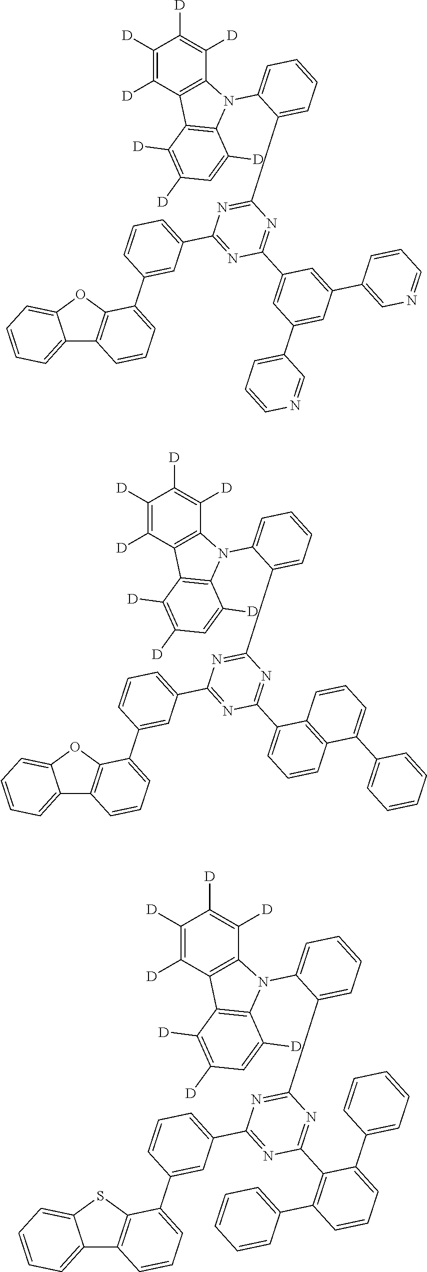 [SMILES: [2H]c1cc([2H])c2c(c1[2H])c1c([2H])c([2H])c([2H])c([2H])c1n2-c1ccccc1-c1nc(-c2cc(-c3cccnc3)cc(-c3cccnc3)c2)nc(-c2cccc(-c3cccc4c3oc3ccccc34)c2)n1 ; [2H]c1cc([2H])c2c(c1[2H])c1c([2H])c([2H])c([2H])c([2H])c1n2-c1ccccc1-c1nc(-c2cccc(-c3cccc4c3oc3ccccc34)c2)nc(-c2cccc3c(-c4ccccc4)cccc23)n1 ; [2H]c1cc([2H])c2c(c1[2H])c1c([2H])c([2H])c([2H])c([2H])c1n2-c1ccccc1-c1nc(-c2cccc(-c3cccc4c3sc3ccccc34)c2)nc(-c2c(-c3ccccc3)cccc2-c2ccccc2)n1]

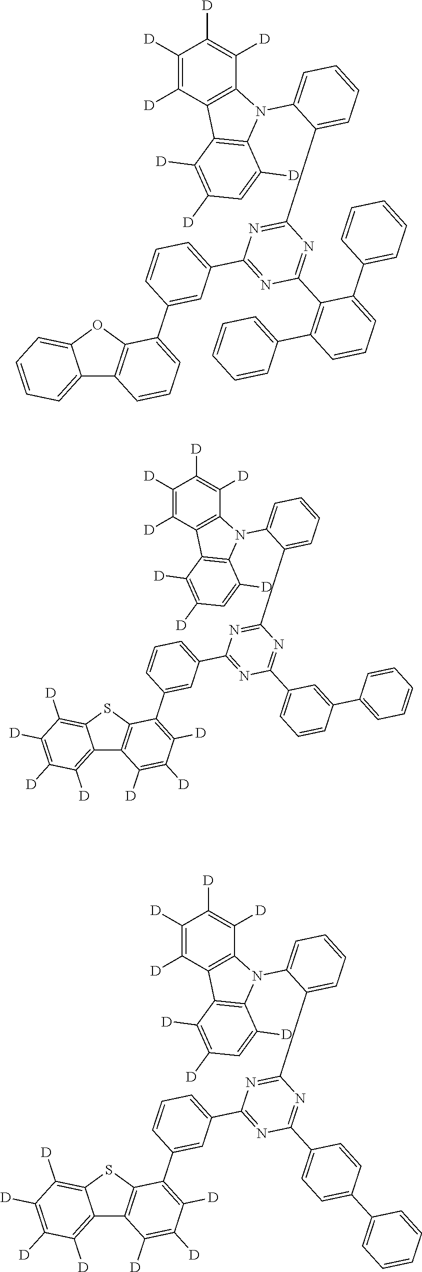 [SMILES: [2H]c1cc([2H])c2c(c1[2H])c1c([2H])c([2H])c([2H])c([2H])c1n2-c1ccccc1-c1nc(-c2cccc(-c3cccc4c3oc3ccccc34)c2)nc(-c2c(-c3ccccc3)cccc2-c2ccccc2)n1 ; [2H]c1cc([2H])c2c(c1[2H])c1c([2H])c([2H])c([2H])c([2H])c1n2-c1ccccc1-c1nc(-c2cccc(-c3ccccc3)c2)nc(-c2cccc(-c3c([2H])c([2H])c([2H])c4c3sc3c([2H])c([2H])c([2H])c([2H])c34)c2)n1 ; [2H]c1cc([2H])c2c(c1[2H])c1c([2H])c([2H])c([2H])c([2H])c1n2-c1ccccc1-c1nc(-c2ccc(-c3ccccc3)cc2)nc(-c2cccc(-c3c([2H])c([2H])c([2H])c4c3sc3c([2H])c([2H])c([2H])c([2H])c34)c2)n1]

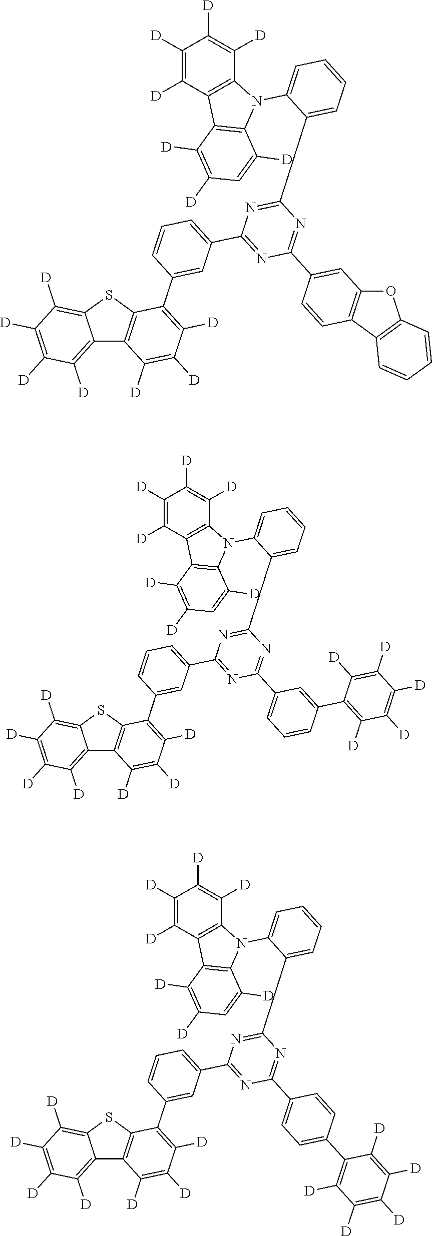

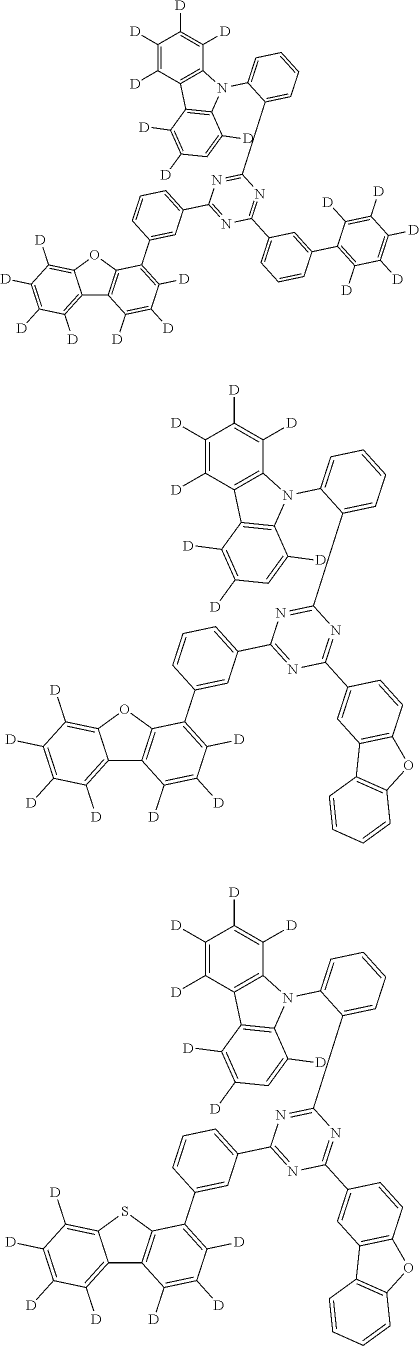

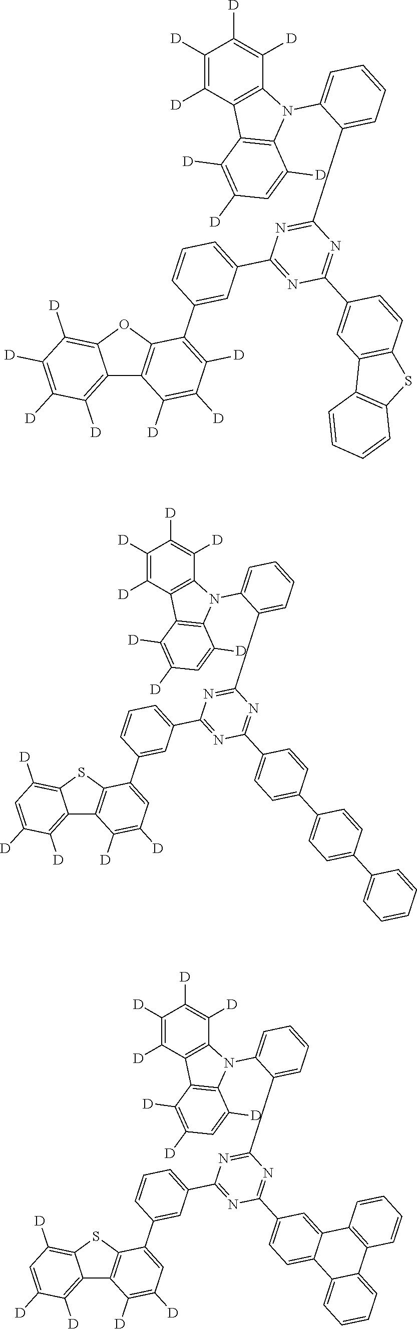

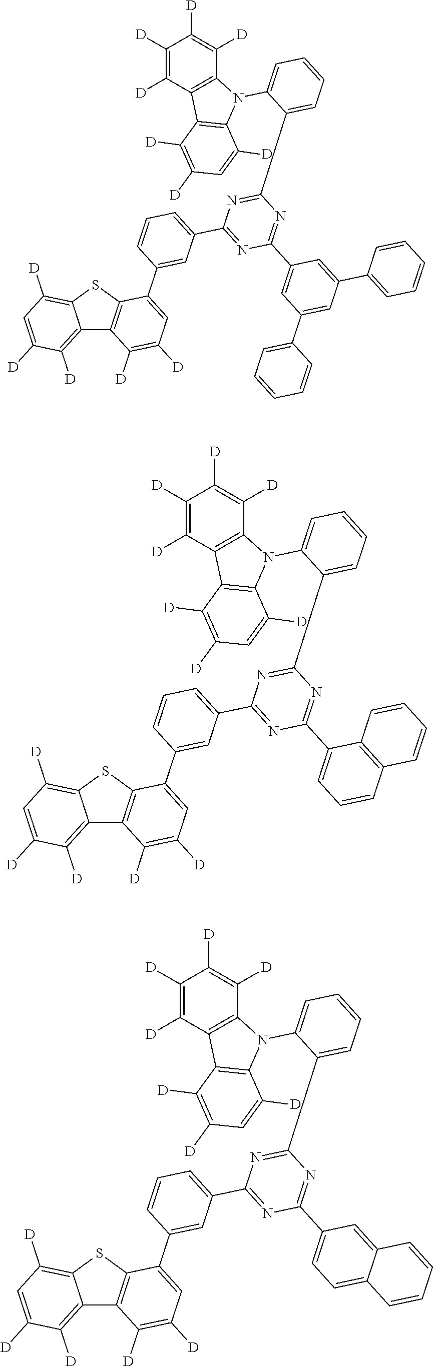 [SMILES: [2H]c1cc([2H])c2sc3c(-c4cccc(-c5nc(-c6cc(-c7ccccc7)cc(-c7ccccc7)c6)nc(-c6ccccc6-n6c7c([2H])cc([2H])c([2H])c7c7c([2H])c([2H])c([2H])c([2H])c76)n5)c4)cc([2H])c([2H])c3c2c1[2H] ; [2H]c1cc([2H])c2sc3c(-c4cccc(-c5nc(-c6ccccc6-n6c7c([2H])cc([2H])c([2H])c7c7c([2H])c([2H])c([2H])c([2H])c76)nc(-c6cccc7ccccc67)n5)c4)cc([2H])c([2H])c3c2c1[2H] ; [2H]c1cc([2H])c2sc3c(-c4cccc(-c5nc(-c6ccc7ccccc7c6)nc(-c6ccccc6-n6c7c([2H])cc([2H])c([2H])c7c7c([2H])c([2H])c([2H])c([2H])c76)n5)c4)cc([2H])c([2H])c3c2c1[2H]]

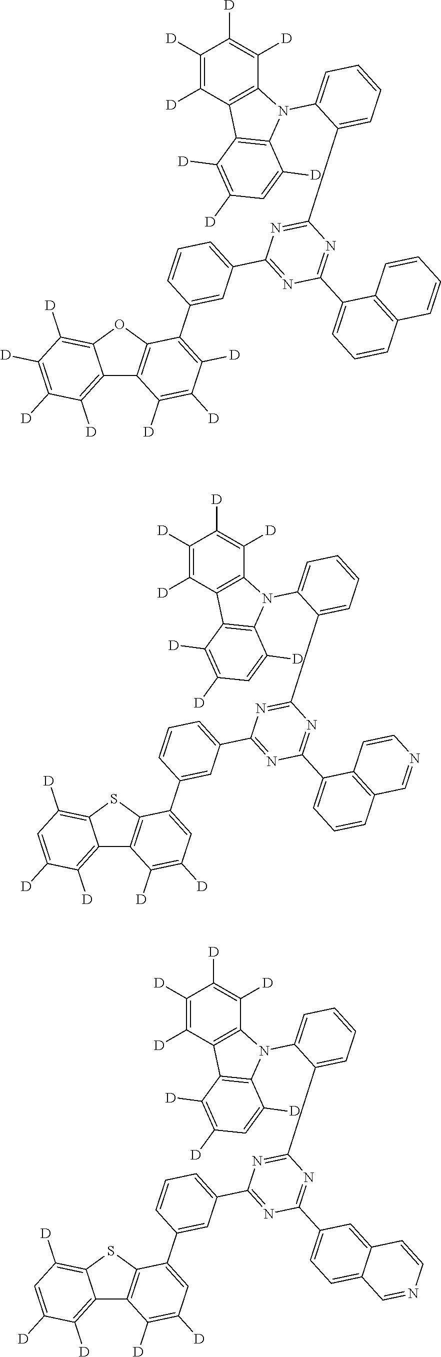 [SMILES: [2H]c1cc([2H])c2c(c1[2H])c1c([2H])c([2H])c([2H])c([2H])c1n2-c1ccccc1-c1nc(-c2cccc(-c3c([2H])c([2H])c([2H])c4c3oc3c([2H])c([2H])c([2H])c([2H])c34)c2)nc(-c2cccc3ccccc23)n1 ; [2H]c1cc([2H])c2sc3c(-c4cccc(-c5nc(-c6ccccc6-n6c7c([2H])cc([2H])c([2H])c7c7c([2H])c([2H])c([2H])c([2H])c76)nc(-c6cccc7cnccc67)n5)c4)cc([2H])c([2H])c3c2c1[2H] ; [2H]c1cc([2H])c2sc3c(-c4cccc(-c5nc(-c6ccc7cnccc7c6)nc(-c6ccccc6-n6c7c([2H])cc([2H])c([2H])c7c7c([2H])c([2H])c([2H])c([2H])c76)n5)c4)cc([2H])c([2H])c3c2c1[2H]]

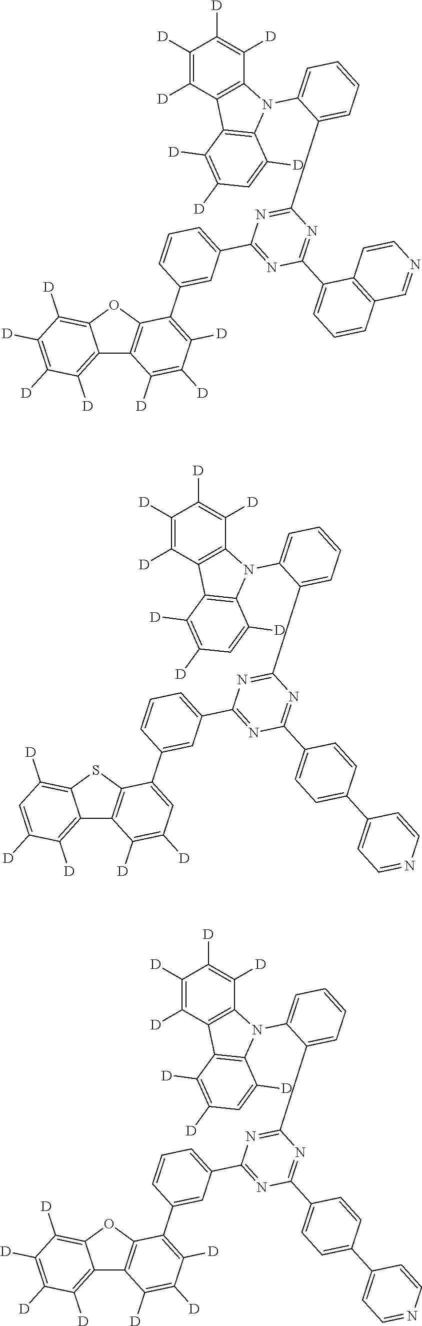

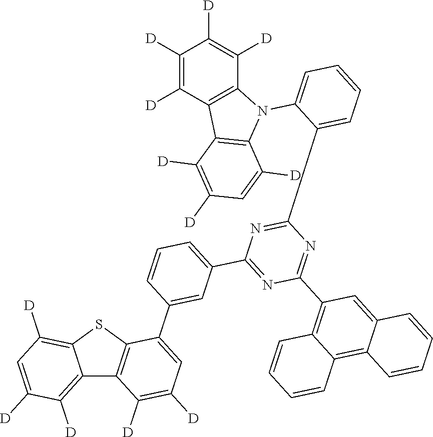 [SMILES: [2H]c1cc([2H])c2sc3c(-c4cccc(-c5nc(-c6ccccc6-n6c7c([2H])cc([2H])c([2H])c7c7c([2H])c([2H])c([2H])c([2H])c76)nc(-c6cc7ccccc7c7ccccc67)n5)c4)cc([2H])c([2H])c3c2c1[2H]]

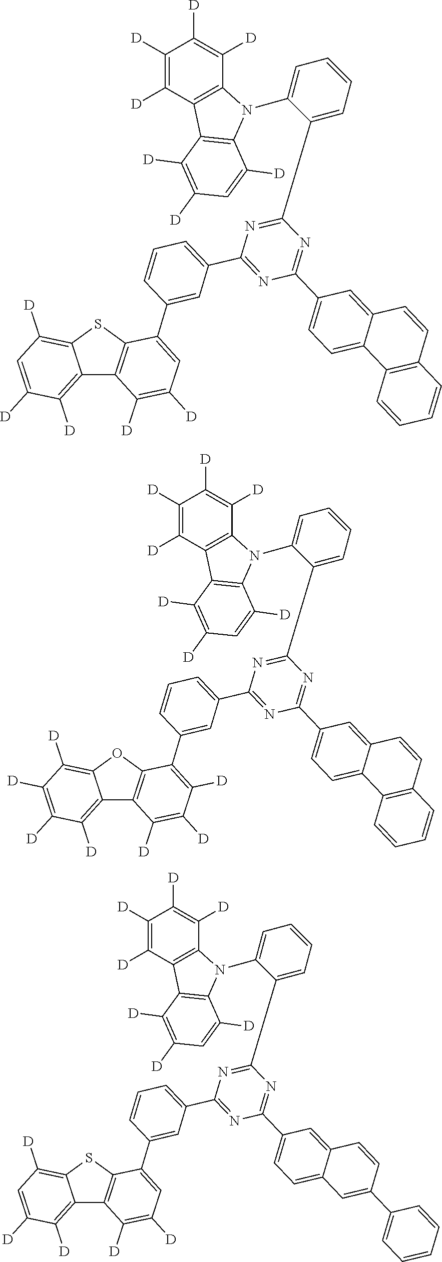

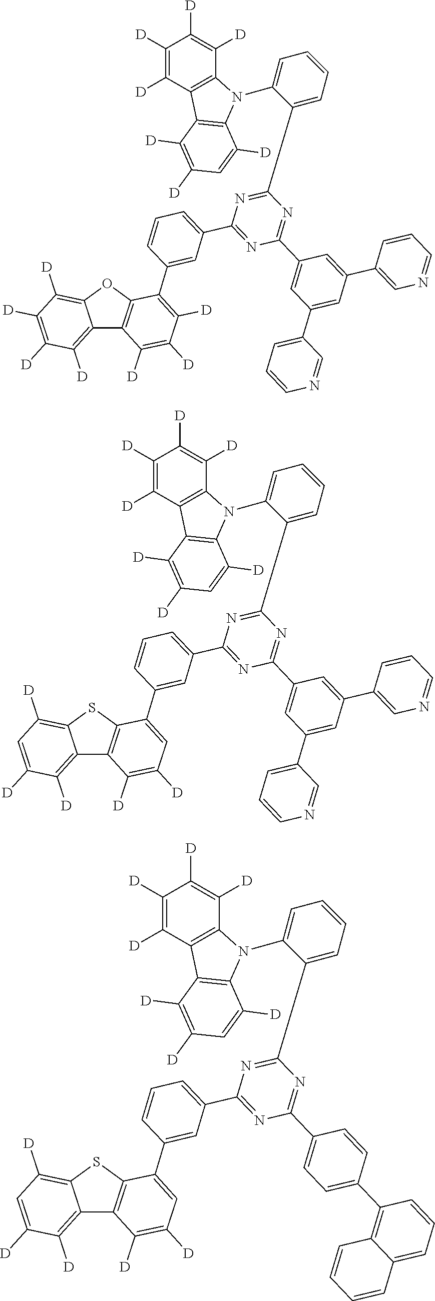

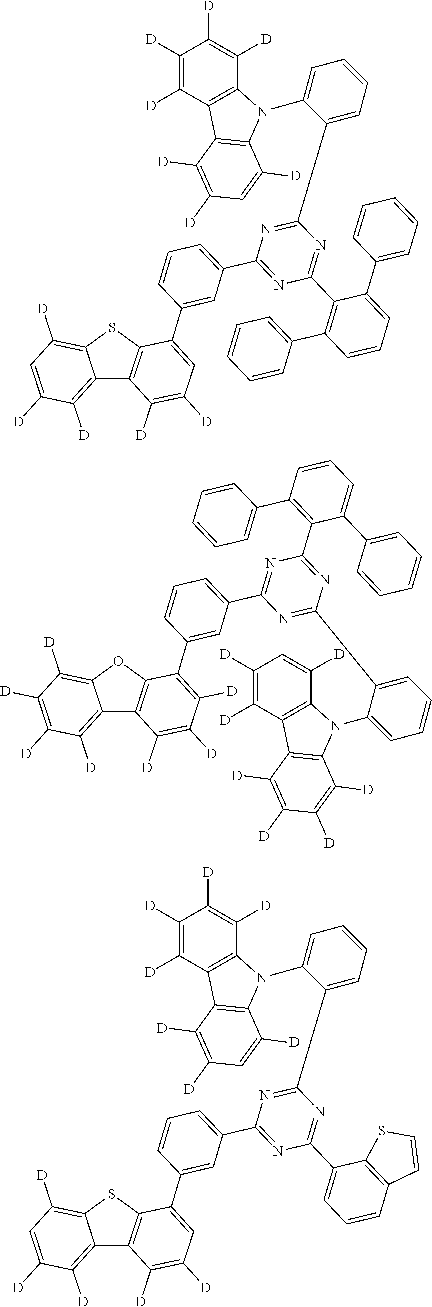

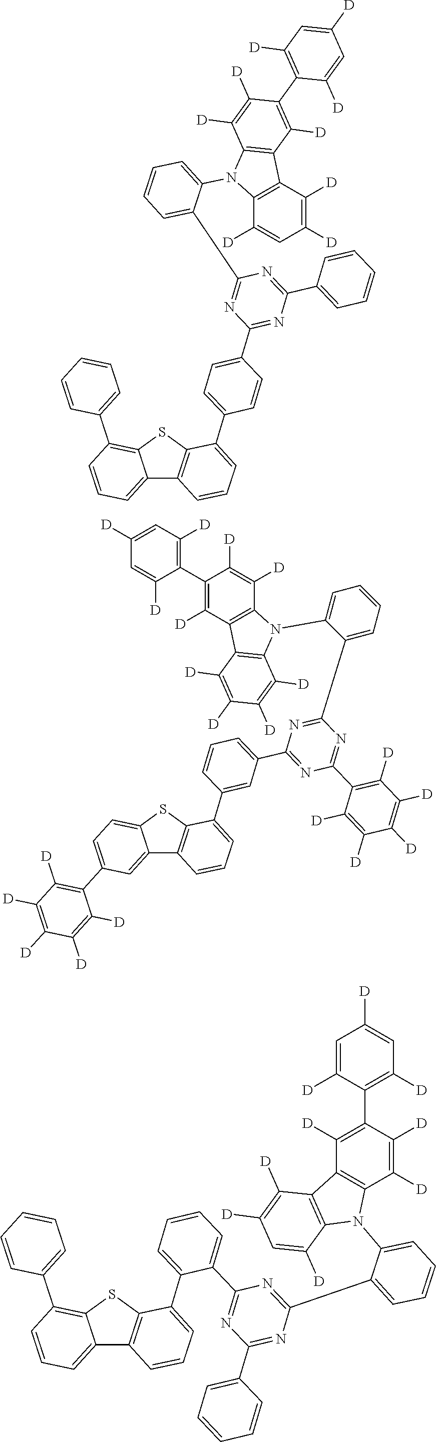

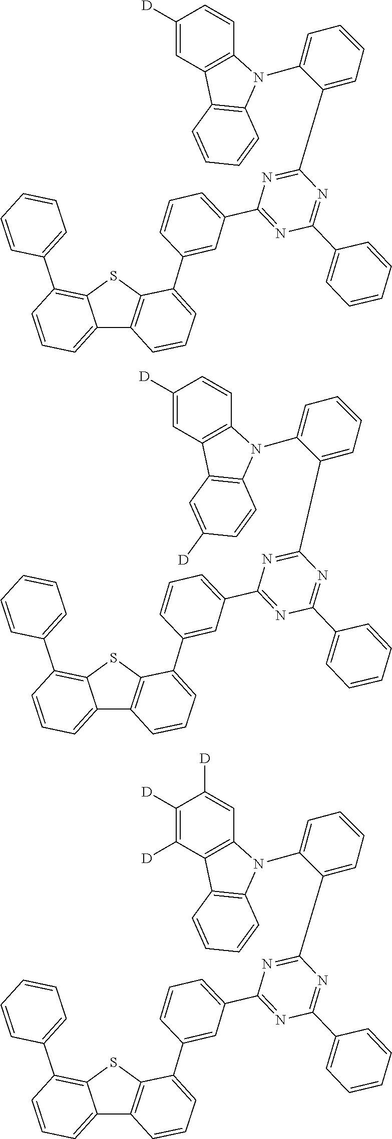

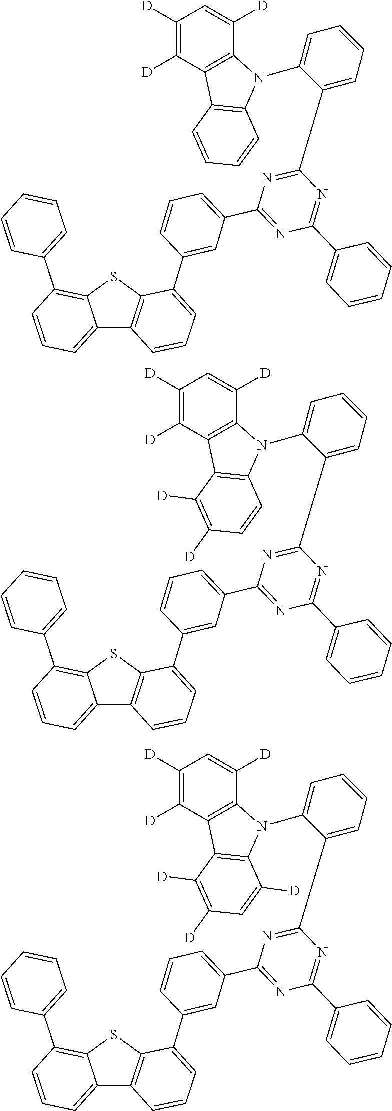

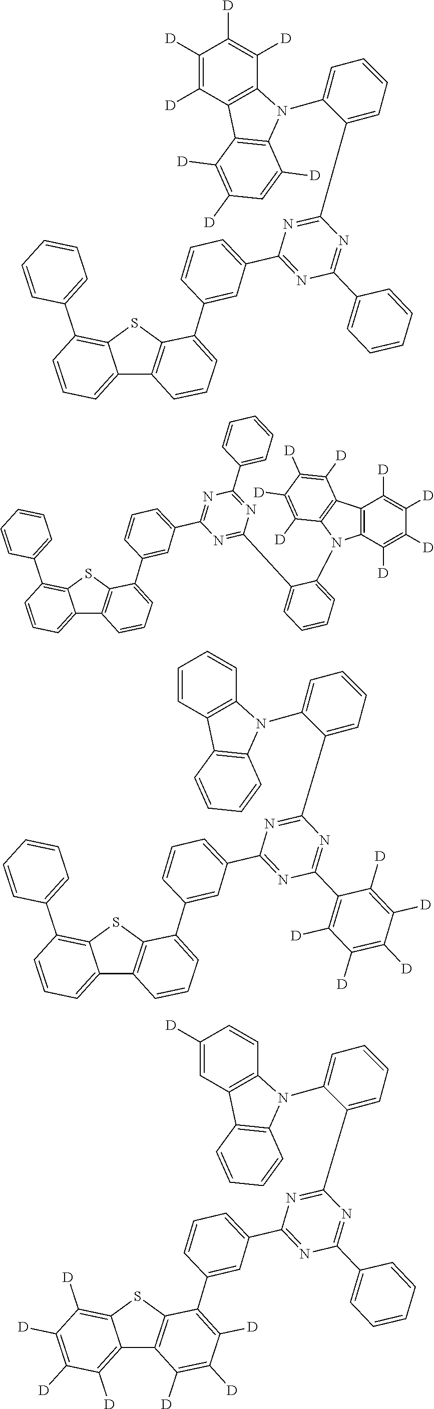

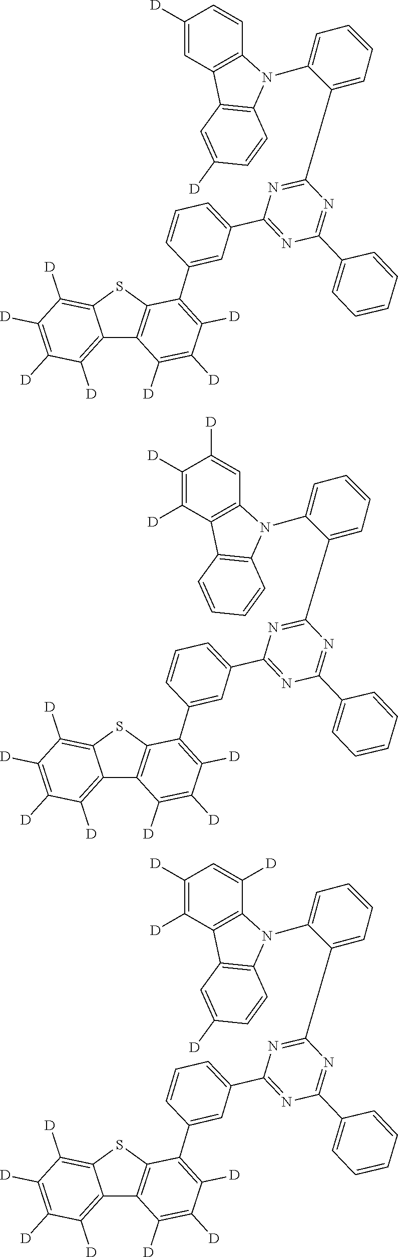

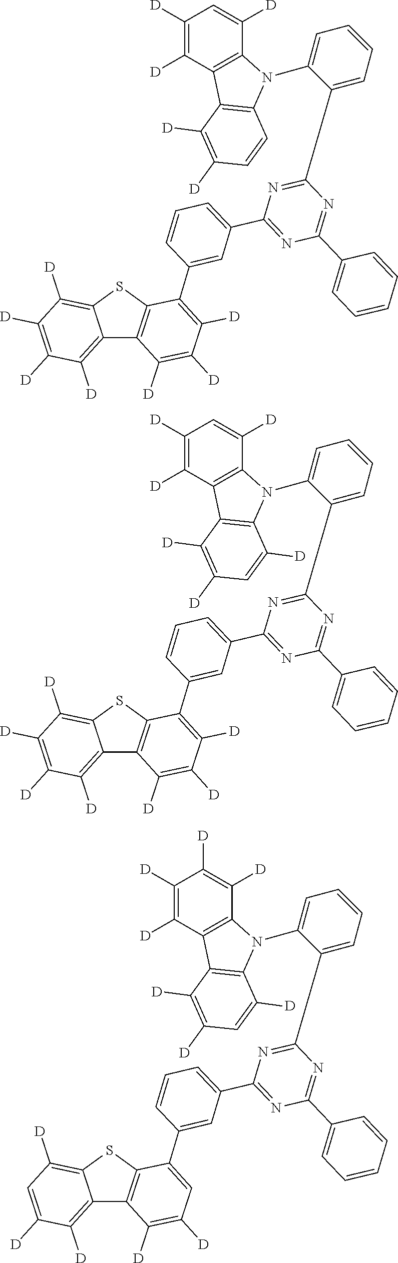

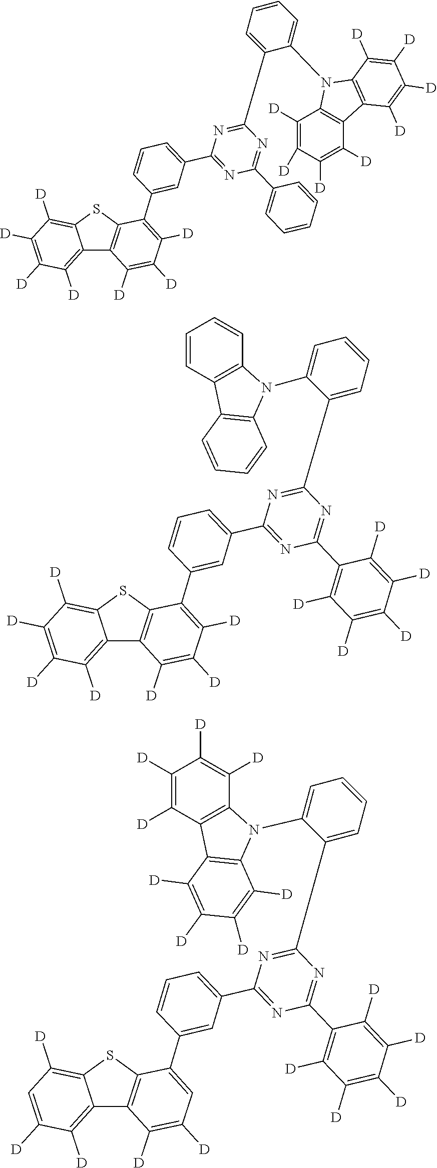

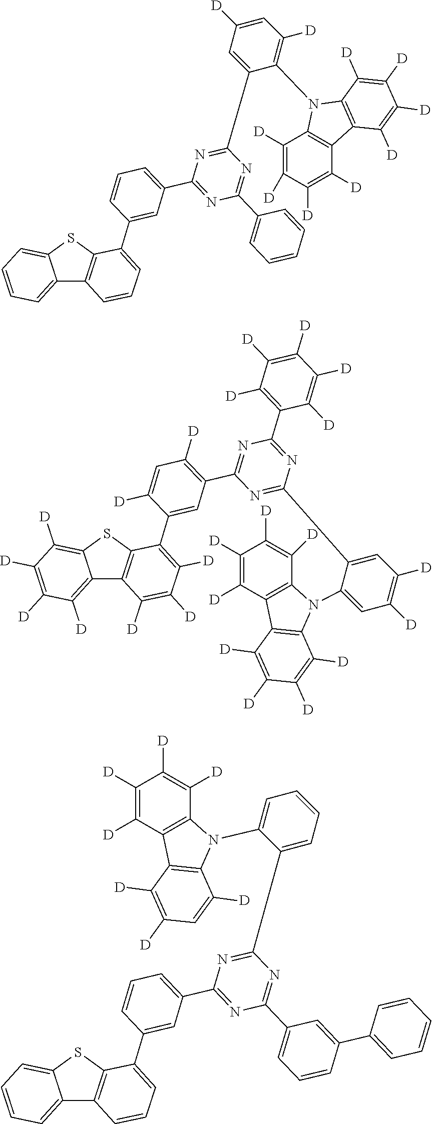

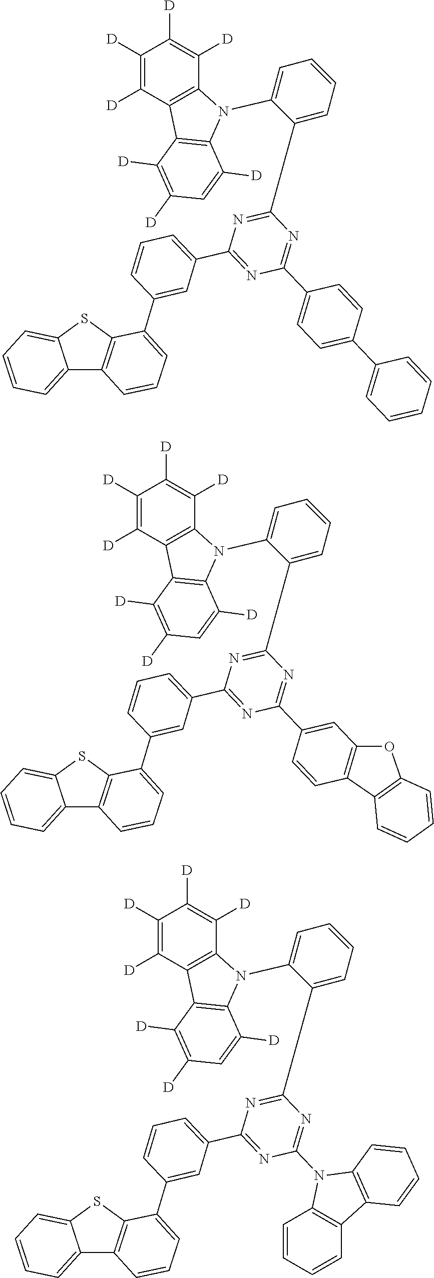 [SMILES: [2H]c1cc([2H])c2c(c1[2H])c1c([2H])c([2H])c([2H])c([2H])c1n2-c1ccccc1-c1nc(-c2ccc(-c3ccccc3)cc2)nc(-c2cccc(-c3cccc4c3sc3ccccc34)c2)n1 ; [2H]c1cc([2H])c2c(c1[2H])c1c([2H])c([2H])c([2H])c([2H])c1n2-c1ccccc1-c1nc(-c2cccc(-c3cccc4c3sc3ccccc34)c2)nc(-c2ccc3c(c2)oc2ccccc23)n1 ; [2H]c1cc([2H])c2c(c1[2H])c1c([2H])c([2H])c([2H])c([2H])c1n2-c1ccccc1-c1nc(-c2cccc(-c3cccc4c3sc3ccccc34)c2)nc(-n2c3ccccc3c3ccccc32)n1]

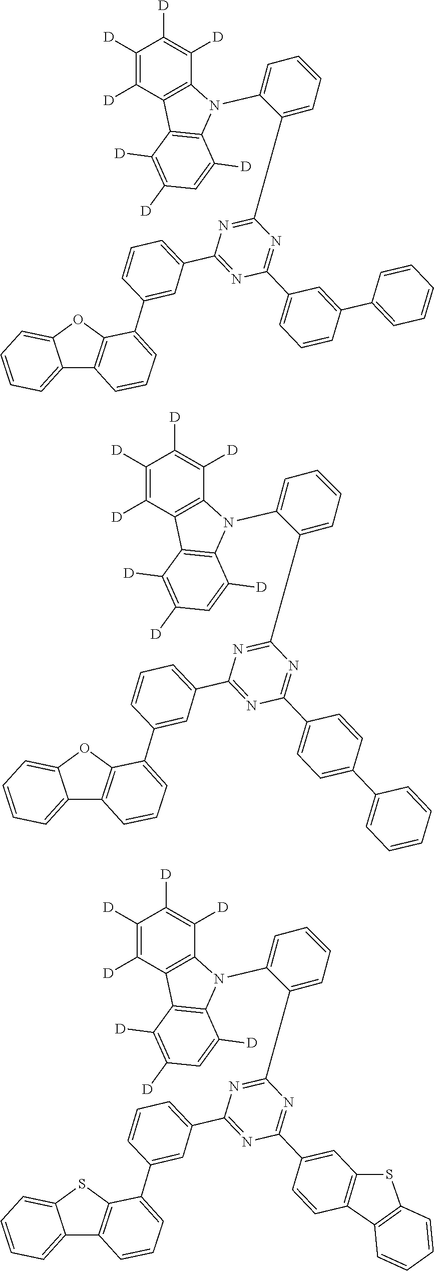

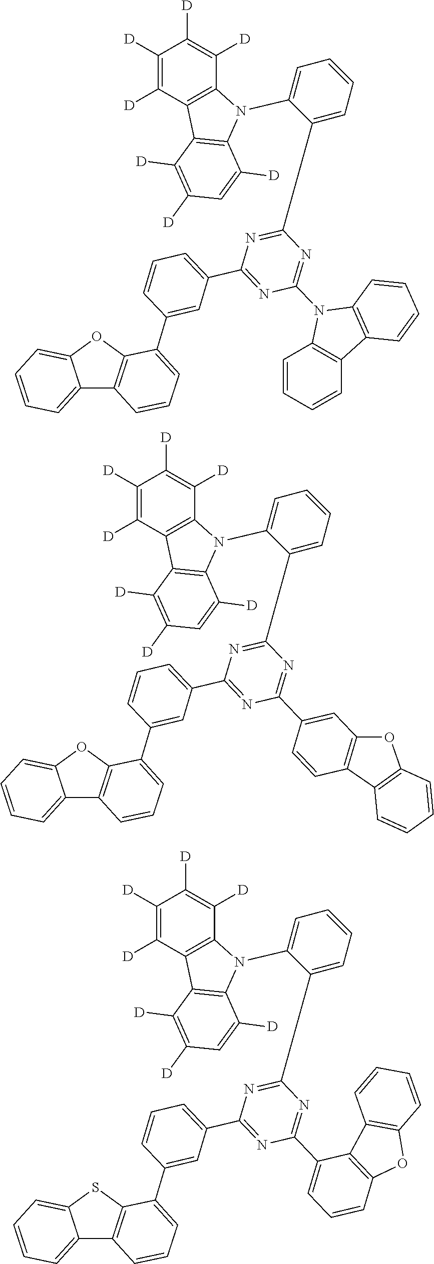

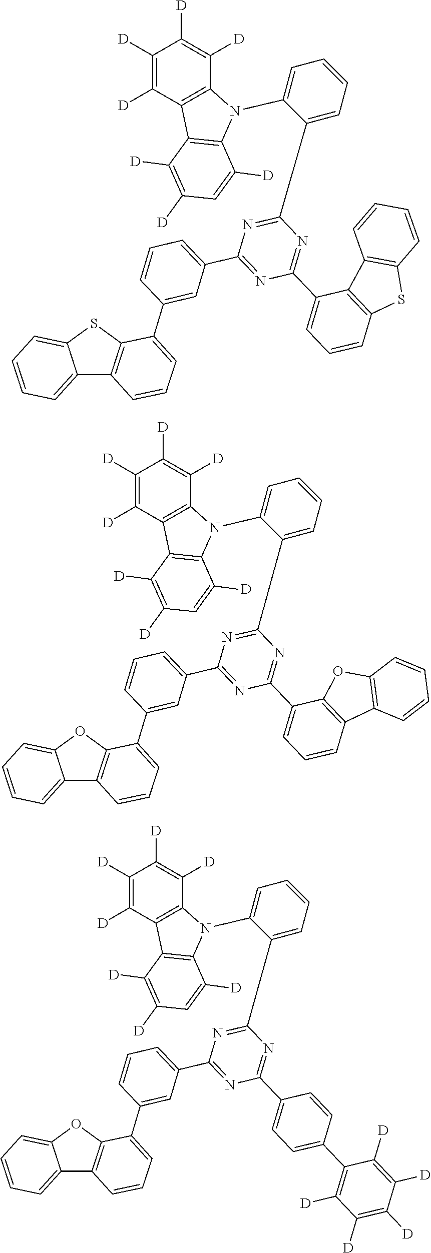

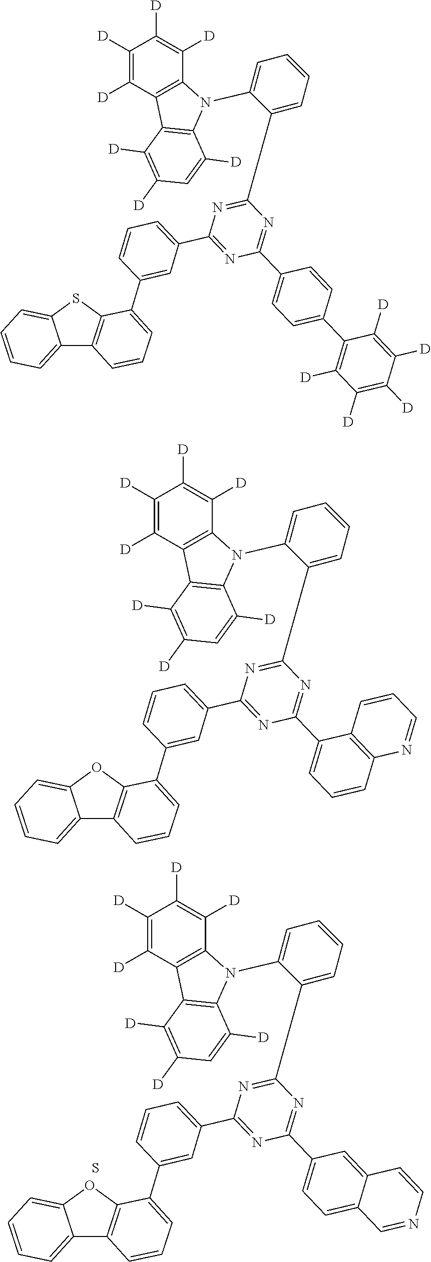

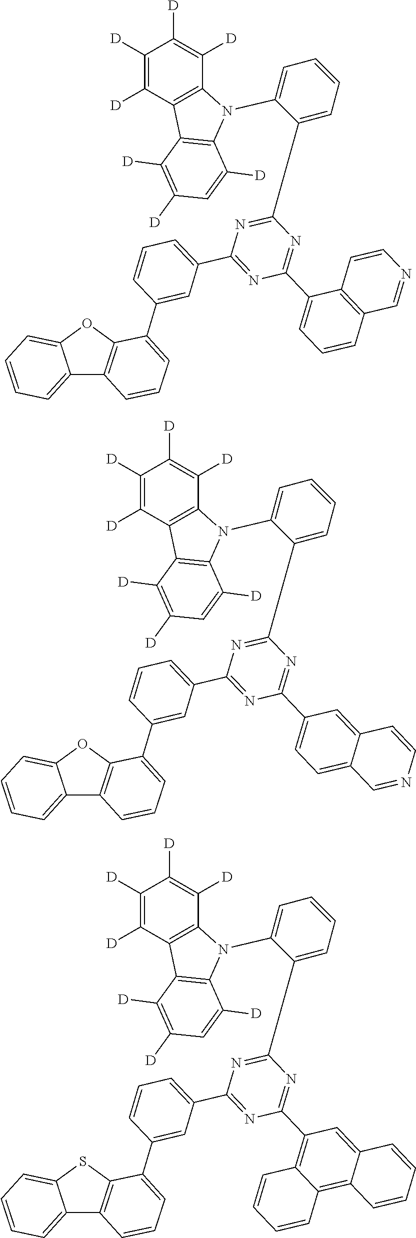

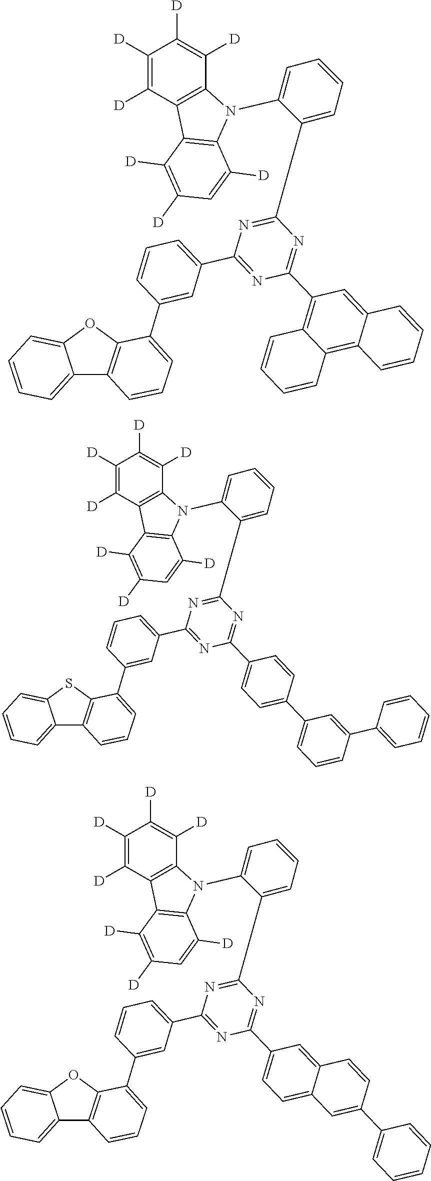

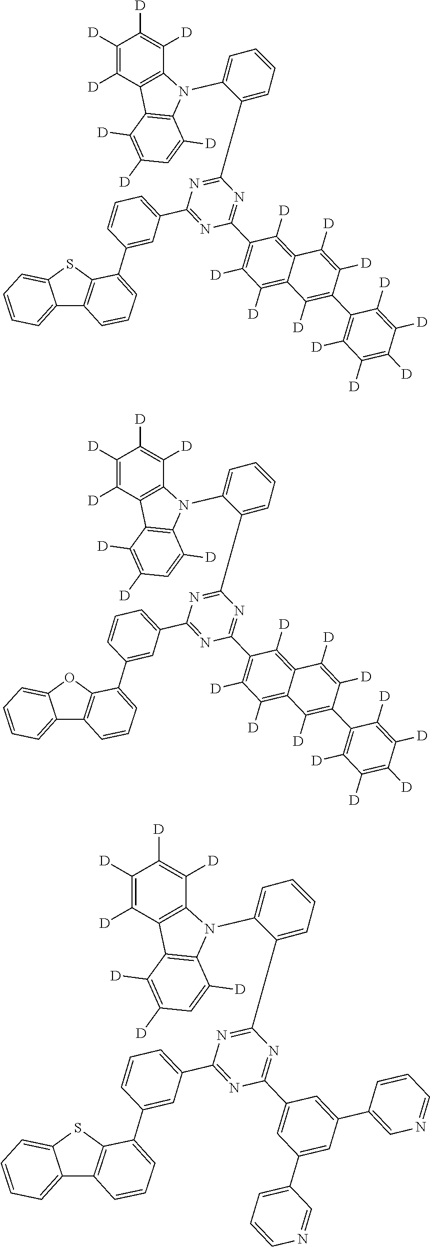

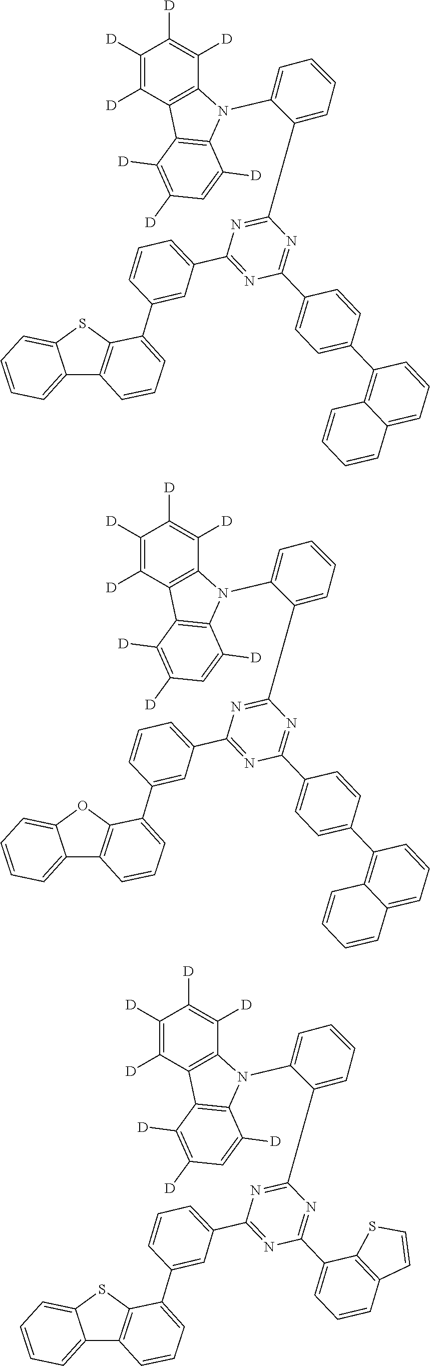 [SMILES: [2H]c1cc([2H])c2c(c1[2H])c1c([2H])c([2H])c([2H])c([2H])c1n2-c1ccccc1-c1nc(-c2ccc(-c3cccc4ccccc34)cc2)nc(-c2cccc(-c3cccc4c3sc3ccccc34)c2)n1 ; [2H]c1cc([2H])c2c(c1[2H])c1c([2H])c([2H])c([2H])c([2H])c1n2-c1ccccc1-c1nc(-c2ccc(-c3cccc4ccccc34)cc2)nc(-c2cccc(-c3cccc4c3oc3ccccc34)c2)n1 ; [2H]c1cc([2H])c2c(c1[2H])c1c([2H])c([2H])c([2H])c([2H])c1n2-c1ccccc1-c1nc(-c2cccc(-c3cccc4c3sc3ccccc34)c2)nc(-c2cccc3ccsc23)n1]

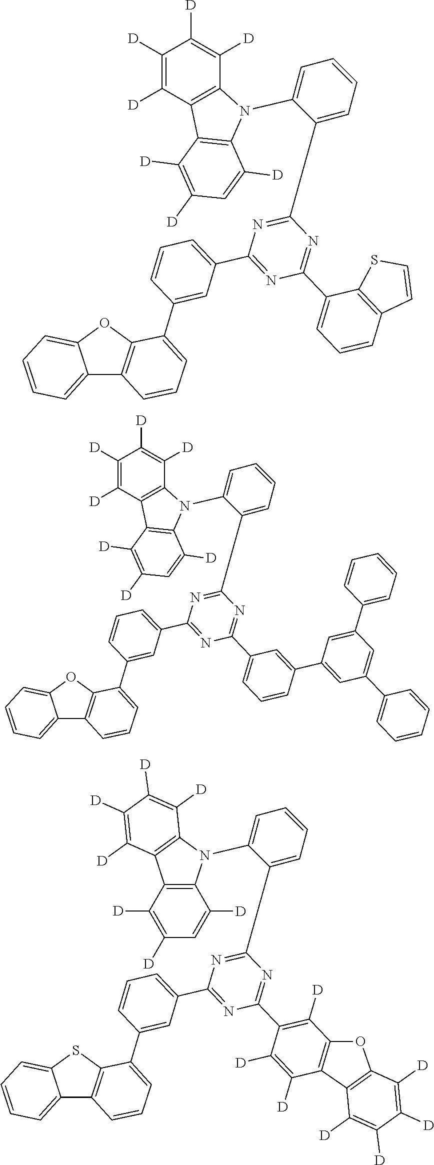

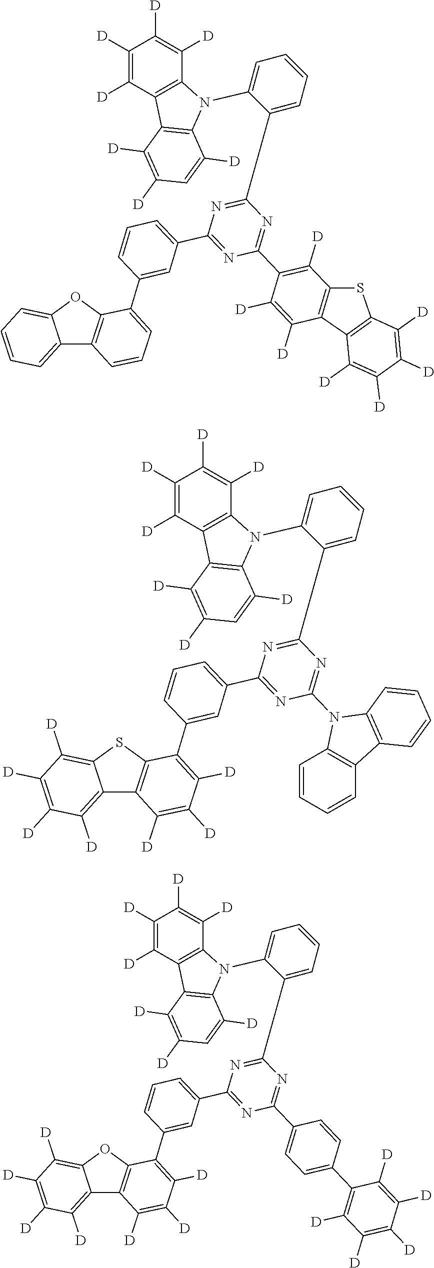

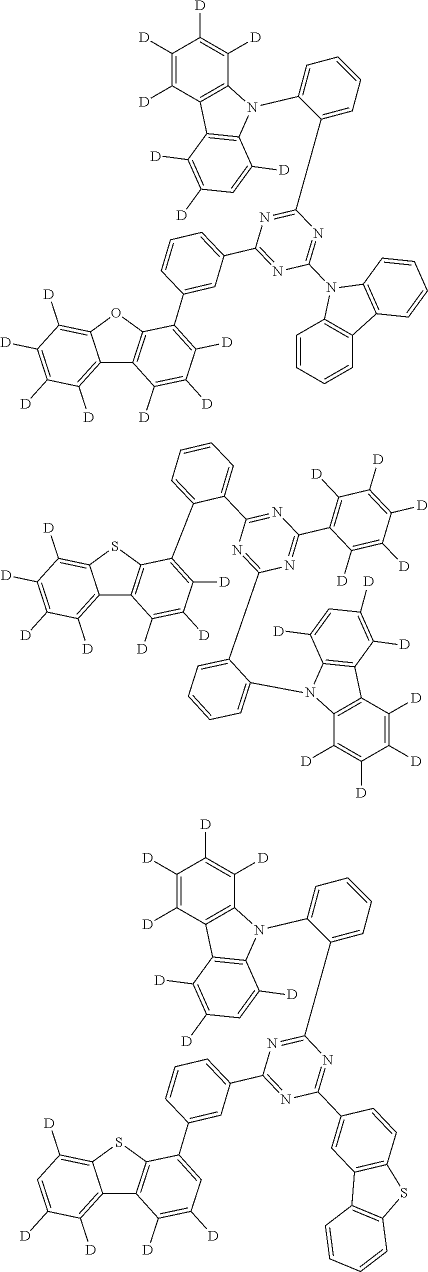

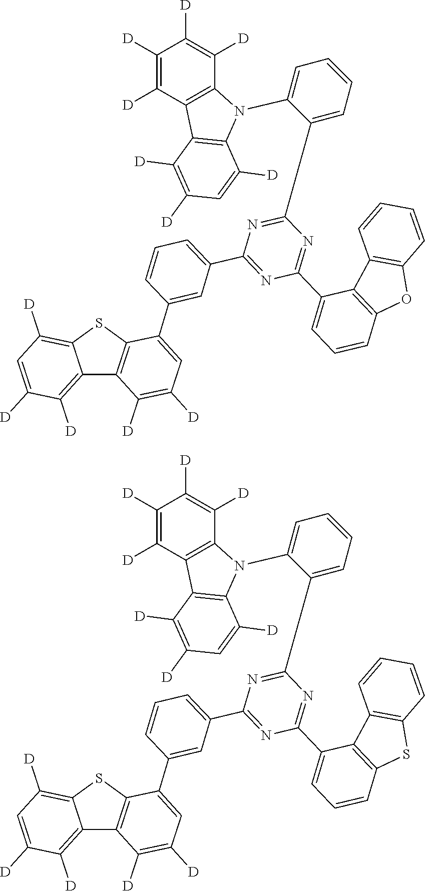

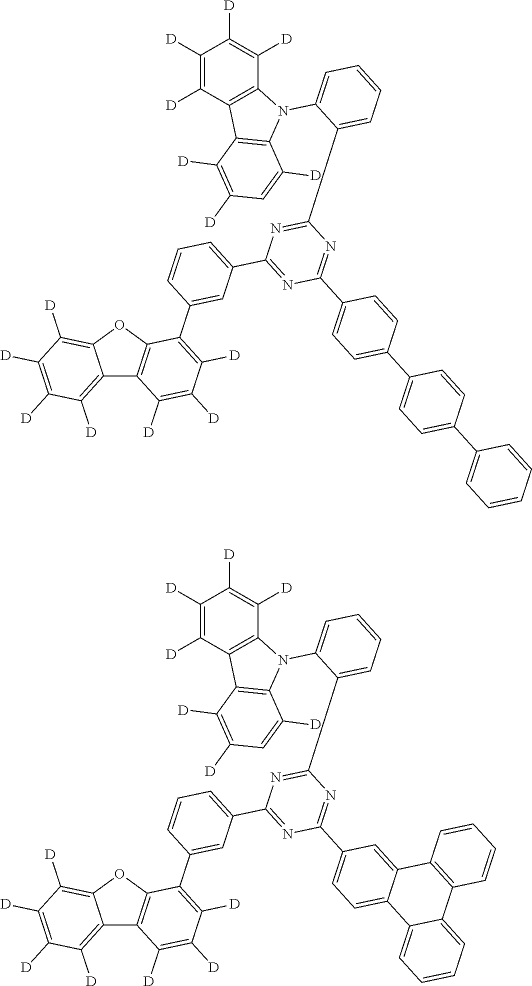 [SMILES: [2H]c1cc([2H])c2c(c1[2H])c1c([2H])c([2H])c([2H])c([2H])c1n2-c1ccccc1-c1nc(-c2ccc(-c3ccc(-c4ccccc4)cc3)cc2)nc(-c2cccc(-c3c([2H])c([2H])c([2H])c4c3oc3c([2H])c([2H])c([2H])c([2H])c34)c2)n1 ; [2H]c1cc([2H])c2c(c1[2H])c1c([2H])c([2H])c([2H])c([2H])c1n2-c1ccccc1-c1nc(-c2cccc(-c3c([2H])c([2H])c([2H])c4c3oc3c([2H])c([2H])c([2H])c([2H])c34)c2)nc(-c2ccc3c4ccccc4c4ccccc4c3c2)n1]

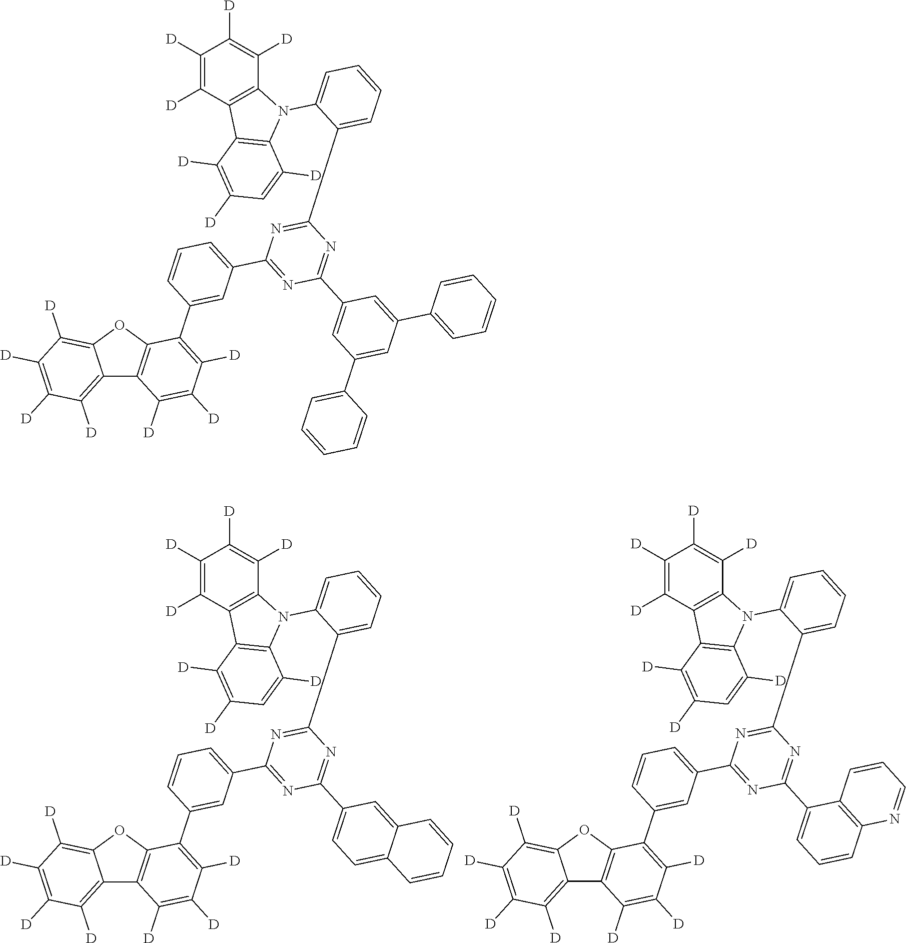

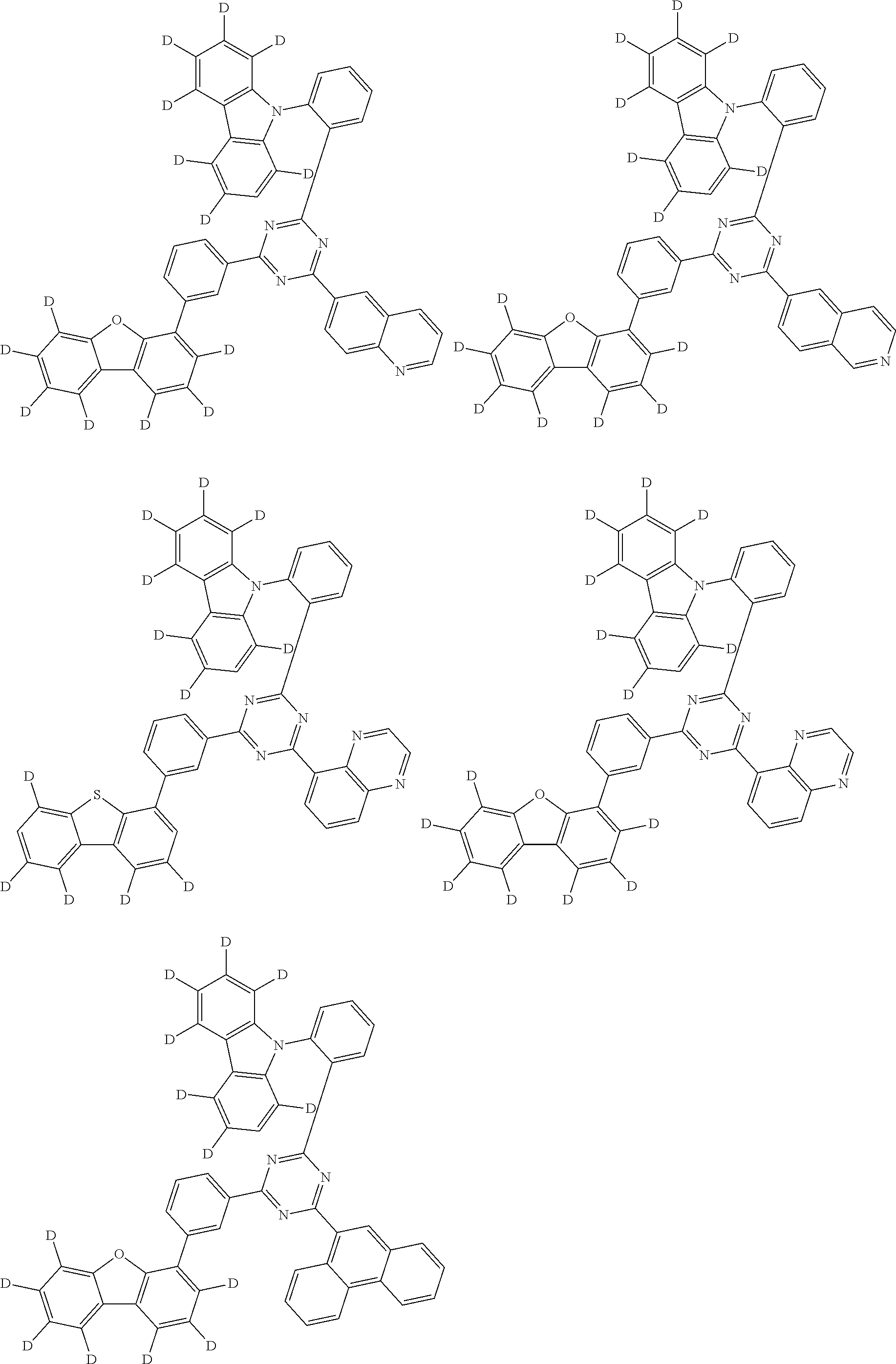

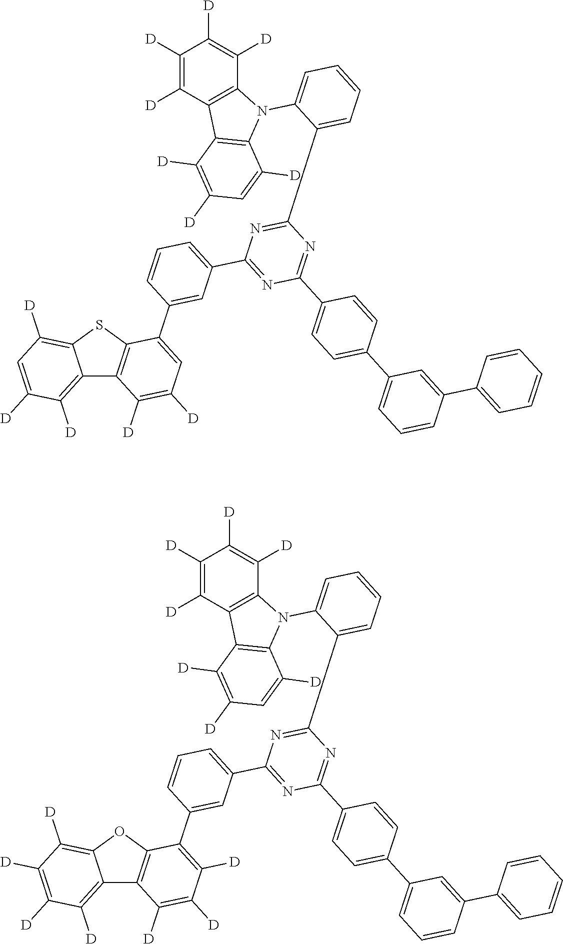 [SMILES: [2H]c1cc([2H])c2sc3c(-c4cccc(-c5nc(-c6ccc(-c7cccc(-c8ccccc8)c7)cc6)nc(-c6ccccc6-n6c7c([2H])cc([2H])c([2H])c7c7c([2H])c([2H])c([2H])c([2H])c76)n5)c4)cc([2H])c([2H])c3c2c1[2H] ; [2H]c1cc([2H])c2c(c1[2H])c1c([2H])c([2H])c([2H])c([2H])c1n2-c1ccccc1-c1nc(-c2ccc(-c3cccc(-c4ccccc4)c3)cc2)nc(-c2cccc(-c3c([2H])c([2H])c([2H])c4c3oc3c([2H])c([2H])c([2H])c([2H])c34)c2)n1]

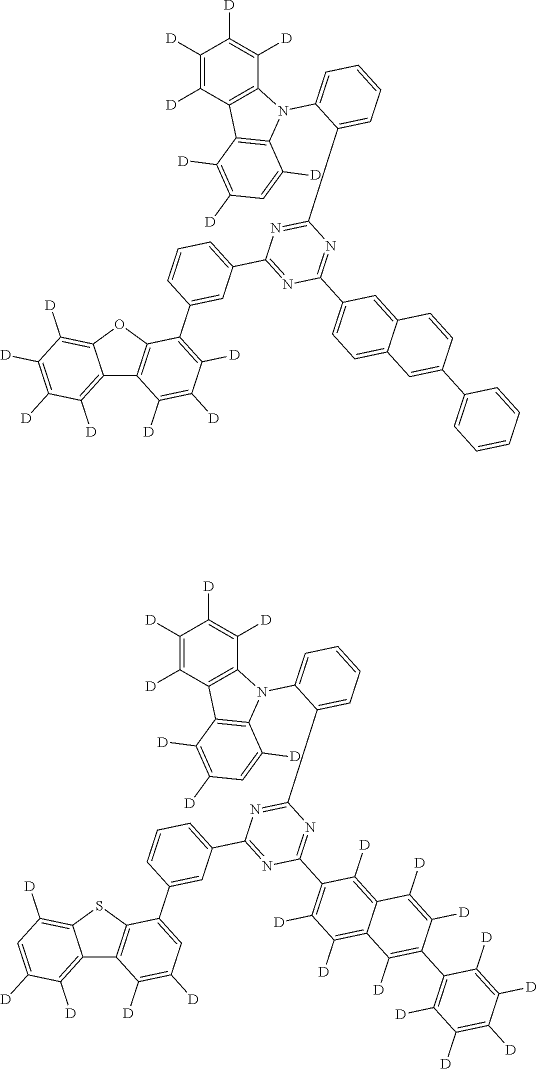 [SMILES: [2H]c1cc([2H])c2c(c1[2H])c1c([2H])c([2H])c([2H])c([2H])c1n2-c1ccccc1-c1nc(-c2cccc(-c3c([2H])c([2H])c([2H])c4c3oc3c([2H])c([2H])c([2H])c([2H])c34)c2)nc(-c2ccc3cc(-c4ccccc4)ccc3c2)n1 ; [2H]c1cc([2H])c2sc3c(-c4cccc(-c5nc(-c6ccccc6-n6c7c([2H])cc([2H])c([2H])c7c7c([2H])c([2H])c([2H])c([2H])c76)nc(-c6c([2H])c([2H])c7c([2H])c(-c8c([2H])c([2H])c([2H])c([2H])c8[2H])c([2H])c([2H])c7c6[2H])n5)c4)cc([2H])c([2H])c3c2c1[2H]]

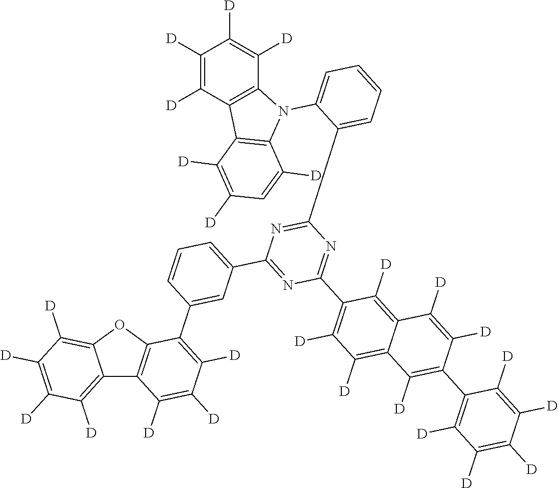 [SMILES: [2H]c1cc([2H])c2c(c1[2H])c1c([2H])c([2H])c([2H])c([2H])c1n2-c1ccccc1-c1nc(-c2cccc(-c3c([2H])c([2H])c([2H])c4c3oc3c([2H])c([2H])c([2H])c([2H])c34)c2)nc(-c2c([2H])c([2H])c3c([2H])c(-c4c([2H])c([2H])c([2H])c([2H])c4[2H])c([2H])c([2H])c3c2[2H])n1]

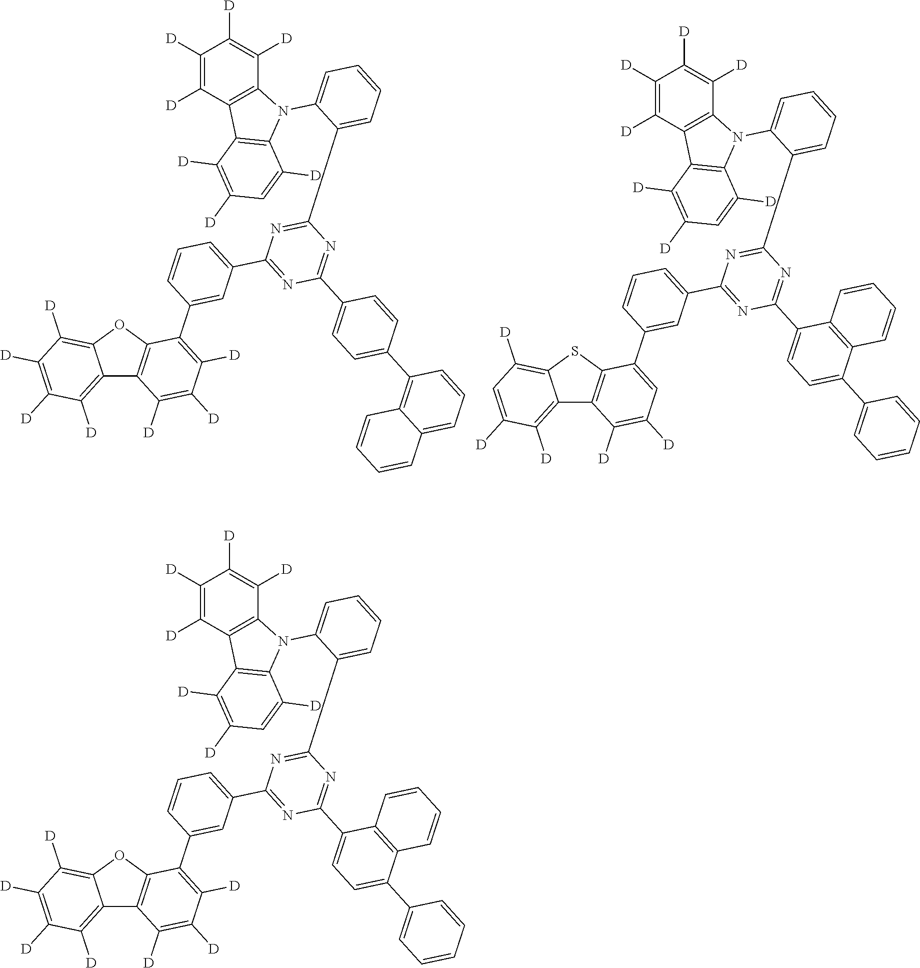

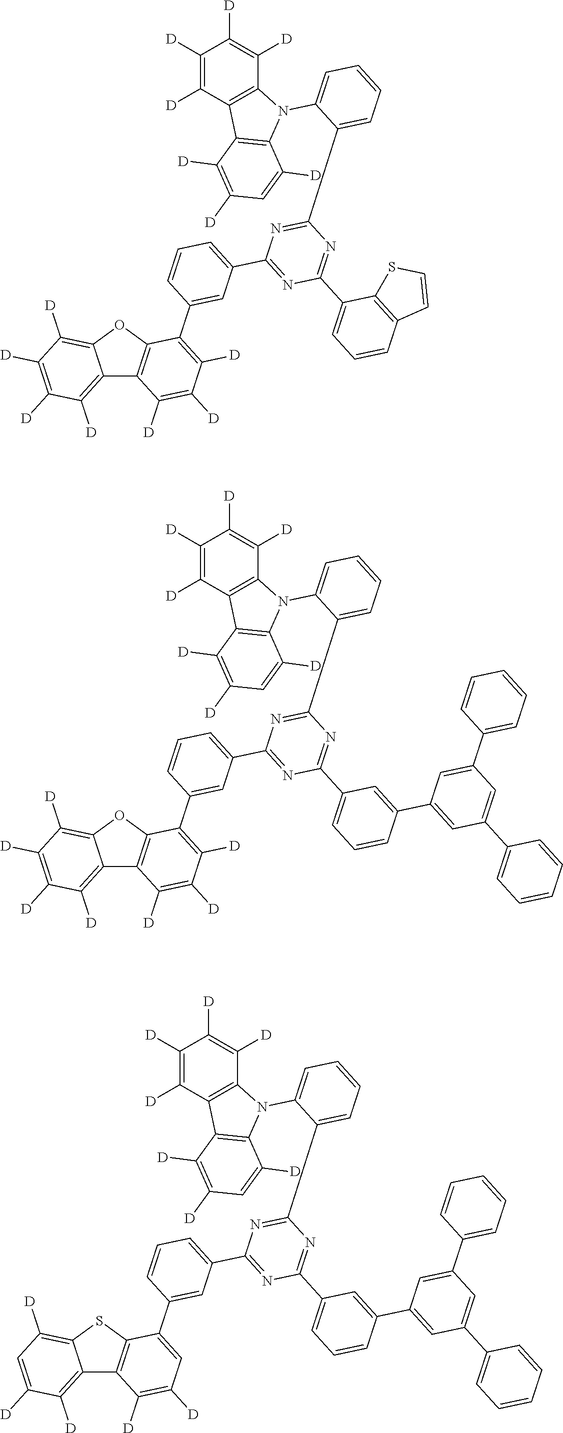

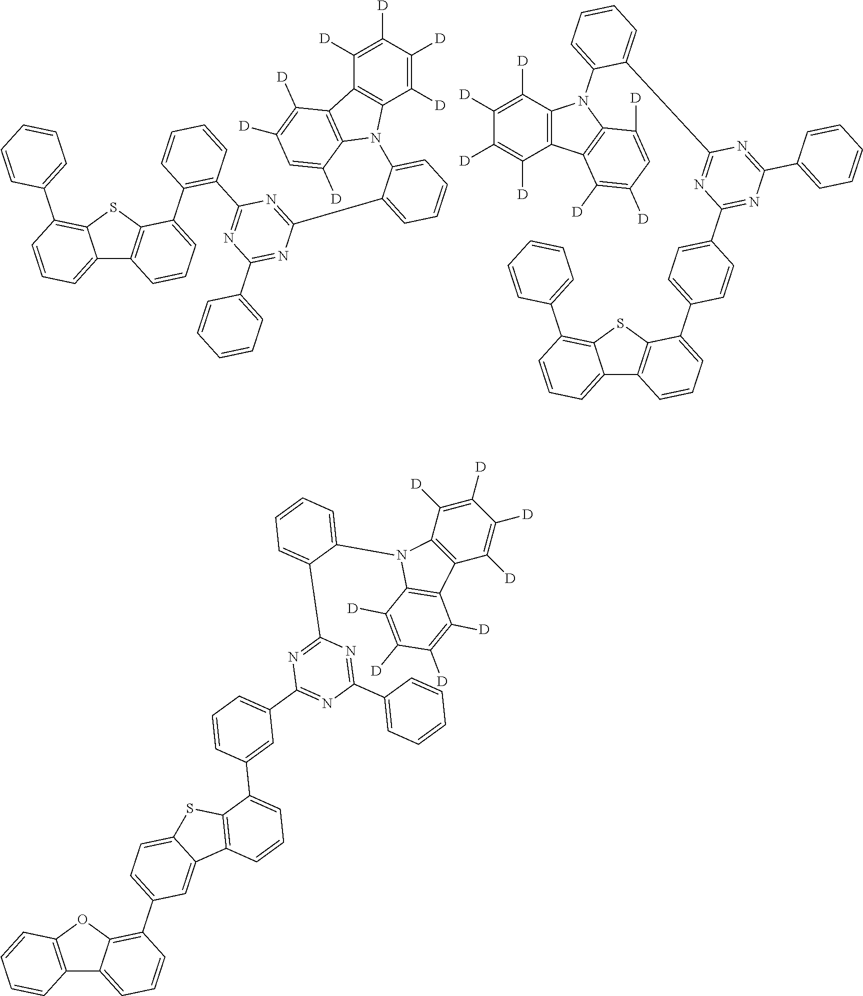

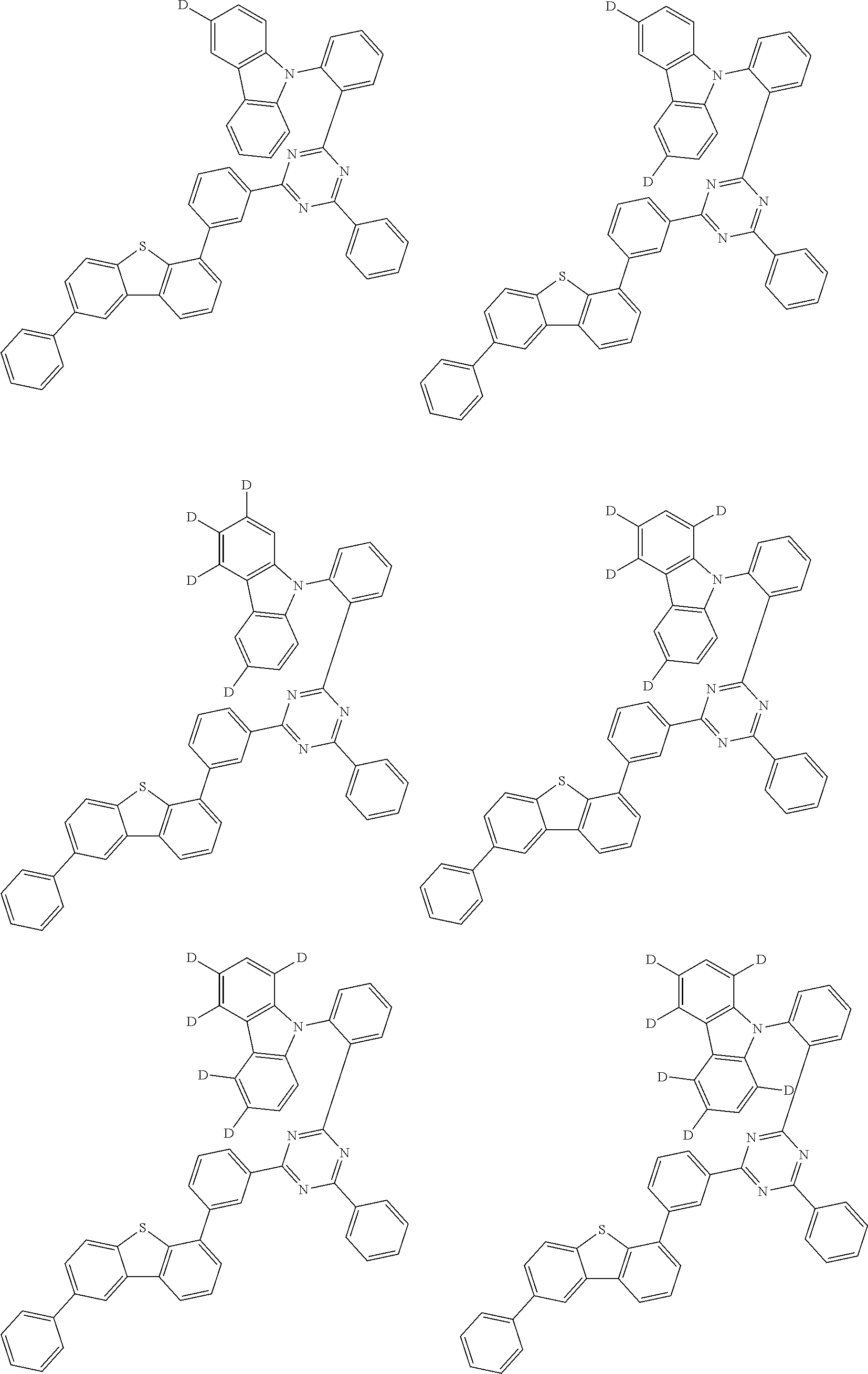

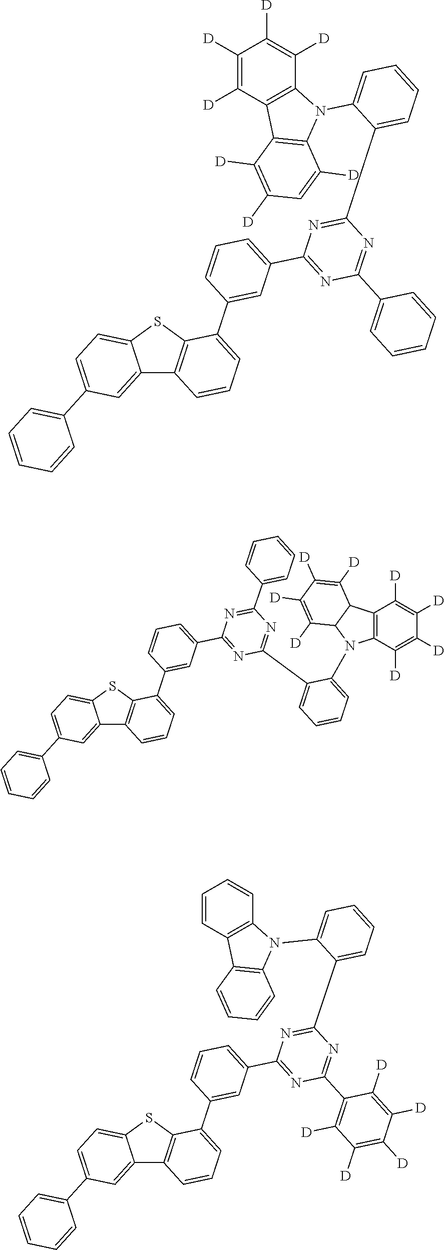 [SMILES: [2H]c1cc([2H])c2c(c1[2H])c1c([2H])c([2H])c([2H])c([2H])c1n2-c1ccccc1-c1nc(-c2ccccc2)nc(-c2cccc(-c3cccc4c3sc3ccc(-c5ccccc5)cc34)c2)n1 ; [2H]C1=C([2H])C2c3c([2H])c([2H])c([2H])c([2H])c3N(c3ccccc3-c3nc(-c4ccccc4)nc(-c4cccc(-c5cccc6c5sc5ccc(-c7ccccc7)cc56)c4)n3)C2C([2H])=C1[2H] ; [2H]c1c([2H])c([2H])c(-c2nc(-c3cccc(-c4cccc5c4sc4ccc(-c6ccccc6)cc45)c3)nc(-c3ccccc3-n3c4ccccc4c4ccccc43)n2)c([2H])c1[2H]]

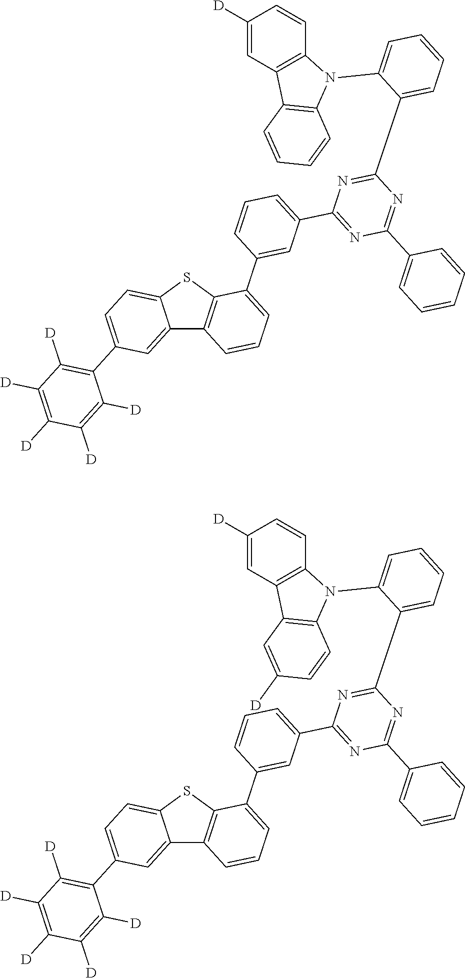 [SMILES: [2H]c1ccc2c(c1)c1ccccc1n2-c1ccccc1-c1nc(-c2ccccc2)nc(-c2cccc(-c3cccc4c3sc3ccc(-c5c([2H])c([2H])c([2H])c([2H])c5[2H])cc34)c2)n1 ; [2H]c1ccc2c(c1)c1cc([2H])ccc1n2-c1ccccc1-c1nc(-c2ccccc2)nc(-c2cccc(-c3cccc4c3sc3ccc(-c5c([2H])c([2H])c([2H])c([2H])c5[2H])cc34)c2)n1]

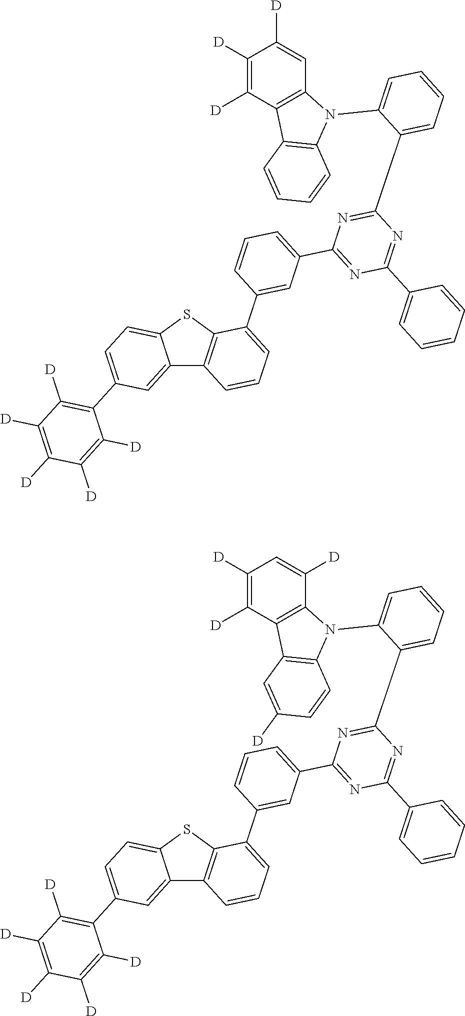 [SMILES: [2H]c1cc2c(c([2H])c1[2H])c1ccccc1n2-c1ccccc1-c1nc(-c2ccccc2)nc(-c2cccc(-c3cccc4c3sc3ccc(-c5c([2H])c([2H])c([2H])c([2H])c5[2H])cc34)c2)n1 ; [2H]c1ccc2c(c1)c1c([2H])c([2H])cc([2H])c1n2-c1ccccc1-c1nc(-c2ccccc2)nc(-c2cccc(-c3cccc4c3sc3ccc(-c5c([2H])c([2H])c([2H])c([2H])c5[2H])cc34)c2)n1]

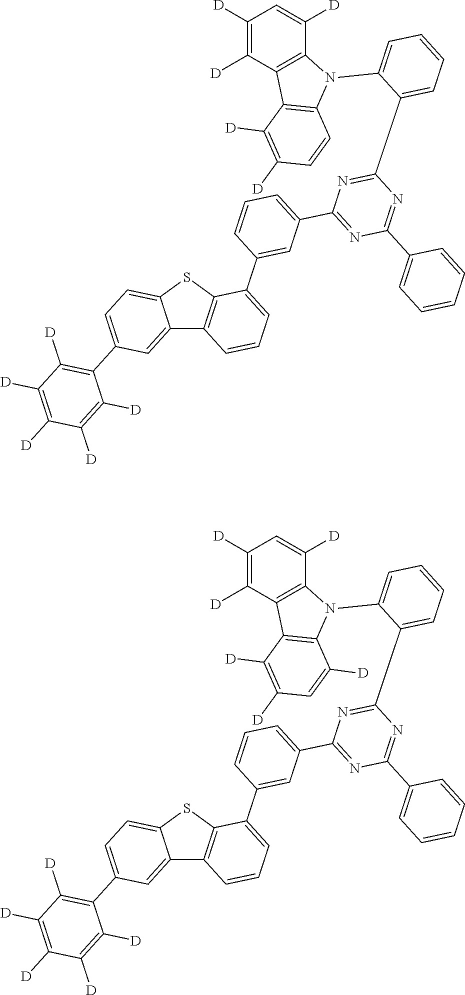 [SMILES: [2H]c1ccc2c(c1[2H])c1c([2H])c([2H])cc([2H])c1n2-c1ccccc1-c1nc(-c2ccccc2)nc(-c2cccc(-c3cccc4c3sc3ccc(-c5c([2H])c([2H])c([2H])c([2H])c5[2H])cc34)c2)n1 ; [2H]c1cc([2H])c2c(c1[2H])c1c([2H])c([2H])cc([2H])c1n2-c1ccccc1-c1nc(-c2ccccc2)nc(-c2cccc(-c3cccc4c3sc3ccc(-c5c([2H])c([2H])c([2H])c([2H])c5[2H])cc34)c2)n1]

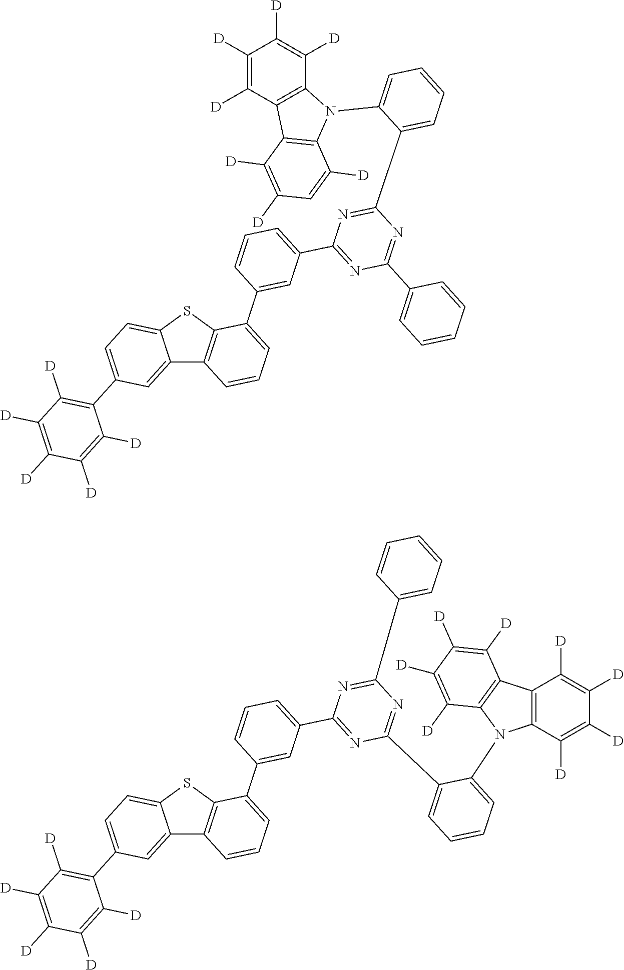 [SMILES: [2H]c1cc([2H])c2c(c1[2H])c1c([2H])c([2H])c([2H])c([2H])c1n2-c1ccccc1-c1nc(-c2ccccc2)nc(-c2cccc(-c3cccc4c3sc3ccc(-c5c([2H])c([2H])c([2H])c([2H])c5[2H])cc34)c2)n1 ; [2H]c1c([2H])c([2H])c(-c2ccc3sc4c(-c5cccc(-c6nc(-c7ccccc7)nc(-c7ccccc7-n7c8c([2H])c([2H])c([2H])c([2H])c8c8c([2H])c([2H])c([2H])c([2H])c87)n6)c5)cccc4c3c2)c([2H])c1[2H]]

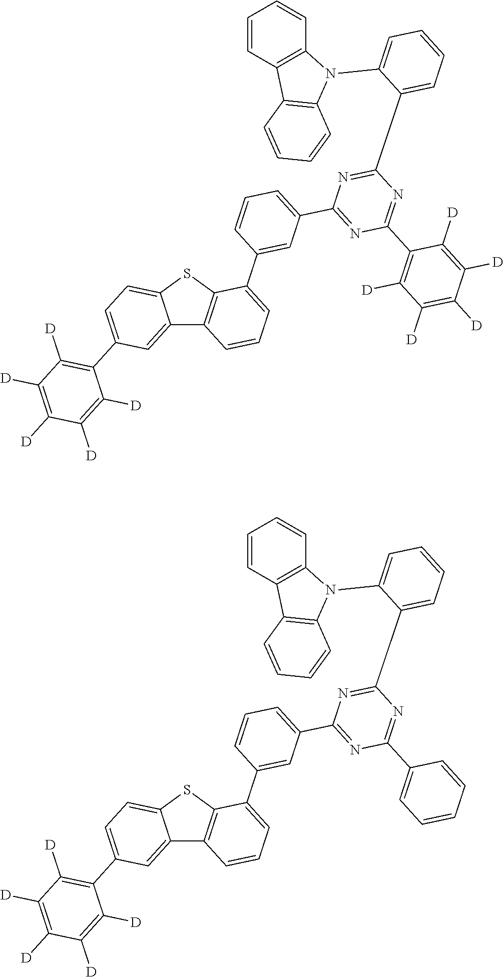 [SMILES: [2H]c1c([2H])c([2H])c(-c2ccc3sc4c(-c5cccc(-c6nc(-c7ccccc7-n7c8ccccc8c8ccccc87)nc(-c7c([2H])c([2H])c([2H])c([2H])c7[2H])n6)c5)cccc4c3c2)c([2H])c1[2H] ; [2H]c1c([2H])c([2H])c(-c2ccc3sc4c(-c5cccc(-c6nc(-c7ccccc7)nc(-c7ccccc7-n7c8ccccc8c8ccccc87)n6)c5)cccc4c3c2)c([2H])c1[2H]]

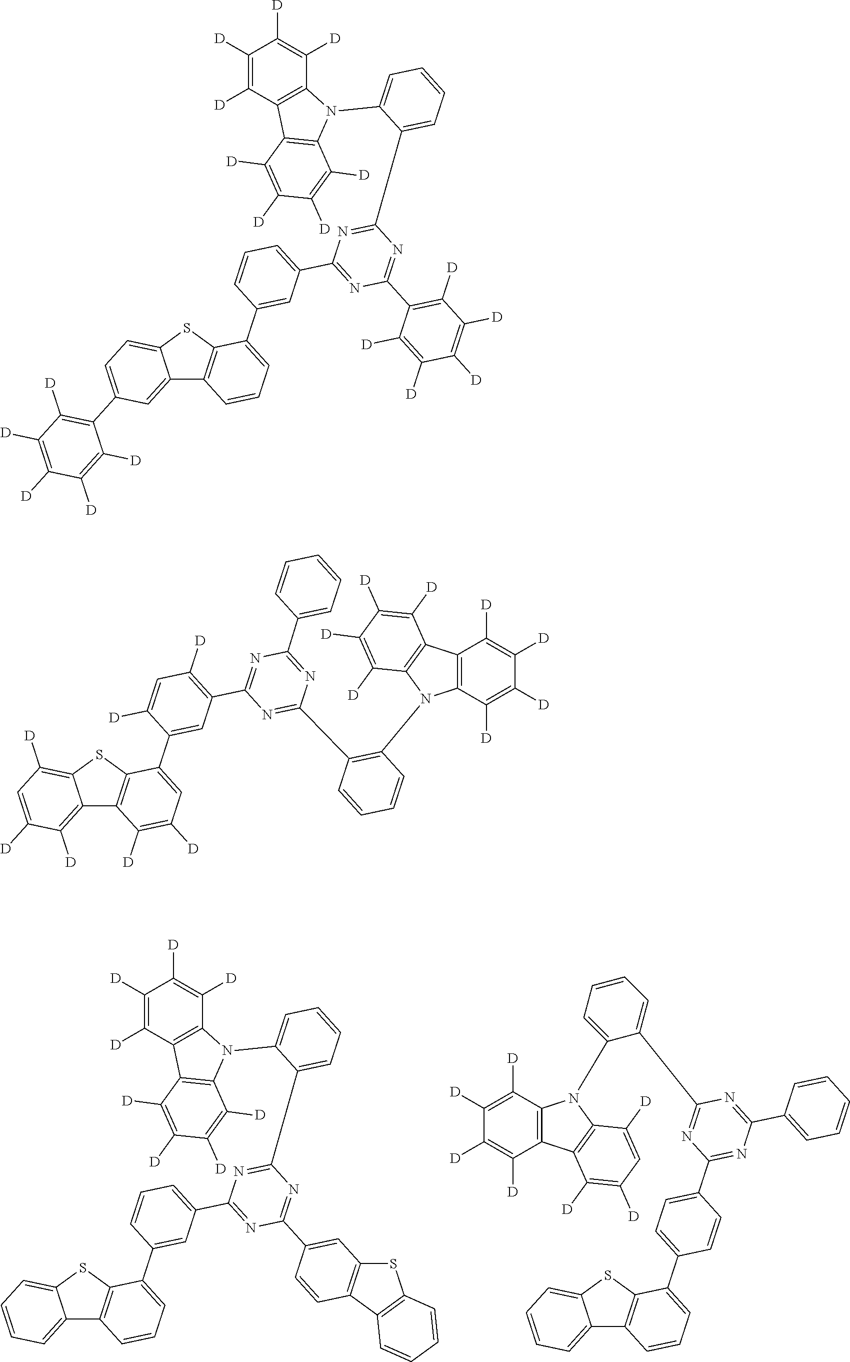

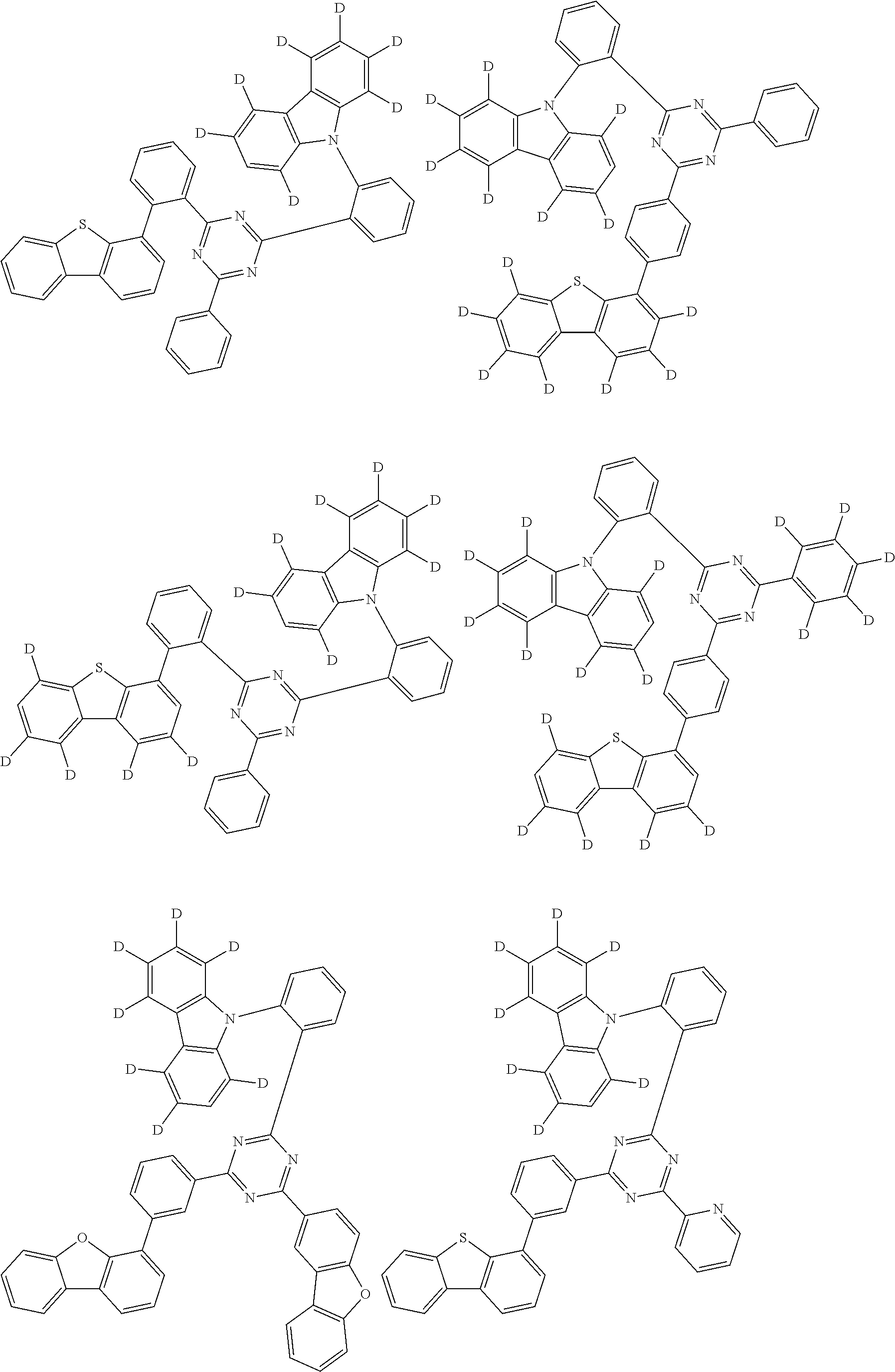

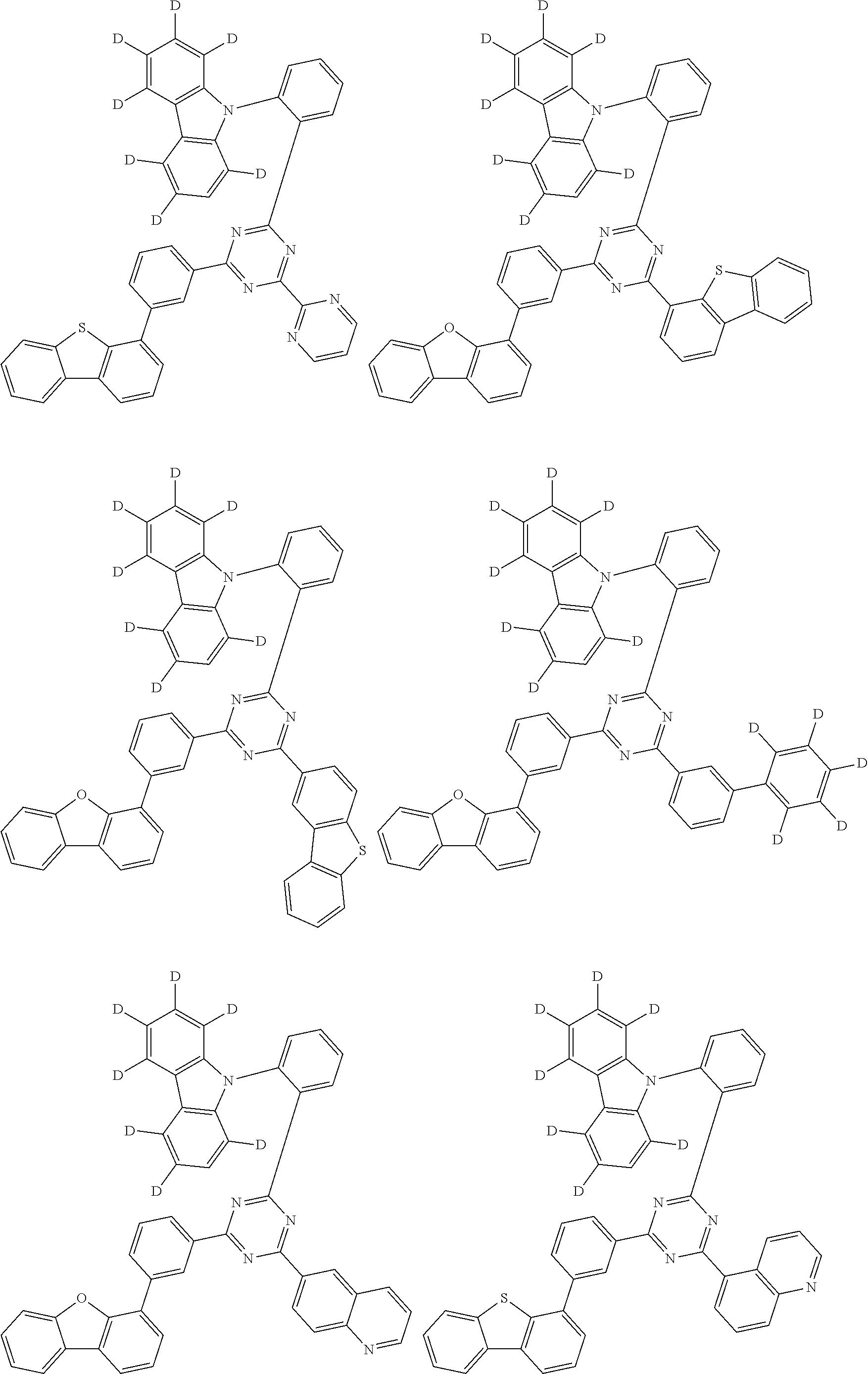

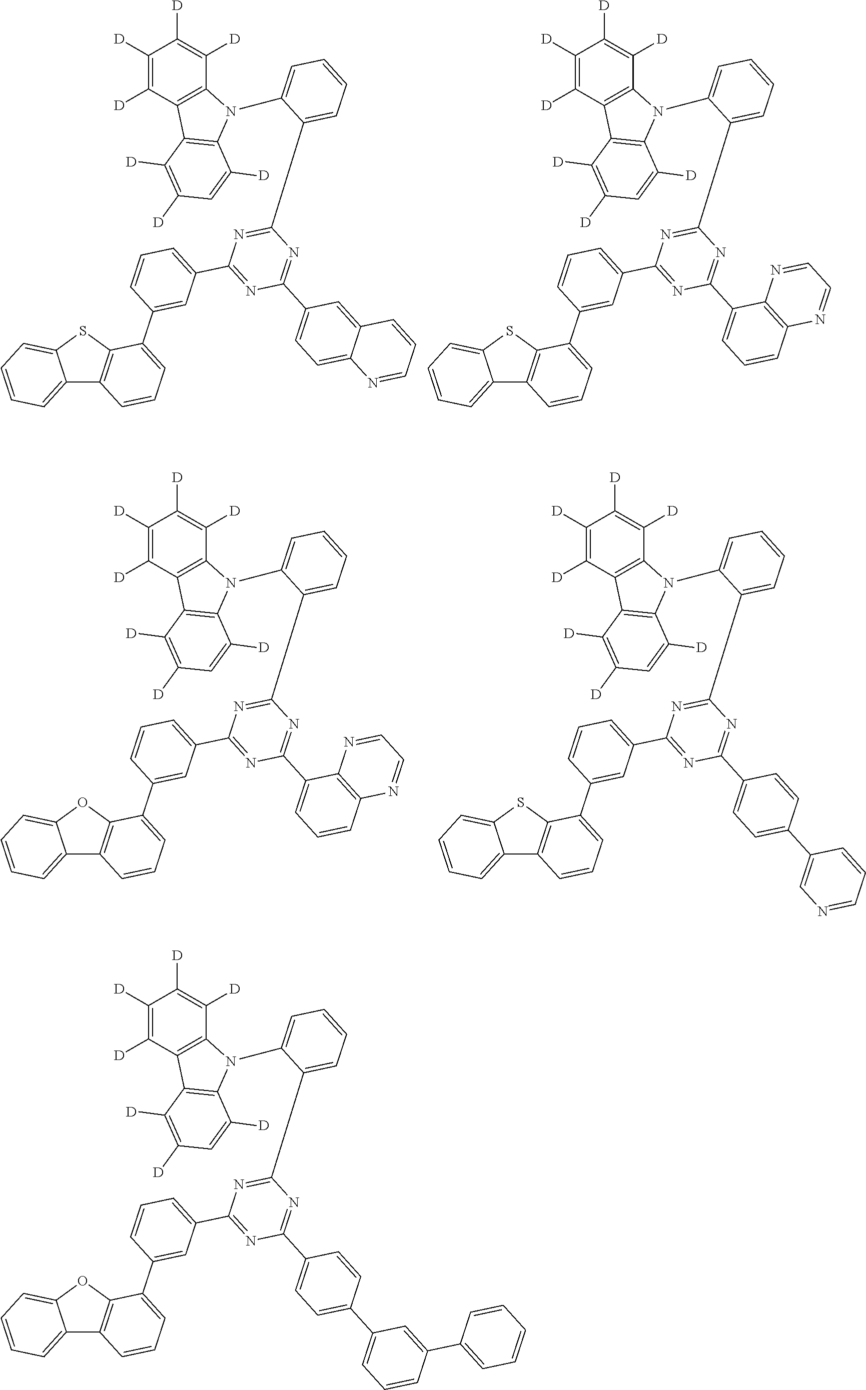

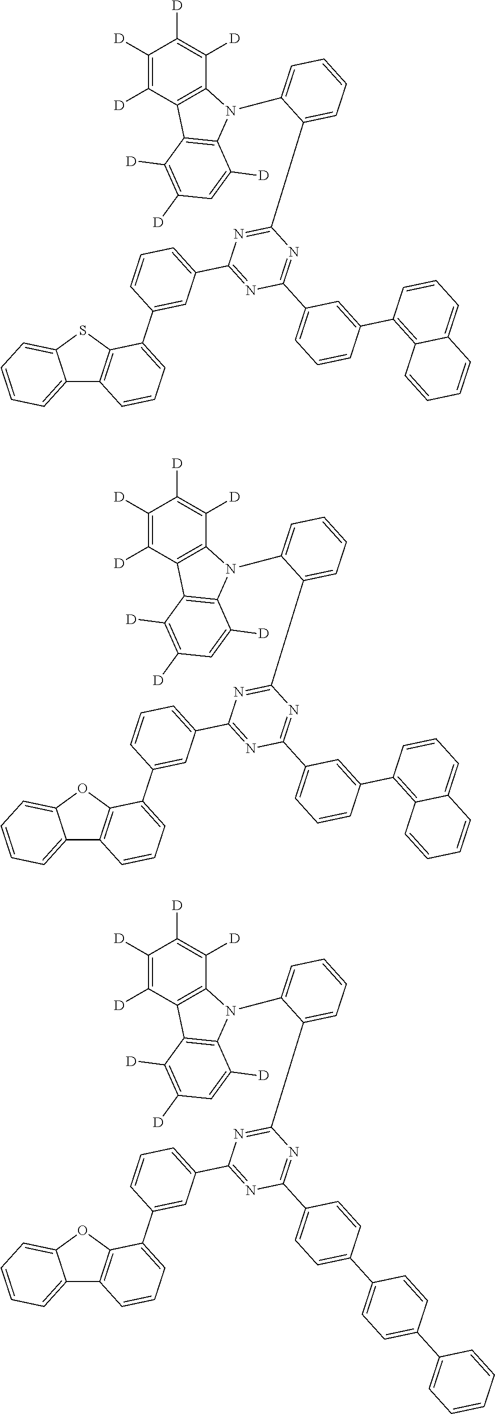 [SMILES: [2H]c1cc([2H])c2c(c1[2H])c1c([2H])c([2H])c([2H])c([2H])c1n2-c1ccccc1-c1nc(-c2cccc(-c3cccc4ccccc34)c2)nc(-c2cccc(-c3cccc4c3sc3ccccc34)c2)n1 ; [2H]c1cc([2H])c2c(c1[2H])c1c([2H])c([2H])c([2H])c([2H])c1n2-c1ccccc1-c1nc(-c2cccc(-c3cccc4ccccc34)c2)nc(-c2cccc(-c3cccc4c3oc3ccccc34)c2)n1 ; [2H]c1cc([2H])c2c(c1[2H])c1c([2H])c([2H])c([2H])c([2H])c1n2-c1ccccc1-c1nc(-c2ccc(-c3ccc(-c4ccccc4)cc3)cc2)nc(-c2cccc(-c3cccc4c3oc3ccccc34)c2)n1]

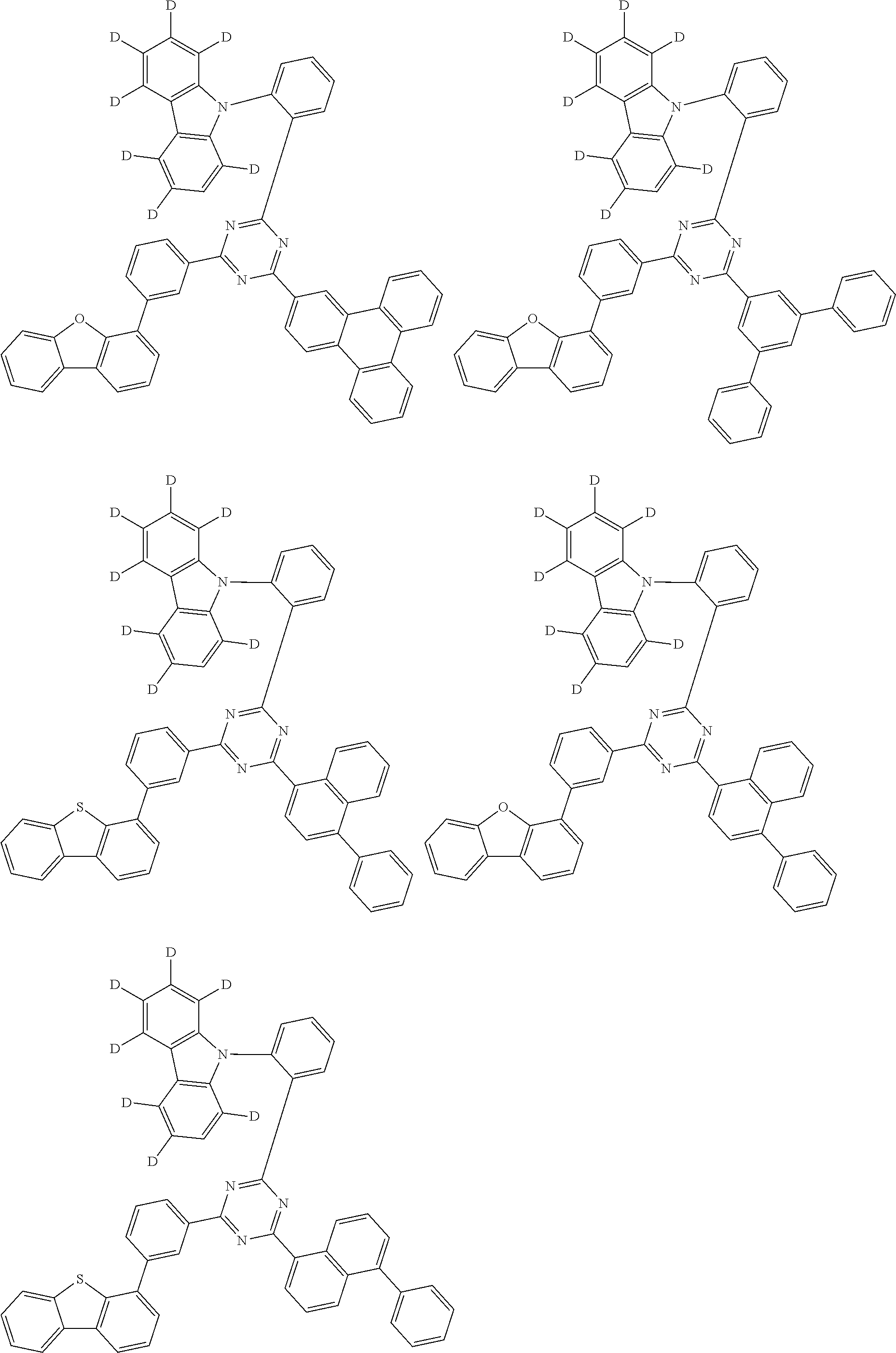

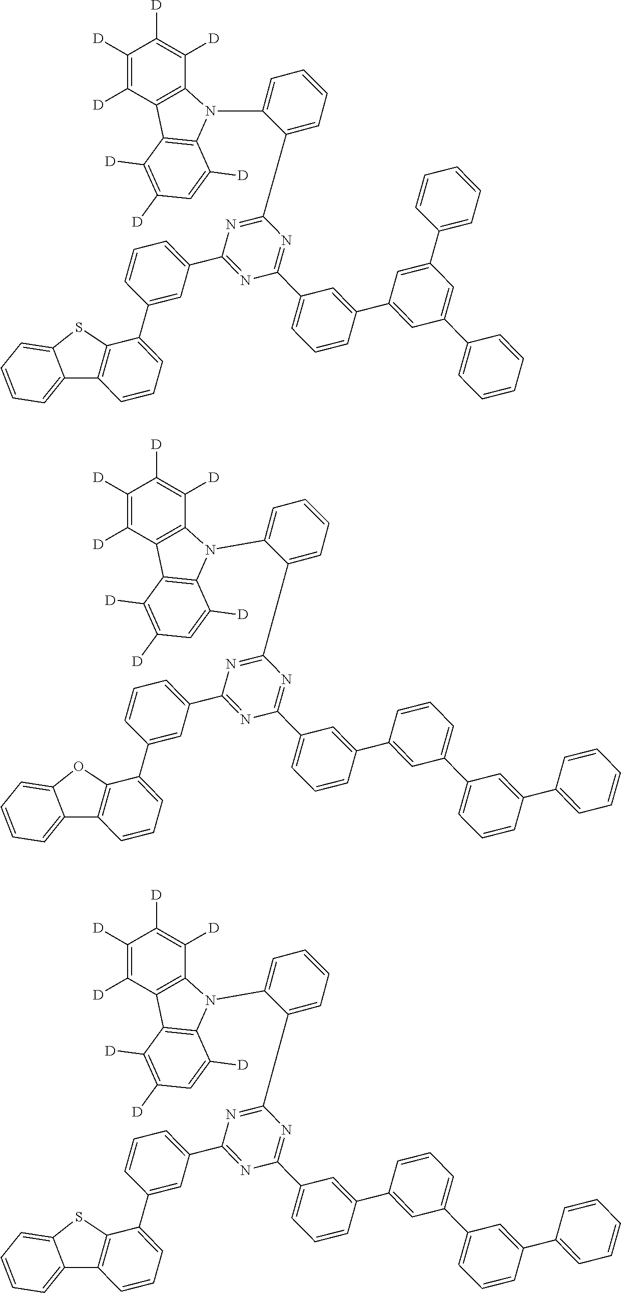 [SMILES: [2H]c1cc([2H])c2c(c1[2H])c1c([2H])c([2H])c([2H])c([2H])c1n2-c1ccccc1-c1nc(-c2cccc(-c3cc(-c4ccccc4)cc(-c4ccccc4)c3)c2)nc(-c2cccc(-c3cccc4c3sc3ccccc34)c2)n1 ; [2H]c1cc([2H])c2c(c1[2H])c1c([2H])c([2H])c([2H])c([2H])c1n2-c1ccccc1-c1nc(-c2cccc(-c3cccc(-c4cccc(-c5ccccc5)c4)c3)c2)nc(-c2cccc(-c3cccc4c3oc3ccccc34)c2)n1 ; [2H]c1cc([2H])c2c(c1[2H])c1c([2H])c([2H])c([2H])c([2H])c1n2-c1ccccc1-c1nc(-c2cccc(-c3cccc(-c4cccc(-c5ccccc5)c4)c3)c2)nc(-c2cccc(-c3cccc4c3sc3ccccc34)c2)n1]

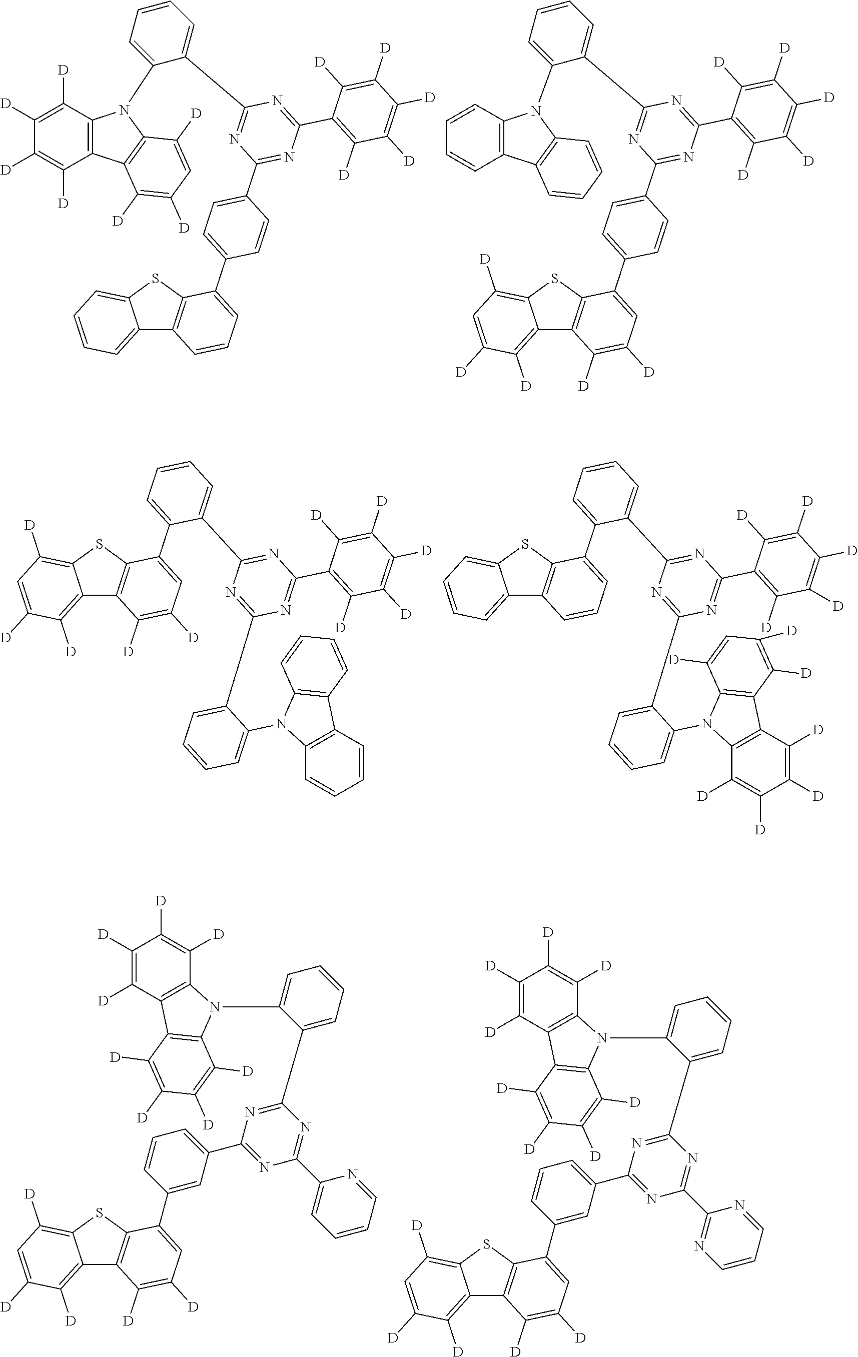

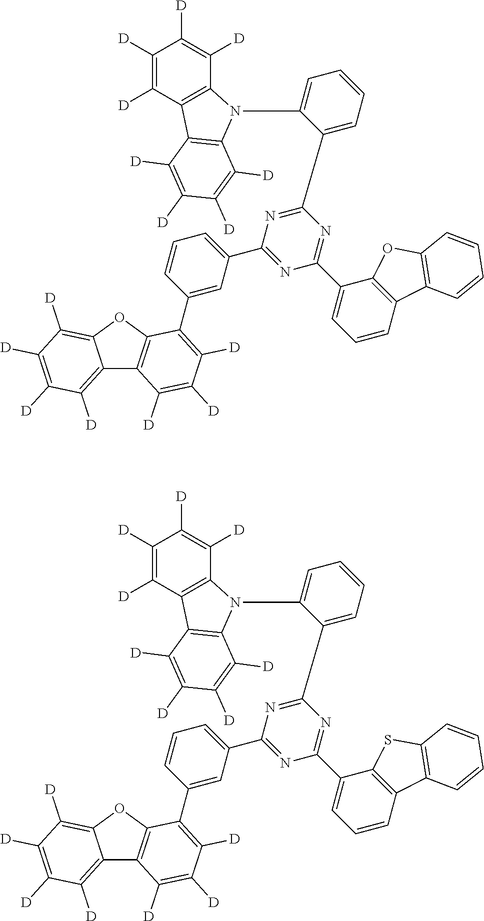 [SMILES: [2H]c1c([2H])c([2H])c2c(oc3c(-c4cccc(-c5nc(-c6ccccc6-n6c7c([2H])c([2H])c([2H])c([2H])c7c7c([2H])c([2H])c([2H])c([2H])c76)nc(-c6cccc7c6oc6ccccc67)n5)c4)c([2H])c([2H])c([2H])c32)c1[2H] ; [2H]c1c([2H])c([2H])c2c(oc3c(-c4cccc(-c5nc(-c6ccccc6-n6c7c([2H])c([2H])c([2H])c([2H])c7c7c([2H])c([2H])c([2H])c([2H])c76)nc(-c6cccc7c6sc6ccccc67)n5)c4)c([2H])c([2H])c([2H])c32)c1[2H]]

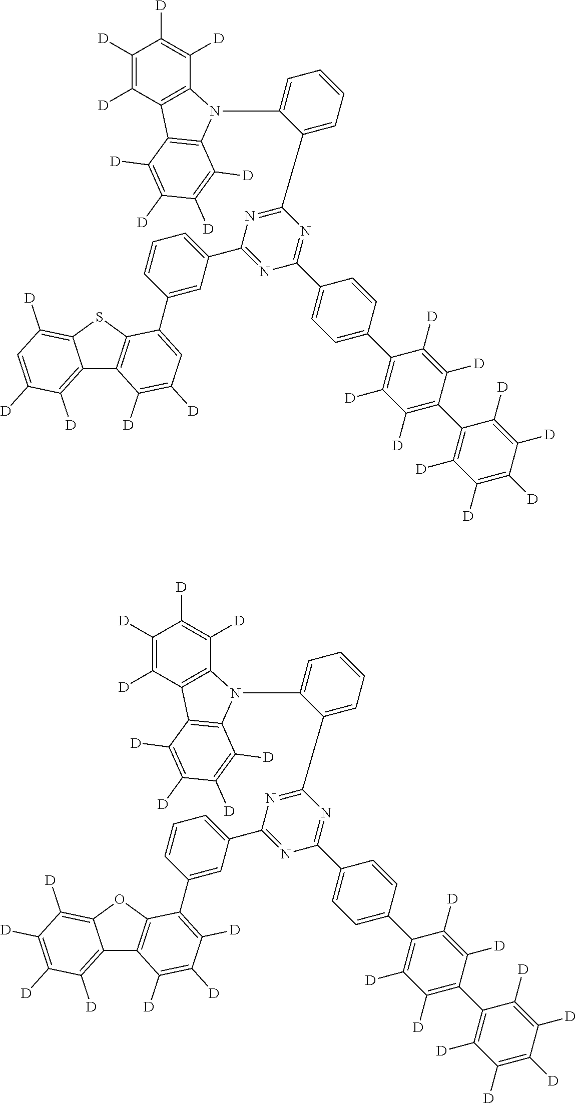 [SMILES: [2H]c1cc([2H])c2sc3c(-c4cccc(-c5nc(-c6ccc(-c7c([2H])c([2H])c(-c8c([2H])c([2H])c([2H])c([2H])c8[2H])c([2H])c7[2H])cc6)nc(-c6ccccc6-n6c7c([2H])c([2H])c([2H])c([2H])c7c7c([2H])c([2H])c([2H])c([2H])c76)n5)c4)cc([2H])c([2H])c3c2c1[2H] ; [2H]c1c([2H])c([2H])c(-c2c([2H])c([2H])c(-c3ccc(-c4nc(-c5cccc(-c6c([2H])c([2H])c([2H])c7c6oc6c([2H])c([2H])c([2H])c([2H])c67)c5)nc(-c5ccccc5-n5c6c([2H])c([2H])c([2H])c([2H])c6c6c([2H])c([2H])c([2H])c([2H])c65)n4)cc3)c([2H])c2[2H])c([2H])c1[2H]]

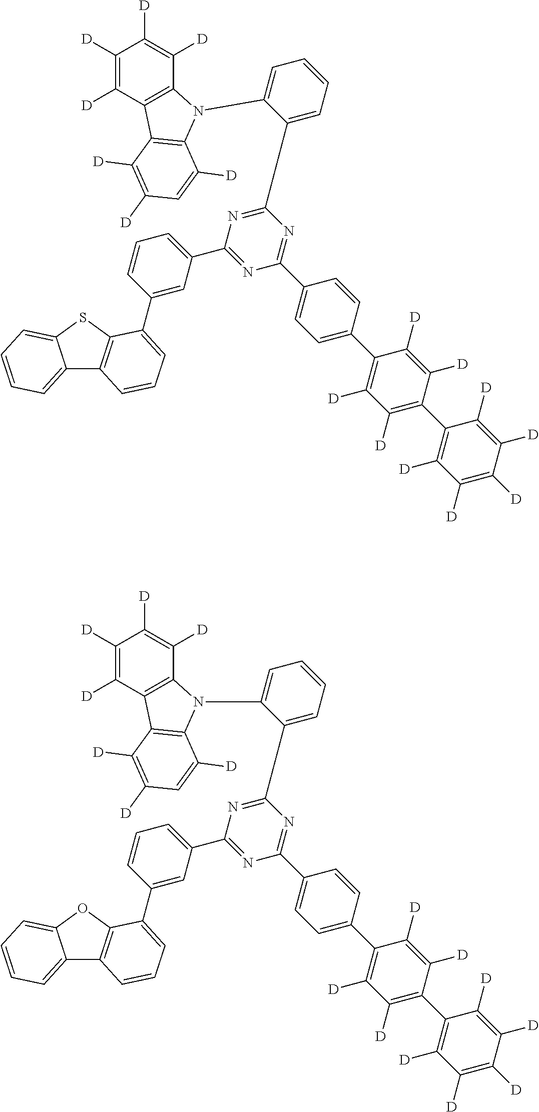 [SMILES: [2H]c1cc([2H])c2c(c1[2H])c1c([2H])c([2H])c([2H])c([2H])c1n2-c1ccccc1-c1nc(-c2ccc(-c3c([2H])c([2H])c(-c4c([2H])c([2H])c([2H])c([2H])c4[2H])c([2H])c3[2H])cc2)nc(-c2cccc(-c3cccc4c3sc3ccccc34)c2)n1 ; [2H]c1cc([2H])c2c(c1[2H])c1c([2H])c([2H])c([2H])c([2H])c1n2-c1ccccc1-c1nc(-c2ccc(-c3c([2H])c([2H])c(-c4c([2H])c([2H])c([2H])c([2H])c4[2H])c([2H])c3[2H])cc2)nc(-c2cccc(-c3cccc4c3oc3ccccc34)c2)n1]

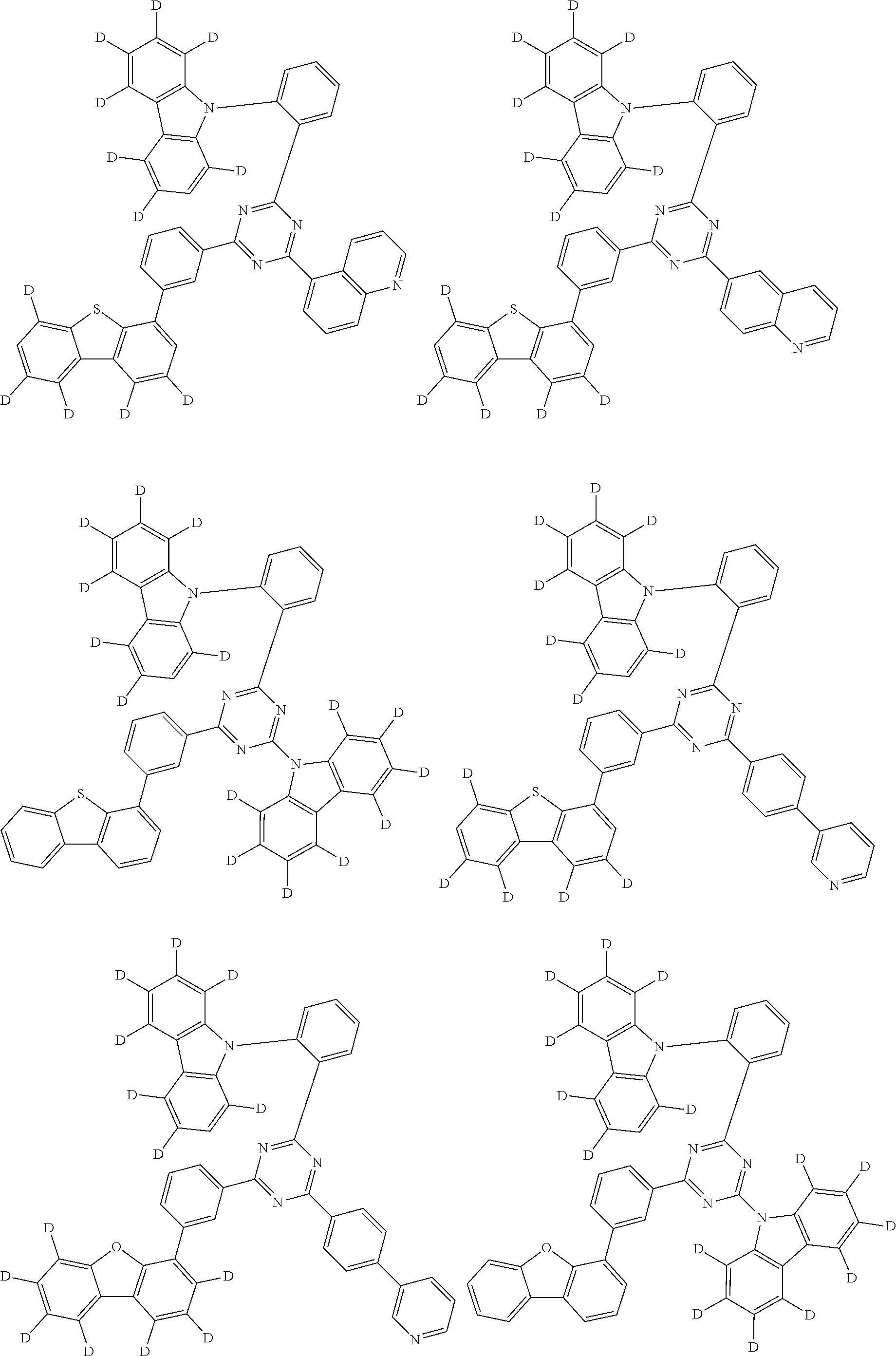

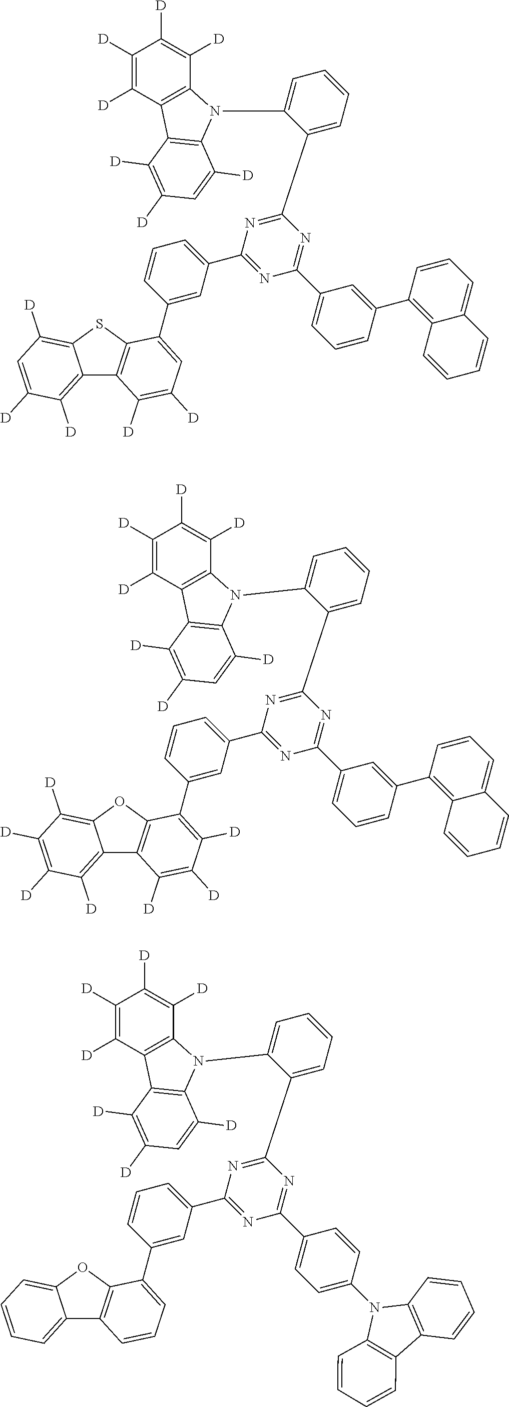

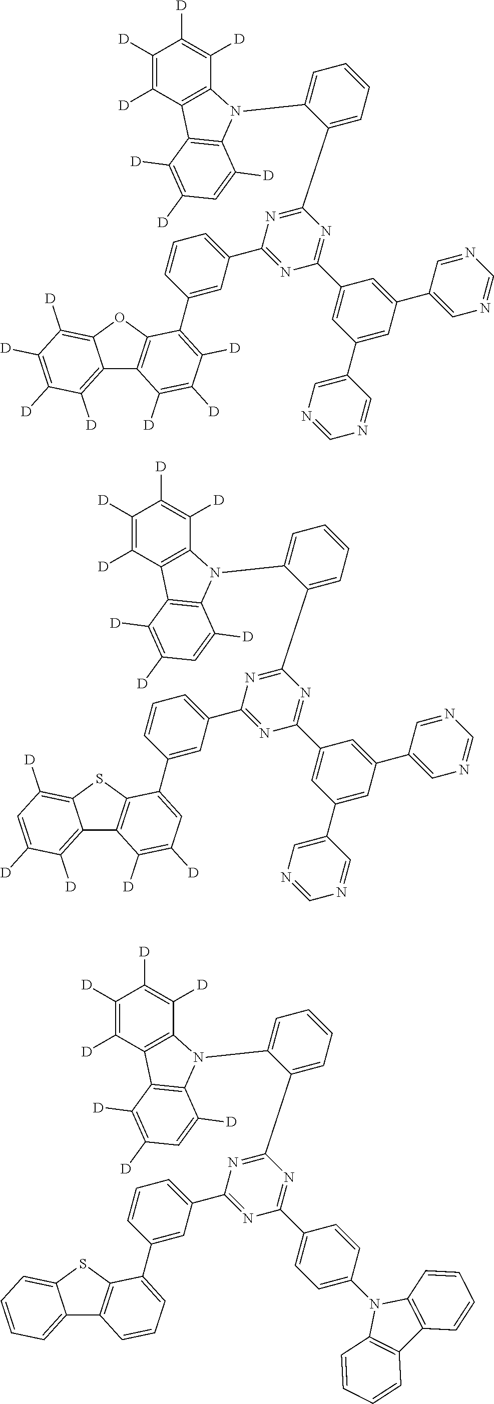

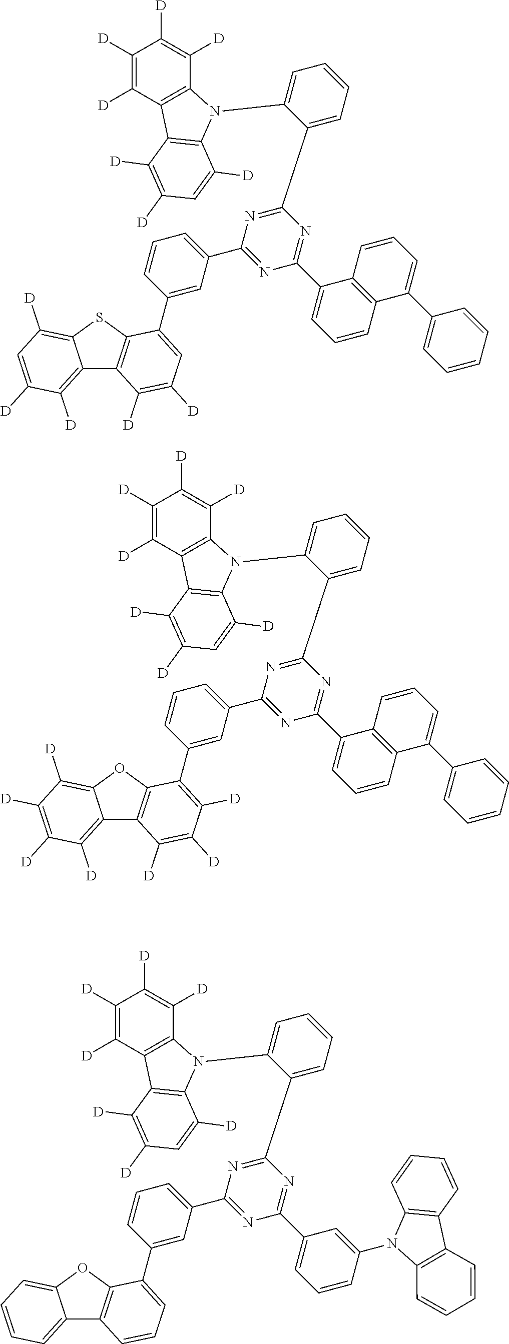

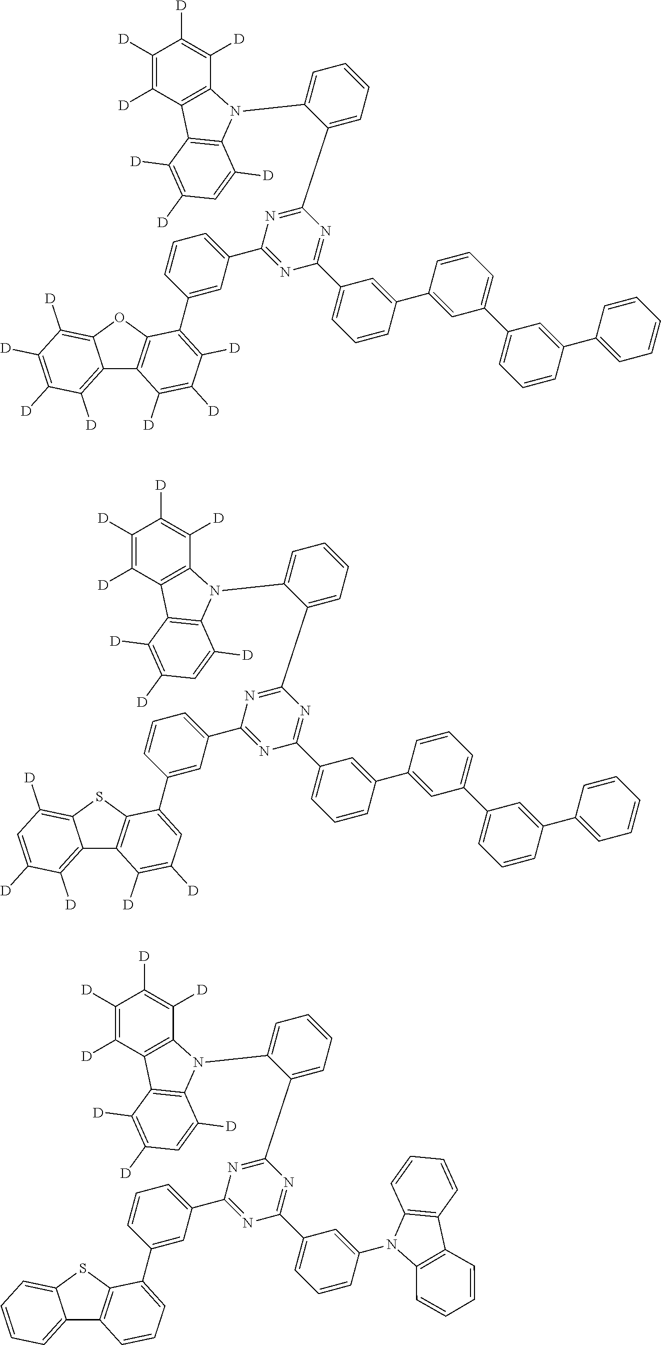

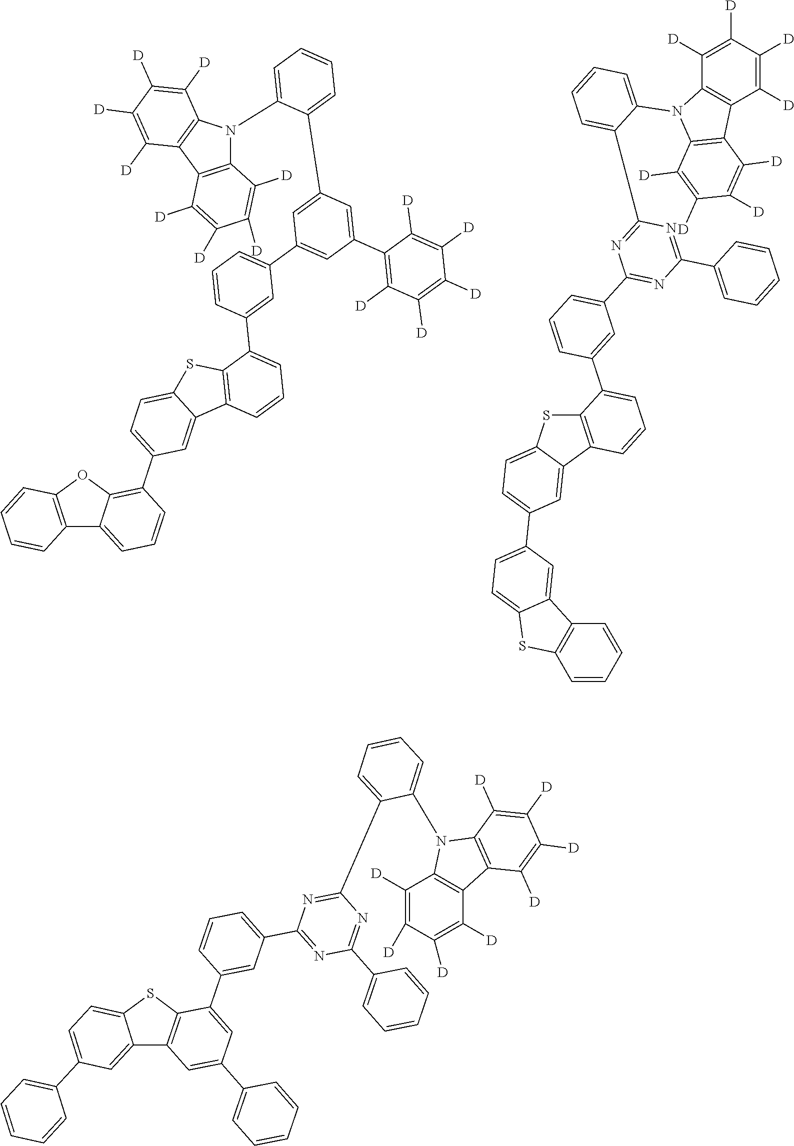 [SMILES: [2H]c1c([2H])c([2H])c(-c2cc(-c3cccc(-c4cccc5c4sc4ccc(-c6cccc7c6oc6ccccc67)cc45)c3)cc(-c3ccccc3-n3c4c([2H])c([2H])c([2H])c([2H])c4c4c([2H])c([2H])c([2H])c([2H])c43)c2)c([2H])c1[2H] ; [2H]c1c([2H])c([2H])c2c(c1[2H])c1c([2H])c([2H])c([2H])c([2H])c1n2-c1ccccc1-c1nc(-c2ccccc2)nc(-c2cccc(-c3cccc4c3sc3ccc(-c5ccc6sc7ccccc7c6c5)cc34)c2)n1 ; [2H]c1c([2H])c([2H])c2c(c1[2H])c1c([2H])c([2H])c([2H])c([2H])c1n2-c1ccccc1-c1nc(-c2ccccc2)nc(-c2cccc(-c3cc(-c4ccccc4)cc4c3sc3ccc(-c5ccccc5)cc34)c2)n1]

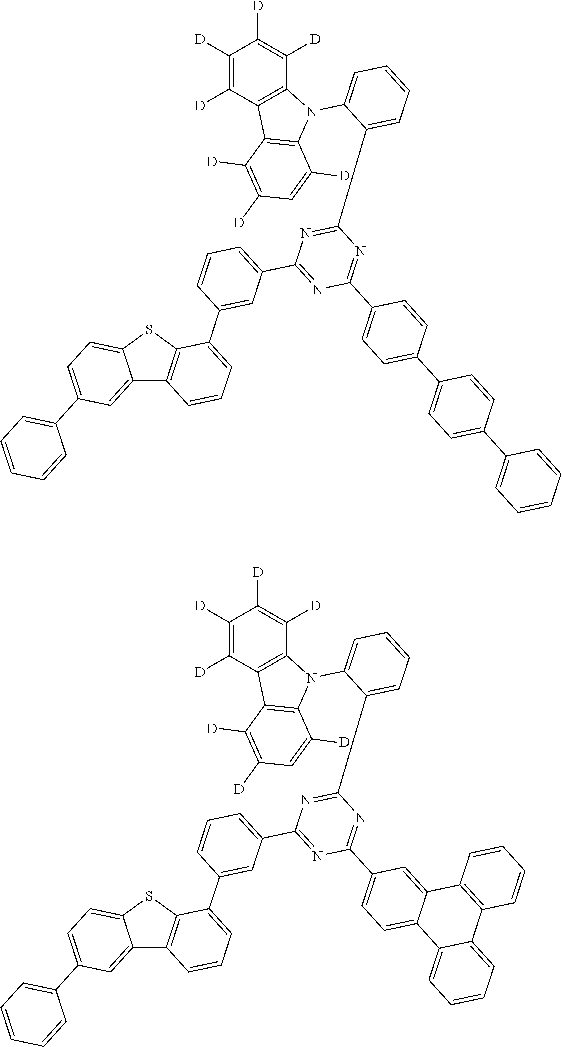 [SMILES: [2H]c1cc([2H])c2c(c1[2H])c1c([2H])c([2H])c([2H])c([2H])c1n2-c1ccccc1-c1nc(-c2ccc(-c3ccc(-c4ccccc4)cc3)cc2)nc(-c2cccc(-c3cccc4c3sc3ccc(-c5ccccc5)cc34)c2)n1 ; [2H]c1cc([2H])c2c(c1[2H])c1c([2H])c([2H])c([2H])c([2H])c1n2-c1ccccc1-c1nc(-c2cccc(-c3cccc4c3sc3ccc(-c5ccccc5)cc34)c2)nc(-c2ccc3c4ccccc4c4ccccc4c3c2)n1]

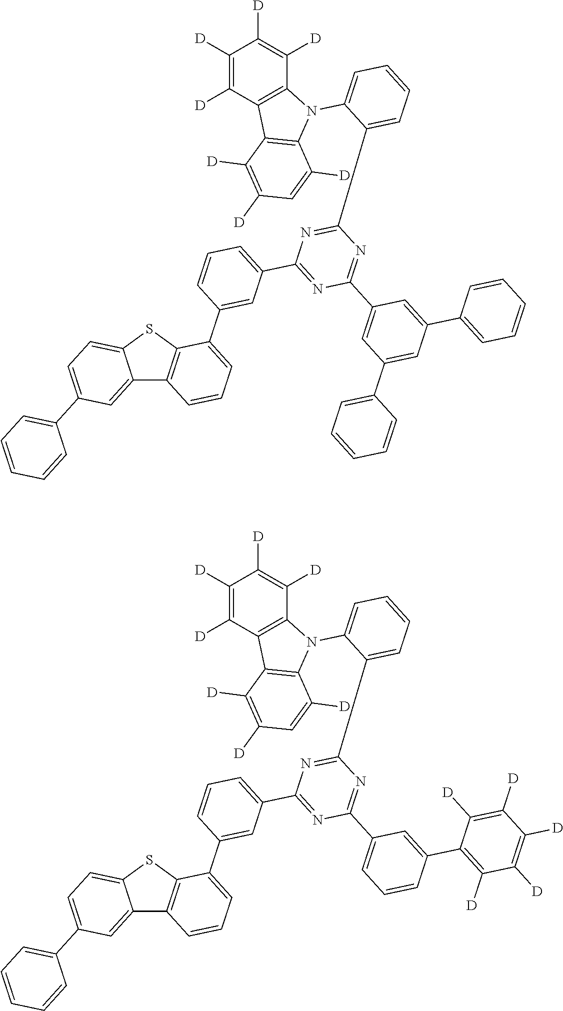 [SMILES: [2H]c1cc([2H])c2c(c1[2H])c1c([2H])c([2H])c([2H])c([2H])c1n2-c1ccccc1-c1nc(-c2cc(-c3ccccc3)cc(-c3ccccc3)c2)nc(-c2cccc(-c3cccc4c3sc3ccc(-c5ccccc5)cc34)c2)n1 ; [2H]c1cc([2H])c2c(c1[2H])c1c([2H])c([2H])c([2H])c([2H])c1n2-c1ccccc1-c1nc(-c2cccc(-c3c([2H])c([2H])c([2H])c([2H])c3[2H])c2)nc(-c2cccc(-c3cccc4c3sc3ccc(-c5ccccc5)cc34)c2)n1]

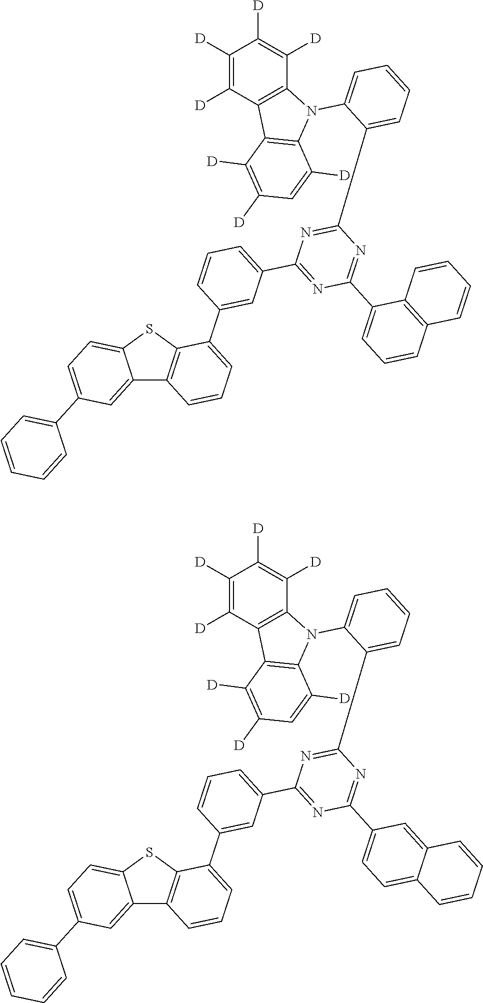 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5ccccc5)cc34)c2)nc(-c2cccc3ccccc23)n1 ; [2H]c1cc([2H])c2c(c1[2H])c1c([2H])c([2H])c([2H])c([2H])c1n2-c1ccccc1-c1nc(-c2cccc(-c3cccc4c3sc3ccc(-c5ccccc5)cc34)c2)nc(-c2ccc3ccccc3c2)n1]

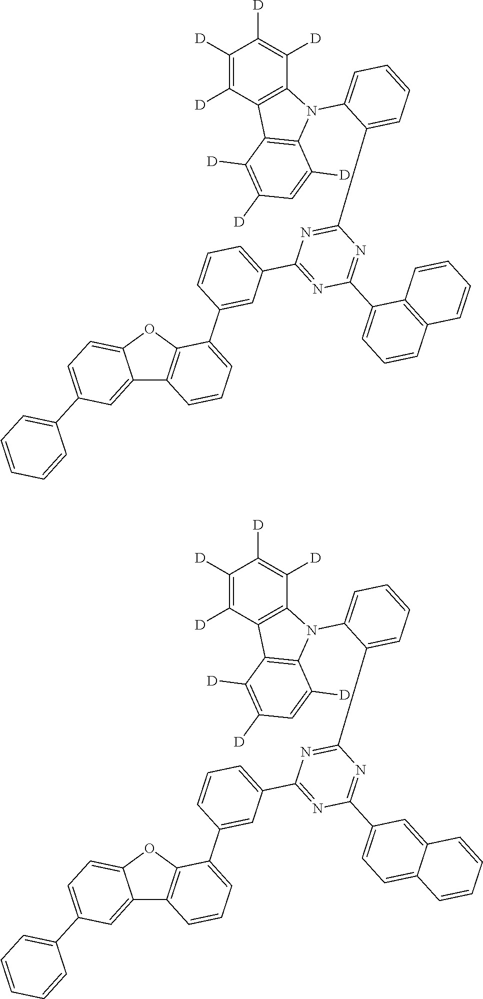 [SMILES: [2H]c1cc([2H])c2c(c1[2H])c1c([2H])c([2H])c([2H])c([2H])c1n2-c1ccccc1-c1nc(-c2cccc(-c3cccc4c3oc3ccc(-c5ccccc5)cc34)c2)nc(-c2cccc3ccccc23)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5ccccc5)cc34)c2)nc(-c2ccc3ccccc3c2)n1]

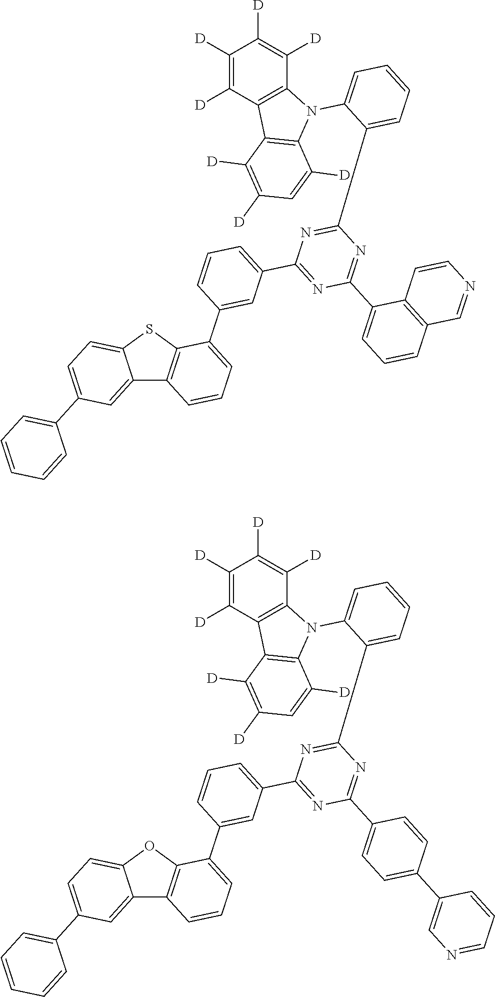 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5ccccc5)cc34)c2)nc(-c2cccc3cnccc23)n1 ; [2H]c1cc([2H])c2c(c1[2H])c1c([2H])c([2H])c([2H])c([2H])c1n2-c1ccccc1-c1nc(-c2ccc(-c3cccnc3)cc2)nc(-c2cccc(-c3cccc4c3oc3ccc(-c5ccccc5)cc34)c2)n1]

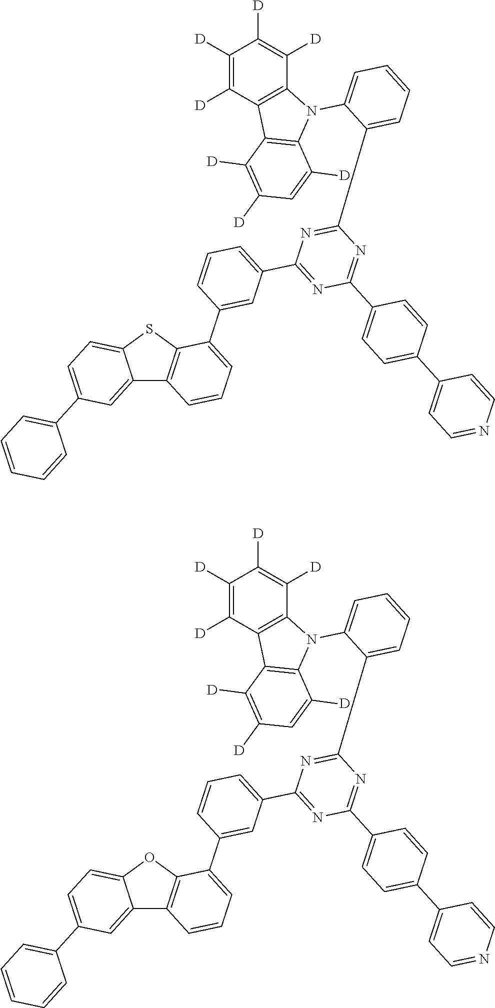 [SMILES: [2H]c1cc([2H])c2c(c1[2H])c1c([2H])c([2H])c([2H])c([2H])c1n2-c1ccccc1-c1nc(-c2ccc(-c3ccncc3)cc2)nc(-c2cccc(-c3cccc4c3sc3ccc(-c5ccccc5)cc34)c2)n1 ; [2H]c1cc([2H])c2c(c1[2H])c1c([2H])c([2H])c([2H])c([2H])c1n2-c1ccccc1-c1nc(-c2ccc(-c3ccncc3)cc2)nc(-c2cccc(-c3cccc4c3oc3ccc(-c5ccccc5)cc34)c2)n1]

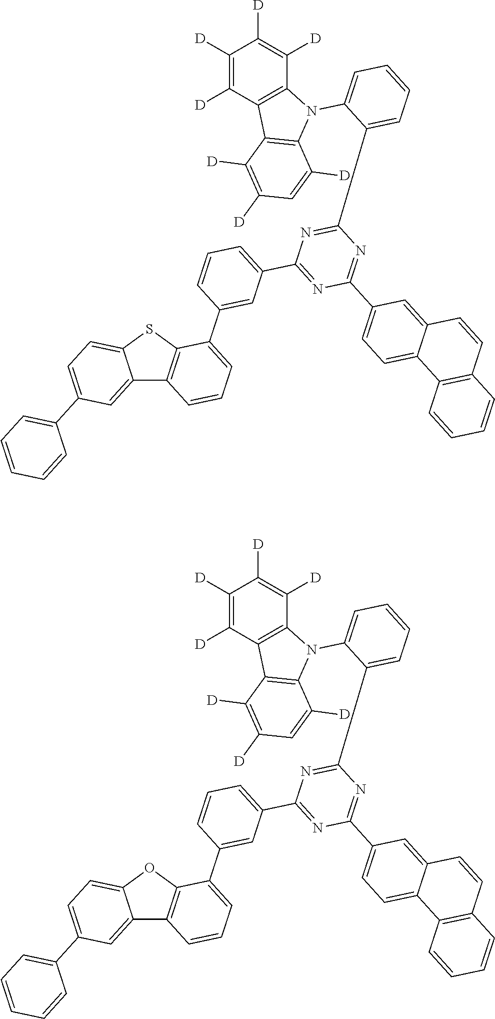 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5ccccc5)cc34)c2)nc(-c2ccc3c(ccc4ccccc43)c2)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5ccccc5)cc34)c2)nc(-c2ccc3c(ccc4ccccc43)c2)n1]

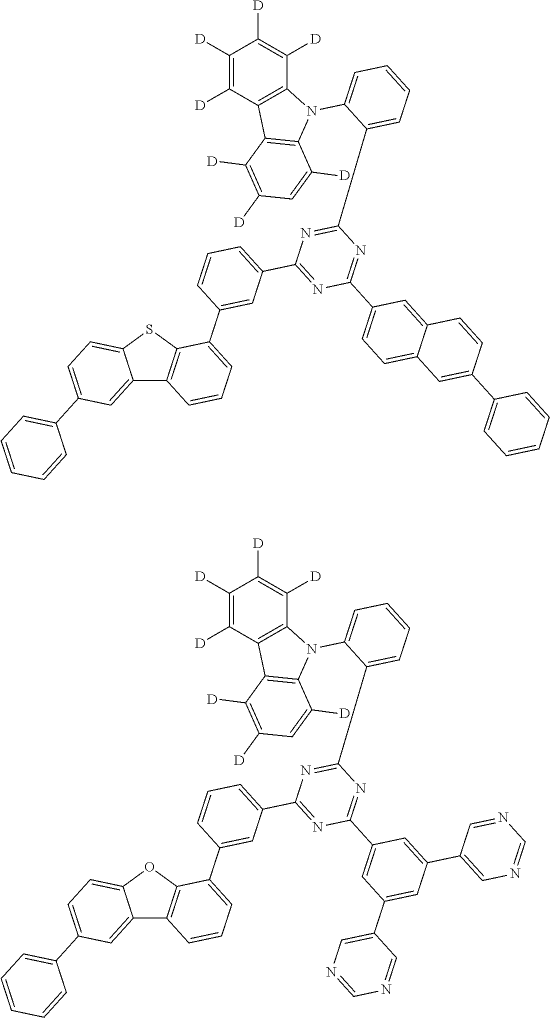 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5ccccc5)cc34)c2)nc(-c2ccc3cc(-c4ccccc4)ccc3c2)n1 ; [2H]c1cc([2H])c2c(c1[2H])c1c([2H])c([2H])c([2H])c([2H])c1n2-c1ccccc1-c1nc(-c2cc(-c3cncnc3)cc(-c3cncnc3)c2)nc(-c2cccc(-c3cccc4c3oc3ccc(-c5ccccc5)cc34)c2)n1]

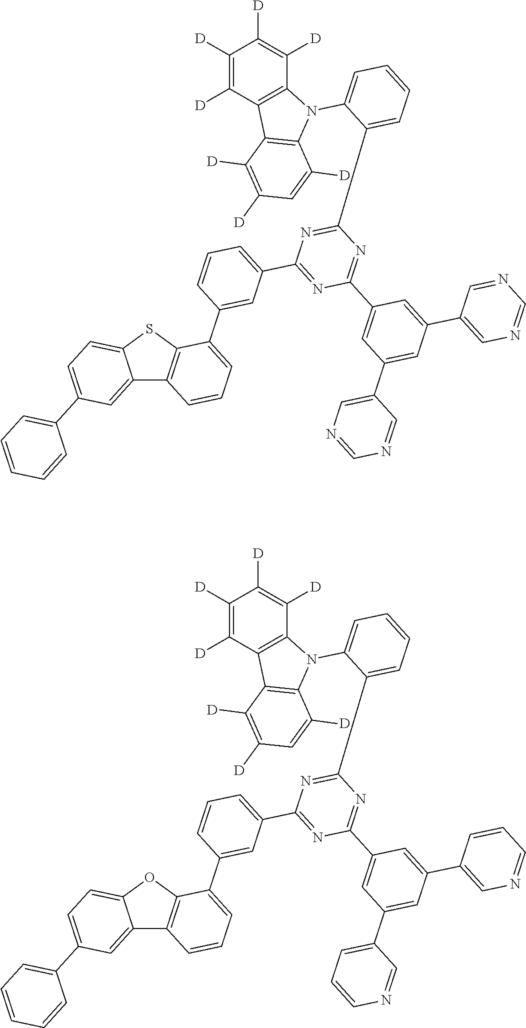 [SMILES: [2H]c1cc([2H])c2c(c1[2H])c1c([2H])c([2H])c([2H])c([2H])c1n2-c1ccccc1-c1nc(-c2cc(-c3cncnc3)cc(-c3cncnc3)c2)nc(-c2cccc(-c3cccc4c3sc3ccc(-c5ccccc5)cc34)c2)n1 ; [2H]c1cc([2H])c2c(c1[2H])c1c([2H])c([2H])c([2H])c([2H])c1n2-c1ccccc1-c1nc(-c2cc(-c3cccnc3)cc(-c3cccnc3)c2)nc(-c2cccc(-c3cccc4c3oc3ccc(-c5ccccc5)cc34)c2)n1]

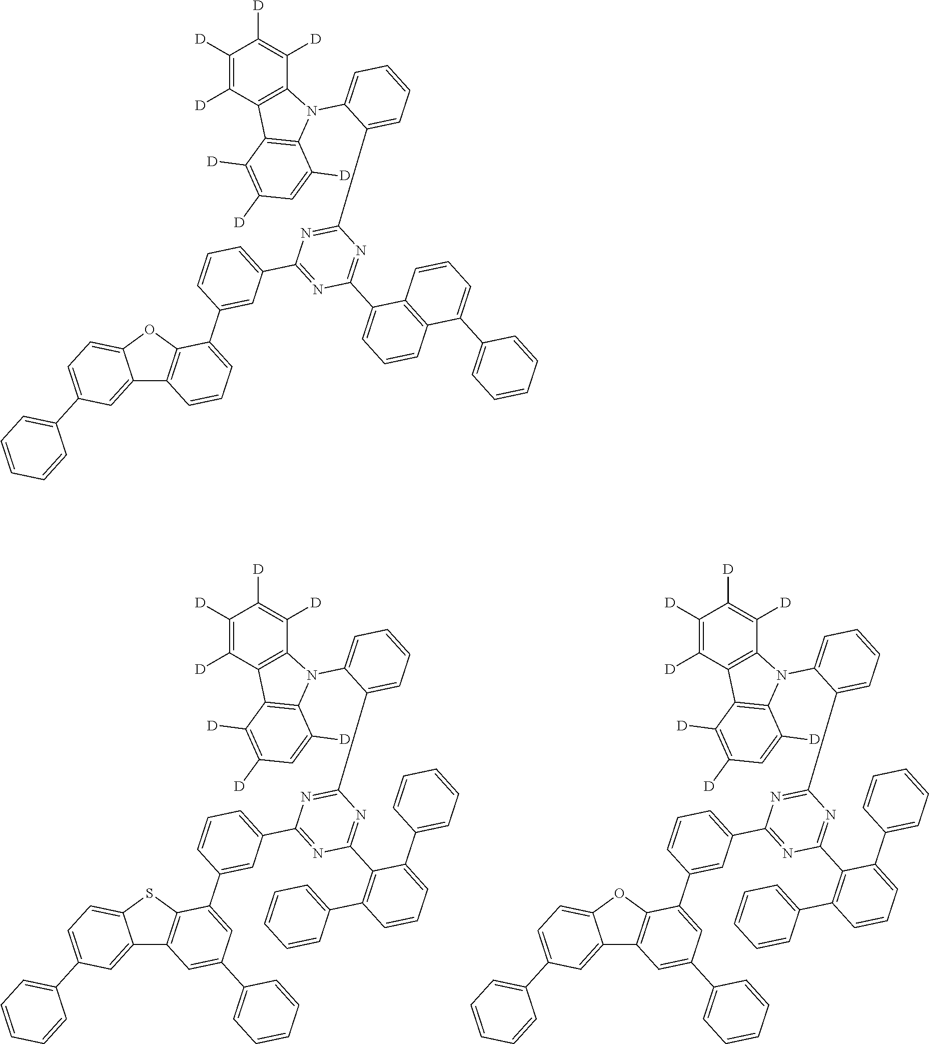 [SMILES: [2H]c1cc([2H])c2c(c1[2H])c1c([2H])c([2H])c([2H])c([2H])c1n2-c1ccccc1-c1nc(-c2cccc(-c3cccc4c3oc3ccc(-c5ccccc5)cc34)c2)nc(-c2cccc3c(-c4ccccc4)cccc23)n1 ; [2H]c1cc([2H])c2c(c1[2H])c1c([2H])c([2H])c([2H])c([2H])c1n2-c1ccccc1-c1nc(-c2cccc(-c3cc(-c4ccccc4)cc4c3sc3ccc(-c5ccccc5)cc34)c2)nc(-c2c(-c3ccccc3)cccc2-c2ccccc2)n1 ; [2H]c1cc([2H])c2c(c1[2H])c1c([2H])c([2H])c([2H])c([2H])c1n2-c1ccccc1-c1nc(-c2cccc(-c3cc(-c4ccccc4)cc4c3oc3ccc(-c5ccccc5)cc34)c2)nc(-c2c(-c3ccccc3)cccc2-c2ccccc2)n1]

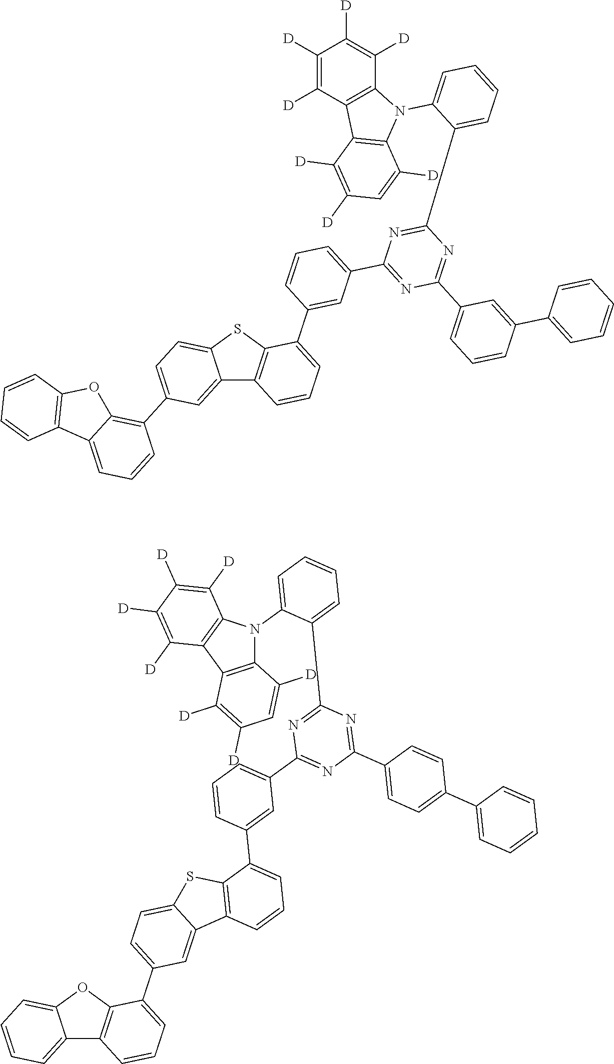 [SMILES: [2H]c1cc([2H])c2c(c1[2H])c1c([2H])c([2H])c([2H])c([2H])c1n2-c1ccccc1-c1nc(-c2cccc(-c3ccccc3)c2)nc(-c2cccc(-c3cccc4c3sc3ccc(-c5cccc6c5oc5ccccc56)cc34)c2)n1 ; [2H]c1cc([2H])c2c(c1[2H])c1c([2H])c([2H])c([2H])c([2H])c1n2-c1ccccc1-c1nc(-c2ccc(-c3ccccc3)cc2)nc(-c2cccc(-c3cccc4c3sc3ccc(-c5cccc6c5oc5ccccc56)cc34)c2)n1]

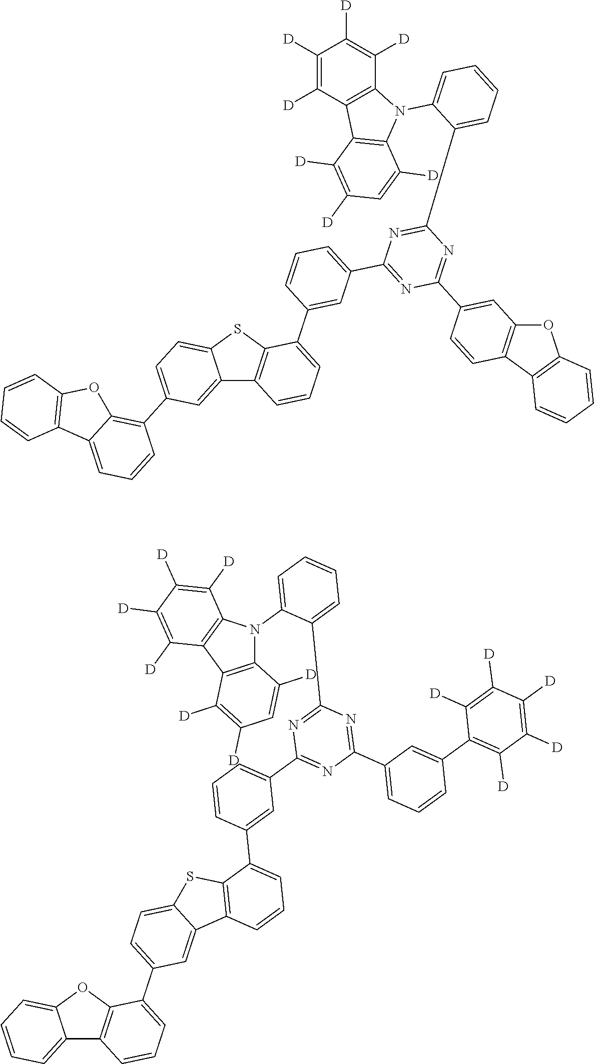 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5cccc6c5oc5ccccc56)cc34)c2)nc(-c2ccc3c(c2)oc2ccccc23)n1 ; [2H]c1cc([2H])c2c(c1[2H])c1c([2H])c([2H])c([2H])c([2H])c1n2-c1ccccc1-c1nc(-c2cccc(-c3c([2H])c([2H])c([2H])c([2H])c3[2H])c2)nc(-c2cccc(-c3cccc4c3sc3ccc(-c5cccc6c5oc5ccccc56)cc34)c2)n1]

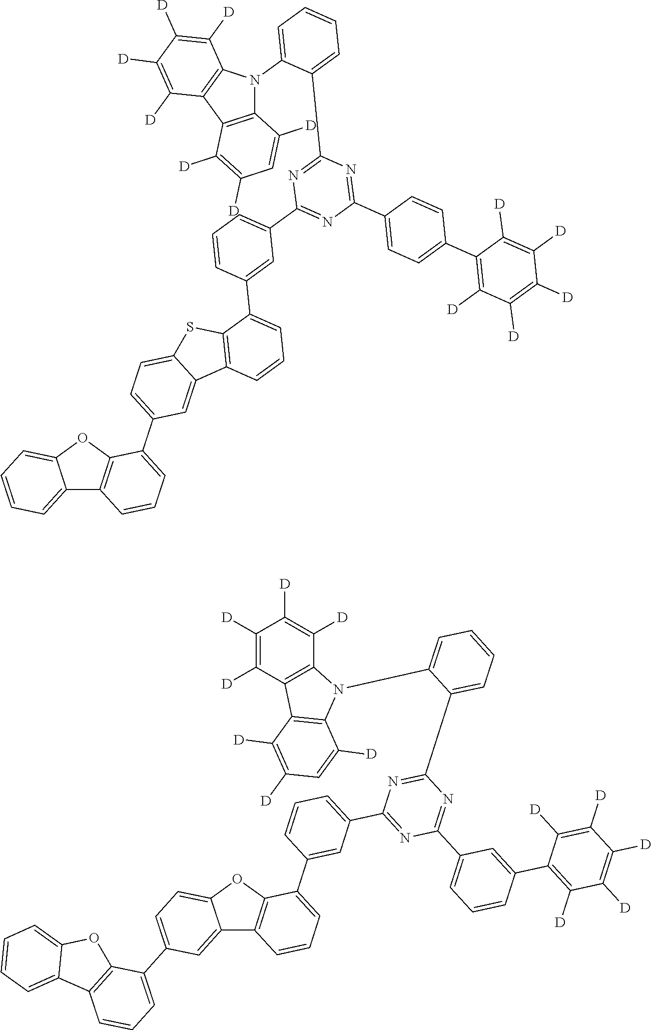 [SMILES: [2H]c1cc([2H])c2c(c1[2H])c1c([2H])c([2H])c([2H])c([2H])c1n2-c1ccccc1-c1nc(-c2ccc(-c3c([2H])c([2H])c([2H])c([2H])c3[2H])cc2)nc(-c2cccc(-c3cccc4c3sc3ccc(-c5cccc6c5oc5ccccc56)cc34)c2)n1 ; [2H]c1cc([2H])c2c(c1[2H])c1c([2H])c([2H])c([2H])c([2H])c1n2-c1ccccc1-c1nc(-c2cccc(-c3c([2H])c([2H])c([2H])c([2H])c3[2H])c2)nc(-c2cccc(-c3cccc4c3oc3ccc(-c5cccc6c5oc5ccccc56)cc34)c2)n1]

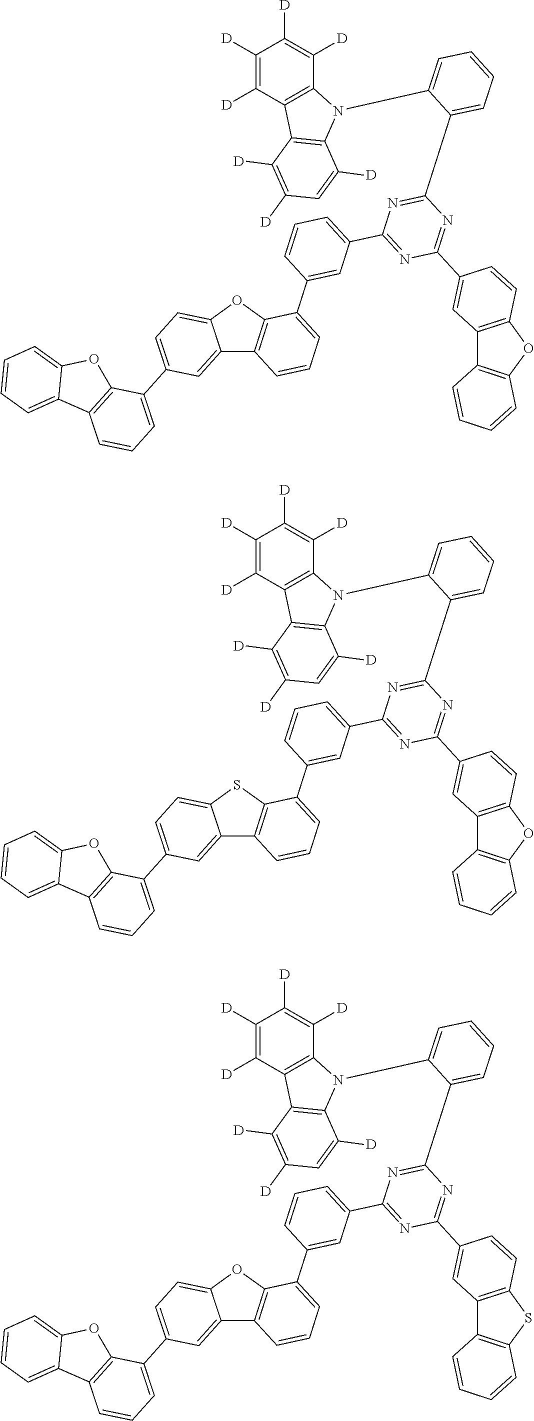 [SMILES: [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-c2ccc3oc4ccccc4c3c2)n1 ; [2H]c1cc([2H])c2c(c1[2H])c1c([2H])c([2H])c([2H])c([2H])c1n2-c1ccccc1-c1nc(-c2cccc(-c3cccc4c3sc3ccc(-c5cccc6c5oc5ccccc56)cc34)c2)nc(-c2ccc3oc4ccccc4c3c2)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-c2ccc3sc4ccccc4c3c2)n1]

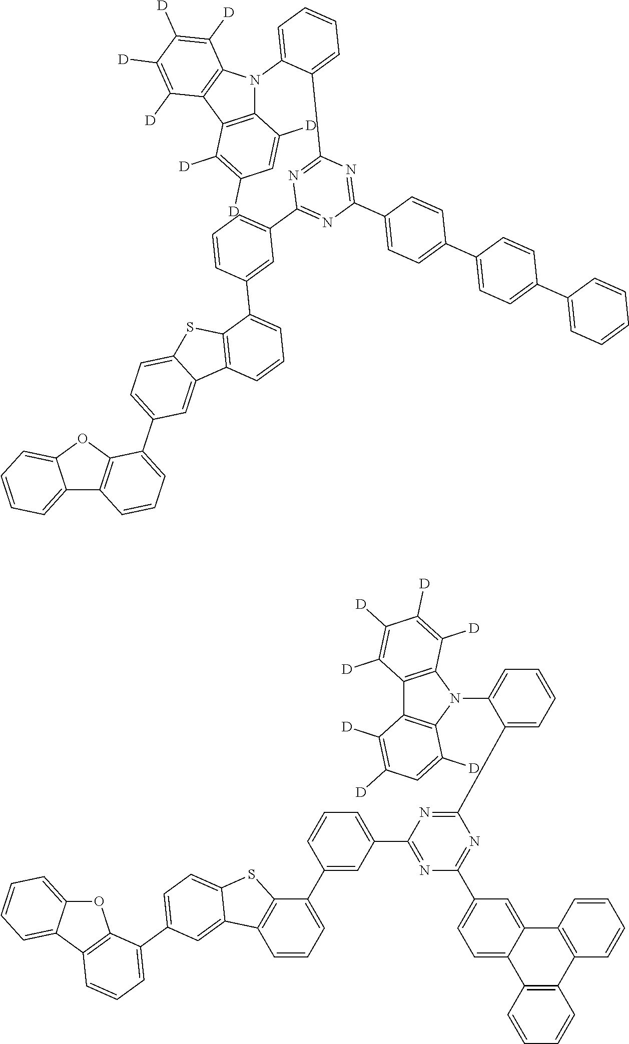 [SMILES: [2H]c1cc([2H])c2c(c1[2H])c1c([2H])c([2H])c([2H])c([2H])c1n2-c1ccccc1-c1nc(-c2ccc(-c3ccc(-c4ccccc4)cc3)cc2)nc(-c2cccc(-c3cccc4c3sc3ccc(-c5cccc6c5oc5ccccc56)cc34)c2)n1 ; [2H]c1cc([2H])c2c(c1[2H])c1c([2H])c([2H])c([2H])c([2H])c1n2-c1ccccc1-c1nc(-c2cccc(-c3cccc4c3sc3ccc(-c5cccc6c5oc5ccccc56)cc34)c2)nc(-c2ccc3c4ccccc4c4ccccc4c3c2)n1]

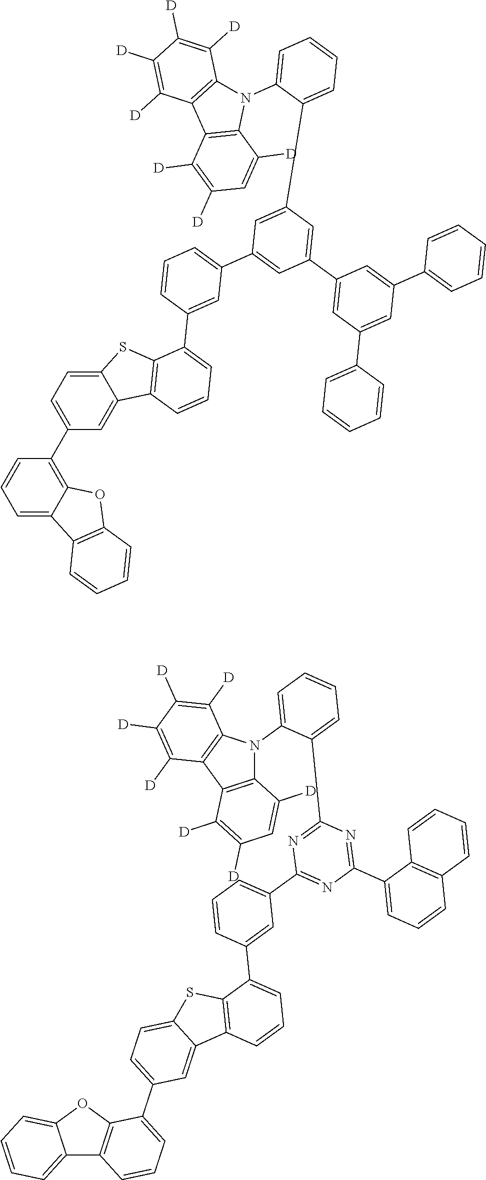 [SMILES: [2H]c1cc([2H])c2c(c1[2H])c1c([2H])c([2H])c([2H])c([2H])c1n2-c1ccccc1-c1cc(-c2cc(-c3ccccc3)cc(-c3ccccc3)c2)cc(-c2cccc(-c3cccc4c3sc3ccc(-c5cccc6c5oc5ccccc56)cc34)c2)c1 ; [2H]c1cc([2H])c2c(c1[2H])c1c([2H])c([2H])c([2H])c([2H])c1n2-c1ccccc1-c1nc(-c2cccc(-c3cccc4c3sc3ccc(-c5cccc6c5oc5ccccc56)cc34)c2)nc(-c2cccc3ccccc23)n1]

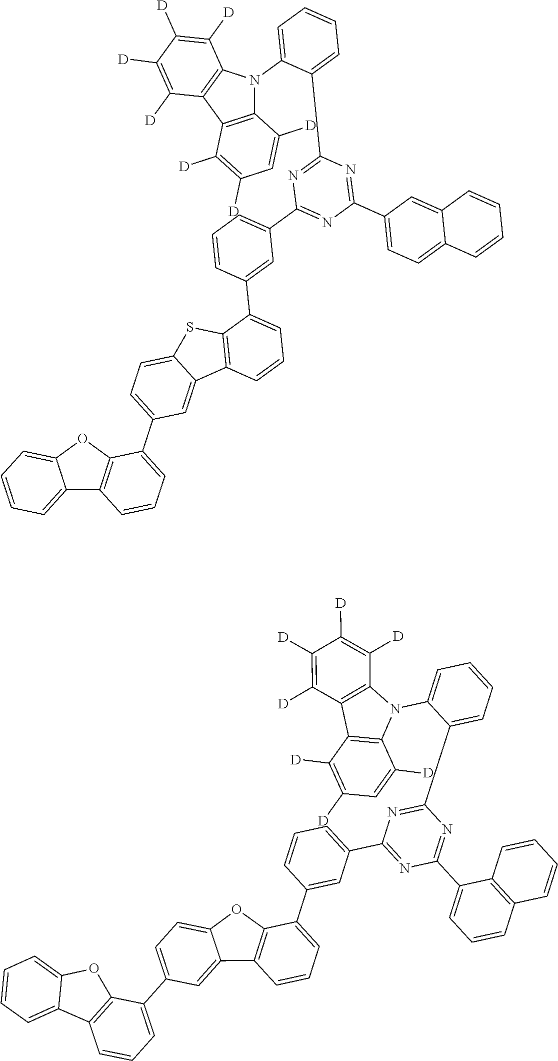 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5cccc6c5oc5ccccc56)cc34)c2)nc(-c2ccc3ccccc3c2)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-c2cccc3ccccc23)n1]

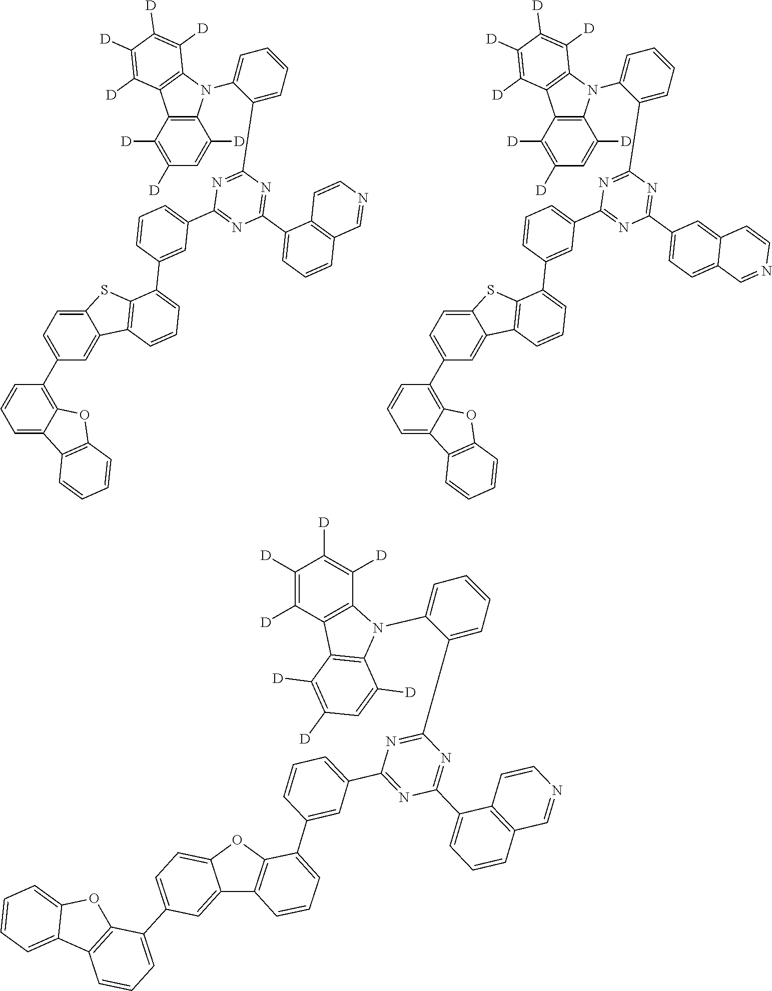 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5cccc6c5oc5ccccc56)cc34)c2)nc(-c2cccc3cnccc23)n1 ; [2H]c1cc([2H])c2c(c1[2H])c1c([2H])c([2H])c([2H])c([2H])c1n2-c1ccccc1-c1nc(-c2cccc(-c3cccc4c3sc3ccc(-c5cccc6c5oc5ccccc56)cc34)c2)nc(-c2ccc3cnccc3c2)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-c2cccc3cnccc23)n1]

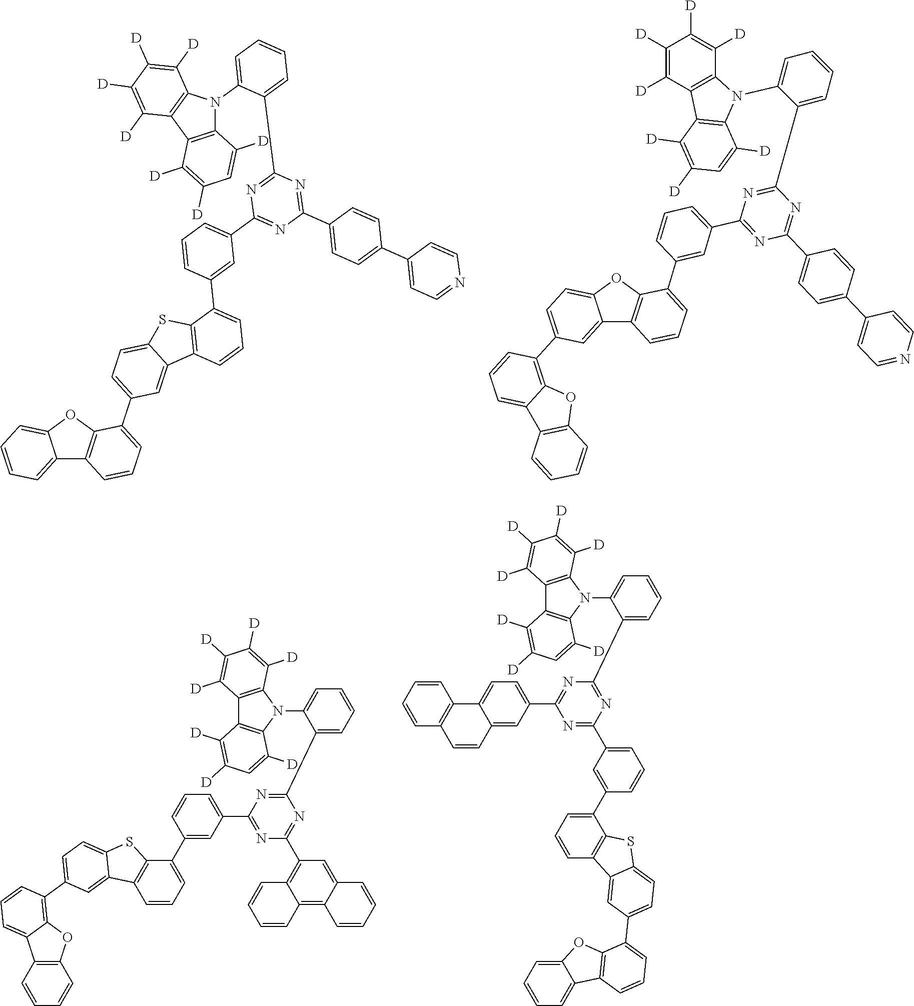

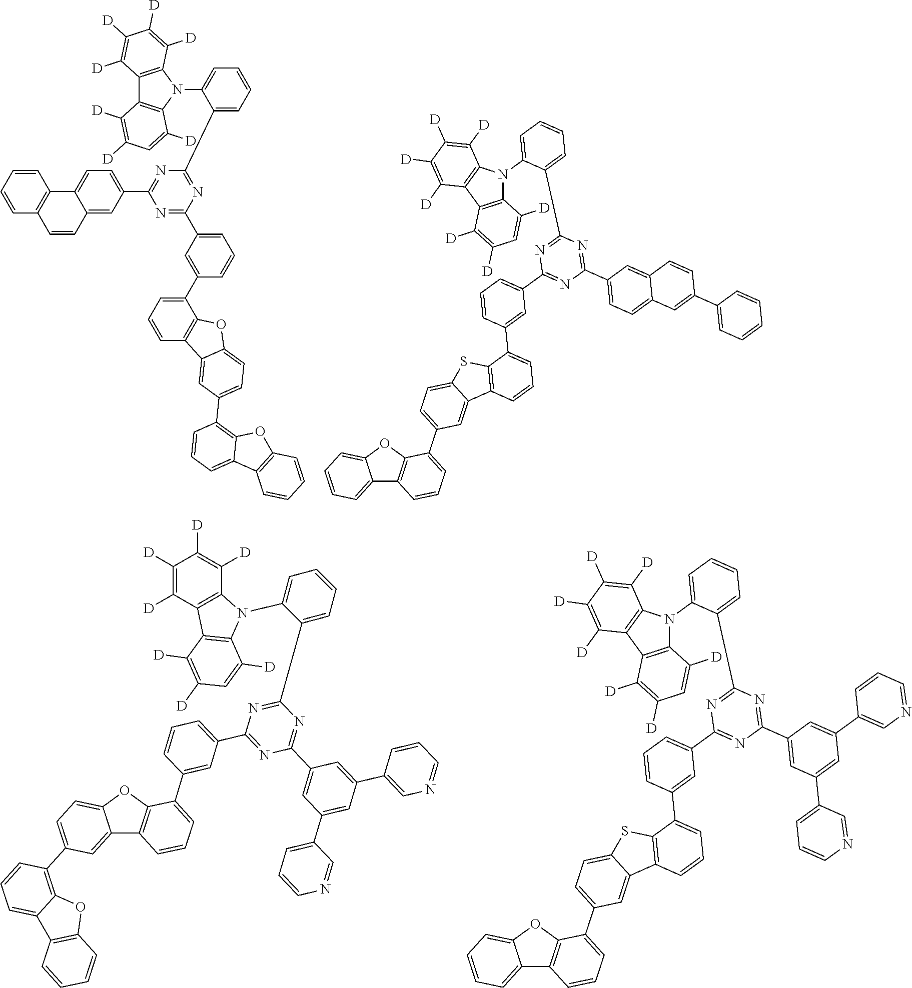

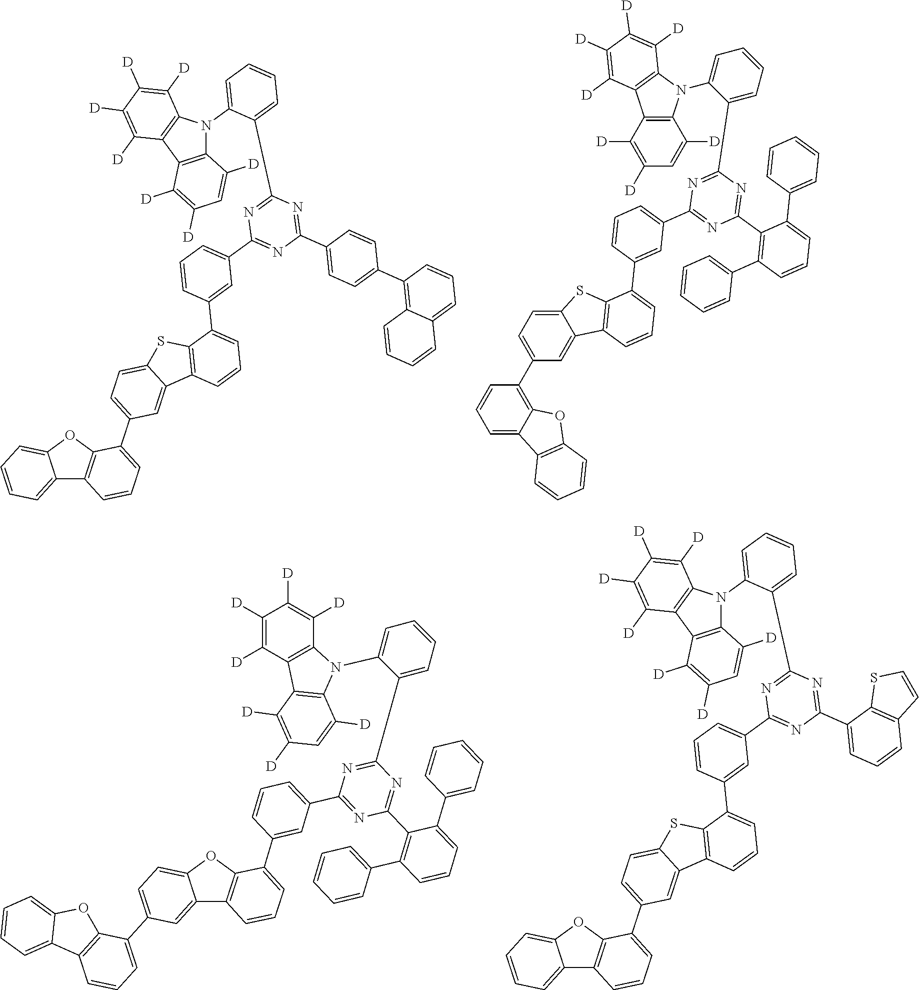

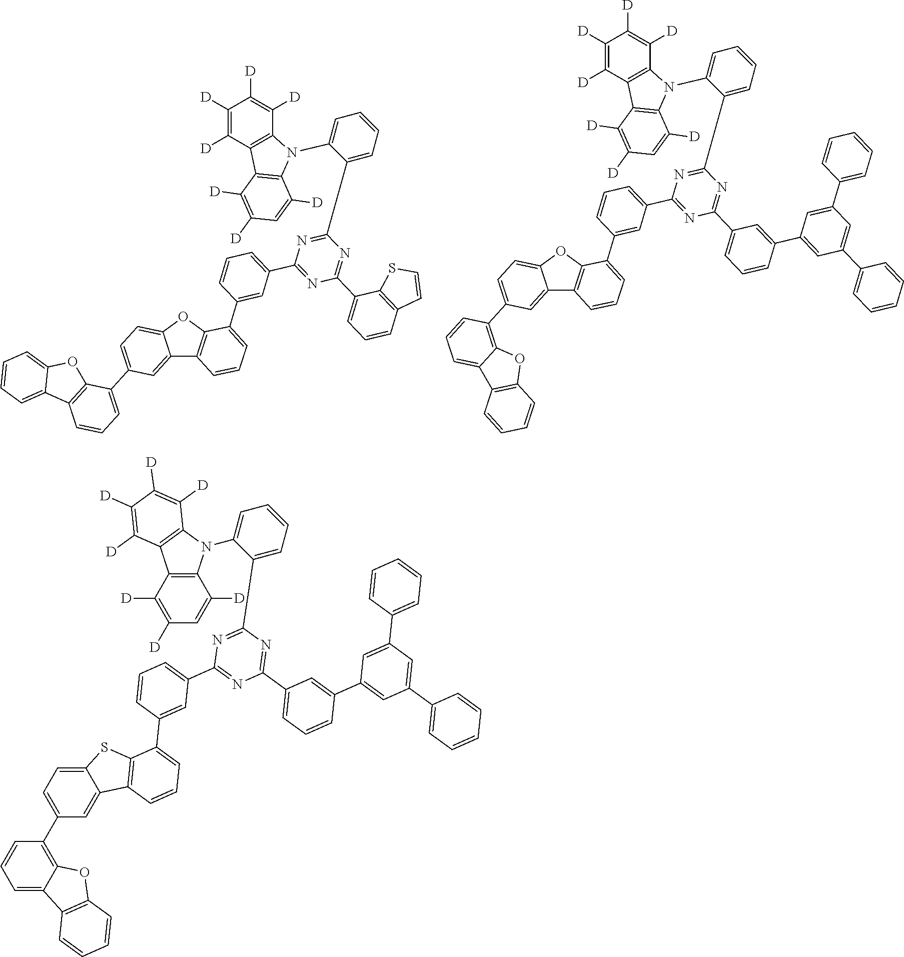

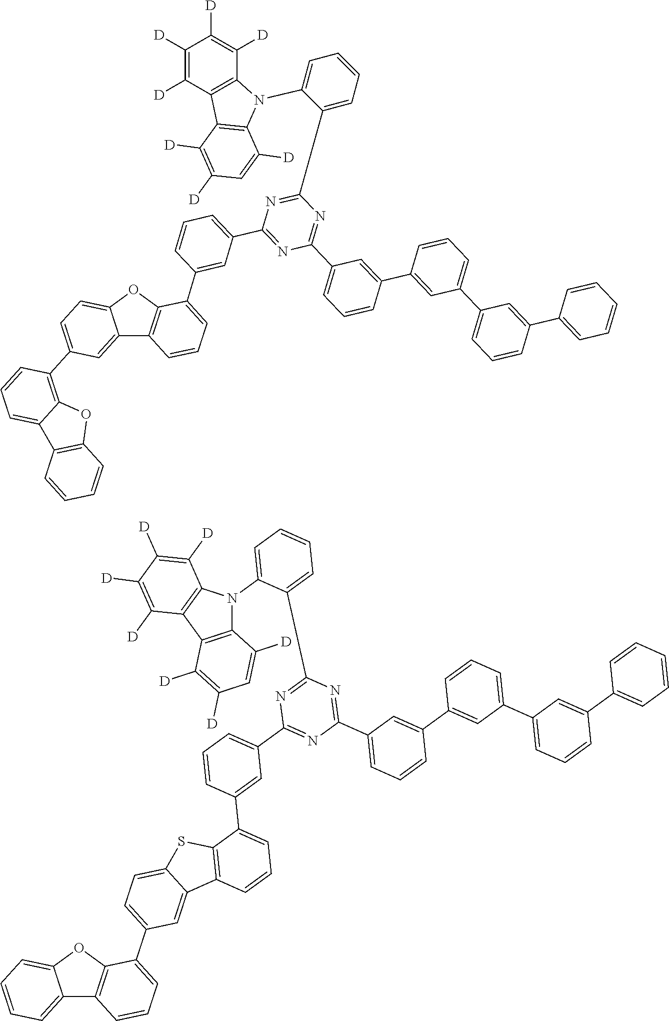 [SMILES: [2H]c1cc([2H])c2c(c1[2H])c1c([2H])c([2H])c([2H])c([2H])c1n2-c1ccccc1-c1nc(-c2cccc(-c3cccc(-c4cccc(-c5ccccc5)c4)c3)c2)nc(-c2cccc(-c3cccc4c3oc3ccc(-c5cccc6c5oc5ccccc56)cc34)c2)n1 ; [2H]c1cc([2H])c2c(c1[2H])c1c([2H])c([2H])c([2H])c([2H])c1n2-c1ccccc1-c1nc(-c2cccc(-c3cccc(-c4cccc(-c5ccccc5)c4)c3)c2)nc(-c2cccc(-c3cccc4c3sc3ccc(-c5cccc6c5oc5ccccc56)cc34)c2)n1]

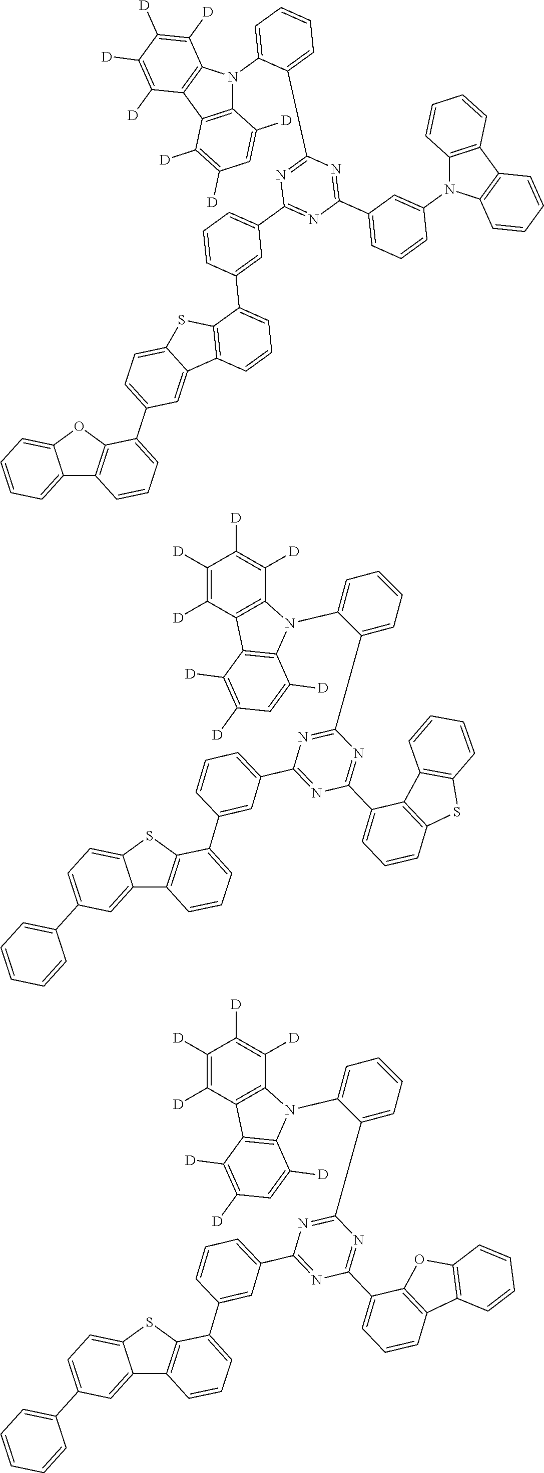 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5cccc6c5oc5ccccc56)cc34)c2)nc(-c2cccc(-n3c4ccccc4c4ccccc43)c2)n1 ; [2H]c1cc([2H])c2c(c1[2H])c1c([2H])c([2H])c([2H])c([2H])c1n2-c1ccccc1-c1nc(-c2cccc(-c3cccc4c3sc3ccc(-c5ccccc5)cc34)c2)nc(-c2cccc3sc4ccccc4c23)n1 ; [2H]c1cc([2H])c2c(c1[2H])c1c([2H])c([2H])c([2H])c([2H])c1n2-c1ccccc1-c1nc(-c2cccc(-c3cccc4c3sc3ccc(-c5ccccc5)cc34)c2)nc(-c2cccc3c2oc2ccccc23)n1]

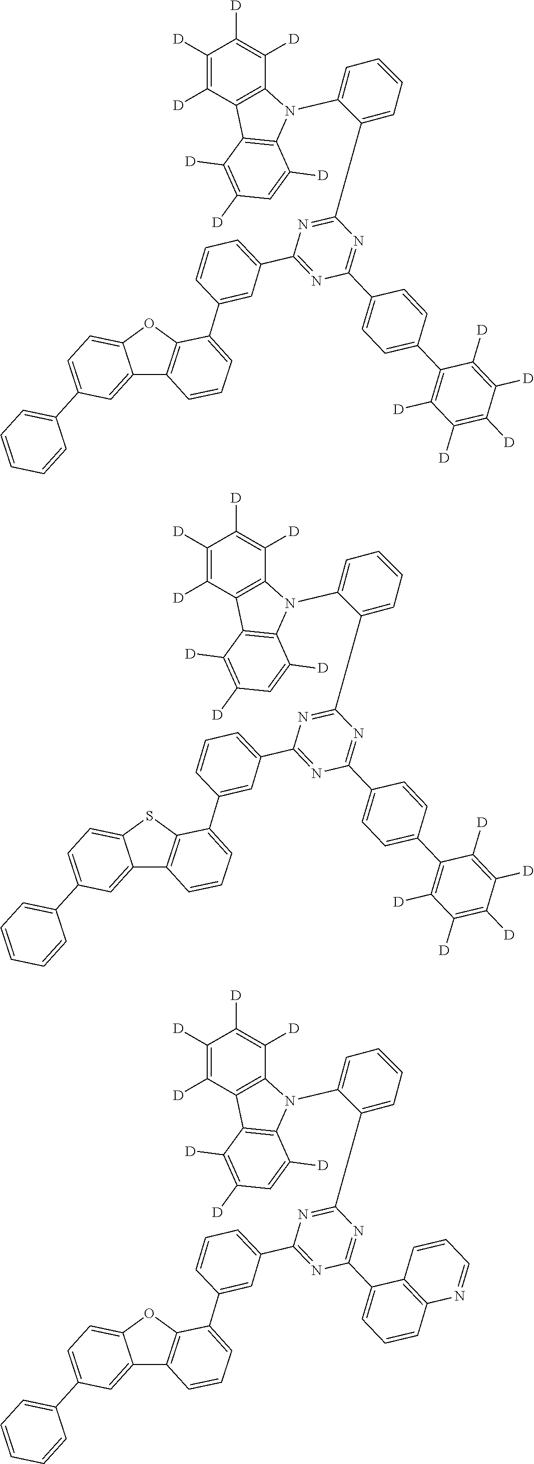 [SMILES: [2H]c1cc([2H])c2c(c1[2H])c1c([2H])c([2H])c([2H])c([2H])c1n2-c1ccccc1-c1nc(-c2ccc(-c3c([2H])c([2H])c([2H])c([2H])c3[2H])cc2)nc(-c2cccc(-c3cccc4c3oc3ccc(-c5ccccc5)cc34)c2)n1 ; [2H]c1cc([2H])c2c(c1[2H])c1c([2H])c([2H])c([2H])c([2H])c1n2-c1ccccc1-c1nc(-c2ccc(-c3c([2H])c([2H])c([2H])c([2H])c3[2H])cc2)nc(-c2cccc(-c3cccc4c3sc3ccc(-c5ccccc5)cc34)c2)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5ccccc5)cc34)c2)nc(-c2cccc3ncccc23)n1]

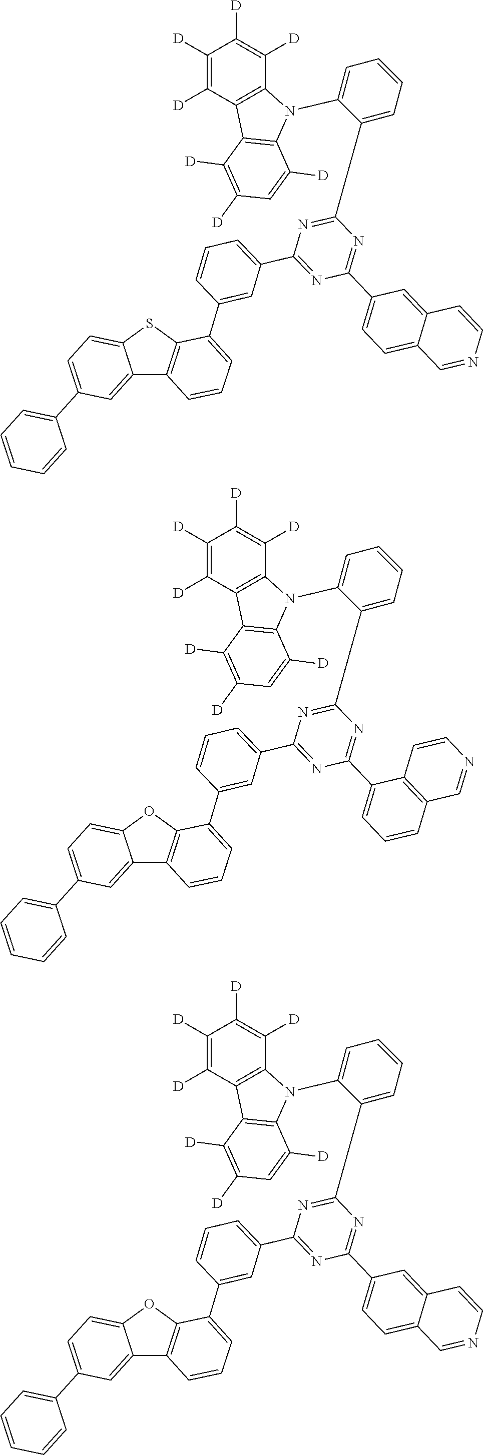 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5ccccc5)cc34)c2)nc(-c2ccc3cnccc3c2)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5ccccc5)cc34)c2)nc(-c2cccc3cnccc23)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5ccccc5)cc34)c2)nc(-c2ccc3cnccc3c2)n1]

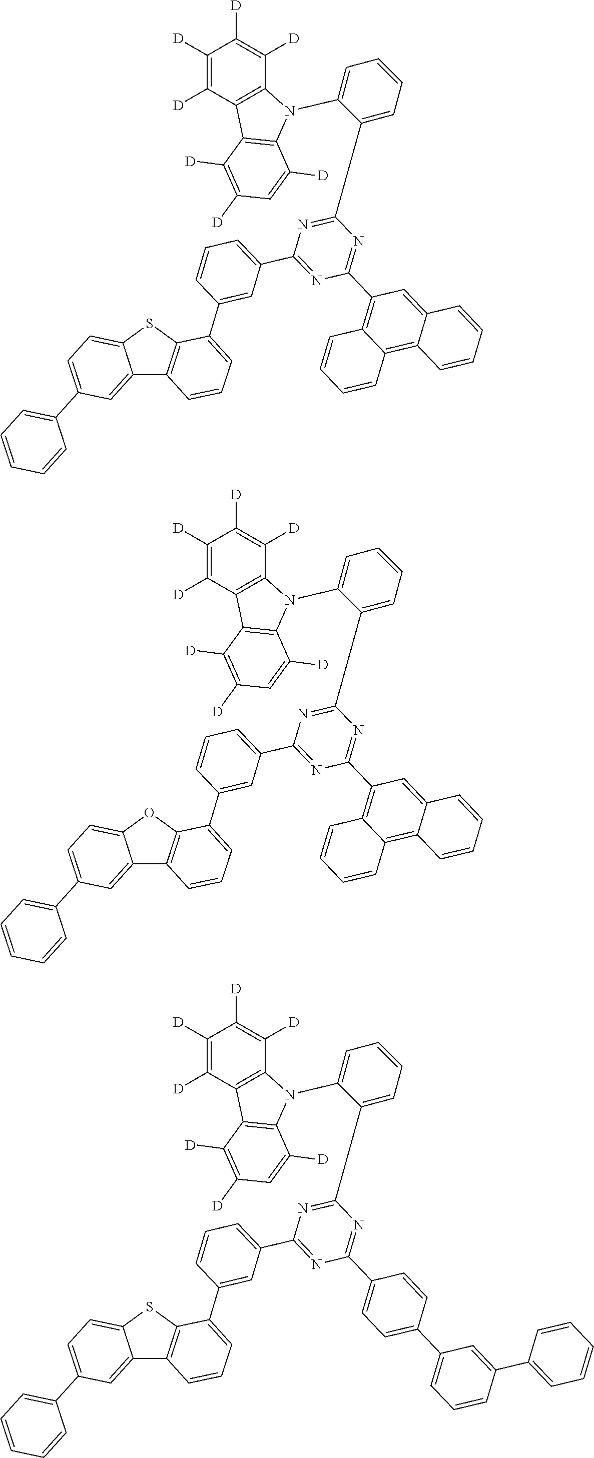 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5ccccc5)cc34)c2)nc(-c2cc3ccccc3c3ccccc23)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5ccccc5)cc34)c2)nc(-c2cc3ccccc3c3ccccc23)n1 ; [2H]c1cc([2H])c2c(c1[2H])c1c([2H])c([2H])c([2H])c([2H])c1n2-c1ccccc1-c1nc(-c2ccc(-c3cccc(-c4ccccc4)c3)cc2)nc(-c2cccc(-c3cccc4c3sc3ccc(-c5ccccc5)cc34)c2)n1]

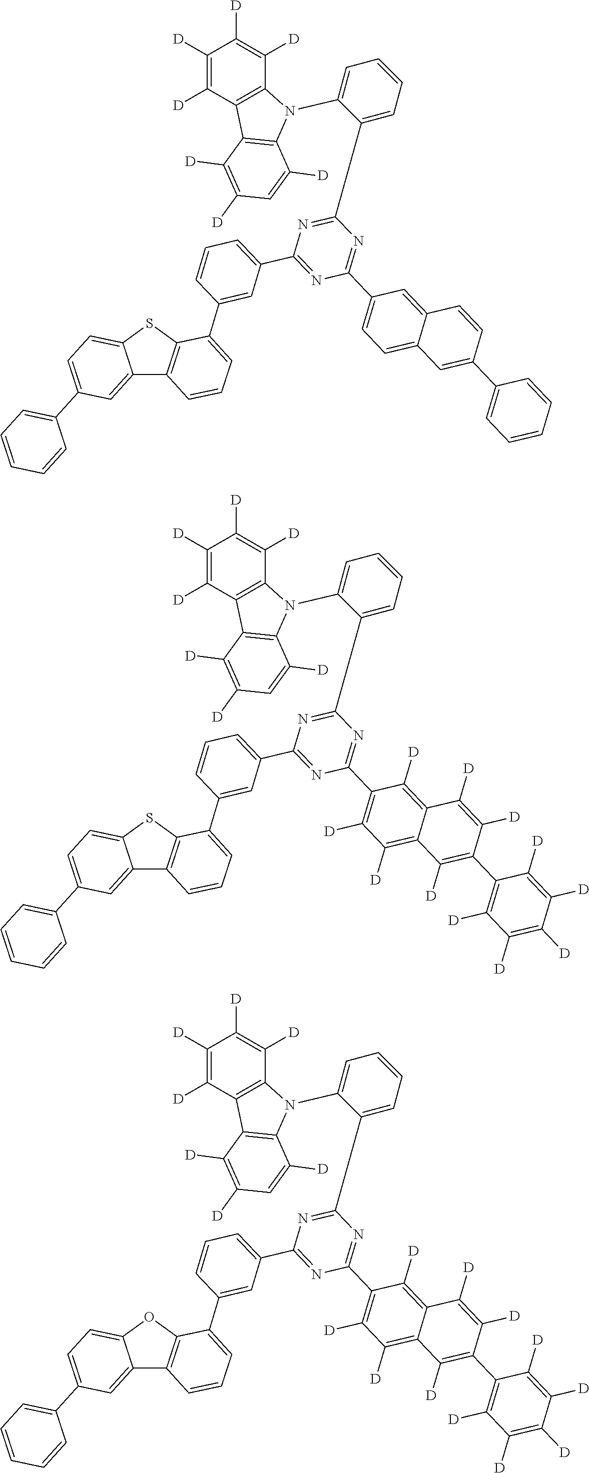

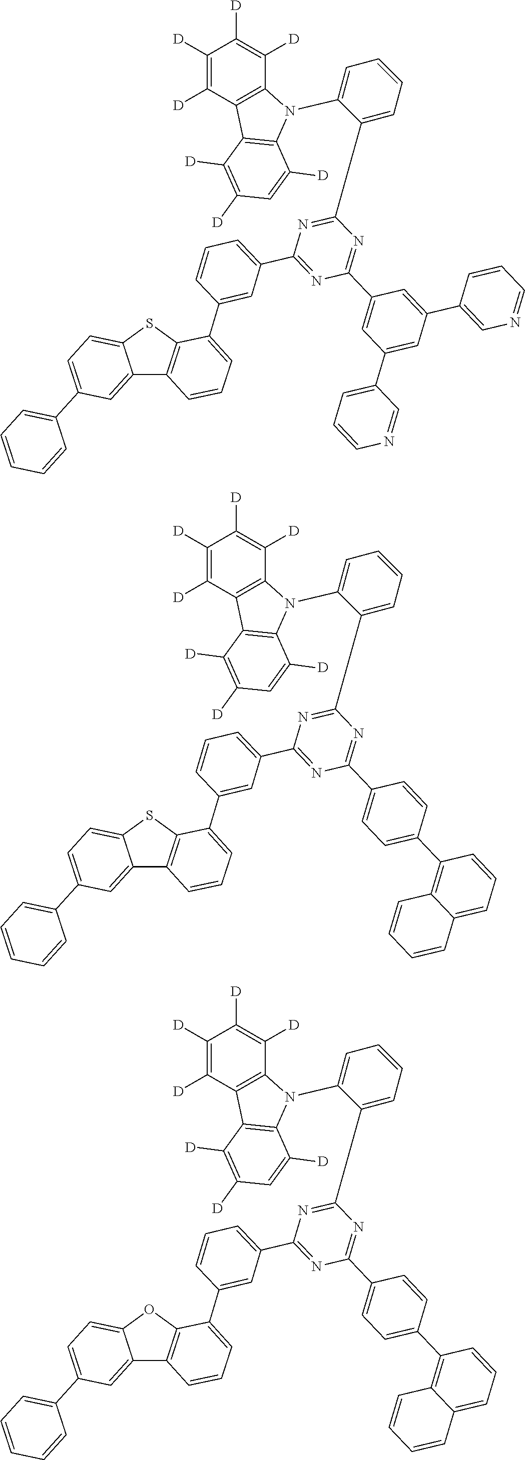 [SMILES: [2H]c1cc([2H])c2c(c1[2H])c1c([2H])c([2H])c([2H])c([2H])c1n2-c1ccccc1-c1nc(-c2cc(-c3cccnc3)cc(-c3cccnc3)c2)nc(-c2cccc(-c3cccc4c3sc3ccc(-c5ccccc5)cc34)c2)n1 ; [2H]c1cc([2H])c2c(c1[2H])c1c([2H])c([2H])c([2H])c([2H])c1n2-c1ccccc1-c1nc(-c2ccc(-c3cccc4ccccc34)cc2)nc(-c2cccc(-c3cccc4c3sc3ccc(-c5ccccc5)cc34)c2)n1 ; [2H]c1cc([2H])c2c(c1[2H])c1c([2H])c([2H])c([2H])c([2H])c1n2-c1ccccc1-c1nc(-c2ccc(-c3cccc4ccccc34)cc2)nc(-c2cccc(-c3cccc4c3oc3ccc(-c5ccccc5)cc34)c2)n1]

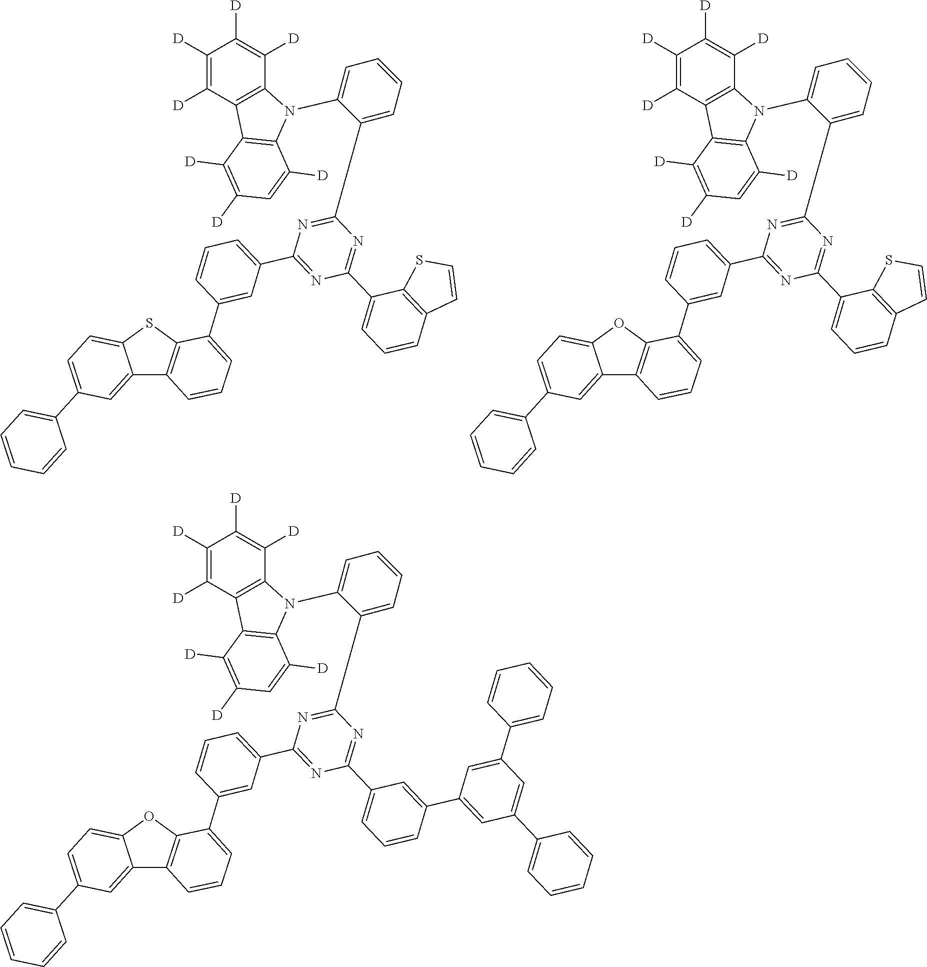 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5ccccc5)cc34)c2)nc(-c2cccc3ccsc23)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5ccccc5)cc34)c2)nc(-c2cccc3ccsc23)n1 ; [2H]c1cc([2H])c2c(c1[2H])c1c([2H])c([2H])c([2H])c([2H])c1n2-c1ccccc1-c1nc(-c2cccc(-c3cc(-c4ccccc4)cc(-c4ccccc4)c3)c2)nc(-c2cccc(-c3cccc4c3oc3ccc(-c5ccccc5)cc34)c2)n1]

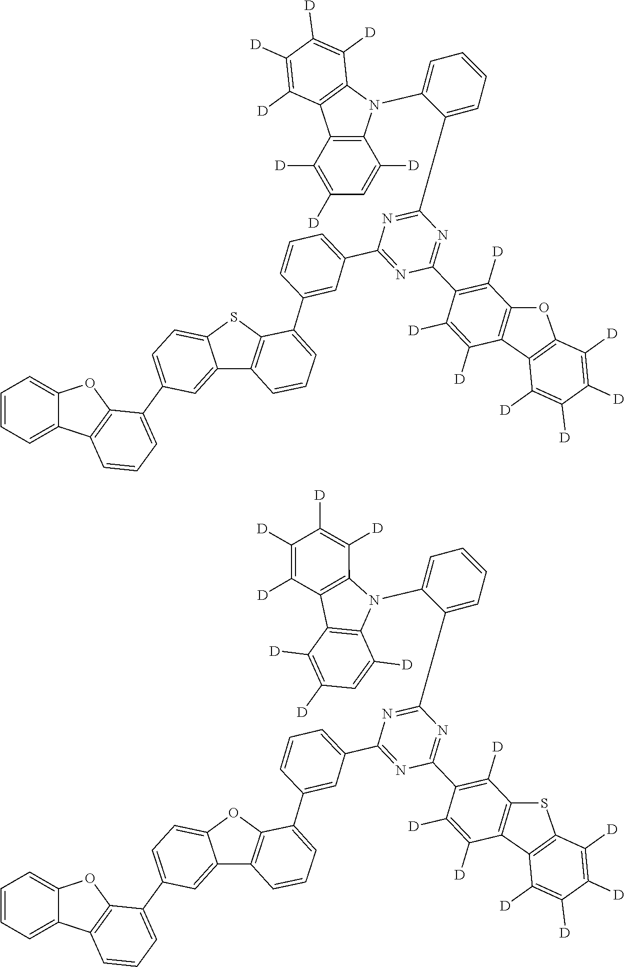 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5cccc6c5oc5ccccc56)cc34)c2)nc(-c2c([2H])c([2H])c3c(oc4c([2H])c([2H])c([2H])c([2H])c43)c2[2H])n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-c2c([2H])c([2H])c3c(sc4c([2H])c([2H])c([2H])c([2H])c43)c2[2H])n1]

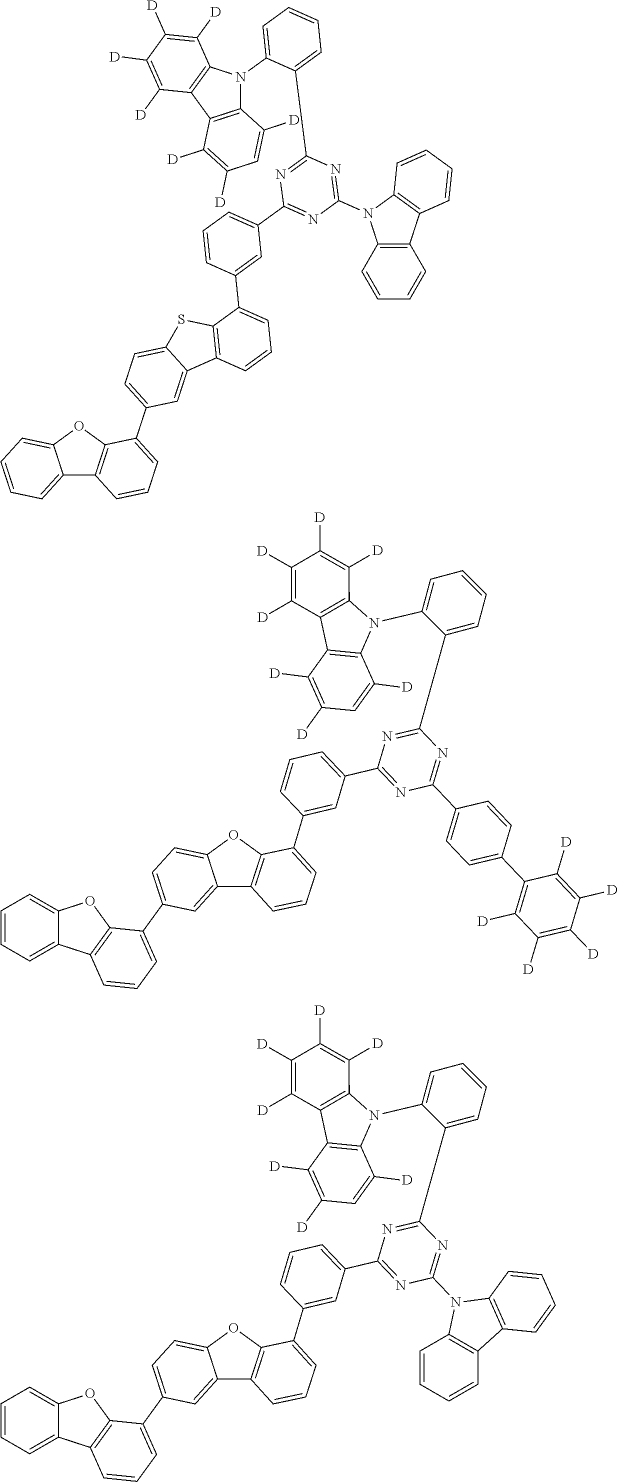 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5cccc6c5oc5ccccc56)cc34)c2)nc(-n2c3ccccc3c3ccccc32)n1 ; [2H]c1cc([2H])c2c(c1[2H])c1c([2H])c([2H])c([2H])c([2H])c1n2-c1ccccc1-c1nc(-c2ccc(-c3c([2H])c([2H])c([2H])c([2H])c3[2H])cc2)nc(-c2cccc(-c3cccc4c3oc3ccc(-c5cccc6c5oc5ccccc56)cc34)c2)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-n2c3ccccc3c3ccccc32)n1]

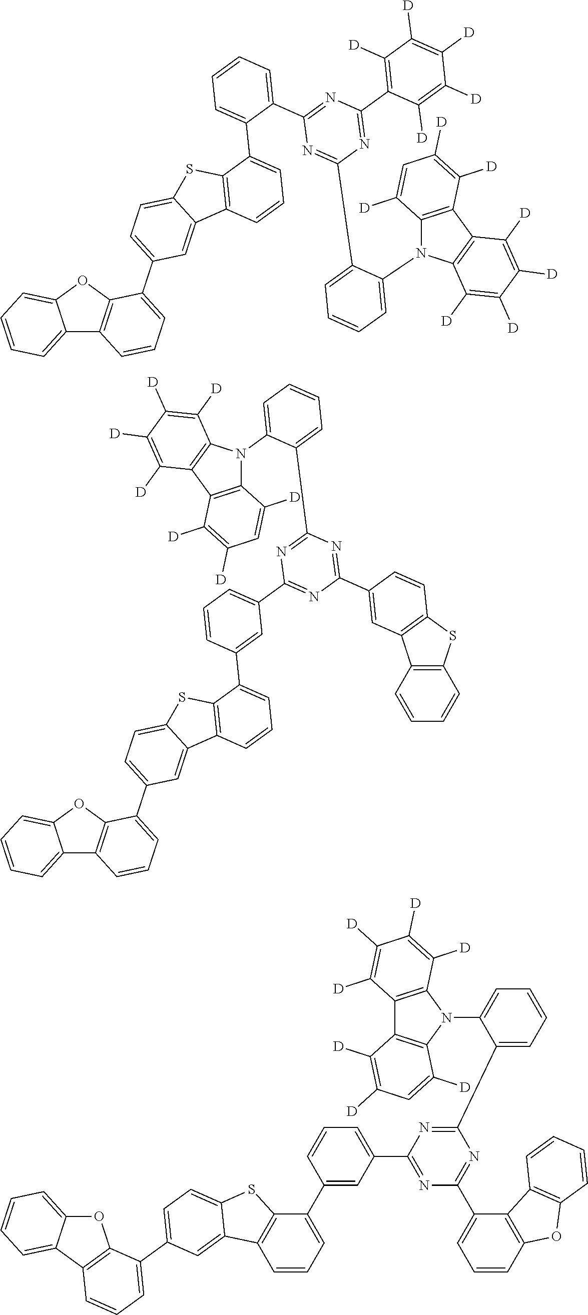

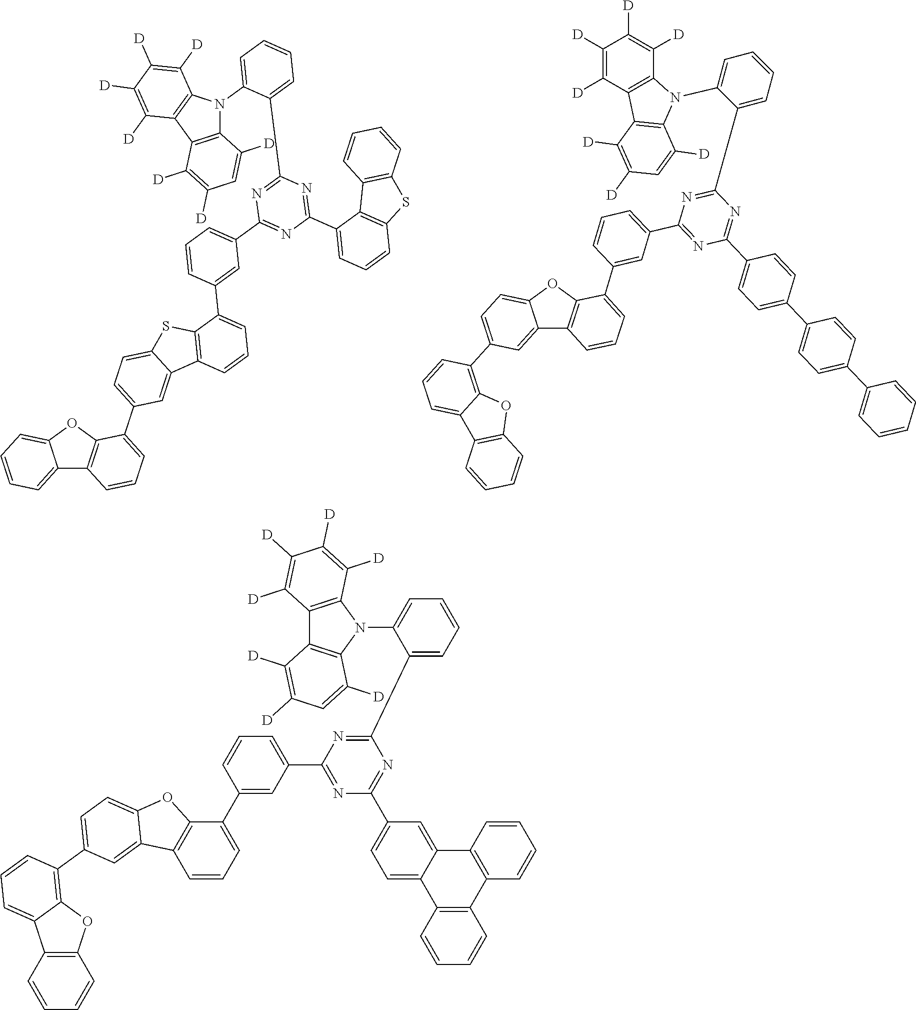 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5cccc6c5oc5ccccc56)cc34)c2)nc(-c2cccc3sc4ccccc4c23)n1 ; [2H]c1cc([2H])c2c(c1[2H])c1c([2H])c([2H])c([2H])c([2H])c1n2-c1ccccc1-c1nc(-c2ccc(-c3ccc(-c4ccccc4)cc3)cc2)nc(-c2cccc(-c3cccc4c3oc3ccc(-c5cccc6c5oc5ccccc56)cc34)c2)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-c2ccc3c4ccccc4c4ccccc4c3c2)n1]

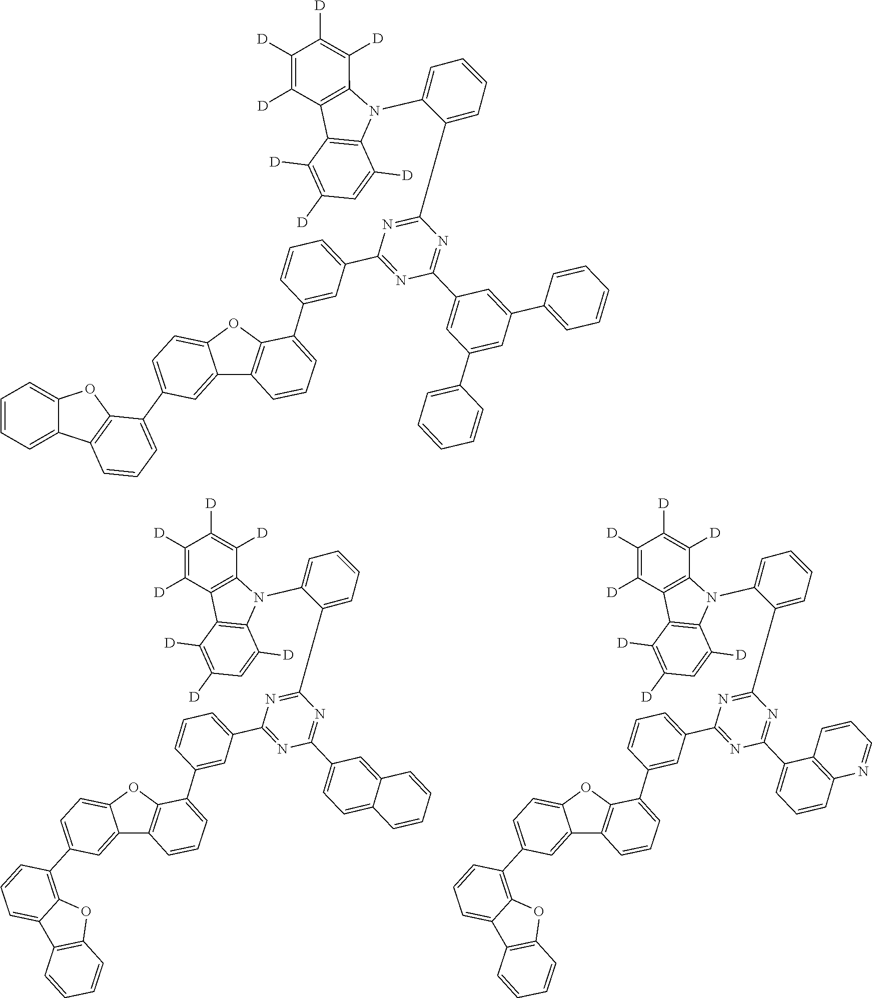 [SMILES: [2H]c1cc([2H])c2c(c1[2H])c1c([2H])c([2H])c([2H])c([2H])c1n2-c1ccccc1-c1nc(-c2cc(-c3ccccc3)cc(-c3ccccc3)c2)nc(-c2cccc(-c3cccc4c3oc3ccc(-c5cccc6c5oc5ccccc56)cc34)c2)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-c2ccc3ccccc3c2)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-c2cccc3ncccc23)n1]

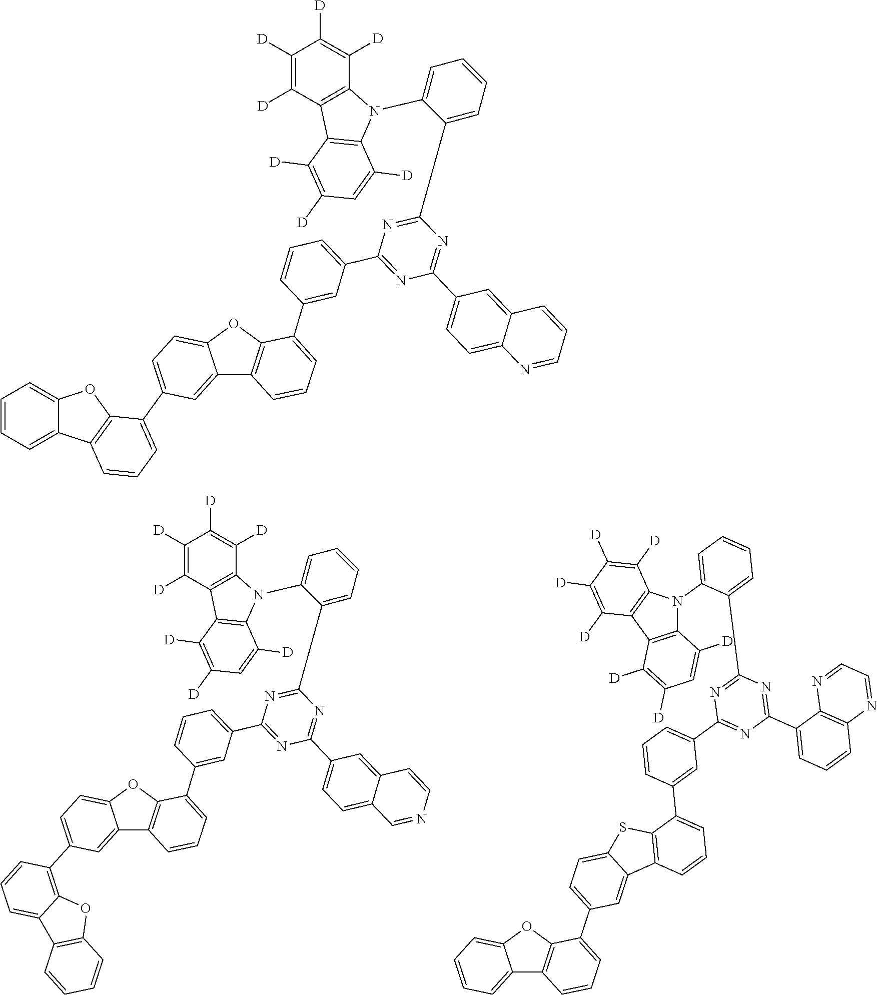 [SMILES: [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-c2ccc3ncccc3c2)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-c2ccc3cnccc3c2)n1 ; [2H]c1cc([2H])c2c(c1[2H])c1c([2H])c([2H])c([2H])c([2H])c1n2-c1ccccc1-c1nc(-c2cccc(-c3cccc4c3sc3ccc(-c5cccc6c5oc5ccccc56)cc34)c2)nc(-c2cccc3nccnc23)n1]

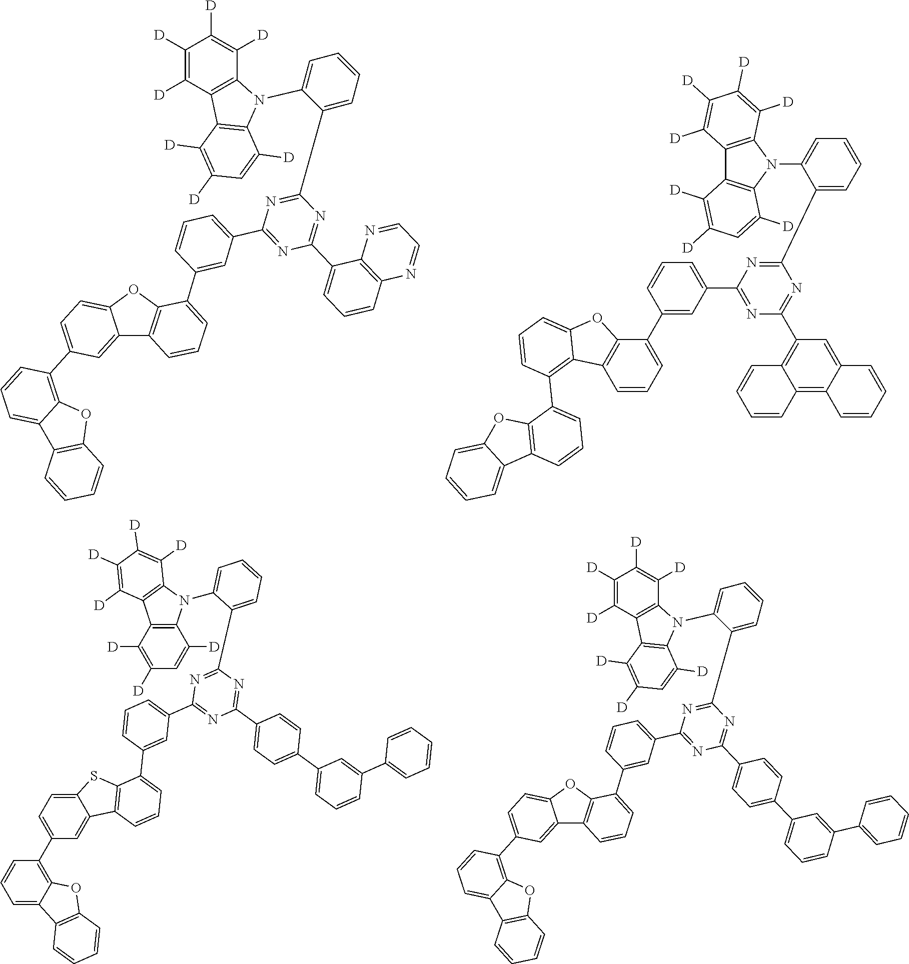

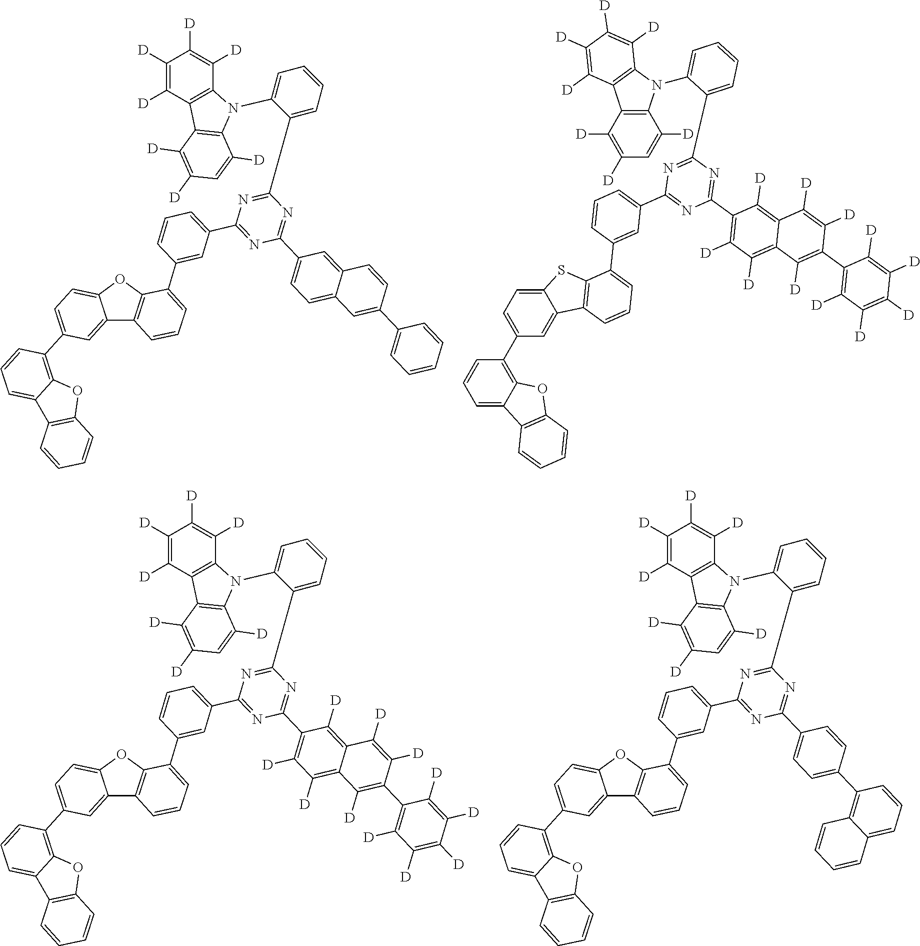

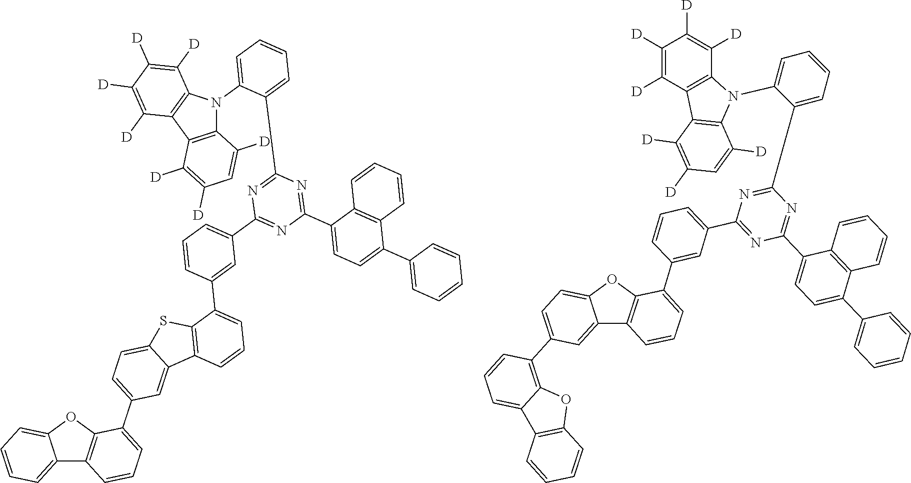 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5cccc6c5oc5ccccc56)cc34)c2)nc(-c2ccc(-c3ccccc3)c3ccccc23)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-c2ccc(-c3ccccc3)c3ccccc23)n1]

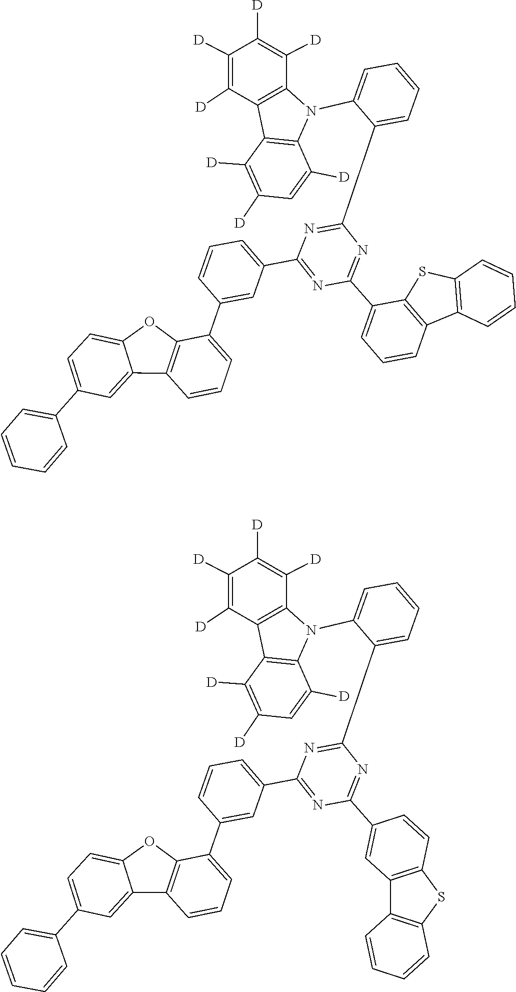 [SMILES: [2H]c1cc([2H])c2c(c1[2H])c1c([2H])c([2H])c([2H])c([2H])c1n2-c1ccccc1-c1nc(-c2cccc(-c3cccc4c3oc3ccc(-c5ccccc5)cc34)c2)nc(-c2cccc3c2sc2ccccc23)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5ccccc5)cc34)c2)nc(-c2ccc3sc4ccccc4c3c2)n1]

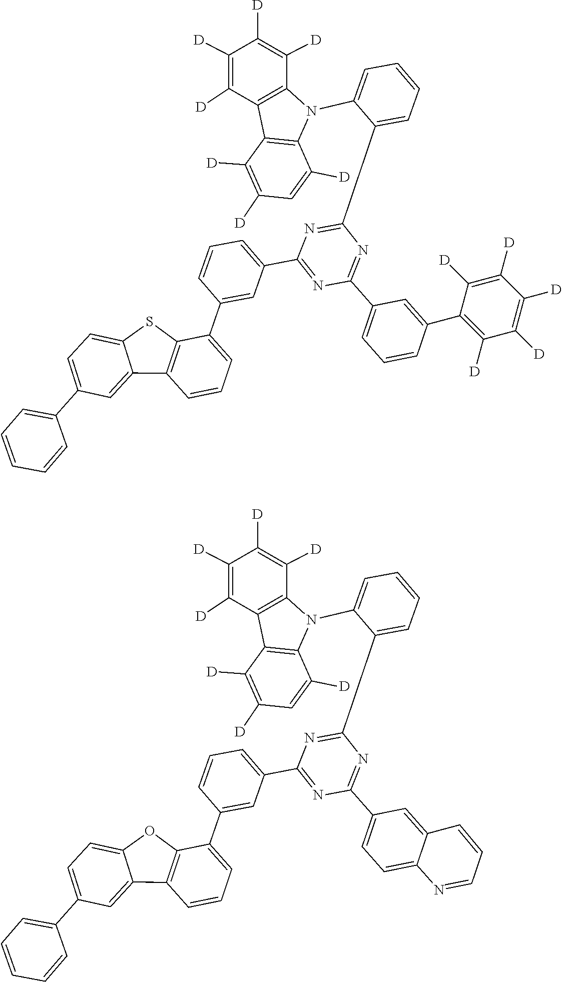 [SMILES: [2H]c1cc([2H])c2c(c1[2H])c1c([2H])c([2H])c([2H])c([2H])c1n2-c1ccccc1-c1nc(-c2cccc(-c3c([2H])c([2H])c([2H])c([2H])c3[2H])c2)nc(-c2cccc(-c3cccc4c3sc3ccc(-c5ccccc5)cc34)c2)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5ccccc5)cc34)c2)nc(-c2ccc3ncccc3c2)n1]

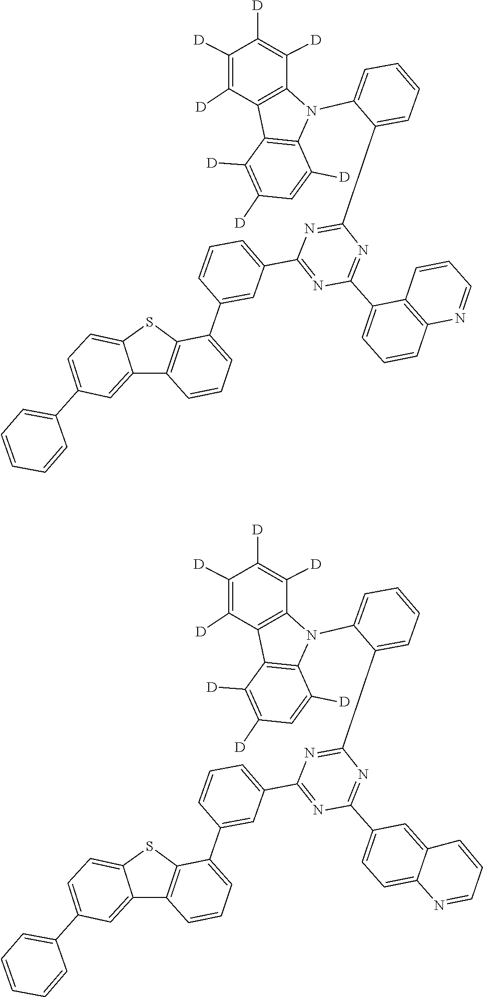 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5ccccc5)cc34)c2)nc(-c2cccc3ncccc23)n1 ; [2H]c1cc([2H])c2c(c1[2H])c1c([2H])c([2H])c([2H])c([2H])c1n2-c1ccccc1-c1nc(-c2cccc(-c3cccc4c3sc3ccc(-c5ccccc5)cc34)c2)nc(-c2ccc3ncccc3c2)n1]

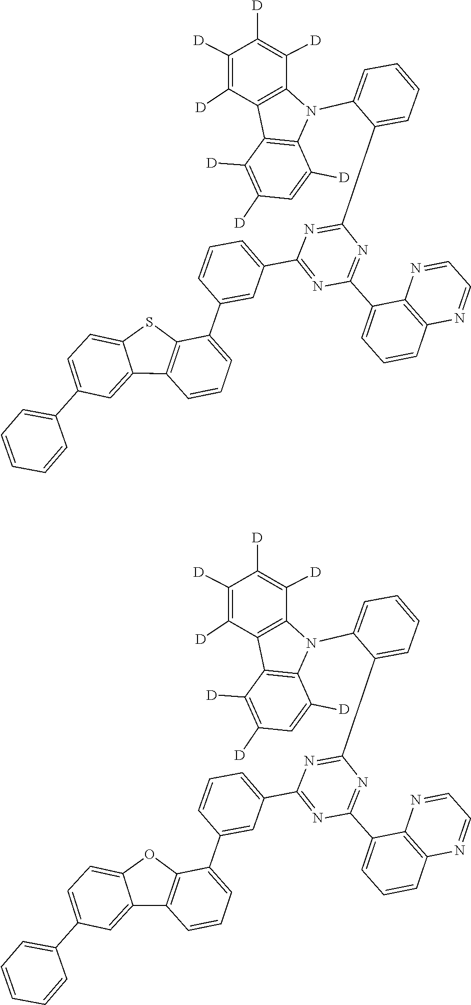 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5ccccc5)cc34)c2)nc(-c2cccc3nccnc23)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5ccccc5)cc34)c2)nc(-c2cccc3nccnc23)n1]

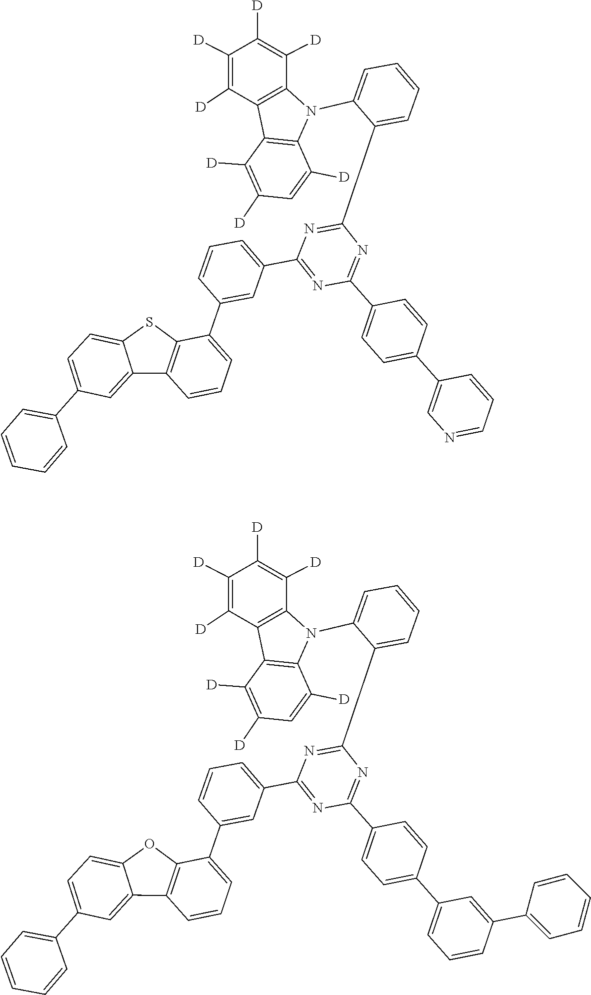 [SMILES: [2H]c1cc([2H])c2c(c1[2H])c1c([2H])c([2H])c([2H])c([2H])c1n2-c1ccccc1-c1nc(-c2ccc(-c3cccnc3)cc2)nc(-c2cccc(-c3cccc4c3sc3ccc(-c5ccccc5)cc34)c2)n1 ; [2H]c1cc([2H])c2c(c1[2H])c1c([2H])c([2H])c([2H])c([2H])c1n2-c1ccccc1-c1nc(-c2ccc(-c3cccc(-c4ccccc4)c3)cc2)nc(-c2cccc(-c3cccc4c3oc3ccc(-c5ccccc5)cc34)c2)n1]

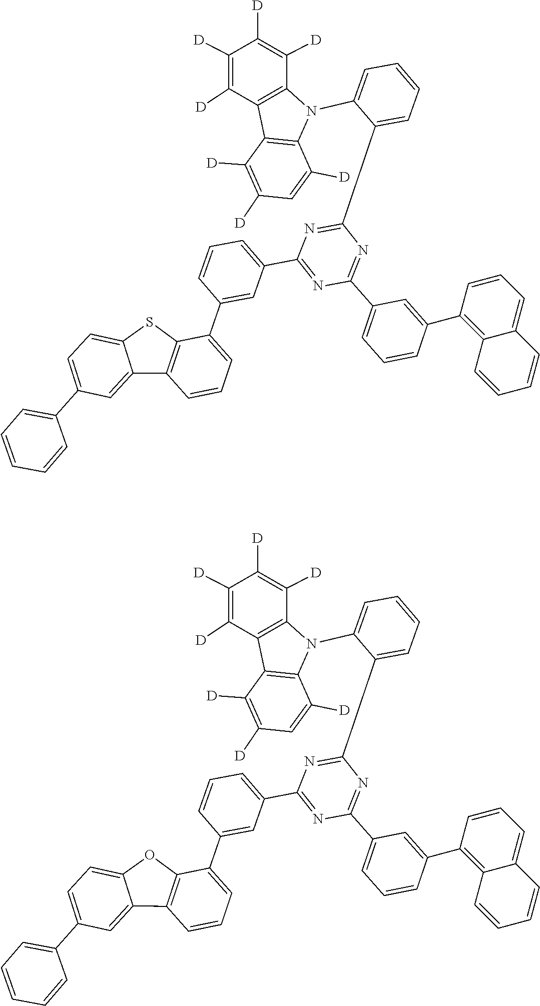 [SMILES: [2H]c1cc([2H])c2c(c1[2H])c1c([2H])c([2H])c([2H])c([2H])c1n2-c1ccccc1-c1nc(-c2cccc(-c3cccc4ccccc34)c2)nc(-c2cccc(-c3cccc4c3sc3ccc(-c5ccccc5)cc34)c2)n1 ; [2H]c1cc([2H])c2c(c1[2H])c1c([2H])c([2H])c([2H])c([2H])c1n2-c1ccccc1-c1nc(-c2cccc(-c3cccc4ccccc34)c2)nc(-c2cccc(-c3cccc4c3oc3ccc(-c5ccccc5)cc34)c2)n1]

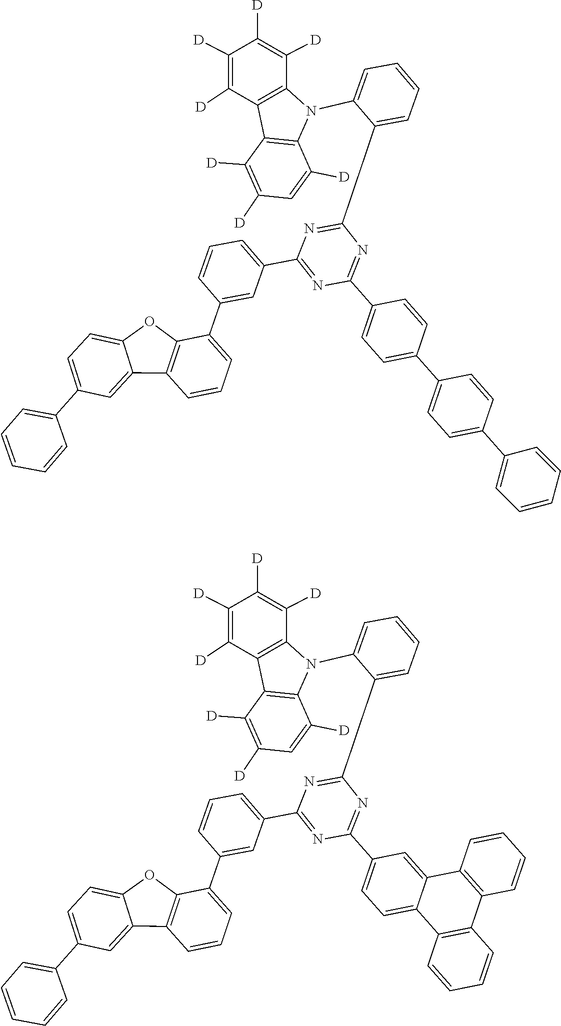 [SMILES: [2H]c1cc([2H])c2c(c1[2H])c1c([2H])c([2H])c([2H])c([2H])c1n2-c1ccccc1-c1nc(-c2ccc(-c3ccc(-c4ccccc4)cc3)cc2)nc(-c2cccc(-c3cccc4c3oc3ccc(-c5ccccc5)cc34)c2)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5ccccc5)cc34)c2)nc(-c2ccc3c4ccccc4c4ccccc4c3c2)n1]

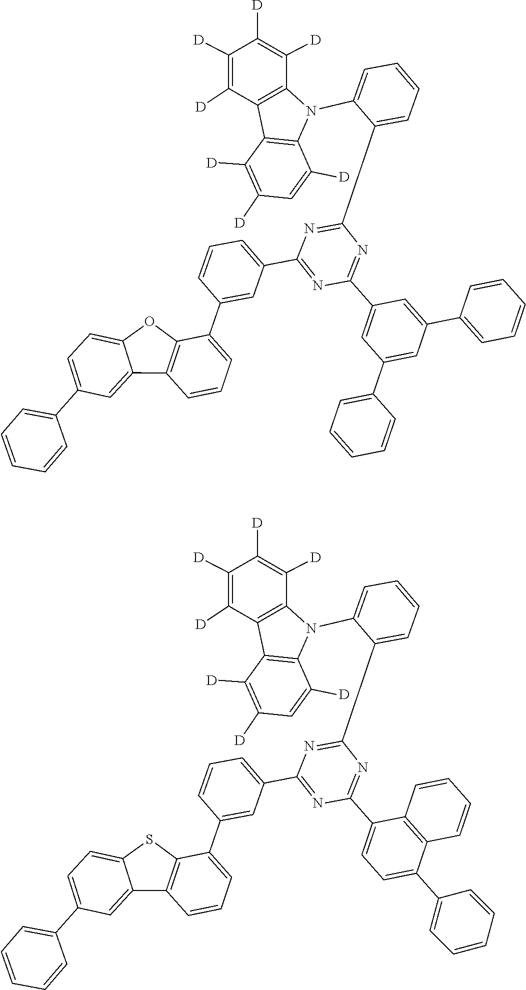 [SMILES: [2H]c1cc([2H])c2c(c1[2H])c1c([2H])c([2H])c([2H])c([2H])c1n2-c1ccccc1-c1nc(-c2cc(-c3ccccc3)cc(-c3ccccc3)c2)nc(-c2cccc(-c3cccc4c3oc3ccc(-c5ccccc5)cc34)c2)n1 ; [2H]c1cc([2H])c2c(c1[2H])c1c([2H])c([2H])c([2H])c([2H])c1n2-c1ccccc1-c1nc(-c2cccc(-c3cccc4c3sc3ccc(-c5ccccc5)cc34)c2)nc(-c2ccc(-c3ccccc3)c3ccccc23)n1]

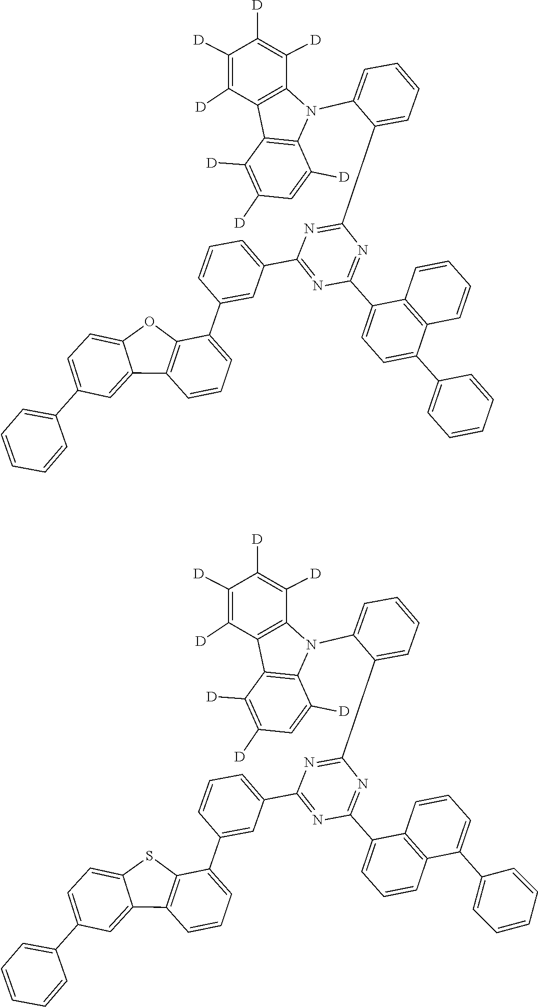 [SMILES: [2H]c1cc([2H])c2c(c1[2H])c1c([2H])c([2H])c([2H])c([2H])c1n2-c1ccccc1-c1nc(-c2cccc(-c3cccc4c3oc3ccc(-c5ccccc5)cc34)c2)nc(-c2ccc(-c3ccccc3)c3ccccc23)n1 ; [2H]c1cc([2H])c2c(c1[2H])c1c([2H])c([2H])c([2H])c([2H])c1n2-c1ccccc1-c1nc(-c2cccc(-c3cccc4c3sc3ccc(-c5ccccc5)cc34)c2)nc(-c2cccc3c(-c4ccccc4)cccc23)n1]

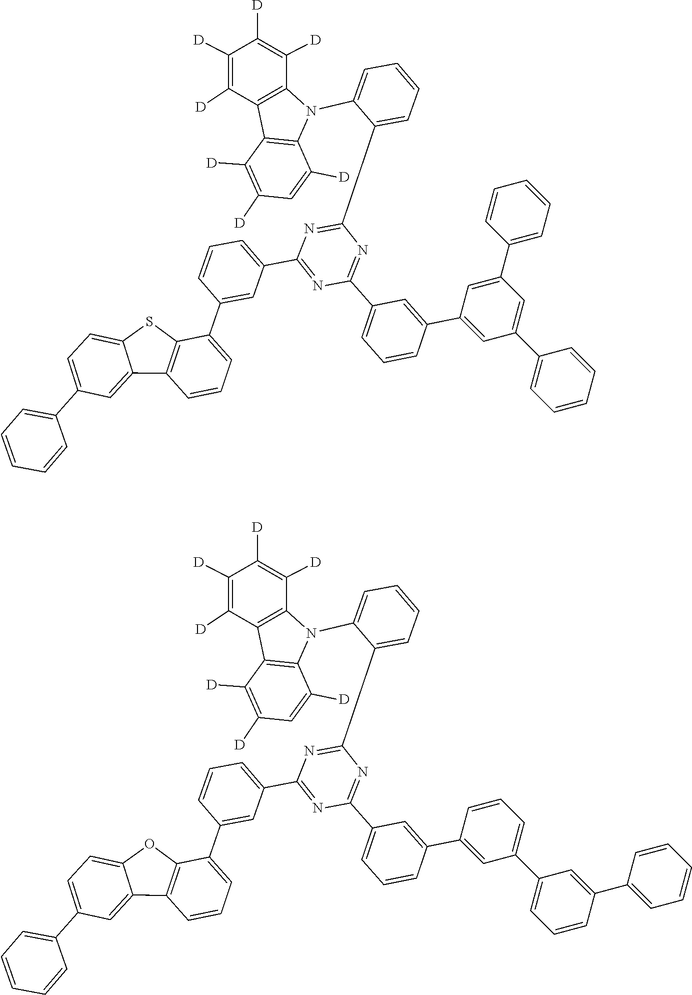 [SMILES: [2H]c1cc([2H])c2c(c1[2H])c1c([2H])c([2H])c([2H])c([2H])c1n2-c1ccccc1-c1nc(-c2cccc(-c3cc(-c4ccccc4)cc(-c4ccccc4)c3)c2)nc(-c2cccc(-c3cccc4c3sc3ccc(-c5ccccc5)cc34)c2)n1 ; [2H]c1cc([2H])c2c(c1[2H])c1c([2H])c([2H])c([2H])c([2H])c1n2-c1ccccc1-c1nc(-c2cccc(-c3cccc(-c4cccc(-c5ccccc5)c4)c3)c2)nc(-c2cccc(-c3cccc4c3oc3ccc(-c5ccccc5)cc34)c2)n1]

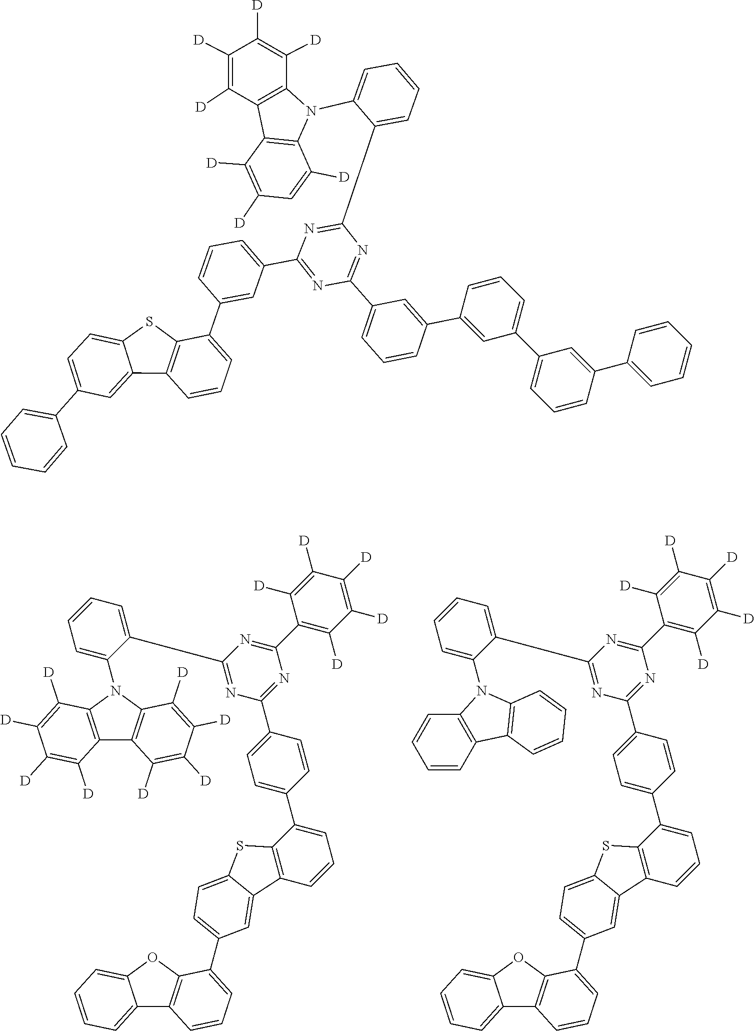 [SMILES: [2H]c1cc([2H])c2c(c1[2H])c1c([2H])c([2H])c([2H])c([2H])c1n2-c1ccccc1-c1nc(-c2cccc(-c3cccc(-c4cccc(-c5ccccc5)c4)c3)c2)nc(-c2cccc(-c3cccc4c3sc3ccc(-c5ccccc5)cc34)c2)n1 ; [2H]c1c([2H])c([2H])c(-c2nc(-c3ccc(-c4cccc5c4sc4ccc(-c6cccc7c6oc6ccccc67)cc45)cc3)nc(-c3ccccc3-n3c4c([2H])c([2H])c([2H])c([2H])c4c4c([2H])c([2H])c([2H])c([2H])c43)n2)c([2H])c1[2H] ; [2H]c1c([2H])c([2H])c(-c2nc(-c3ccc(-c4cccc5c4sc4ccc(-c6cccc7c6oc6ccccc67)cc45)cc3)nc(-c3ccccc3-n3c4ccccc4c4ccccc43)n2)c([2H])c1[2H]]

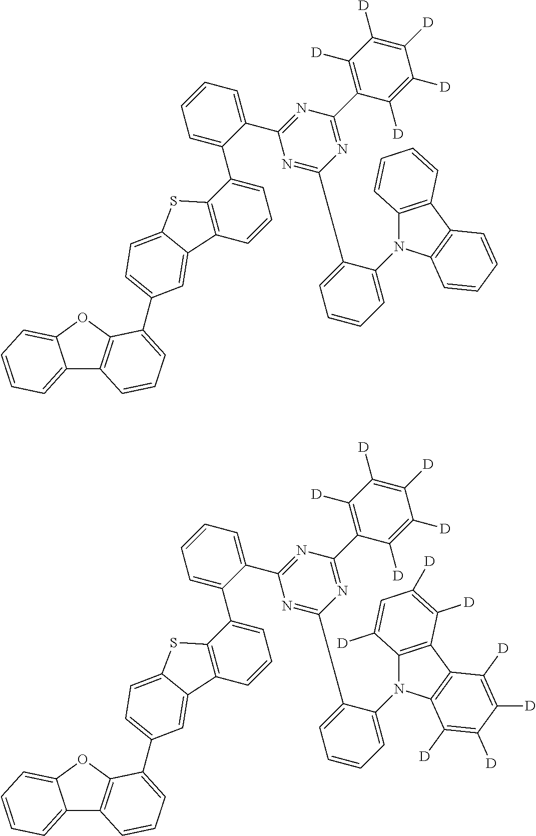 [SMILES: [2H]c1c([2H])c([2H])c(-c2nc(-c3ccccc3-c3cccc4c3sc3ccc(-c5cccc6c5oc5ccccc56)cc34)nc(-c3ccccc3-n3c4ccccc4c4ccccc43)n2)c([2H])c1[2H] ; [2H]c1cc([2H])c2c(c1[2H])c1c([2H])c([2H])c([2H])c([2H])c1n2-c1ccccc1-c1nc(-c2ccccc2-c2cccc3c2sc2ccc(-c4cccc5c4oc4ccccc45)cc23)nc(-c2c([2H])c([2H])c([2H])c([2H])c2[2H])n1]

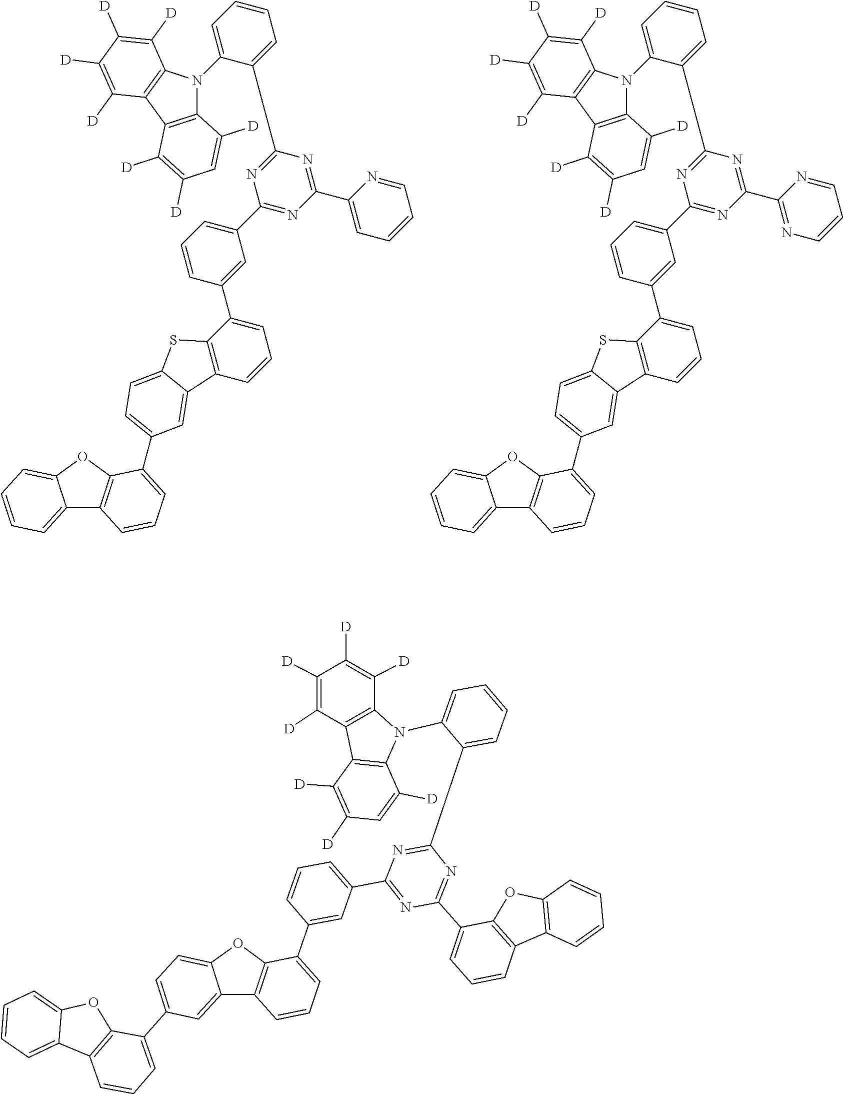 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccc(-c5cccc6c5oc5ccccc56)cc34)c2)nc(-c2ccccn2)n1 ; [2H]c1cc([2H])c2c(c1[2H])c1c([2H])c([2H])c([2H])c([2H])c1n2-c1ccccc1-c1nc(-c2cccc(-c3cccc4c3sc3ccc(-c5cccc6c5oc5ccccc56)cc34)c2)nc(-c2ncccn2)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-c2cccc3c2oc2ccccc23)n1]

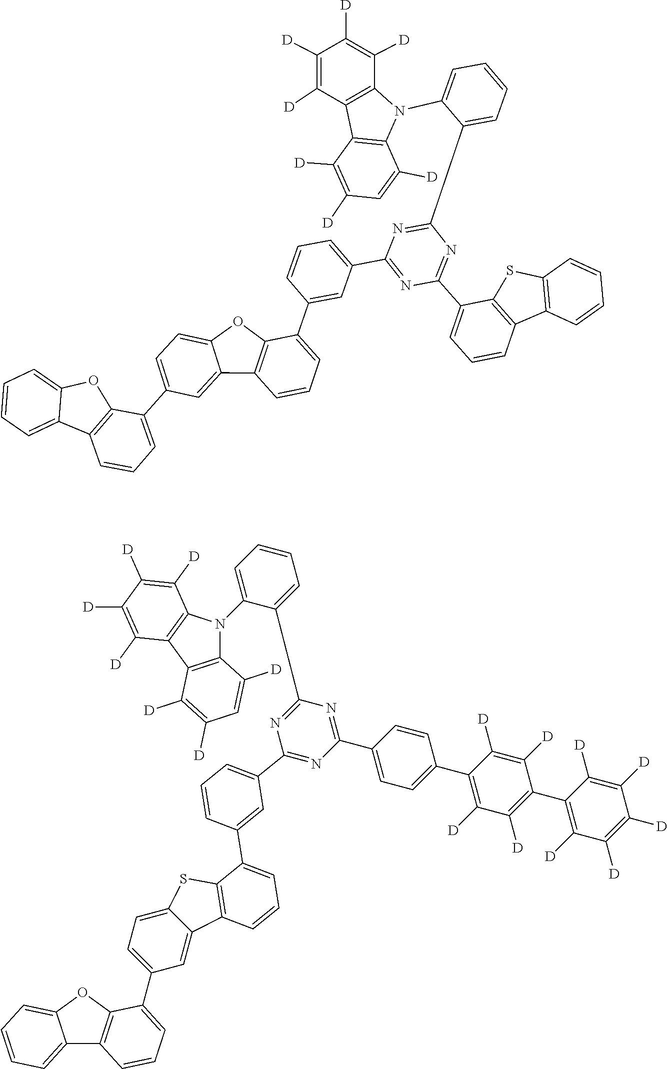 [SMILES: [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-c2cccc3c2sc2ccccc23)n1 ; [2H]c1cc([2H])c2c(c1[2H])c1c([2H])c([2H])c([2H])c([2H])c1n2-c1ccccc1-c1nc(-c2ccc(-c3c([2H])c([2H])c(-c4c([2H])c([2H])c([2H])c([2H])c4[2H])c([2H])c3[2H])cc2)nc(-c2cccc(-c3cccc4c3sc3ccc(-c5cccc6c5oc5ccccc56)cc34)c2)n1]

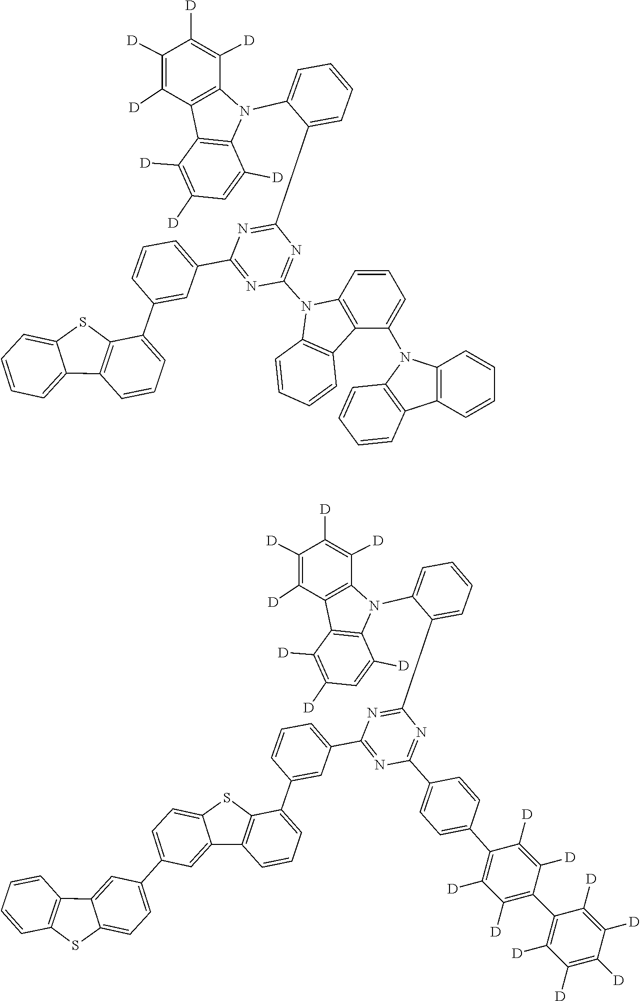 [SMILES: [2H]c1cc([2H])c2c(c1[2H])c1c([2H])c([2H])c([2H])c([2H])c1n2-c1ccccc1-c1nc(-c2cccc(-c3cccc4c3sc3ccccc34)c2)nc(-n2c3ccccc3c3c(-n4c5ccccc5c5ccccc54)cccc32)n1 ; [2H]c1cc([2H])c2c(c1[2H])c1c([2H])c([2H])c([2H])c([2H])c1n2-c1ccccc1-c1nc(-c2ccc(-c3c([2H])c([2H])c(-c4c([2H])c([2H])c([2H])c([2H])c4[2H])c([2H])c3[2H])cc2)nc(-c2cccc(-c3cccc4c3sc3ccc(-c5ccc6sc7ccccc7c6c5)cc34)c2)n1]

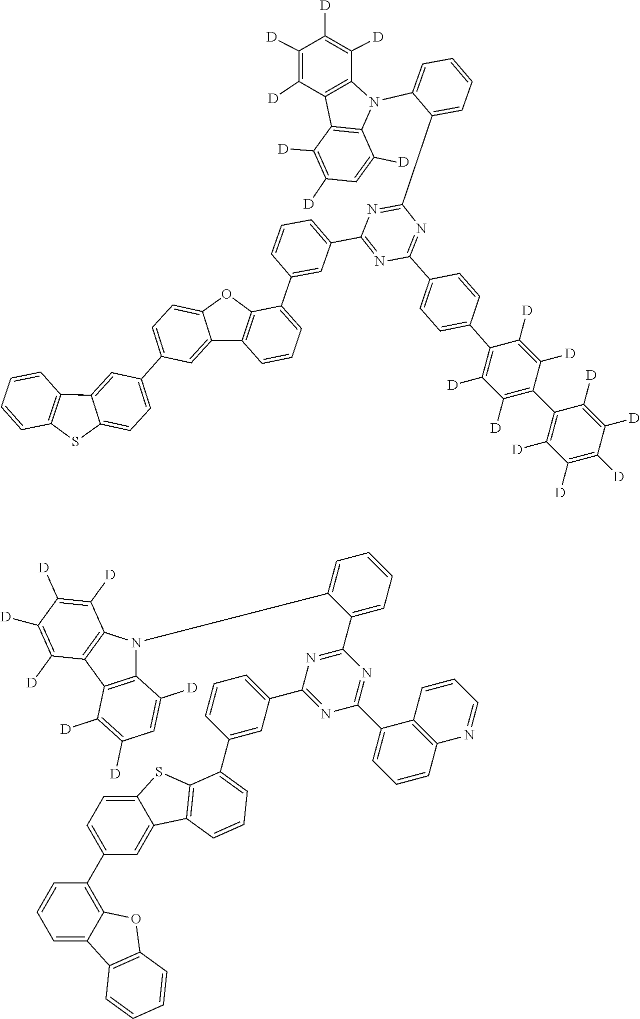 [SMILES: [2H]c1cc([2H])c2c(c1[2H])c1c([2H])c([2H])c([2H])c([2H])c1n2-c1ccccc1-c1nc(-c2ccc(-c3c([2H])c([2H])c(-c4c([2H])c([2H])c([2H])c([2H])c4[2H])c([2H])c3[2H])cc2)nc(-c2cccc(-c3cccc4c3oc3ccc(-c5ccc6sc7ccccc7c6c5)cc34)c2)n1 ; [2H]c1cc([2H])c2c(c1[2H])c1c([2H])c([2H])c([2H])c([2H])c1n2-c1ccccc1-c1nc(-c2cccc(-c3cccc4c3sc3ccc(-c5cccc6c5oc5ccccc56)cc34)c2)nc(-c2cccc3ncccc23)n1]

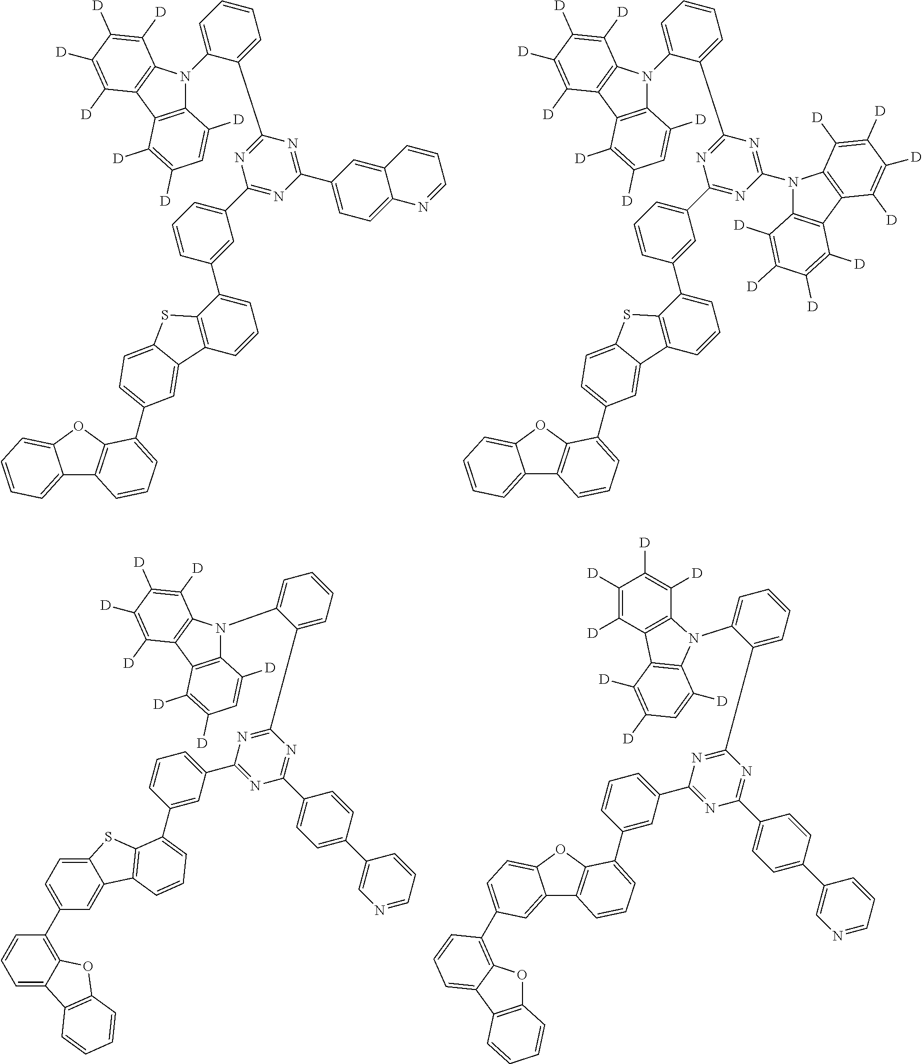

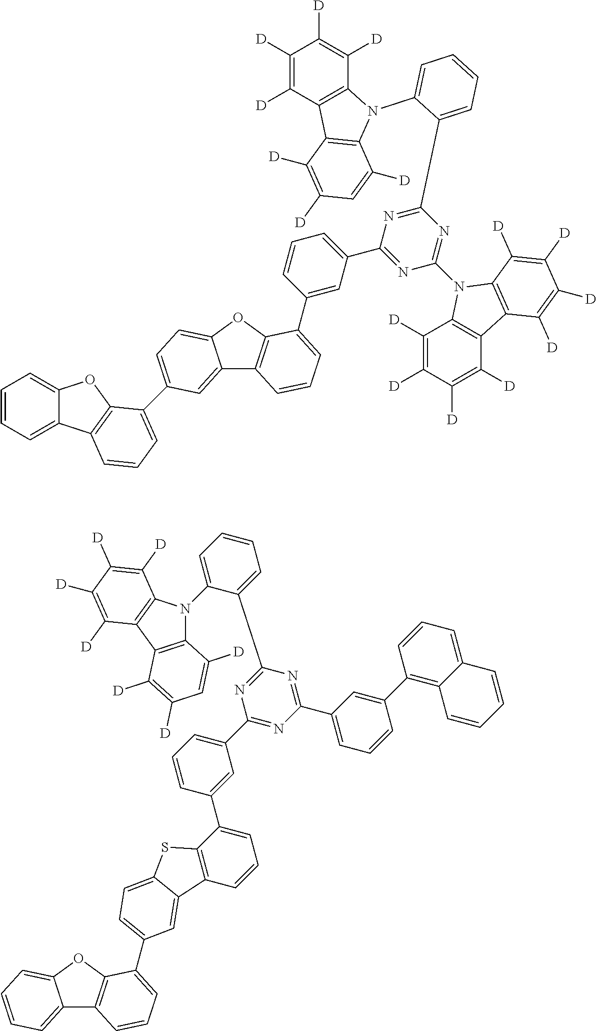 [SMILES: [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-n2c3c([2H])c([2H])c([2H])c([2H])c3c3c([2H])c([2H])c([2H])c([2H])c32)n1 ; [2H]c1cc([2H])c2c(c1[2H])c1c([2H])c([2H])c([2H])c([2H])c1n2-c1ccccc1-c1nc(-c2cccc(-c3cccc4ccccc34)c2)nc(-c2cccc(-c3cccc4c3sc3ccc(-c5cccc6c5oc5ccccc56)cc34)c2)n1]

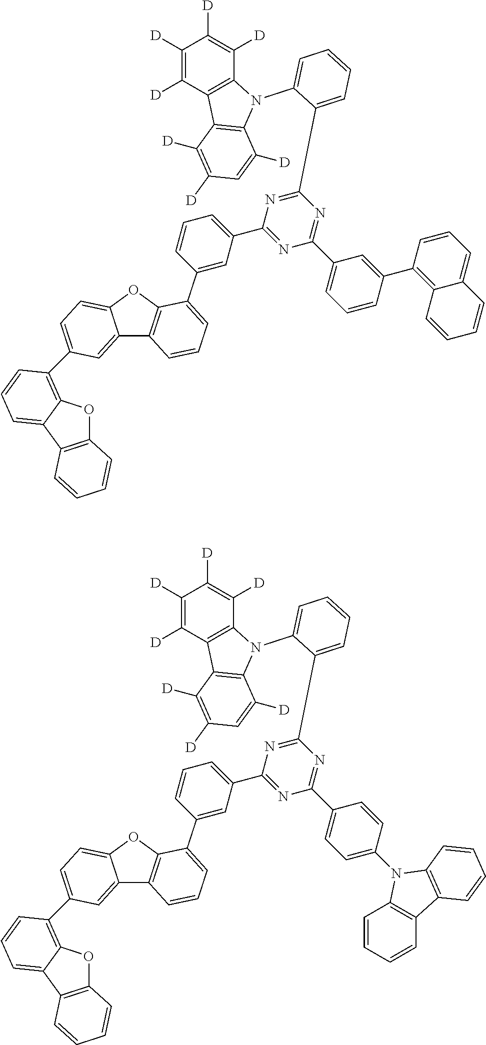 [SMILES: [2H]c1cc([2H])c2c(c1[2H])c1c([2H])c([2H])c([2H])c([2H])c1n2-c1ccccc1-c1nc(-c2cccc(-c3cccc4ccccc34)c2)nc(-c2cccc(-c3cccc4c3oc3ccc(-c5cccc6c5oc5ccccc56)cc34)c2)n1 ; [2H]c1cc([2H])c2c(c1[2H])c1c([2H])c([2H])c([2H])c([2H])c1n2-c1ccccc1-c1nc(-c2ccc(-n3c4ccccc4c4ccccc43)cc2)nc(-c2cccc(-c3cccc4c3oc3ccc(-c5cccc6c5oc5ccccc56)cc34)c2)n1]

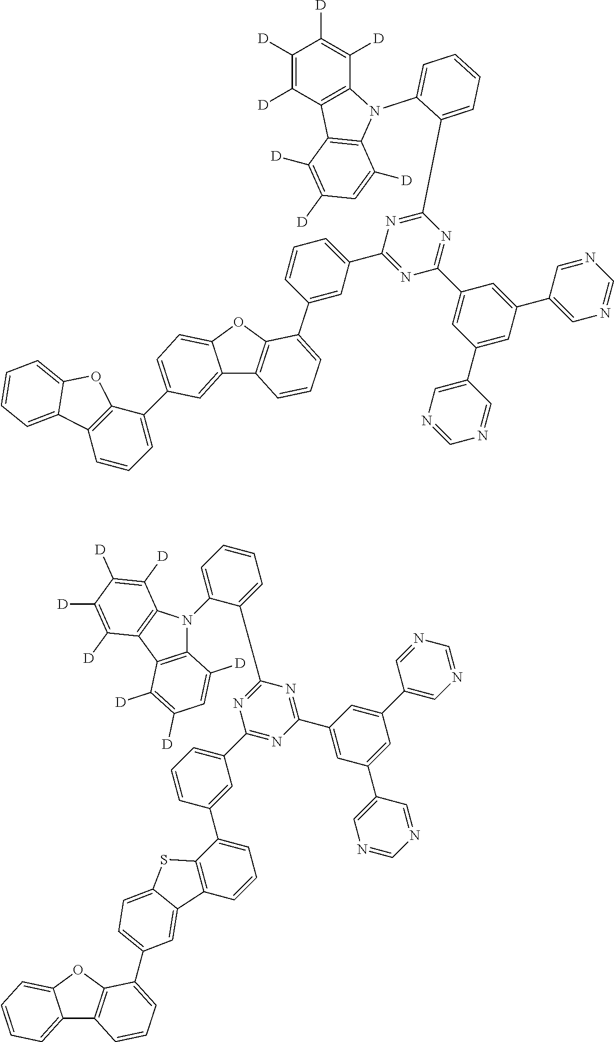 [SMILES: [2H]c1cc([2H])c2c(c1[2H])c1c([2H])c([2H])c([2H])c([2H])c1n2-c1ccccc1-c1nc(-c2cc(-c3cncnc3)cc(-c3cncnc3)c2)nc(-c2cccc(-c3cccc4c3oc3ccc(-c5cccc6c5oc5ccccc56)cc34)c2)n1 ; [2H]c1cc([2H])c2c(c1[2H])c1c([2H])c([2H])c([2H])c([2H])c1n2-c1ccccc1-c1nc(-c2cc(-c3cncnc3)cc(-c3cncnc3)c2)nc(-c2cccc(-c3cccc4c3sc3ccc(-c5cccc6c5oc5ccccc56)cc34)c2)n1]

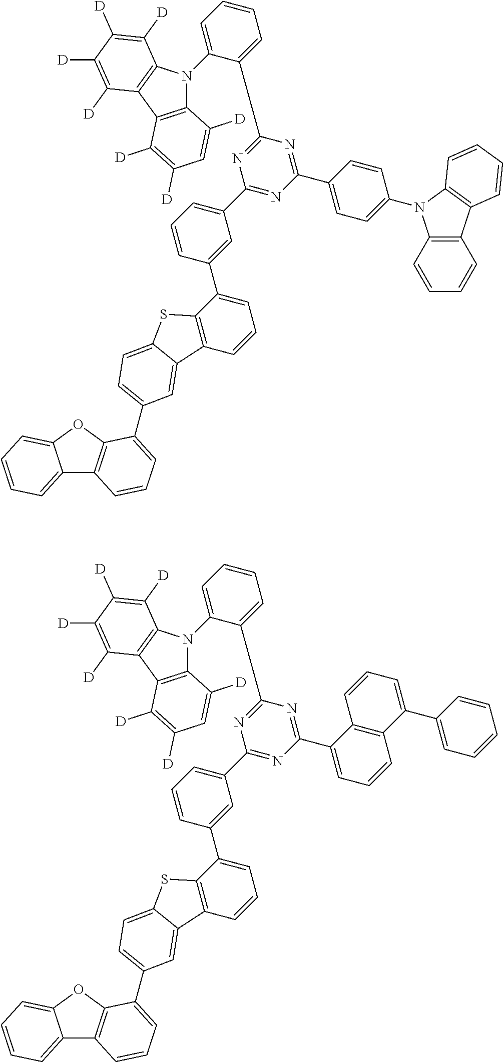 [SMILES: [2H]c1cc([2H])c2c(c1[2H])c1c([2H])c([2H])c([2H])c([2H])c1n2-c1ccccc1-c1nc(-c2ccc(-n3c4ccccc4c4ccccc43)cc2)nc(-c2cccc(-c3cccc4c3sc3ccc(-c5cccc6c5oc5ccccc56)cc34)c2)n1 ; [2H]c1cc([2H])c2c(c1[2H])c1c([2H])c([2H])c([2H])c([2H])c1n2-c1ccccc1-c1nc(-c2cccc(-c3cccc4c3sc3ccc(-c5cccc6c5oc5ccccc56)cc34)c2)nc(-c2cccc3c(-c4ccccc4)cccc23)n1]

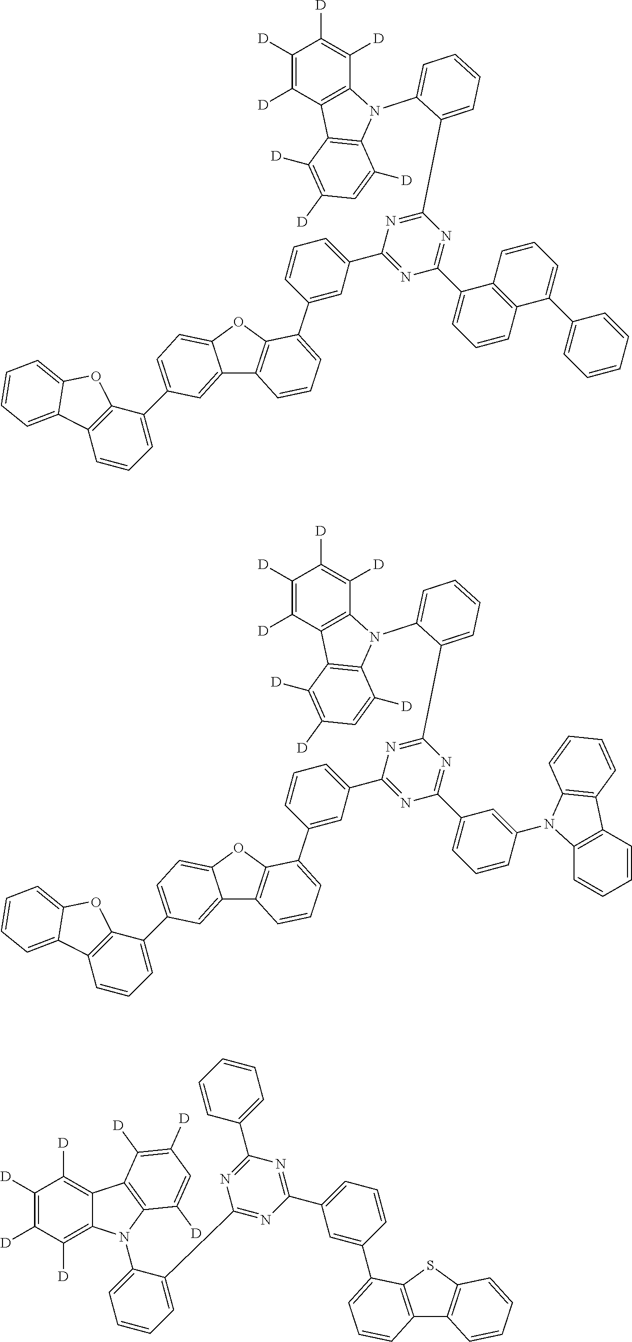 [SMILES: [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-c2cccc3c(-c4ccccc4)cccc23)n1 ; [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-c2cccc(-n3c4ccccc4c4ccccc43)c2)n1 ; [2H]c1cc([2H])c2c(c1[2H])c1c([2H])c([2H])c([2H])c([2H])c1n2-c1ccccc1-c1nc(-c2ccccc2)nc(-c2cccc(-c3cccc4c3sc3ccccc34)c2)n1]

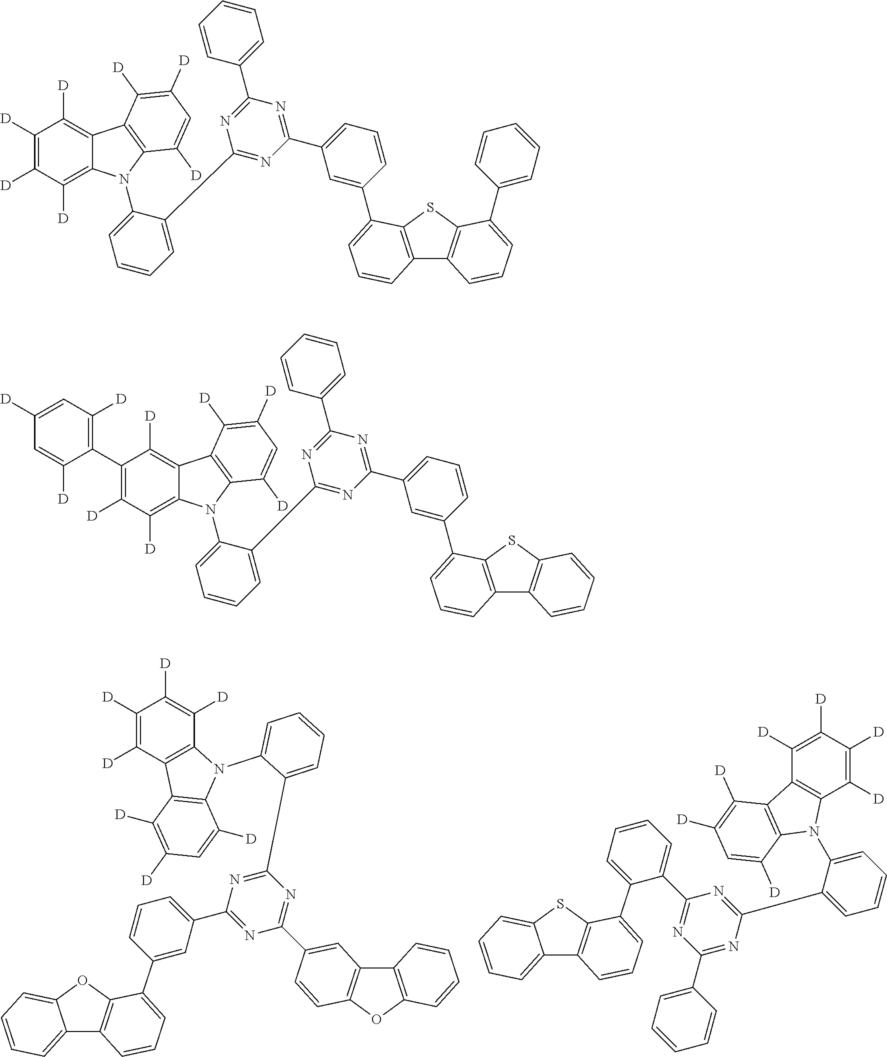

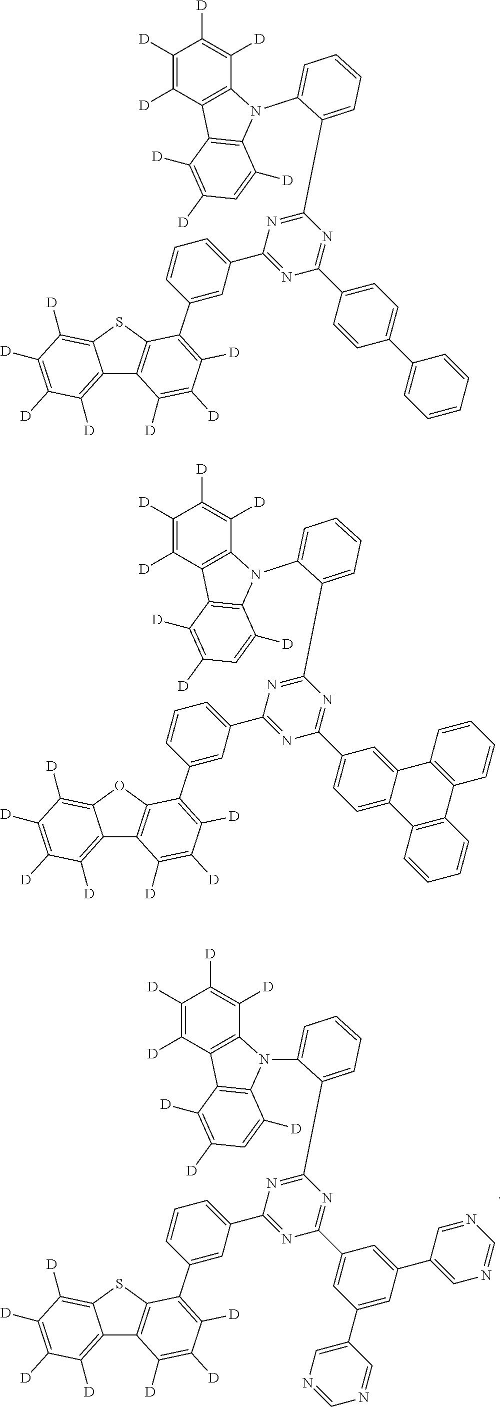

The compound represented by Chemical Formula 1 can be prepared by a preparation method as shown in the following Reaction Scheme 1 as an example, and other remaining compounds can also be prepared in a similar manner.

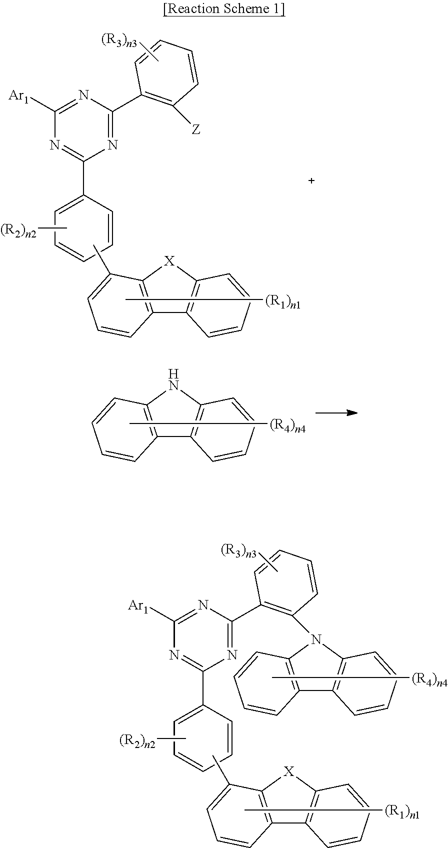

In Reaction Scheme 1, Ar₁, X, R₁ to R₄ and n1 to n4 are as defined in Chemical Formula 1, Z is halogen, preferably Z is fluoro, chloro or bromo.

Reaction Scheme 1 is an amine substitution reaction, which is preferably carried out in the presence of a palladium catalyst and a base, and a reactive group for the amine substitution reaction can be modified as known in the art. The preparation method can be further embodied in Preparation Examples described hereinafter.

Further, according to the present disclosure, provided is an organic light emitting device comprising a compound represented by Chemical Formula 1. In one example, the present disclosure provides an organic light emitting device comprising: a first electrode; a second electrode that is provided opposite to the first electrode; and one or more organic material layers that are provided between the first electrode and the second electrode, wherein at least one layer of the organic material layers includes the compound represented by Chemical Formula 1.

The organic material layer of the organic light emitting device of the present disclosure may have a single-layer structure, or it may have a multilayered structure in which two or more organic material layers are stacked. For example, the organic light emitting device of the present disclosure may have a structure comprising a hole injection layer, a hole transport layer, an electron blocking layer, a light emitting layer, a hole blocking layer, an electron transport layer, an electron injection layer and the like as the organic material layer. However, the structure of the organic light emitting device is not limited thereto, and it may include a smaller number of organic layers.

Further, the organic material layer may include a light emitting layer, wherein the light emitting layer may include the compound represented by Chemical Formula 1.

Further, the organic material layer may include a hole transport layer, a hole injection layer, or a layer that simultaneously performs hole transport and hole injection, wherein the hole transport layer, the hole injection layer, or the layer that simultaneously performs hole transport and hole injection may include the compound represented by Chemical Formula 1.

Further, the organic material layer may include an electron transport layer, an electron injection layer, or an electron injection and transport layer, wherein the electron transport layer, the electron injection layer, or the electron injection and transport layer may include the compound represented by Chemical Formula 1.

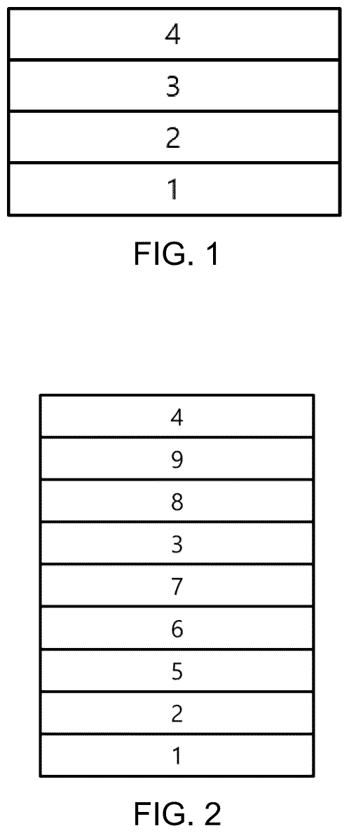

Further, the organic light emitting device according to the present disclosure may be a normal type organic light emitting device in which an anode, one or more organic material layers, and a cathode are sequentially stacked on a substrate. Further, the organic light emitting device according to the present disclosure may be an inverted type organic light emitting device in which a cathode, one or more organic material layers, and an anode are sequentially stacked on a substrate. For example, the structure of the organic light emitting device according to an embodiment of the present disclosure is illustrated in FIGS. 1 and 2.

FIG. 1 shows an example of an organic light emitting device comprising a substrate 1, an anode 2, a light emitting layer 3, and a cathode 4. FIG. 2 shows an example of an organic light emitting device comprising a substrate 1, an anode 2, a hole injection layer 5, a hole transport layer 6, an electron blocking layer 7, a light emitting layer 3, a hole blocking layer 8, an electron transport and injection layer 9, and a cathode 4. In such a structure, the compound represented by Chemical Formula 1 may be included in the light emitting layer.

The organic light emitting device according to the present disclosure may be manufactured by materials and methods known in the art, except that at least one of the organic material layers includes the compound represented by Chemical Formula 1. Further, when the organic light emitting device includes a plurality of organic material layers, the organic material layers can be formed of the same material or different materials.

For example, the organic light emitting device according to the present disclosure can be manufactured by sequentially stacking a first electrode, an organic material layer and a second electrode on a substrate. In this case, the organic light emitting device may be manufactured by depositing a metal, metal oxides having conductivity, or an alloy thereof on the substrate using a PVD (physical vapor deposition) method such as a sputtering method or an e-beam evaporation method to form an anode, forming organic material layers including the hole injection layer, the hole transport layer, the light emitting layer and the electron transport layer thereon, and then depositing a material that can be used as the cathode thereon. In addition to such a method, the organic light emitting device can be manufactured by sequentially depositing a cathode material, an organic material layer and an anode material on a substrate.

Further, the compound represented by Chemical Formula 1 can be formed into an organic layer by a solution coating method as well as a vacuum deposition method at the time of manufacturing an organic light emitting device. Herein, the solution coating method means a spin coating, a dip coating, a doctor blading, an inkjet printing, a screen printing, a spray method, a roll coating, or the like, but is not limited thereto.

In addition to such a method, the organic light emitting device may be manufactured by sequentially depositing a cathode material, an organic material layer and an anode material on a substrate (International Publication WO2003/012890). However, the manufacturing method is not limited thereto.

As an example, the first electrode is an anode, and the second electrode is a cathode, or alternatively, the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive compounds such as poly(3-methylthiophene), poly[3,4-(ethylene-1,2-dioxy)thiophene](PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

Further, the hole injection layer is a layer for injecting holes from the electrode, and the hole injection material is preferably a compound which has a capability of transporting the holes, thus has a hole injecting effect in the anode and an excellent hole injecting effect to the light emitting layer or the light emitting material, prevents excitons produced in the light emitting layer from moving to a hole injection layer or the electron injection material, and further is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrin, oligothiophene, an arylamine-based organic material, a hexanitrilehexaazatriphenylene-based organic material, a quinacridone-based organic material, a perylene-based organic material, anthraquinone, polyaniline and polythiophene-based conductive polymer, and the like, but are not limited thereto.

The hole transport layer is a layer that receives holes from a hole injection layer and transports the holes to the light emitting layer. The hole transport material is suitably a material having large mobility to the holes, which may receive holes from the anode or the hole injection layer and transfer the holes to the light emitting layer. Specific examples thereof include an arylamine-based organic material, a conductive polymer, a block copolymer in which a conjugate portion and a non-conjugate portion are present together, and the like, but are not limited thereto.

The electron blocking layer means a layer provided between the hole transport layer and the light emitting layer in order to prevent the electrons injected in the cathode from being transferred to the hole transport layer without being recombined in the light emitting layer, which may also be referred to as an electron inhibition layer or an electron stopping layer. The electron blocking layer is preferably a material having the smaller electron affinity than the electron transport layer.

The light emitting material is preferably a material which may receive holes and electrons transported from a hole transport layer and an electron transport layer, respectively, and combine the holes and the electrons to emit light in a visible ray region, and has good quantum efficiency to fluorescence or phosphorescence. Specific examples of the light emitting material include an 8-hydroxy-quinoline aluminum complex (Alq₃); a carbazole-based compound; a dimerized styryl compound; BAlq; a 10-hydroxybenzoquinoline-metal compound; a benzoxazole, benzothiazole and benzimidazole-based compound; a poly(p-phenylenevinylene)(PPV)-based polymer; a spiro compound; polyfluorene, rubrene, and the like, but are not limited thereto.

The light emitting layer may include a host material and a dopant material. The host material includes a fused aromatic ring derivative, a heterocycle-containing compound, or the like. Specific examples of the fused aromatic ring derivatives include anthracene derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

Examples of the dopant material include an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracene, chrysene, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

The hole blocking layer is a layer provided between the electron transport layer and the light emitting layer in order to prevent the holes injected in the anode from being transferred to the electron transport layer without being recombined in the light emitting layer, which may also be referred to as a hole inhibition layer or a hole stopping layer. The hole blocking layer is preferably a material having the large ionization energy.

The electron transport layer is a layer that may receive the electrons from the electron injection layer and transport the electrons to the light emitting layer, and an electron transport material is suitably a material which may receive well injection of electrons from a cathode and transfer the electrons to a light emitting layer, and has a large mobility for electrons. Specific examples of the electron transport material include: an Al complex of 8-hydroxyquinoline; a complex including Alq₃; an organic radical compound; a hydroxyflavone-metal complex, and the like, but are not limited thereto. The electron transport layer may be used with any desired cathode material, as used according to a conventional technique. In particular, appropriate examples of the cathode material are a typical material which has a low work function, followed by an aluminum layer or a silver layer. Specific examples thereof include cesium, barium, calcium, ytterbium, and samarium, in each case followed by an aluminum layer or a silver layer.

The electron injection layer is a layer which injects electrons from an electrode, and is preferably a compound which has a capability of transporting electrons, has an effect of injecting electrons from a cathode and an excellent effect of injecting electrons into a light emitting layer or a light emitting material, prevents excitons produced from the light emitting layer from moving to a hole injection layer, and is also excellent in the ability to form a thin film. Specific examples of the electron injection layer include fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

Examples of the metal complex compound include 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-quinolinato)chlorogallium, bis(2-methyl-8-quinolinato)(o-cresolato)gallium, bis(2-methyl-8-quinolinato)(1-naphtholato)aluminum, bis(2-methyl-8-quinolinato)(2-naphtholato)gallium, and the like, but are not limited thereto.

Meanwhile, in the present disclosure, the “electron injection and transport layer” or “electron transport or injection layer” is a layer that performs both the roles of the electron injection layer and the electron transport layer, and the materials that perform the roles of each layer may be used alone or in combination, but are not limited thereto.

The organic light emitting device according to the present disclosure may be a bottom emission type device, a top emission type device, or a double side emission type device, and in particular, it may be a bottom emission type light emitting device that requires relatively high luminous efficiency.

In addition, the compound represented by Chemical Formula 1 may be included in an organic solar cell or an organic transistor in addition to an organic light emitting device.

Below, embodiments are described in more detail to assist in the understanding of the present disclosure. However, the following Examples are for illustrative purposes only, and are not intended to limit the scope of the present disclosure.

### Preparation Example

### Preparation Example 1: Preparation of Compound 1

### Step 1) Preparation of Compound 1-a

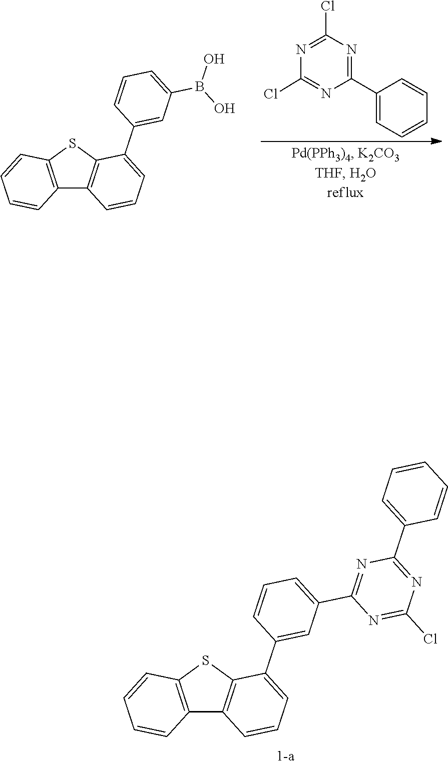 [SMILES: OB(O)c1cccc(-c2cccc3c2sc2ccccc23)c1 ; Clc1nc(Cl)nc(-c2ccccc2)n1 ; Clc1nc(-c2ccccc2)nc(-c2cccc(-c3cccc4c3sc3ccccc34)c2)n1]

(3-(Dibenzo[b,d]thiophen-4-yl)phenyl)boronic acid (30 g, 98.6 mmol) and 2,4-dichloro-6-phenyl-1,3,5-triazine (22.3 g, 98.6 mmol) were added to 360 ml of tetrahydrofuran under a nitrogen atmosphere, and the mixture was stirred and refluxed. Then, potassium carbonate (40.9 g, 295.9 mmol) was dissolved in 123 ml of water and added thereto, the mixture was sufficiently stirred and then tetrakistriphenyl-phosphinopalladium (2.3 g, 2 mmol) was added. After the reaction for 8 hours, the reaction mixture was cooled to room temperature, and the resulting solid was filtered. The solid was added to and dissolved in 1331 mL of chloroform, and washed twice with water. The organic layer was then separated, anhydrous magnesium sulfate was added thereto, stirred, then filtered, and the filtrate was distilled under reduced pressure. The concentrated compound was recrystallized from chloroform and ethyl acetate to prepare Compound 1-a as a gray solid (31.1 g, 70%, MS: [M+H]⁺=451).

### Step 2) Preparation of Compound 1-b

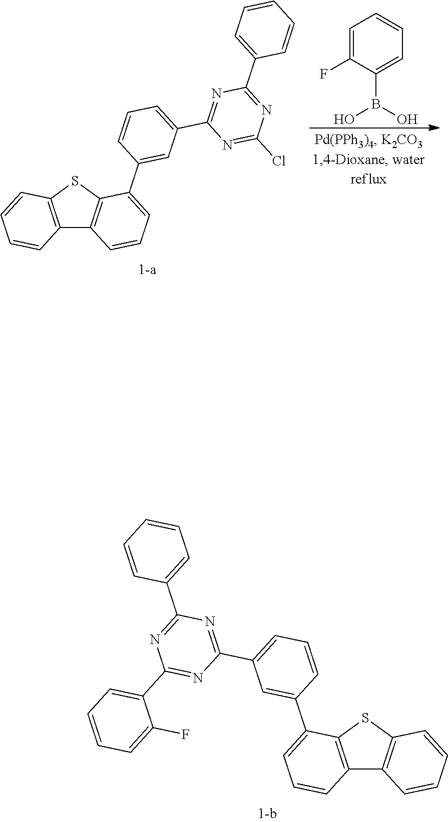 [SMILES: Clc1nc(-c2ccccc2)nc(-c2cccc(-c3cccc4c3sc3ccccc34)c2)n1 ; OB(O)c1ccccc1F ; Fc1ccccc1-c1nc(-c2ccccc2)nc(-c2cccc(-c3cccc4c3sc3ccccc34)c2)n1]

Compound 1-a (30 g, 66.7 mmol) and (2-fluorophenyl)boronic acid (9.3 g, 66.7 mmol) were added to 450 ml of 1,4-dioxane under a nitrogen atmosphere, and the mixture was stirred and refluxed. Then, potassium carbonate (27.6 g, 200 mmol) was dissolved in 90 ml of water and added thereto, and the mixture was sufficiently stirred and then tetrakistriphenyl-phosphinopalladium (1.5 g, 1.3 mmol) was added. After the reaction for 9 hours, the reaction mixture was cooled to room temperature, and the resulting solid was filtered. The solid was added to and dissolved in 1019 mL of chloroform, and washed twice with water. The organic layer was then separated, anhydrous magnesium sulfate was added thereto, stirred, then filtered, and the filtrate was distilled under reduced pressure. The concentrated compound was recrystallized from chloroform and ethyl acetate to prepare Compound 1-b as a white solid (29.9 g, 88%, MS: [M+H]⁺=510.6).

### Step 3) Preparation of Compound 1

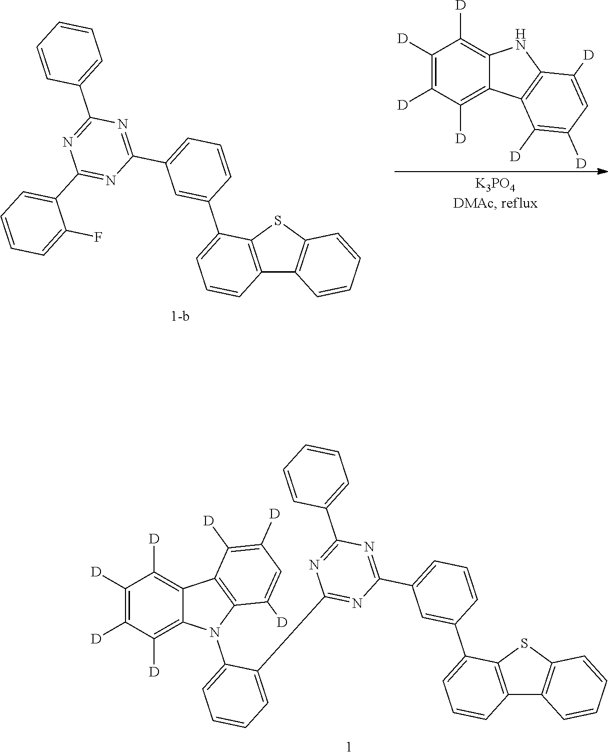 [SMILES: Fc1ccccc1-c1nc(-c2ccccc2)nc(-c2cccc(-c3cccc4c3sc3ccccc34)c2)n1 ; [2H]c1cc([2H])c2[nH]c3c([2H])c([2H])c([2H])c([2H])c3c2c1[2H] ; [2H]c1cc([2H])c2c(c1[2H])c1c([2H])c([2H])c([2H])c([2H])c1n2-c1ccccc1-c1nc(-c2ccccc2)nc(-c2cccc(-c3cccc4c3sc3ccccc34)c2)n1]

Compound 1-b (25 g, 49.1 mmol) and 9H-carbazole-1,2,3,4,5,6,8-d7 (8.5 g, 49.1 mmol) were added to 200 ml of dimethylacetamide under a nitrogen atmosphere, and the mixture was stirred and refluxed. Then, potassium phosphate tribasic (31.2 g, 147.2 mmol) was added thereto, and the mixture was sufficiently stirred, and then reacted for 3 hours. The reaction mixture was then cooled to room temperature, the organic layer was filtered to remove salt, and then the filtered organic layer was distilled. This was again added to and dissolved in 977 mL of chloroform, washed twice with water, and the organic layer was separated. Anhydrous magnesium sulfate was added thereto, stirred, filtered, and the filtrate was distilled under reduced pressure. The concentrated compound was purified by a silica column using chloroform and ethyl acetate to prepare Compound 1 as a yellow solid (22.1 g, 68%, MS: [M+H]+=644.9).

### Preparation Example 2: Preparation of Compound 2

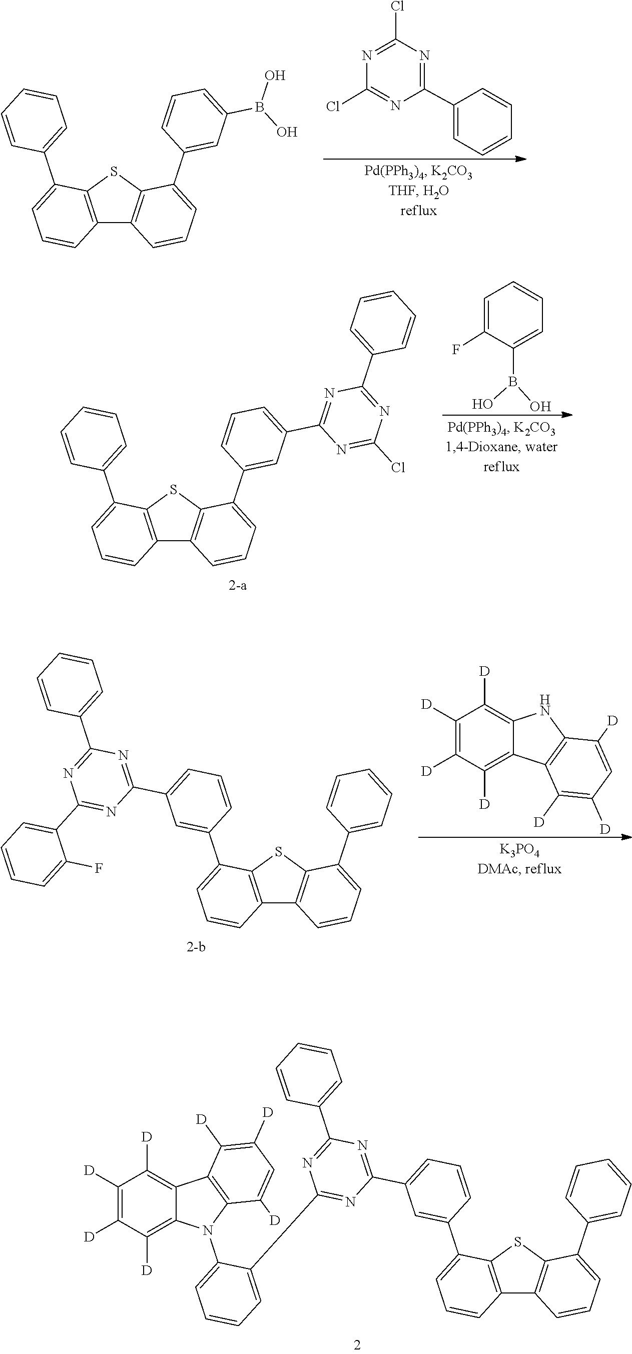

Compound 2 was prepared in the same manner as in the preparation method of Compound 1, except that in Preparation Example 1, (3-(6-phenyldibenzo[b,d]thiophen-4-yl)phenyl)boronic acid was used instead of (3-(dibenzo[b,d]thiophen-4-yl)phenyl)boronic acid. (MS: [M+H]⁺=741)

### Preparation Example 3: Preparation of Compound 3

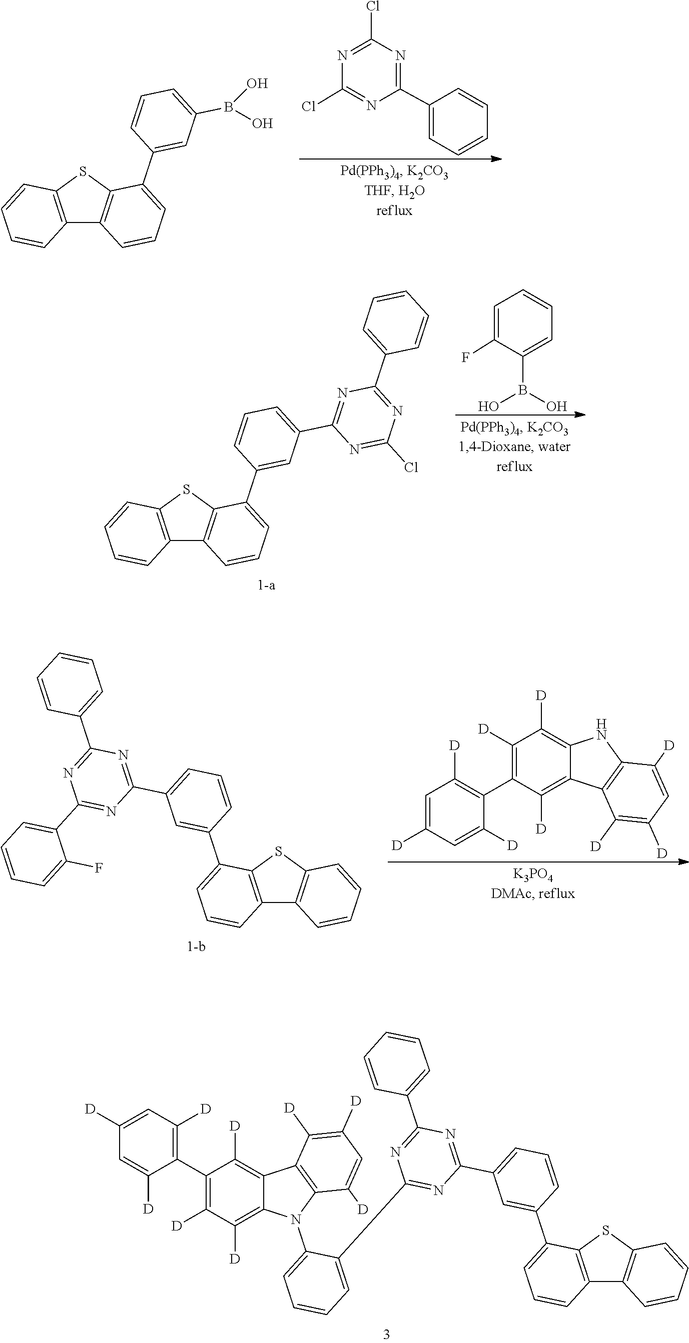

Compound 3 was prepared in the same manner as in the preparation method of Compound 1, except that in Preparation Example 1, 3-(phenyl-2,4,6-d3)-9H-carbazole-1,2,4,5,6,8-d6 was used instead of 9H-carbazole-1,2,3,4,5,6,8-d7. (MS: [M+H]⁺=743)

### Preparation Example 4: Preparation of Compound 4

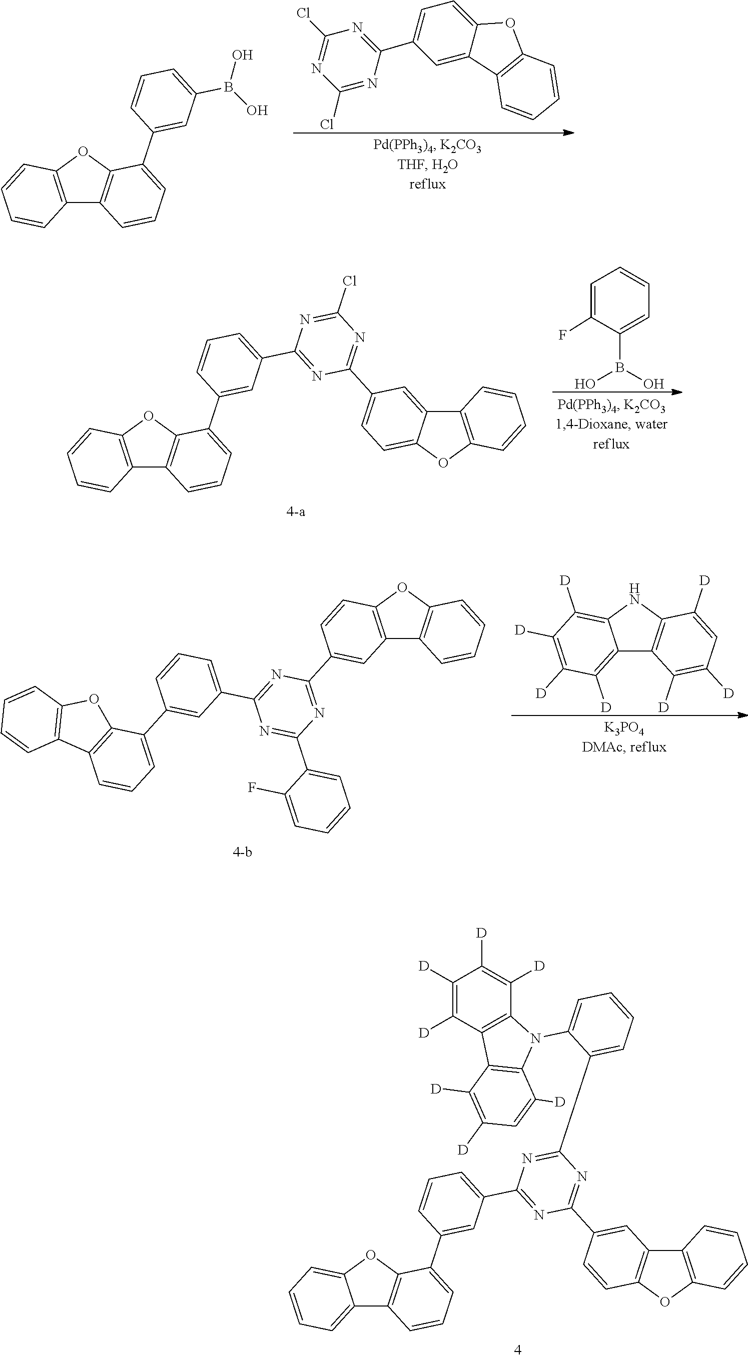

Compound 4 was prepared in the same manner as in the preparation method of Compound 1, except that in Preparation Example 1, (3-(dibenzo[b,d]furan-4-yl)phenyl)boronic acid and 2,4-dichloro-6-(dibenzo[b,d]furan-2-yl)-1,3,5-triazine were used instead of (3-(dibenzo[b,d]thiophen-4-yl)phenyl)boronic acid and 2,4-dichloro-6-phenyl-1,3,5-triazine. (MS: [M+H]⁺=738.9)

### Preparation Example 5: Preparation of Compound 5

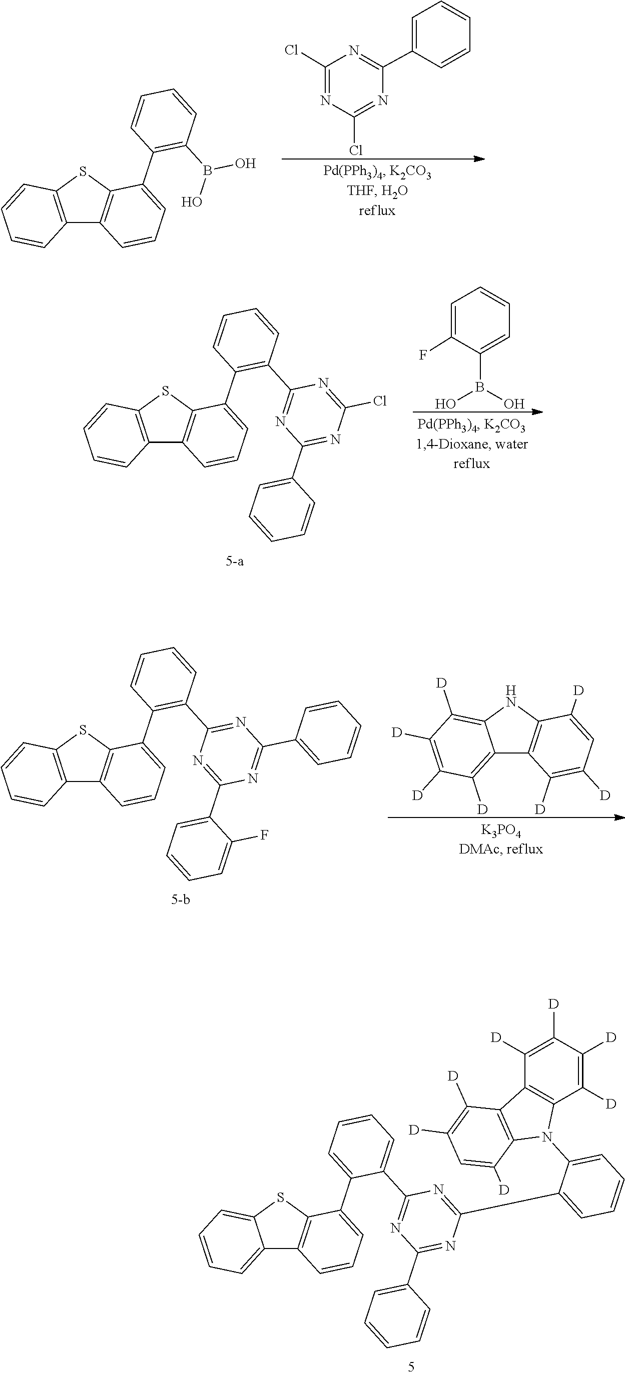

Compound 5 was prepared in the same manner as in the preparation method of Compound 1, except that in Preparation Example 1, (2-(dibenzo[b,d]thiophen-4-yl)phenyl)boronic acid was used instead of (3-(dibenzo[b,d]thiophen-4-yl)phenyl)boronic acid. (MS: [M+H]⁺=664.9)

### Preparation Example 6: Preparation of Compound 6

### Step 1) Preparation of Compound 6-a

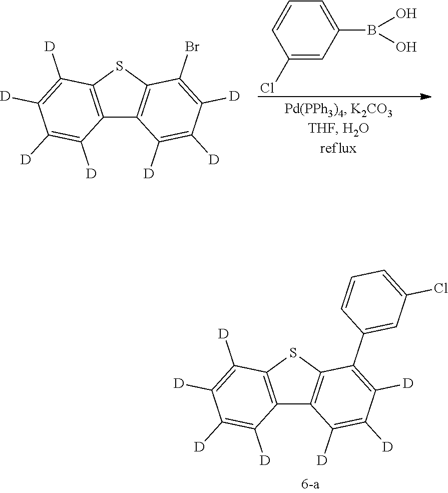

4-Bromodibenzo[b,d]thiophene-1,2,3,6,7,8,9-d7 (30 g, 111 mmol) and (3-chlorophenyl)boronic acid (17.4 g, 111 mmol) were added to 450 ml of tetrahydrofuran under a nitrogen atmosphere, and the mixture was stirred and refluxed. Then, potassium carbonate (46 g, 333.1 mmol) was dissolved in 140 ml of water and added thereto, and the mixture was sufficiently stirred and then tetrakistriphenyl-phosphinopalladium (2.6 g, 2.2 mmol) was added. After the reaction for 6 hours, the reaction mixture was cooled to room temperature, and the resulting solid was filtered. The solid was added to and dissolved in 1005 mL of chloroform, and washed twice with water. The organic layer was then separated, anhydrous magnesium sulfate was added thereto, stirred, then filtered, and the filtrate was distilled under reduced pressure. The concentrated compound was recrystallized from chloroform and ethyl acetate to prepare Compound 6-a as a gray solid (26.8 g, 80%, MS: [M+H]⁺=302.8).

### Step 2) Preparation of Compound 6-b

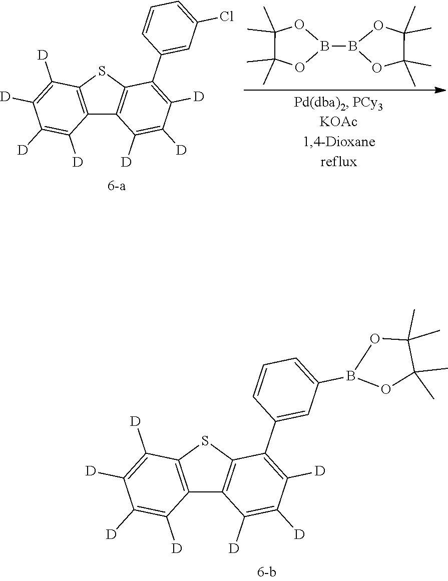

6-a (30 g, 99.4 mmol) and bis(pinacolato)diboron (30.3 g, 119.3 mmol) were added to 450 ml of 1,4-dioxane under a nitrogen atmosphere, and the mixture was stirred and refluxed. Then, potassium acetate (28.7 g, 298.2 mmol) was added thereto, the mixture was sufficiently stirred and then palladiumdibenzylideneacetonepalladium (1.7 g, 3 mmol) and tricyclohexylphosphine (1.7 g, 6 mmol) were added. After the reaction for 4 hours, the reaction mixture was cooled to room temperature, the organic layer was filtered to remove salt, and then the filtered organic layer was distilled. This was again added to and dissolved in 391 mL of chloroform, washed twice with water, and the organic layer was separated. Anhydrous magnesium sulfate was added thereto, stirred, filtered, and the filtrate was distilled under reduced pressure. The concentrated compound was purified by a silica column using chloroform and ethyl acetate to prepare Compound 6-b as a yellow solid (29.3 g, 75%, MS: [M+H]+=394.4).

### Step 3) Preparation of Compound 6-c

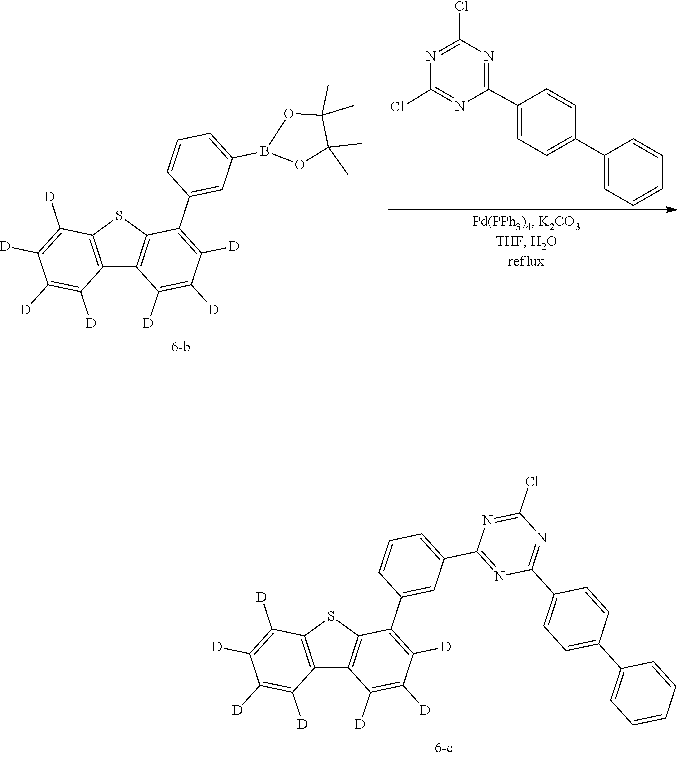 [SMILES: [2H]c1c([2H])c([2H])c2c(sc3c(-c4cccc(B5OC(C)(C)C(C)(C)O5)c4)c([2H])c([2H])c([2H])c32)c1[2H] ; Clc1nc(Cl)nc(-c2ccc(-c3ccccc3)cc2)n1 ; [2H]c1c([2H])c([2H])c2c(sc3c(-c4cccc(-c5nc(Cl)nc(-c6ccc(-c7ccccc7)cc6)n5)c4)c([2H])c([2H])c([2H])c32)c1[2H]]

6-b (30 g, 76.3 mmol) and 2-([1,1′-diphenyl]-4-yl)-4,6-dichloro-1,3,5-triazine (23 g, 76.3 mmol) were added to 450 ml of tetrahydrofuran under a nitrogen atmosphere, and the mixture was stirred and refluxed. Then, potassium carbonate (31.6 g, 228.8 mmol) was dissolved in 130 ml of water and added thereto, the mixture was sufficiently stirred and then tetrakistriphenyl-phosphinopalladium (1.8 g, 1.5 mmol) was added. After the reaction for 8 hours, the reaction mixture was cooled to room temperature, and the resulting solid was filtered. The solid was added to and dissolved in 1220 mL of chloroform, and washed twice with water. The organic layer was then separated, anhydrous magnesium sulfate was added thereto, stirred, then filtered, and the filtrate was distilled under reduced pressure. The concentrated compound was recrystallized from chloroform and ethyl acetate to prepare Compound 6-c as a gray solid (32.5 g, 80%, MS: [M+H]⁺=534.1).

### Step 4) Preparation of Compound 6-d

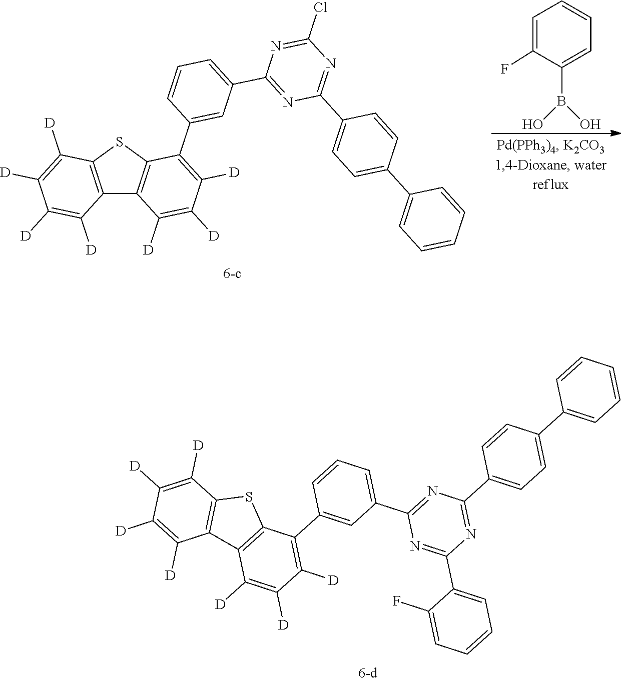 [SMILES: [2H]c1c([2H])c([2H])c2c(sc3c(-c4cccc(-c5nc(Cl)nc(-c6ccc(-c7ccccc7)cc6)n5)c4)c([2H])c([2H])c([2H])c32)c1[2H] ; OB(O)c1ccccc1F ; [2H]c1c([2H])c([2H])c2c(sc3c(-c4cccc(-c5nc(-c6ccc(-c7ccccc7)cc6)nc(-c6ccccc6F)n5)c4)c([2H])c([2H])c([2H])c32)c1[2H]]

6-c (30 g, 56.3 mmol) and (2-fluorophenyl)boronic acid (7.9 g, 56.3 mmol) were added to 450 ml of 1,4-dioxane under a nitrogen atmosphere, and the mixture was stirred and refluxed. Then, potassium carbonate (23.3 g, 168.8 mmol) was dissolved in 70 ml of water and added thereto, the mixture was sufficiently stirred and then tetrakistriphenyl-phosphinopalladium (1.3 g, 1.1 mmol) was added. After the reaction for 4 hours, the reaction mixture was cooled to room temperature, and the resulting solid was filtered. The solid was added to and dissolved in 1001 mL of chloroform, and washed twice with water. The organic layer was then separated, anhydrous magnesium sulfate was added thereto, stirred, then filtered, and the filtrate was distilled under reduced pressure. The concentrated compound was recrystallized from chloroform and ethyl acetate to prepare Compound 6-d as a gray solid (23.7 g, 71%, MS: [M+H]⁺=593.7).

### Step 5) Preparation of Compound 6

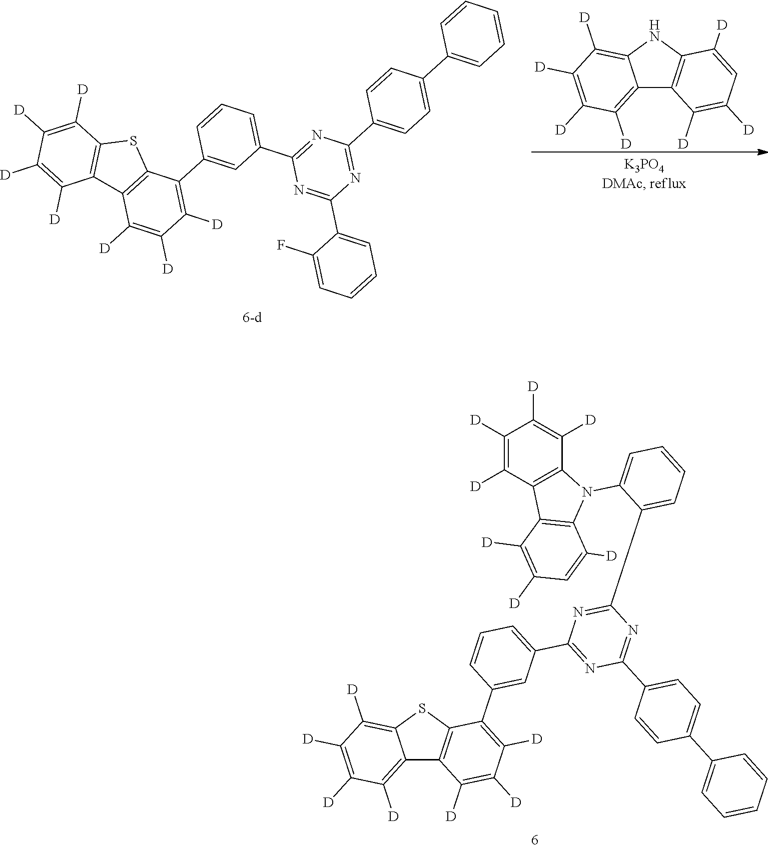

6-d (25 g, 42.2 mmol) and 9H-carbazole-1,2,3,4,5,6,8-d7 (7.3 g, 42.2 mmol) were added to 200 ml of dimethylacetamide under a nitrogen atmosphere, and the mixture was stirred and refluxed. Then, potassium phosphate tribasic (26.9 g, 126.5 mmol) was added thereto, the mixture was sufficiently stirred and then reacted for 3 hours. The reaction mixture was cooled to room temperature, the organic layer was filtered to remove salt, and then the filtered organic layer was distilled. This was again added to and dissolved in 945 mL of chloroform, washed twice with water, and the organic layer was separated. Anhydrous magnesium sulfate was added thereto, stirred, filtered, and the filtrate was distilled under reduced pressure. The concentrated compound was purified by a silica column using chloroform and ethyl acetate to prepare Compound 6 as a yellow solid (20.2 g, 64%, MS: [M+H]+=748).

### Preparation Example 7: Preparation of Compound 7

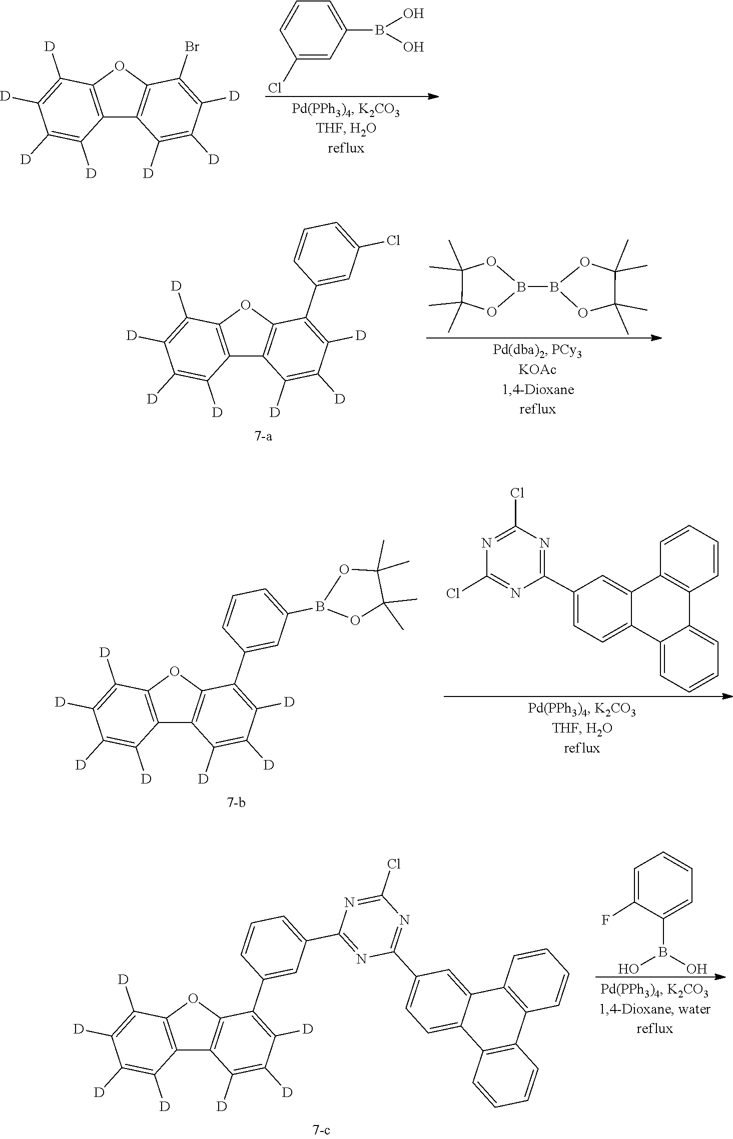

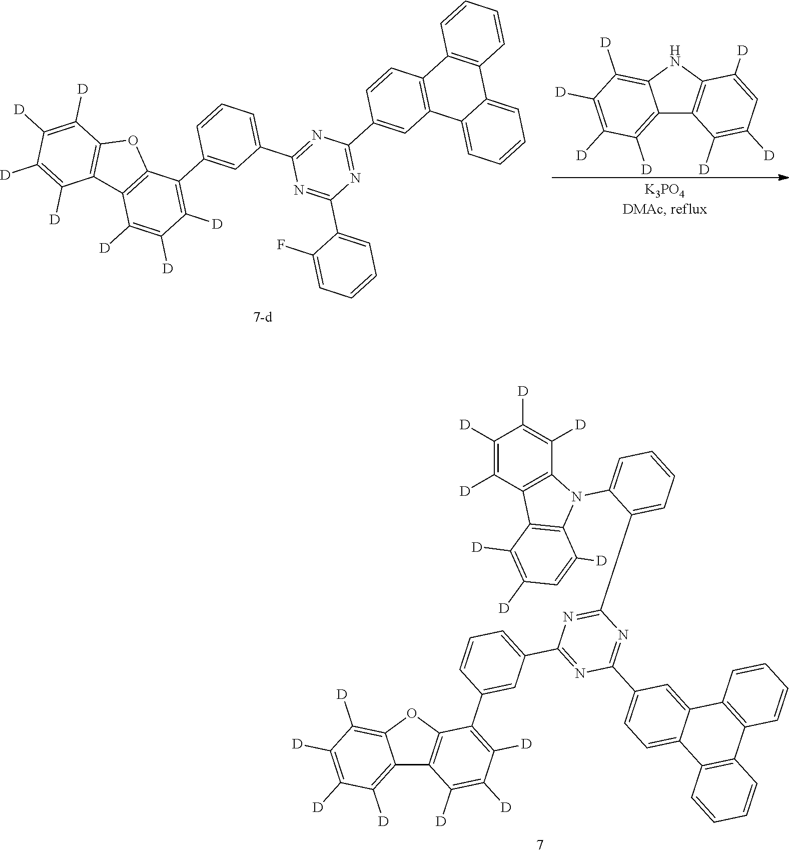

Compound 7 was prepared in the same manner as in the preparation method of Compound 1, except that in the preparation method of Compound 6, except that in Preparation Example 6, 4-bromodibenzo[b,d]furan-d7 and 2,4-dichloro-6-triphenylen-2-yl)-1,3,5-triazine were used instead of 4-bromodibenzo[b,d]thiophene-d7 and 2-([1,1′-diphenyl]-4-yl)-4,6-dichloro-1,3,5-triazine. (MS: [M+H]⁺=806)

### Preparation Example 8: Preparation of Compound 8

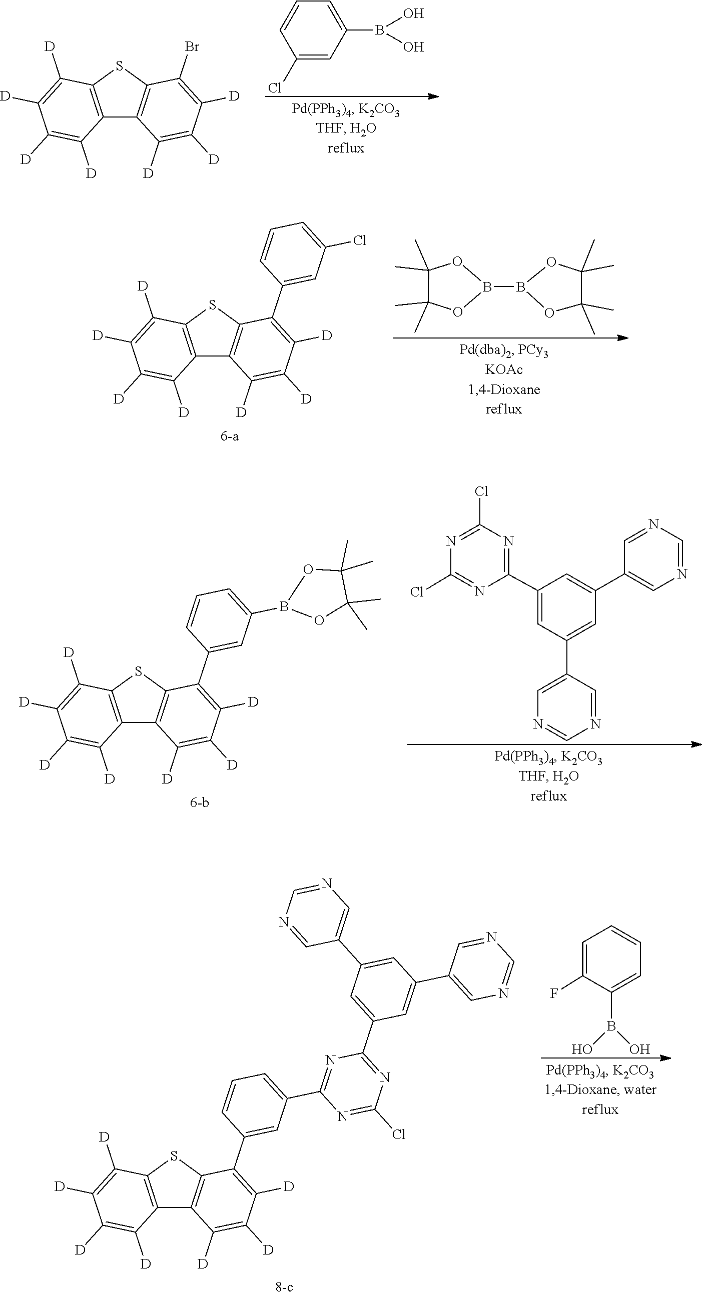

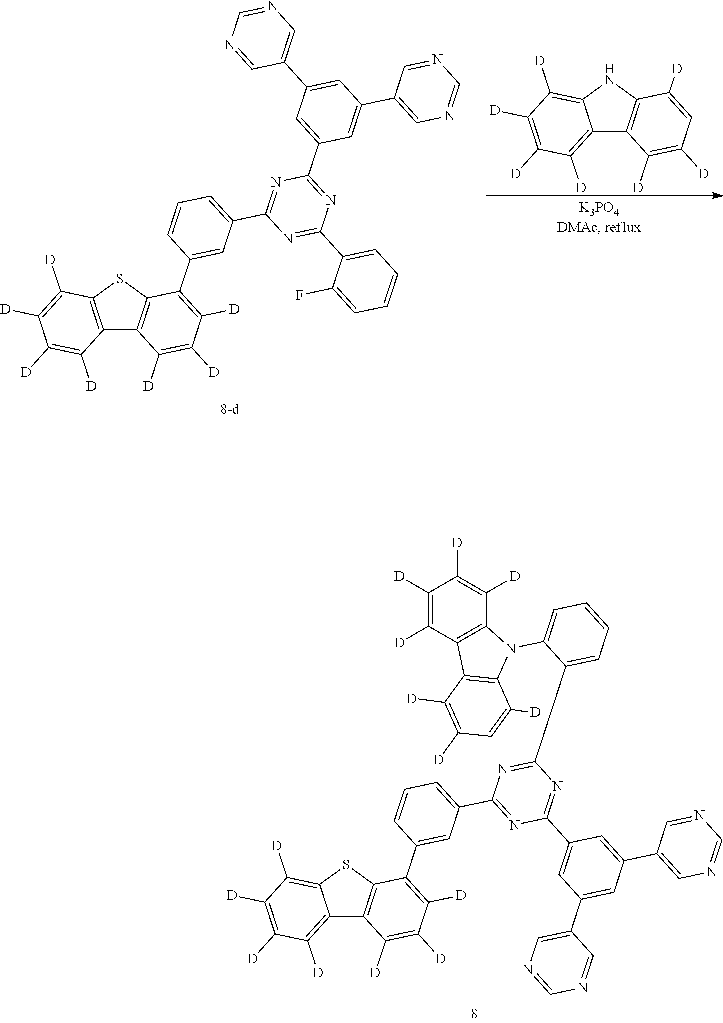

Compound 8 was prepared in the same manner as in the preparation method of Compound 1, except that in the preparation method of Compound 6, except that in Preparation Example 6, 2,4-dichloro-6-3,5-di(pyrimidin-5-yl)phenyl)-1,3,5-triazine was used instead of 2-([1,1′-diphenyl]-4-yl)-4,6-dichloro-1,3,5-triazine. (MS: [M+H]⁺=826)

### Examples

### Example 1: Manufacture of Organic Light Emitting Device

A glass substrate on which ITO (indium tin oxide) was coated as a thin film to a thickness of 1400 Å was put into distilled water in which a detergent was dissolved, and ultrasonically cleaned. At this time, a product manufactured by Fischer Co. was used as the detergent, and as the distilled water, distilled water filtered twice using a filter manufactured by Millipore Co. was used. After the ITO was cleaned for 30 minutes, ultrasonic cleaning was repeated twice using distilled water for 10 minutes. After the cleaning with distilled water was completed, the substrate was ultrasonically cleaned with solvents of isopropyl alcohol, acetone, and methanol, dried, and then transferred to a plasma cleaner. In addition, the substrate was cleaned for 5 minutes using oxygen plasma and then transferred to a vacuum depositor.

On the ITO transparent electrode thus prepared, the following compound HT-A and the following compound PD were thermally vacuum-deposited at a weight ratio of 95:5 to a thickness of 100 Å, and then only the following compound HT-A was deposited to a thickness of 1150 Å to form a hole transport layer. The following compound HT-B was thermally vacuum-deposited to a thickness of 450 Å on the hole transport layer to form an electron blocking layer. The previously prepared compound 1 and the following compound GD were vacuum-deposited at a weight ratio of 85:15 to a thickness of 400 Å on the electron blocking layer to form a light emitting layer. The following compound ET-A was vacuum-deposited to a thickness of 50 Å on the light emitting layer to form a hole blocking layer. The following compound ET-B and the following compound Liq were thermally vacuum-deposited at a weight ratio of 2:1 to a thickness of 250 Å on the hole blocking layer, and then Lithium fluoride (LiF) and magnesium were vacuum-deposited at a weight ratio of 1:1 to a thickness of 30 Å to form an electron transport and injection layer. Magnesium and silver were deposited at a weight ratio of 1:4 to a thickness of 160 Å on the electron injection layer to form a cathode, thereby completing the manufacture of an organic light emitting device.

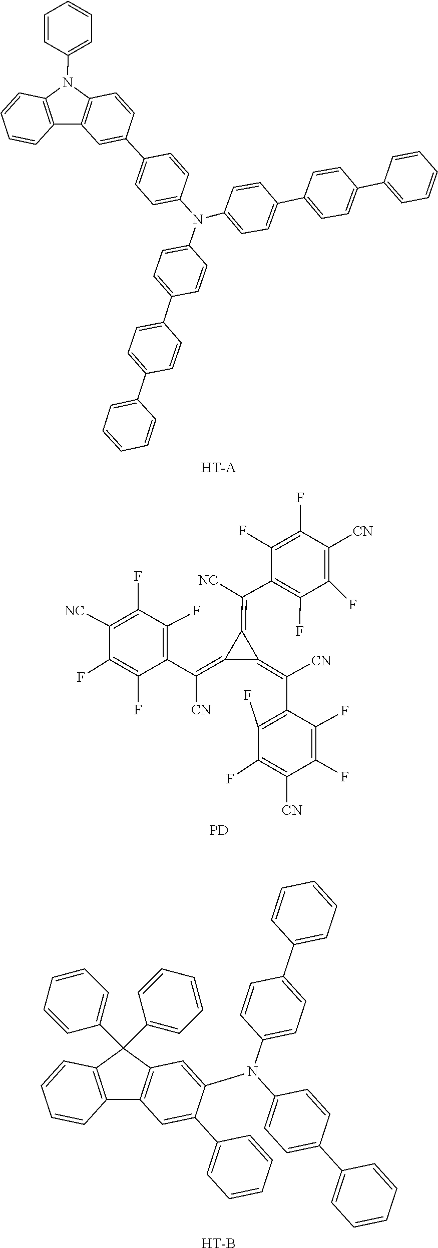 [SMILES: c1ccc(-c2ccc(-c3ccc(N(c4ccc(-c5ccc(-c6ccccc6)cc5)cc4)c4ccc(-c5ccc6c(c5)c5ccccc5n6-c5ccccc5)cc4)cc3)cc2)cc1 ; "HT-A" ; N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; "PD" ; c1ccc(-c2ccc(N(c3ccc(-c4ccccc4)cc3)c3cc4c(cc3-c3ccccc3)-c3ccccc3C4(c3ccccc3)c3ccccc3)cc2)cc1 ; "HT-B"]

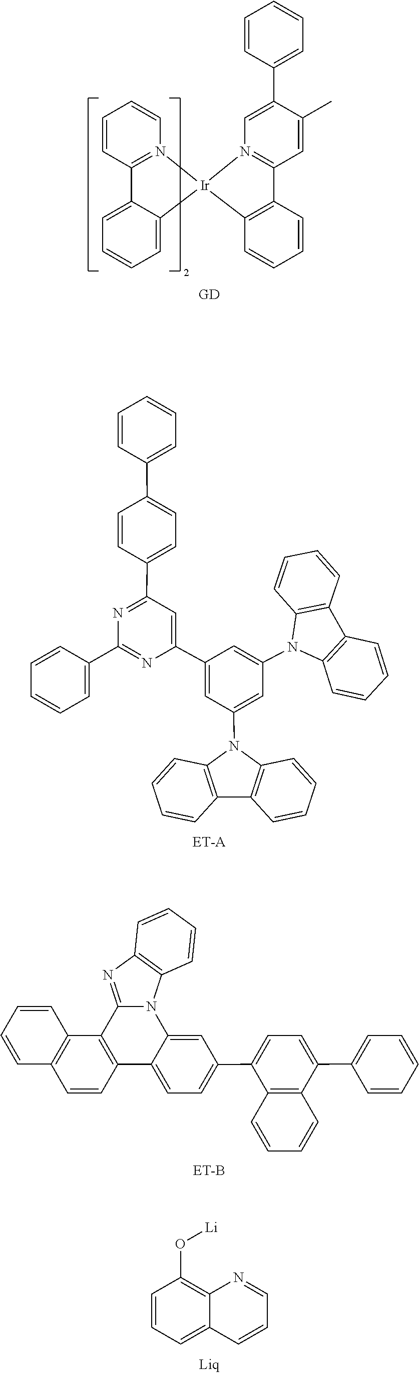

In the above-mentioned processes, the vapor deposition rate of the organic material was maintained at 0.4˜0.7 Å/sec, the deposition rates of lithium fluoride of the cathode was maintained at 0.3 Å/sec and 2 Å/sec, the deposition rate of magnesium and silver was maintained at 2 Å/sec, and the degree of vacuum during the deposition was maintained at 2×10⁻⁷˜5×10⁻⁶ torr, thereby manufacturing an organic light emitting device.

### Examples 2 to 8 and Comparative Examples 1 to 5

The organic light emitting devices of Examples 2 to 8 and Comparative Examples 1 to 5 were respectively manufactured in the same manner as in Example 1, except that the compounds listed in Table 1 below were used instead of Compound 1 in Example 1. The compounds GH-A to GH-E listed in Table 1 below are as follows.

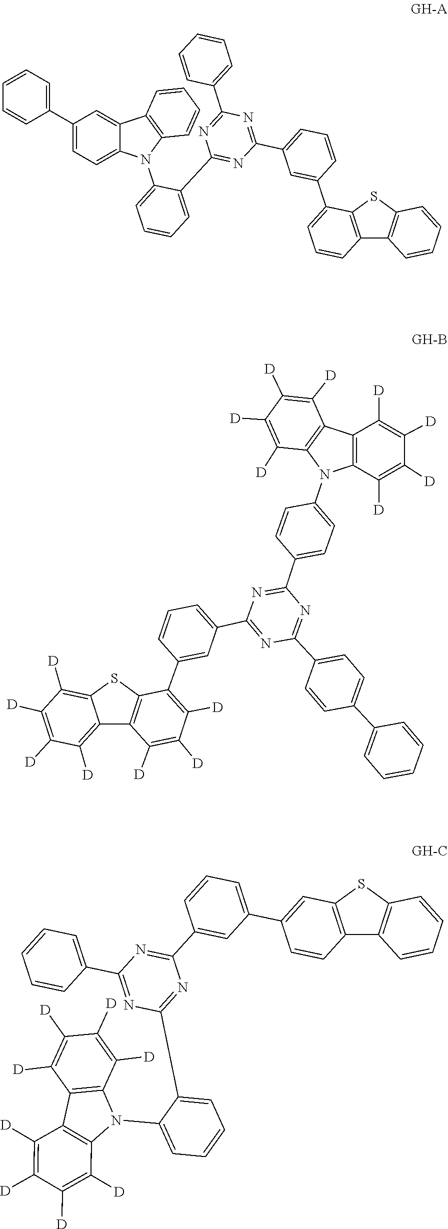 [SMILES: c1ccc(-c2ccc3c(c2)c2ccccc2n3-c2ccccc2-c2nc(-c3ccccc3)nc(-c3cccc(-c4cccc5c4sc4ccccc45)c3)n2)cc1 ; [2H]c1c([2H])c([2H])c2c(sc3c(-c4cccc(-c5nc(-c6ccc(-c7ccccc7)cc6)nc(-c6ccc(-n7c8c([2H])c([2H])c([2H])c([2H])c8c8c([2H])c([2H])c([2H])c([2H])c87)cc6)n5)c4)c([2H])c([2H])c([2H])c32)c1[2H] ; [2H]c1c([2H])c([2H])c2c(c1[2H])c1c([2H])c([2H])c([2H])c([2H])c1n2-c1ccccc1-c1nc(-c2ccccc2)nc(-c2cccc(-c3ccc4c(c3)sc3ccccc34)c2)n1]

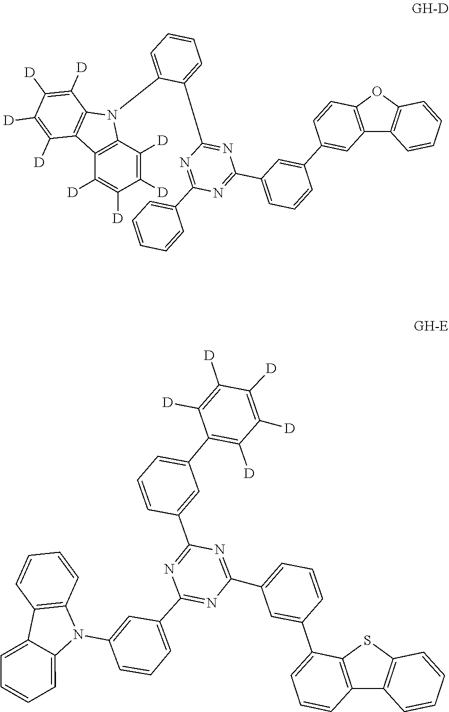 [SMILES: [2H]c1c([2H])c([2H])c2c(c1[2H])c1c([2H])c([2H])c([2H])c([2H])c1n2-c1ccccc1-c1nc(-c2ccccc2)nc(-c2cccc(-c3ccc4oc5ccccc5c4c3)c2)n1 ; [2H]c1c([2H])c([2H])c(-c2cccc(-c3nc(-c4cccc(-c5cccc6c5sc5ccccc56)c4)nc(-c4cccc(-n5c6ccccc6c6ccccc65)c4)n3)c2)c([2H])c1[2H]]

### Experimental Example

The voltage, efficiency and lifetime (T95) were measured by applying a current to the organic light emitting devices manufactured in the Examples and Comparative Examples, and the results are shown in Table 1 below. At this time, the voltage and efficiency were measured by applying a current density of 10 mA/cm². Further, T95 shown in Table 1 means the time required for the luminance to be reduced to 95% of the initial luminance at a current density of 20 mA/cm².

TABLE 1

| | Light emitting | Voltage | Efficiency | Lifetime |
|---|---|---|---|---|
| | layer (host) | (V) | (cd/A) | (T95, hr) |
| Example 1 | Compound 1 | 3.67 | 72.5 | 85 |
| Example 2 | Compound 2 | 3.75 | 67 | 77 |
| Example 3 | Compound 3 | 3.72 | 66.1 | 87 |
| Example 4 | Compound 4 | 3.50 | 60.4 | 81 |
| Example 5 | Compound 5 | 4.01 | 57.4 | 78 |
| Example 6 | Compound 6 | 3.99 | 69.1 | 77 |
| Example 7 | Compound 7 | 4.12 | 66.5 | 69 |
| Example 8 | Compound 8 | 3.74 | 62.9 | 82 |
| Comparative | GH-A | 3.87 | 61.0 | 64 |
| Example 1 | | | | |
| Comparative | GH-B | 3.85 | 35.6 | 45 |
| Example 2 | | | | |
| Comparative | GH-C | 4.58 | 28.2 | 57.5 |
| Example 3 | | | | |
| Comparative | GH-D | 4.51 | 22.1 | 59.4 |
| Example 4 | | | | |
| Comparative | GH-E | 4.01 | 33.5 | 41.4 |
| Example 5 | | | | |

It can be confirmed that the devices of Examples 1 to 8 using the compounds of the present disclosure have improved lifetime characteristics due to deuterium substitution compared to the device of Comparative Example 1, and that the devices of Comparative Examples 2 and 5 have a structure in which triazine and carbazole are adjacent to each other in para- or meta-positions, and thus intra charge transfer does not easily occur, thereby exhibiting lower efficiency and lifetime characteristics than the devices of Examples 1 to 8. In addition, it can be confirmed that the devices of Comparative Examples 3 and 4 are compounds substituted at positions 2 and 3 of dibenzofuran and dibenzothiophene, and have higher voltages, shorter lifetimes, and especially lower efficiency characteristics than the devices of Examples 1 to 8.

Therefore, as shown in Table 1, it can be confirmed that when the compounds of Chemical Formulas 1 to 8 are used as hosts for organic light emitting devices, they exhibit low voltage, high efficiency, and long lifetime characteristics.

[Description of Symbols]

| | |
|---|---|
| 1: substrate | 2: anode |
| 3: light emitting layer | 4: cathode |
| 5: hole injection layer | 6: hole transport layer |
| 7: electron blocking layer | 8: hole blocking layer |
| 9: electron transport and injection layer | |

1. A compound represented by the following Chemical Formula 1: 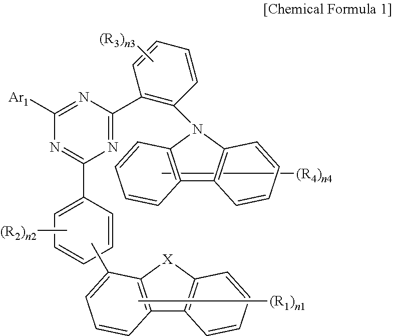
wherein in Chemical Formula 1,
Ar₁ is a substituted or unsubstituted C₆₋₆₀ aryl; or a substituted or unsubstituted C₂₋₆₀ heteroaryl containing at least one selected from the group consisting of N, O and S,
X is O or S,
R₁ is hydrogen; deuterium; a substituted or unsubstituted C₆₋₆₀ aryl; or a substituted or unsubstituted C₂₋₆₀ heteroaryl containing at least one selected from the group consisting of N, O and S,
each R₂ is independently hydrogen; deuterium; or a substituted or unsubstituted C₆₋₆₀ aryl,
each R₃ is independently hydrogen; deuterium; or a substituted or unsubstituted C₆₋₆₀ aryl,
each R₄ is independently hydrogen; deuterium; or a substituted or unsubstituted C₆₋₆₀ aryl,
n1 is an integer of 1 to 7,
n2 and n3 are each independently an integer of 1 to 4, and
n4 is an integer of 1 to 8,
wherein the compound represented by Chemical Formula 1 is substituted with at least one deuterium.

2. The compound of claim 1, wherein:
Ar₁ is phenyl, biphenylyl, terphenylyl, quaterphenylyl, naphthyl, phenanthrenyl, triphenylenyl, dibenzofuranyl, dibenzothiophenyl, benzothiophenyl, carbazolyl, pyridinyl, pyrimidinyl, quinolinyl, isoquinolinyl, quinoxalinyl, naphthyl phenyl, terphenylyl phenyl, phenyl substituted with one or two pyridinyls, phenyl substituted with two pyrimidinyls, carbazolyl phenyl, carbazolyl carbazolyl, or phenyl naphthyl,
wherein the Ar₁ is substituted or unsubstituted with at least one deuterium.

3. The compound of claim 1, wherein:
Ar₁ is any one selected from among the following:

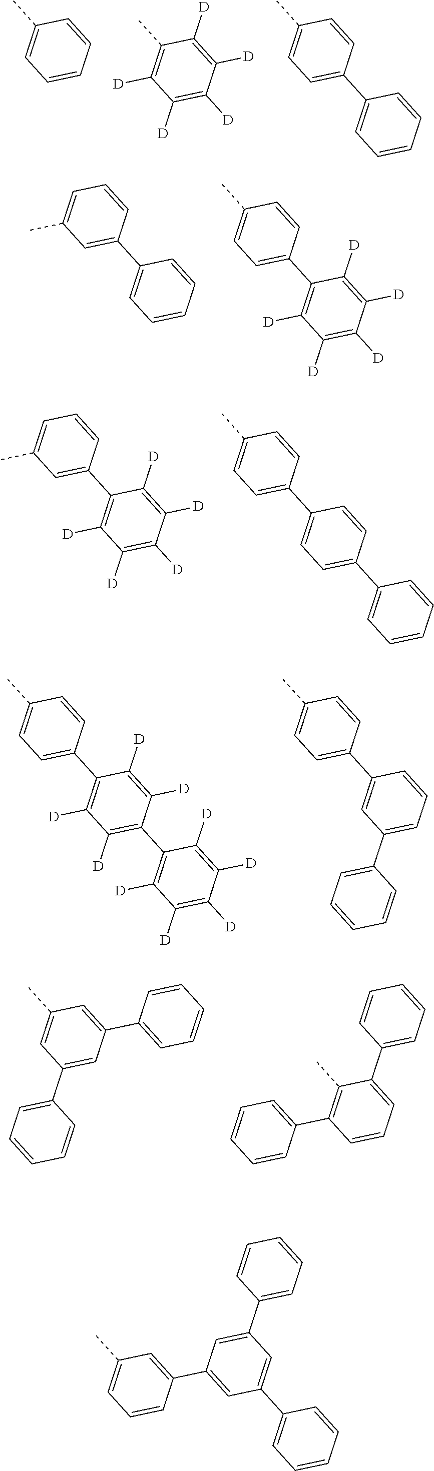

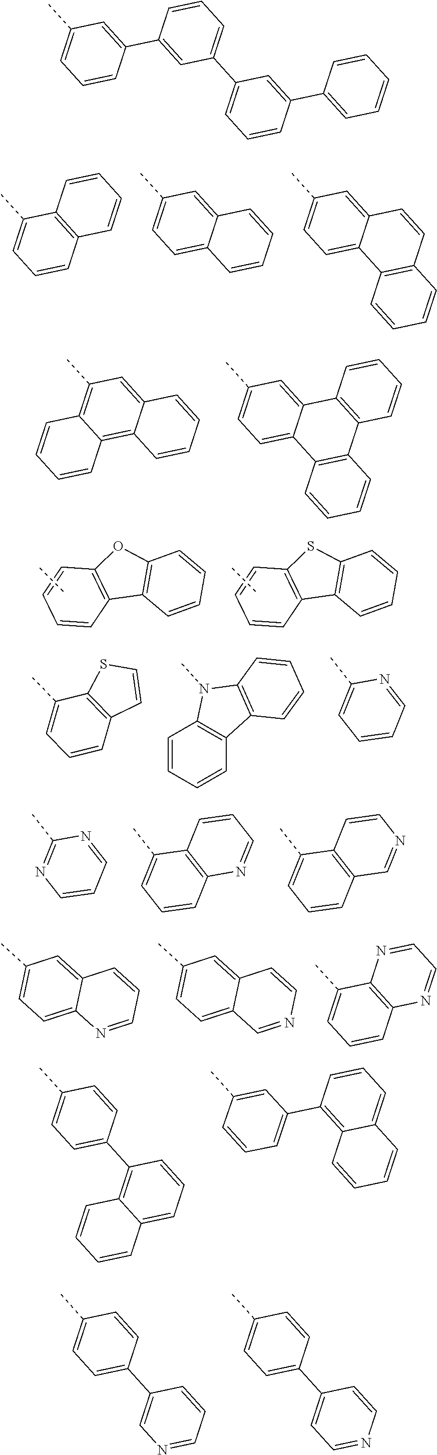

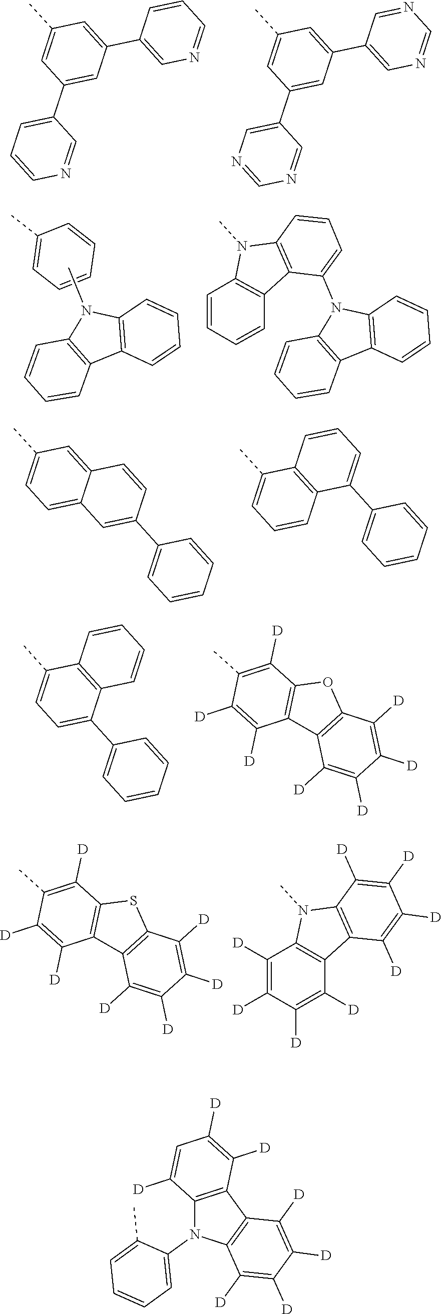

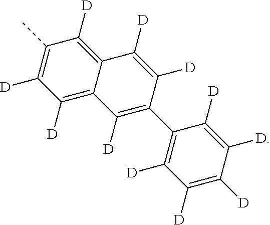

4. The compound of claim 1, wherein:
each R₁ is independently hydrogen, deuterium, a substituted or unsubstituted phenyl, a substituted or unsubstituted dibenzofuranyl, or a substituted or unsubstituted dibenzothiophenyl.

5. The compound of claim 1, wherein:
each R₁ is independently hydrogen, deuterium, phenyl, phenyl substituted with 3 deuterium, phenyl substituted with 5 deuterium, dibenzofuranyl, or dibenzothiophenyl.

6. The compound of claim 1, wherein:
each R₂ is independently hydrogen, deuterium, or a substituted or unsubstituted phenyl,
each R₃ is independently hydrogen, deuterium, or a substituted or unsubstituted phenyl, and
each R₄ is independently hydrogen, deuterium, or a substituted or unsubstituted phenyl.

7. The compound of claim 1, wherein:
each R₂ is independently hydrogen, deuterium, phenyl, phenyl substituted with 3 deuterium, or phenyl substituted with 5 deuterium,
each R₃ is independently hydrogen, deuterium, phenyl, phenyl substituted with 3 deuterium, or phenyl substituted with 5 deuterium, and
each R₄ is independently hydrogen, deuterium, phenyl, phenyl substituted with 3 deuterium, or phenyl substituted with 5 deuterium,

8. The compound of claim 1, wherein:
the compound represented by Chemical Formula 1 is any one compound selected from among the following compounds:

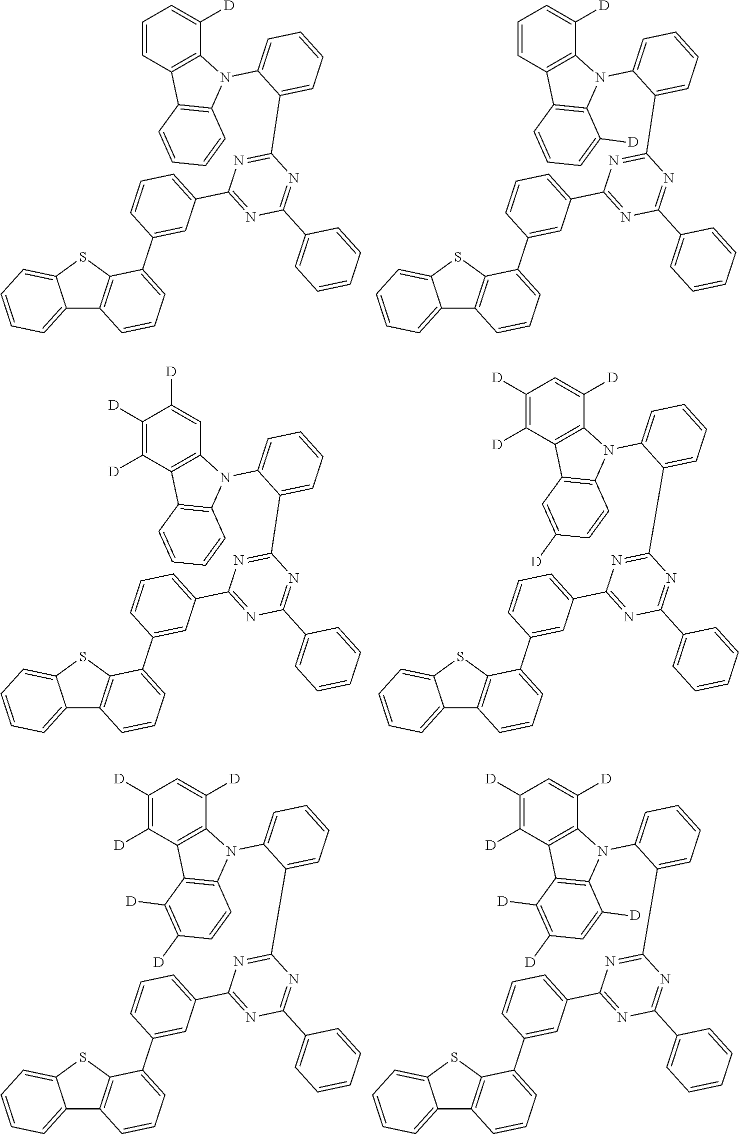

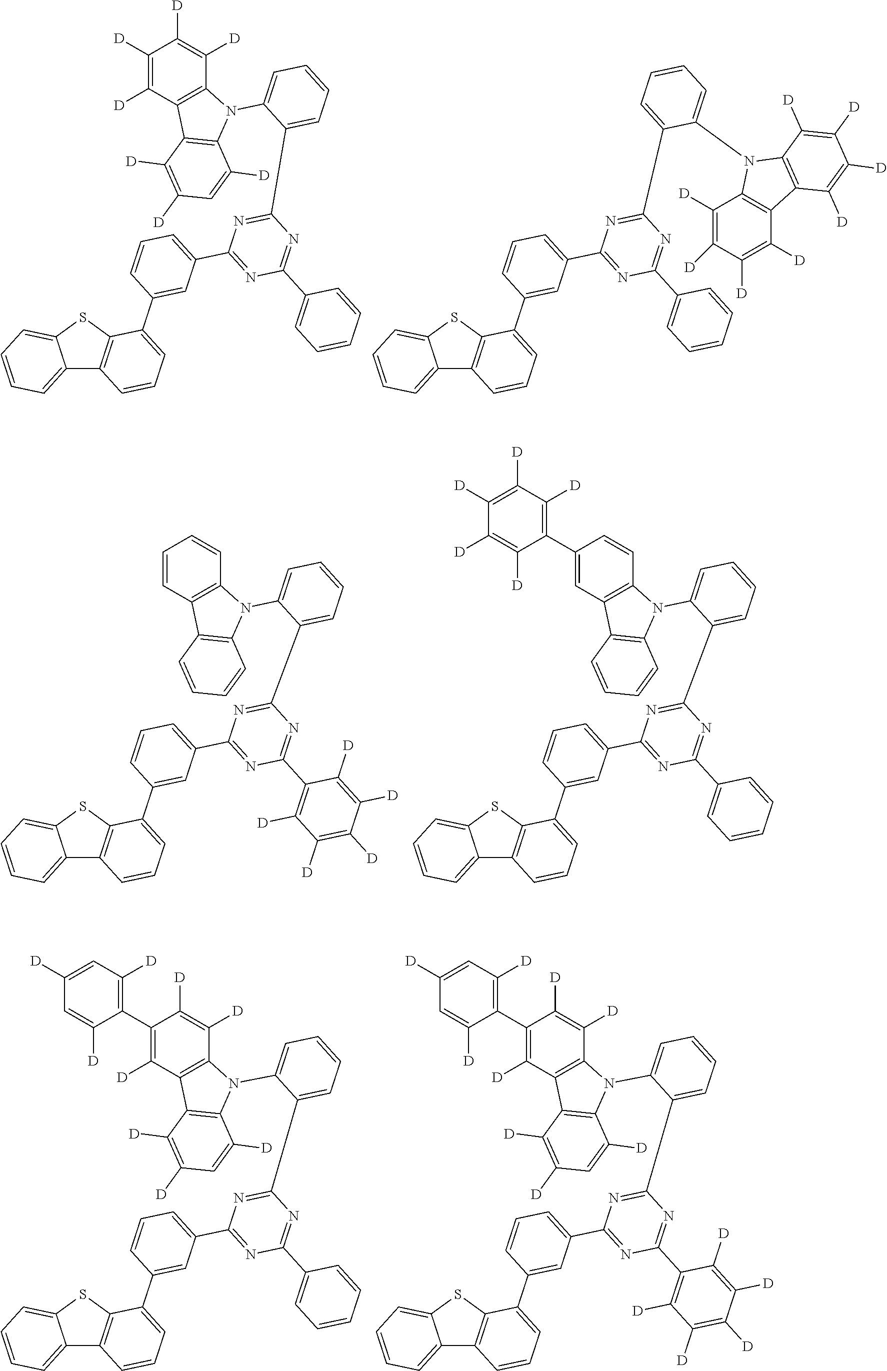

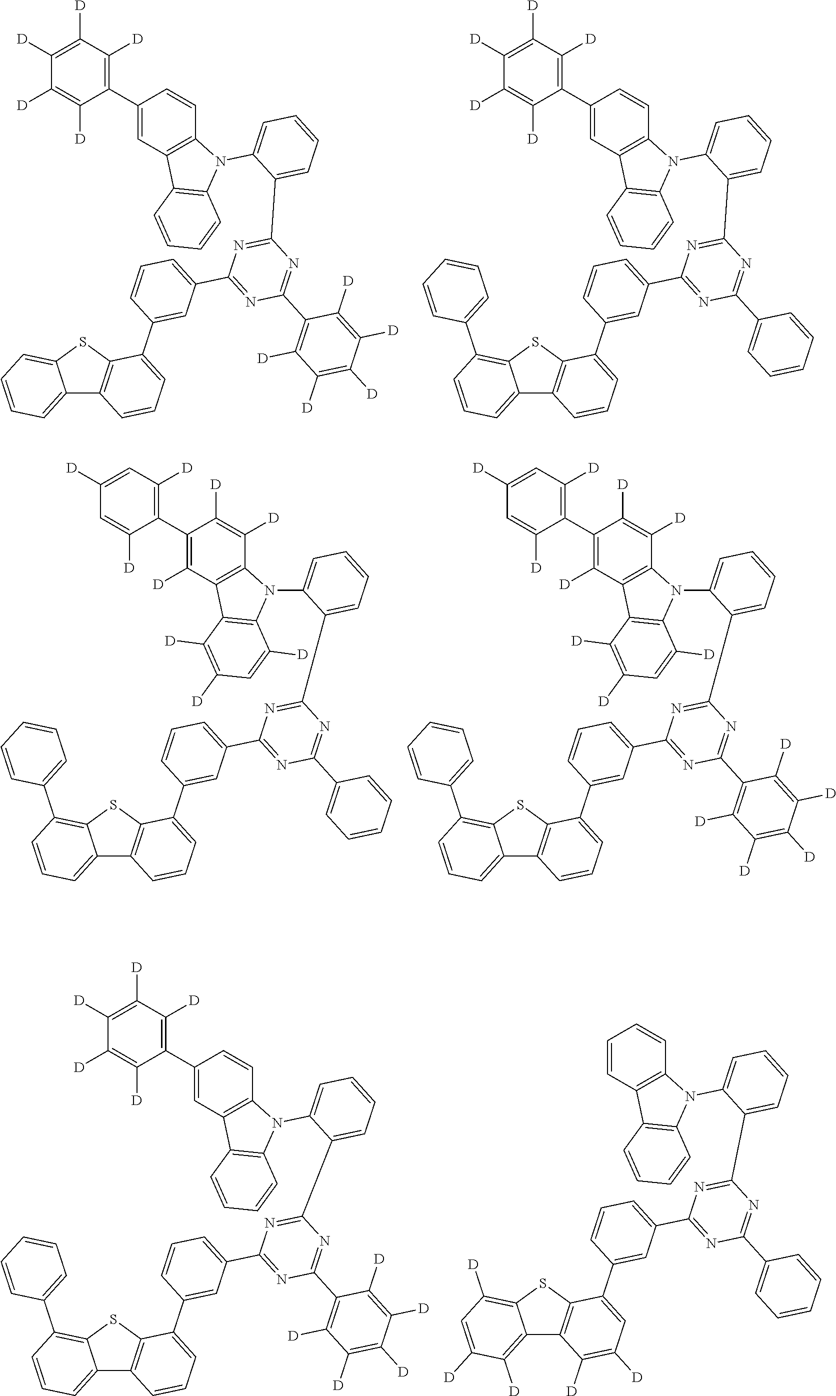

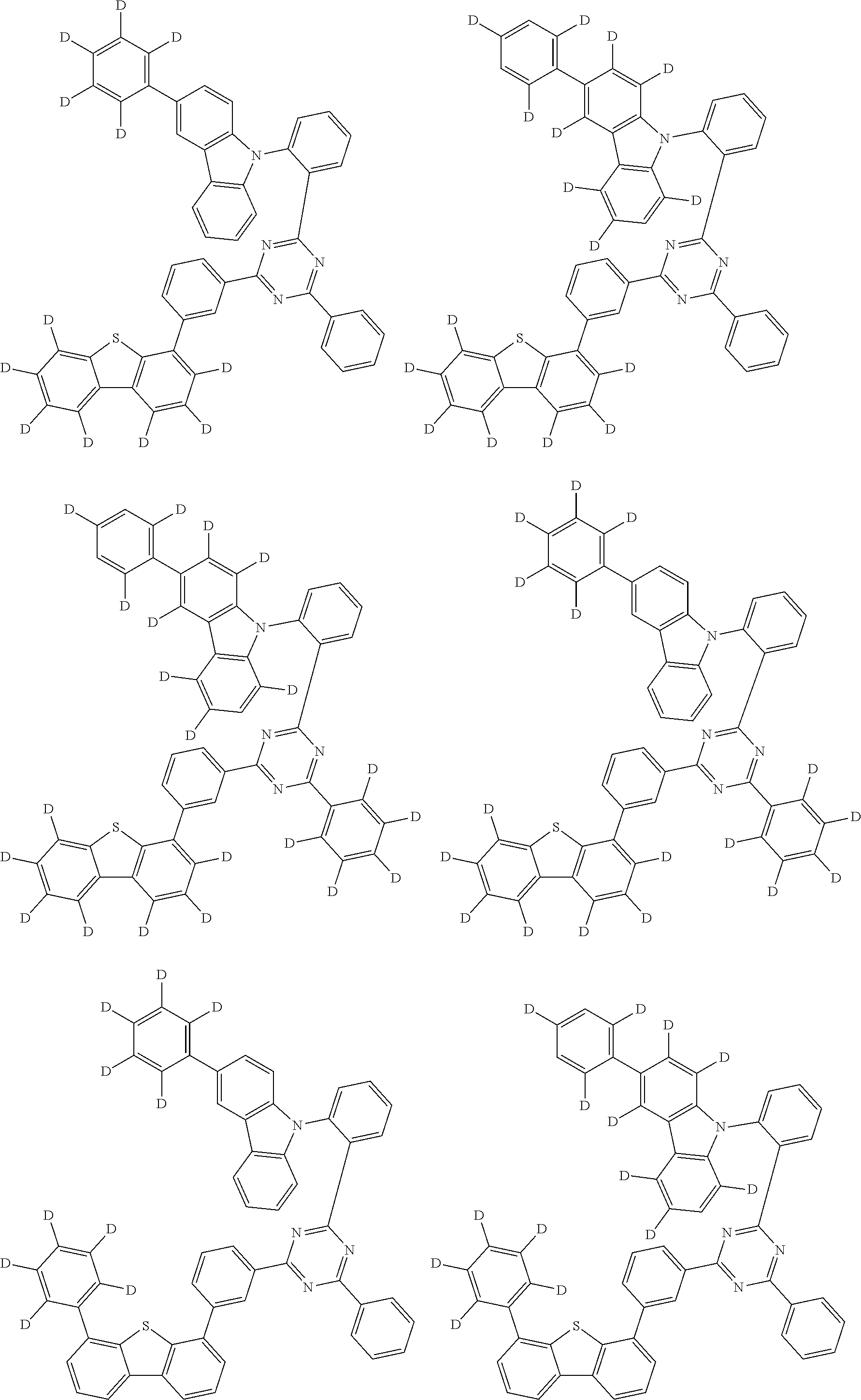

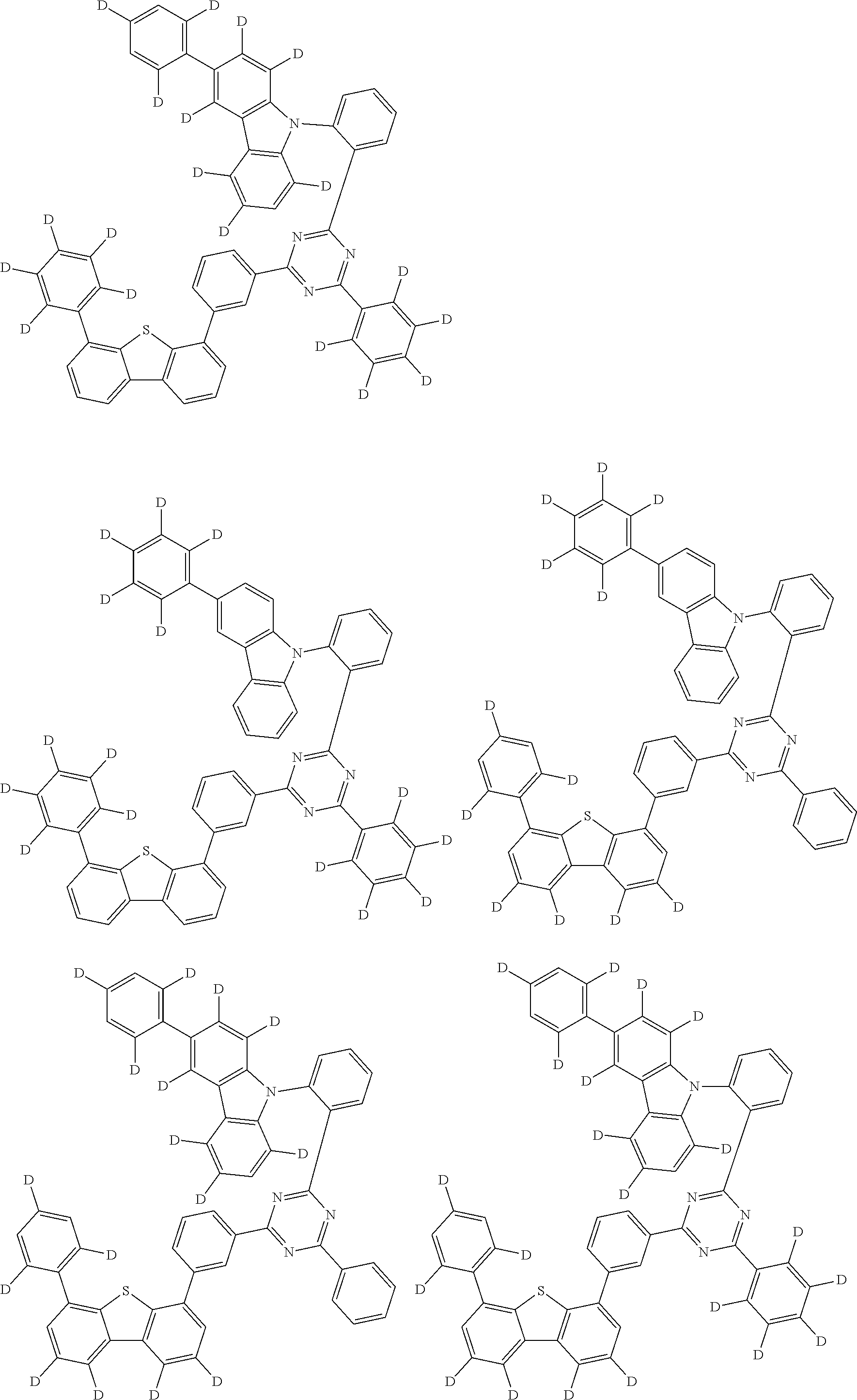

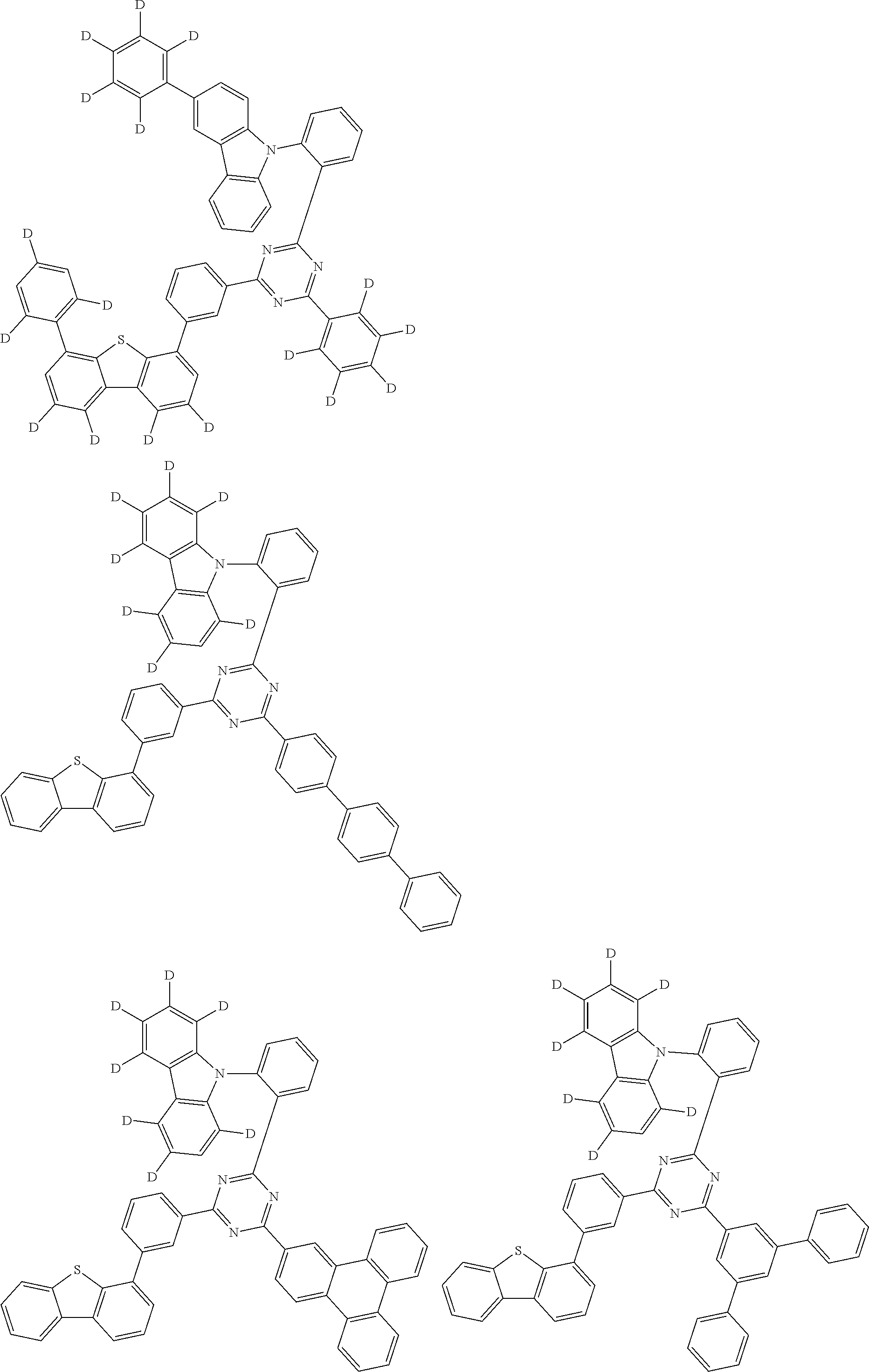

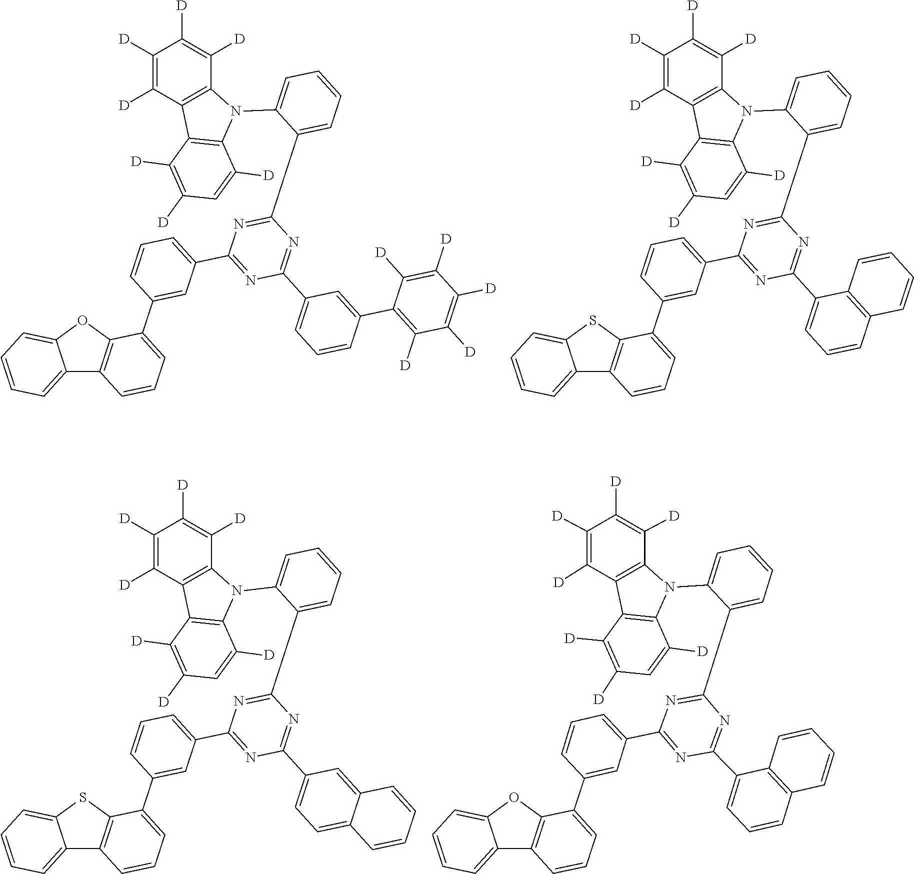

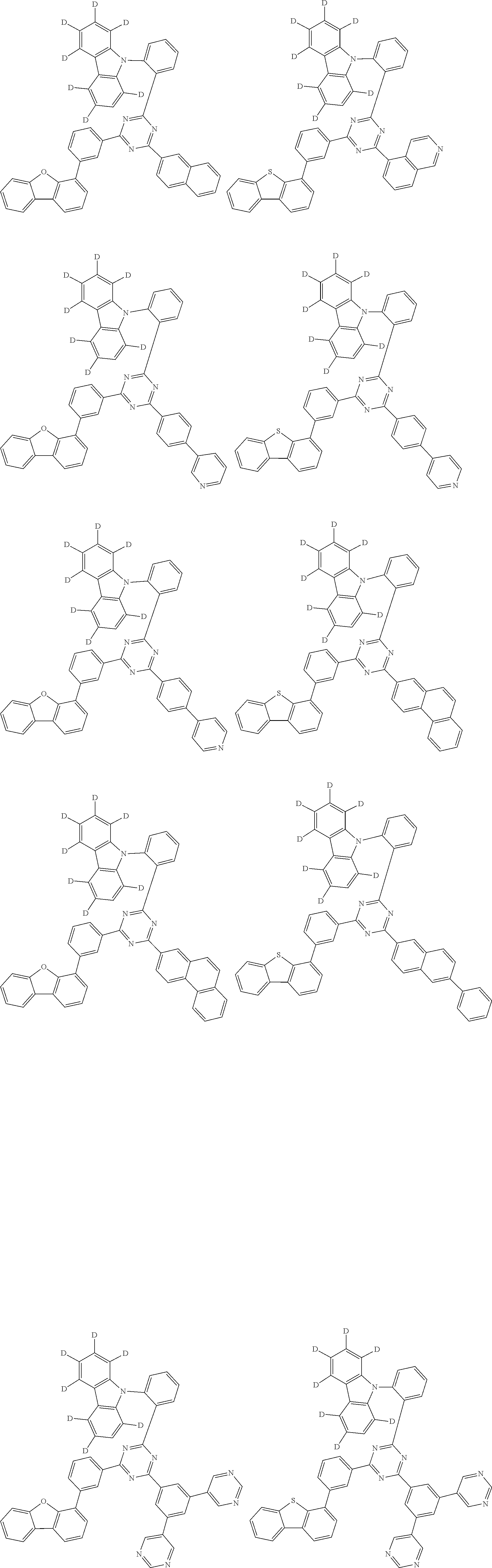

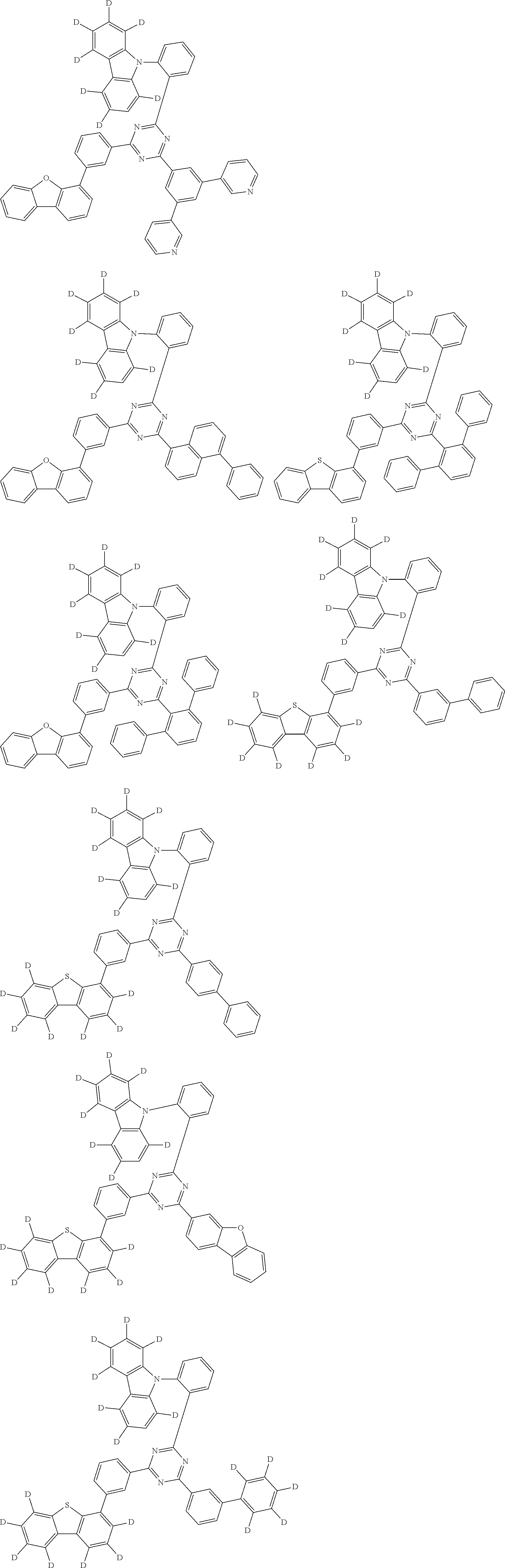

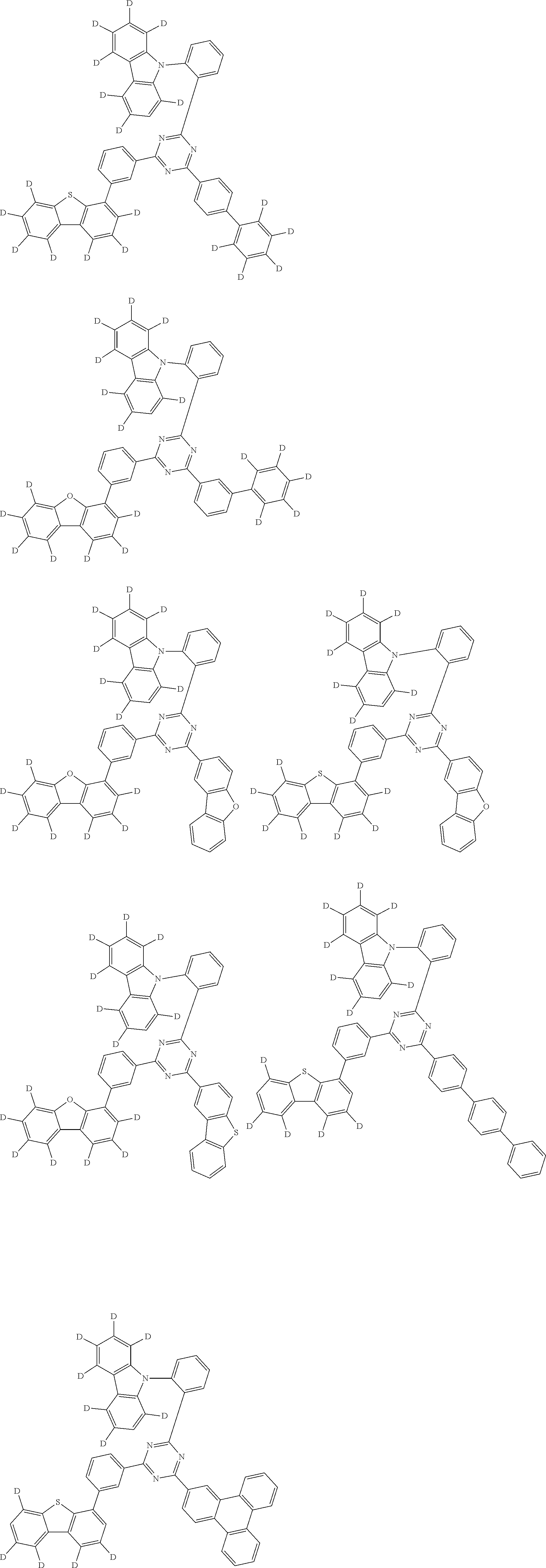

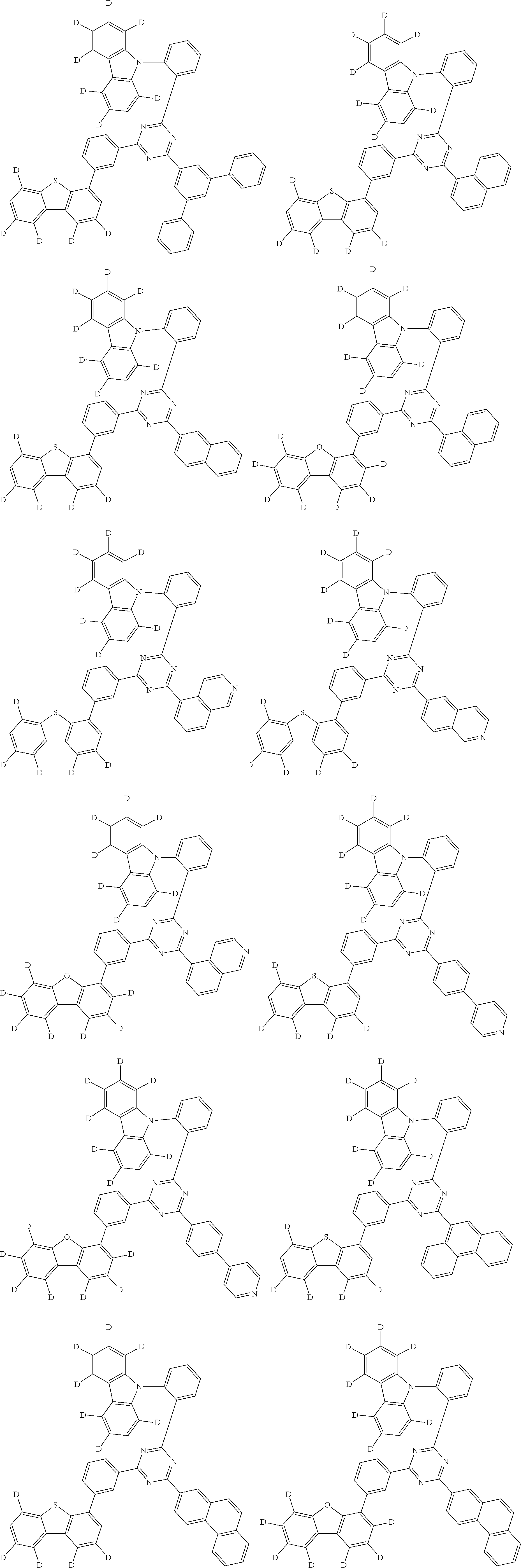

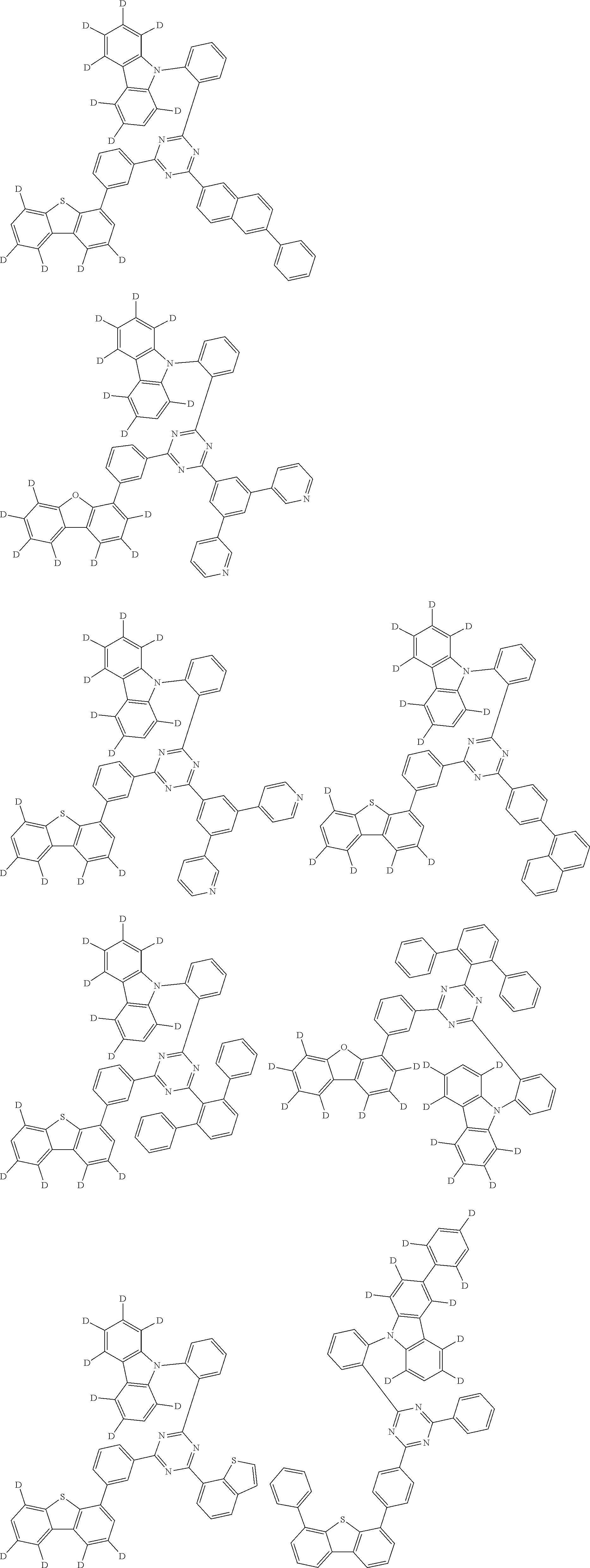

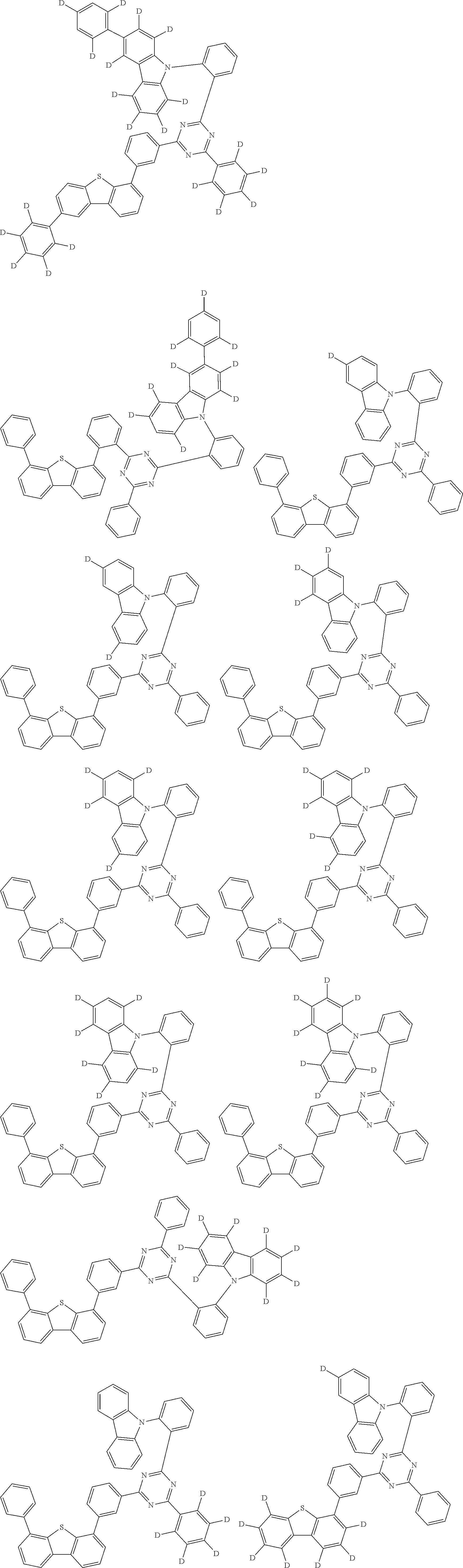

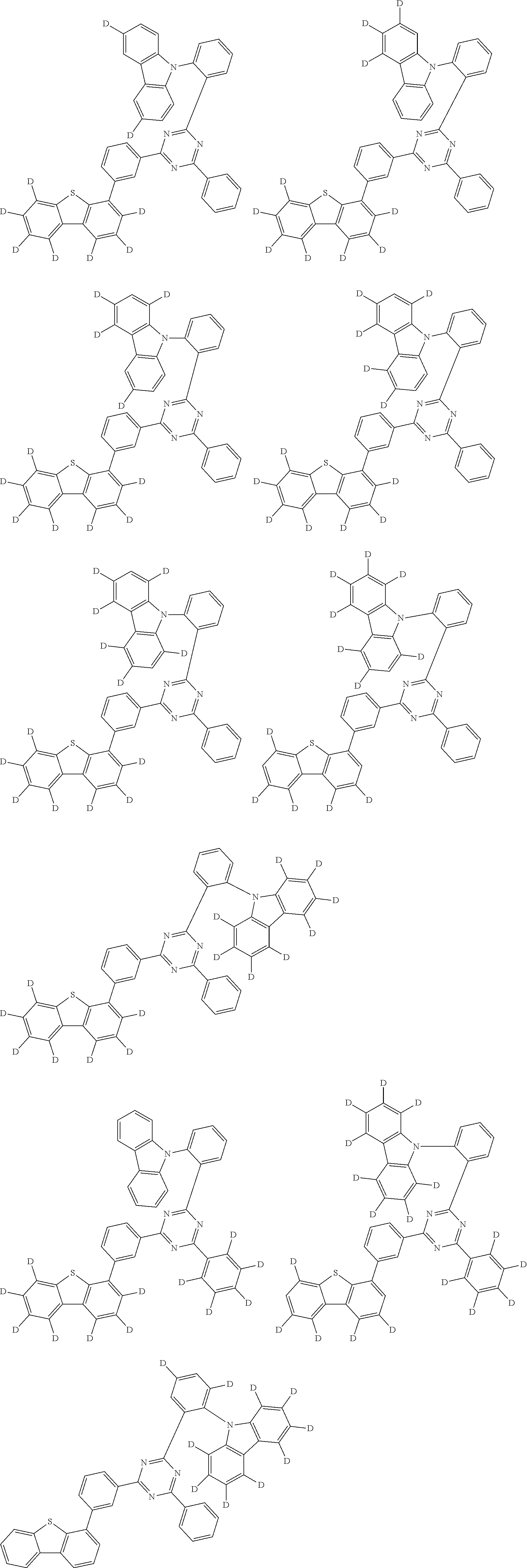

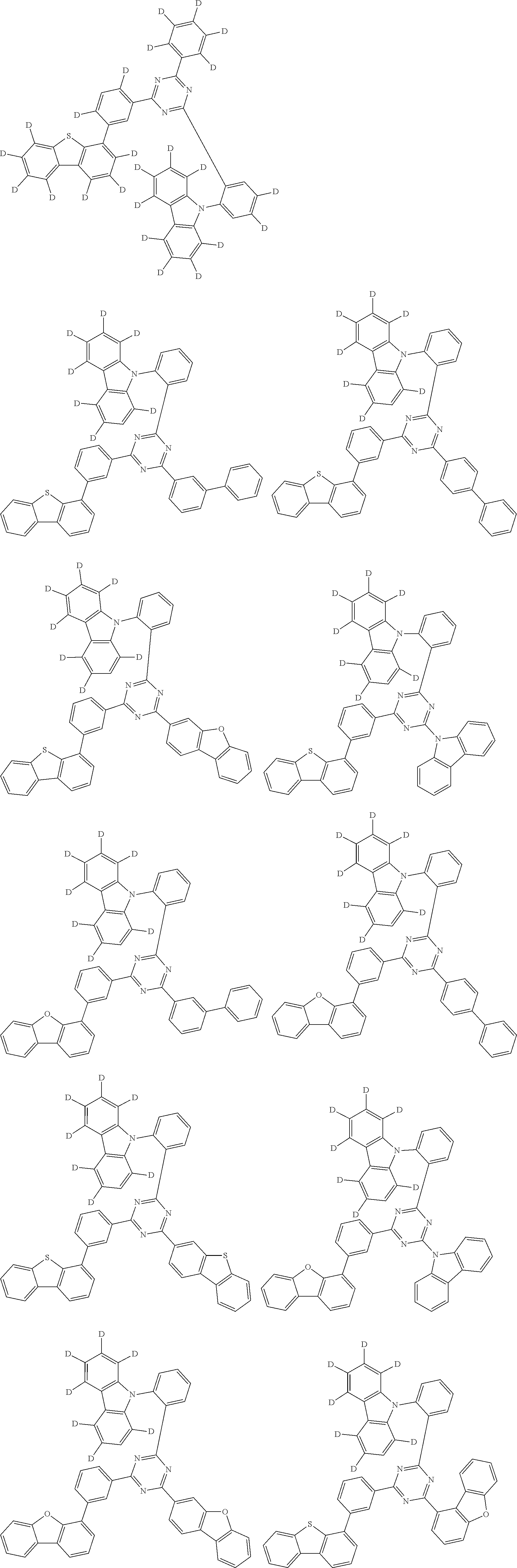

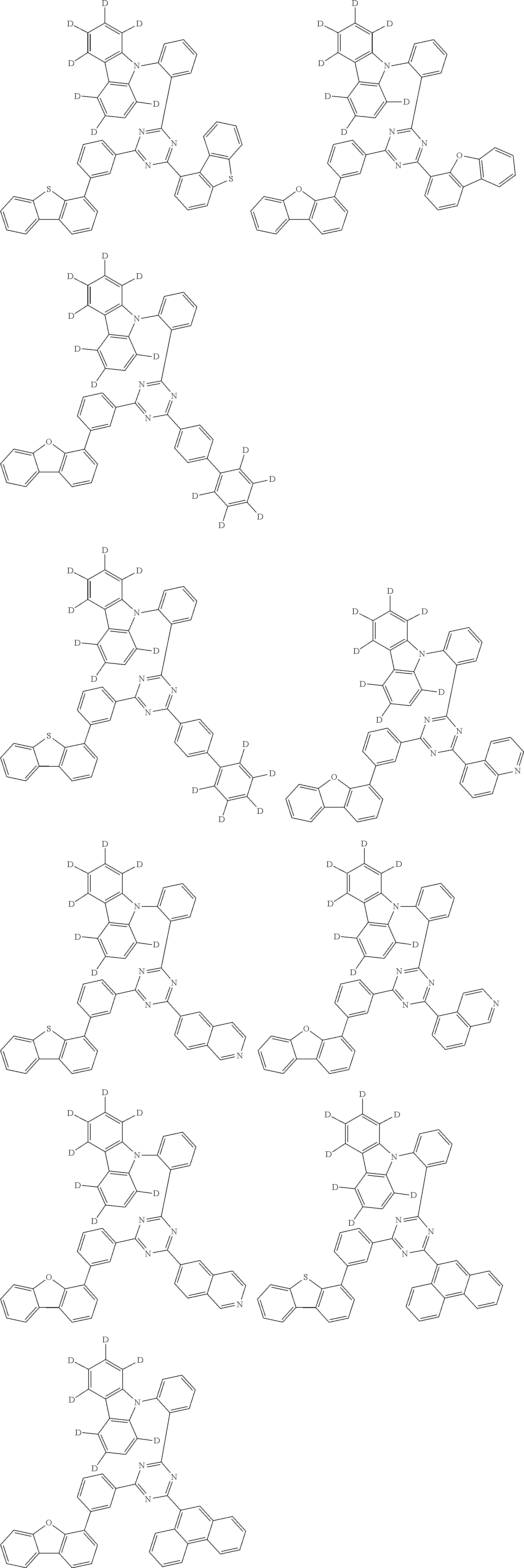

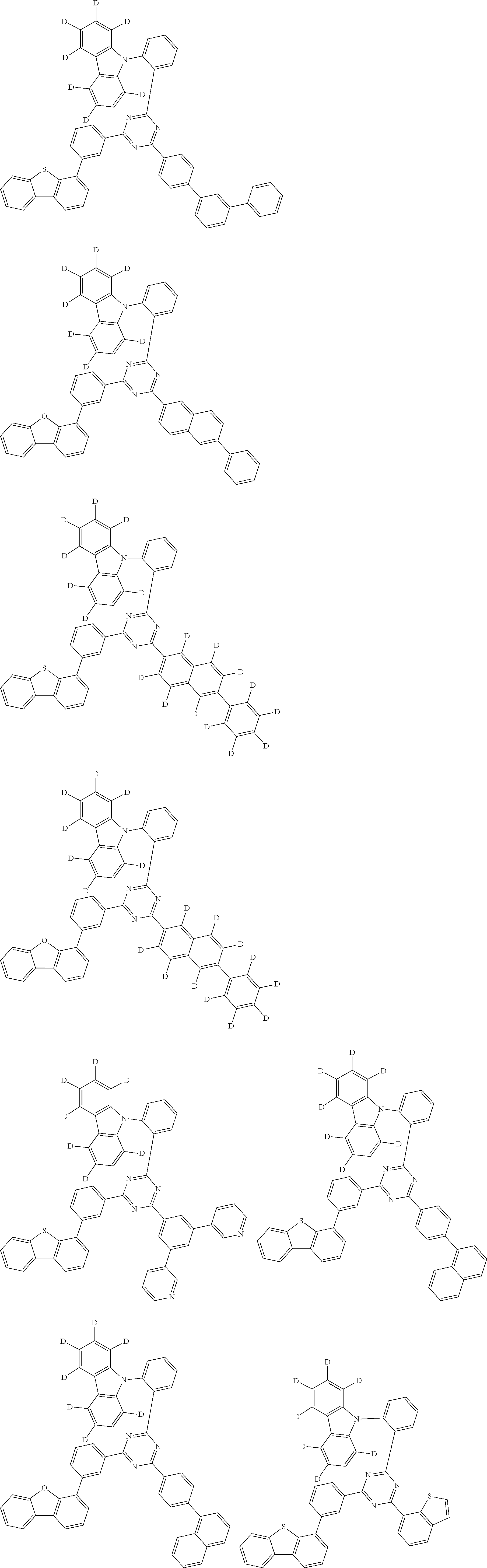

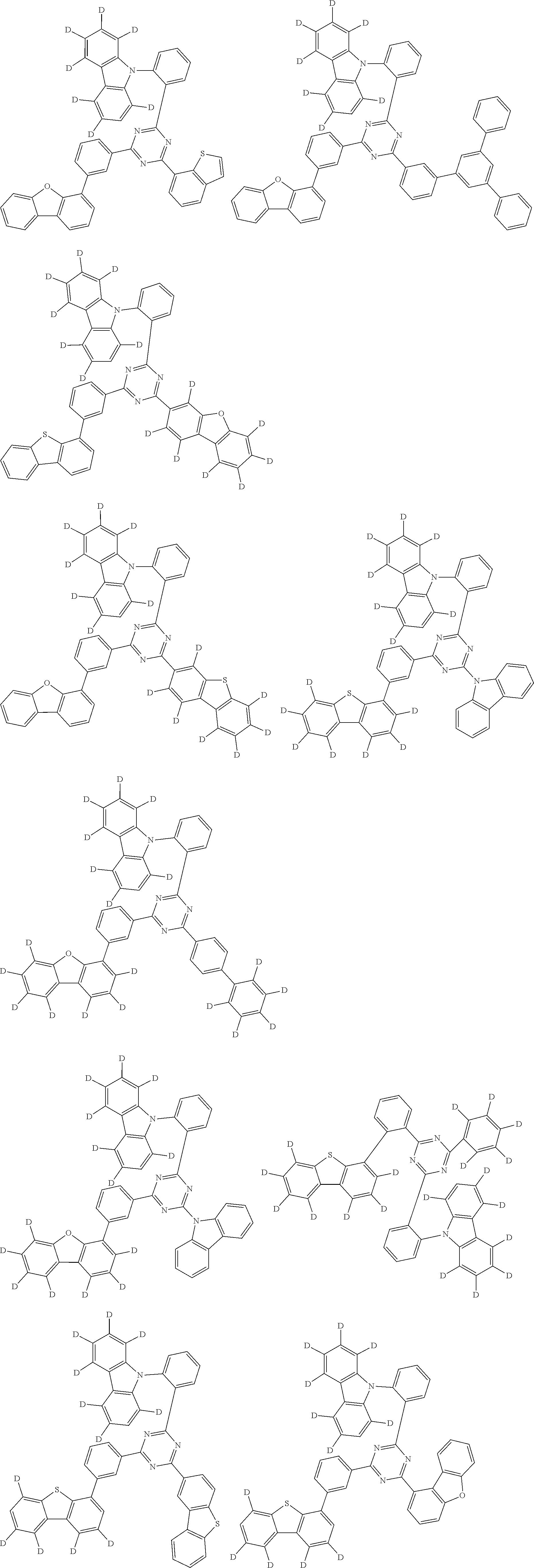

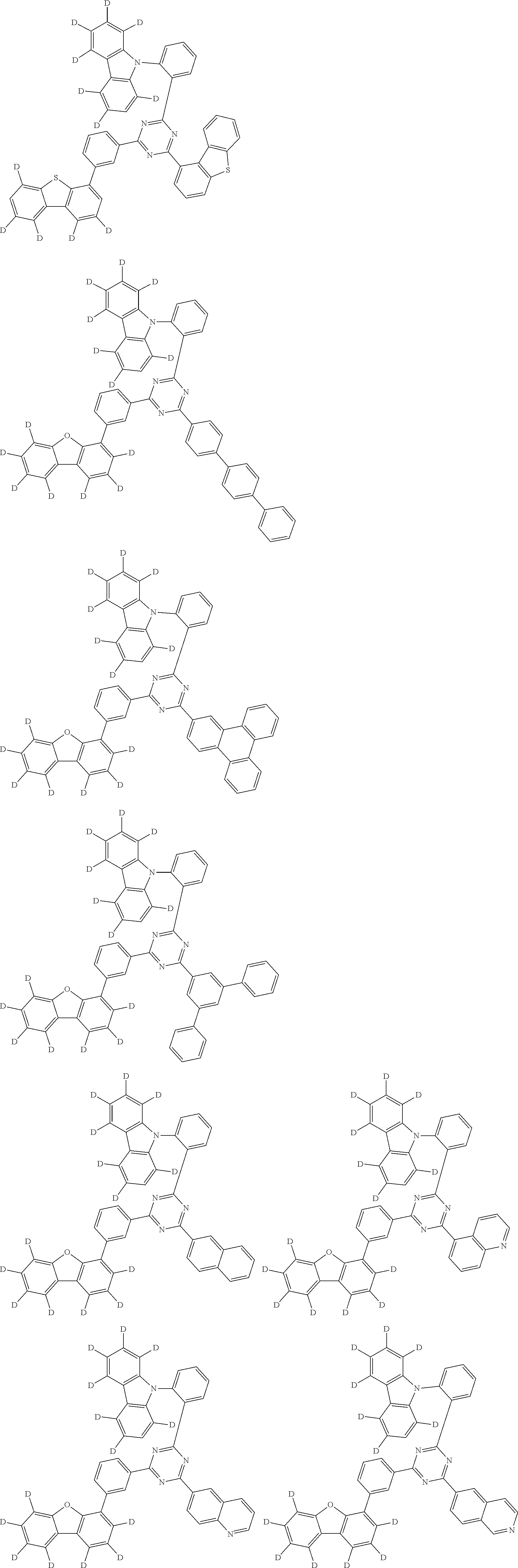

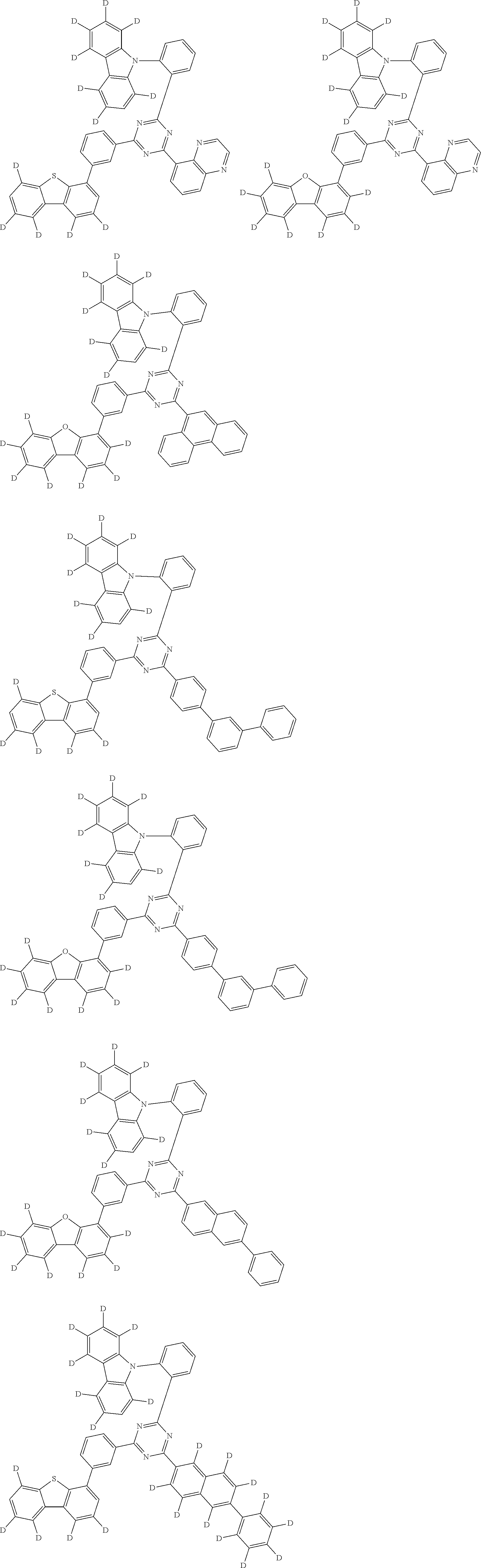

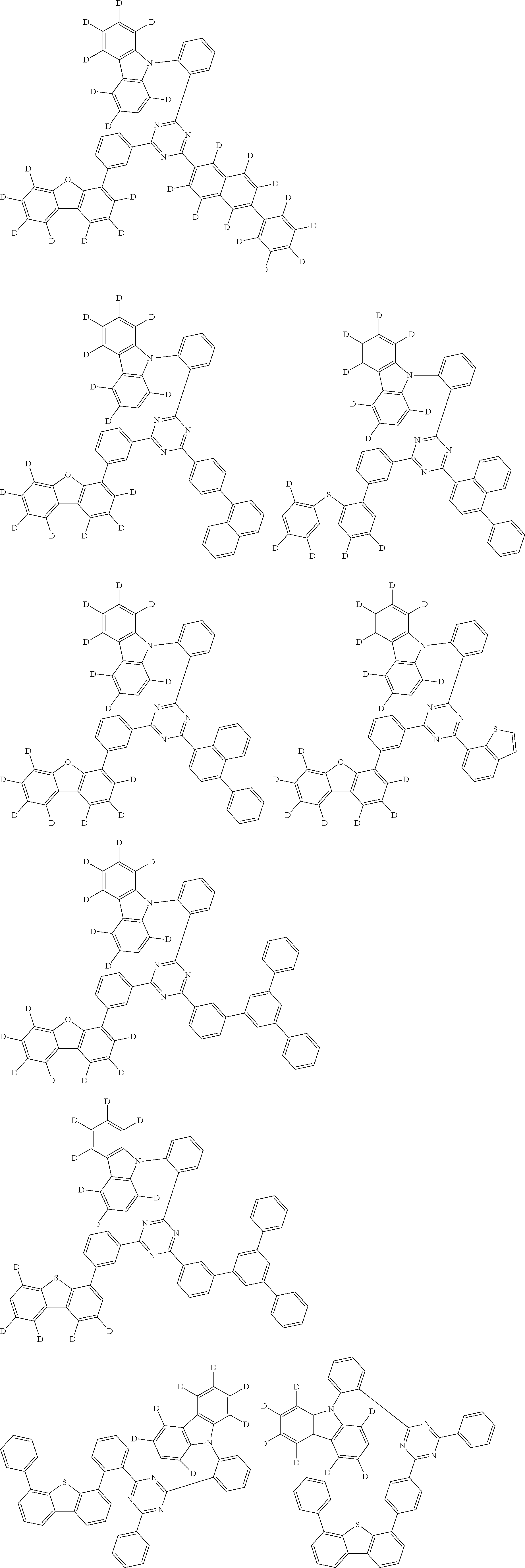

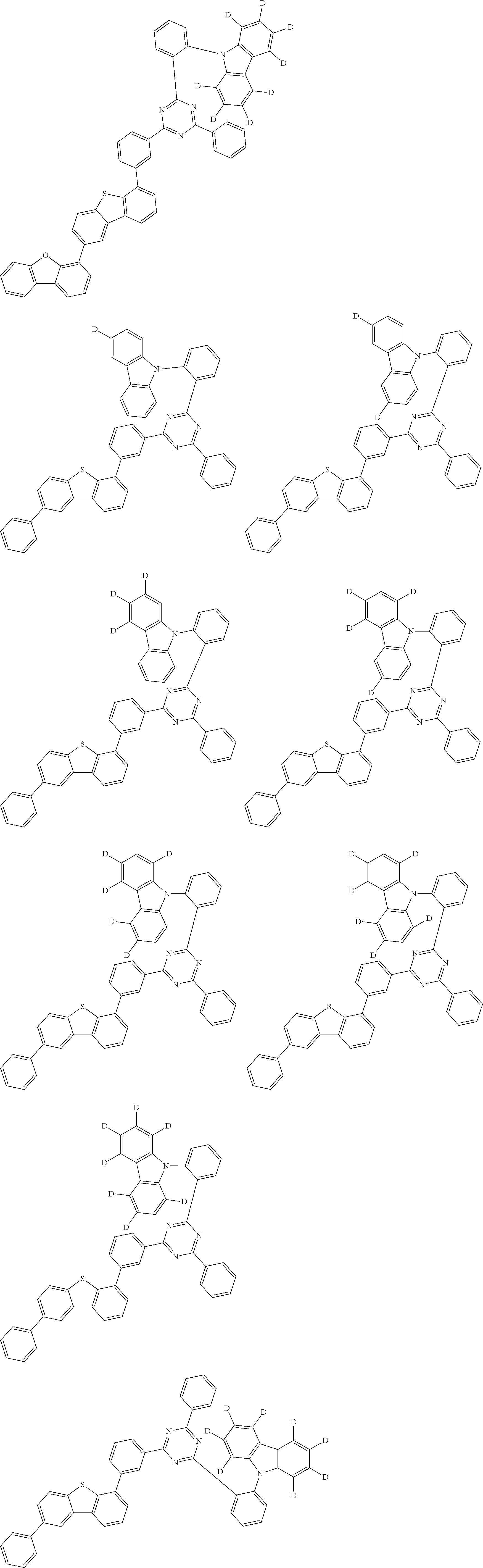

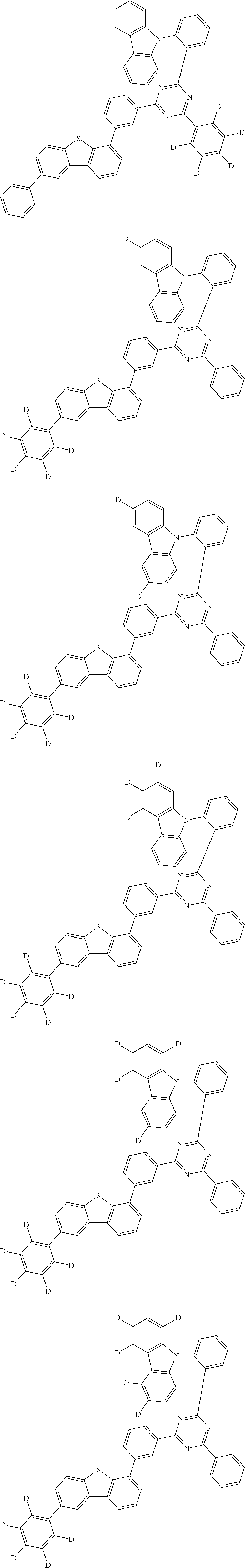

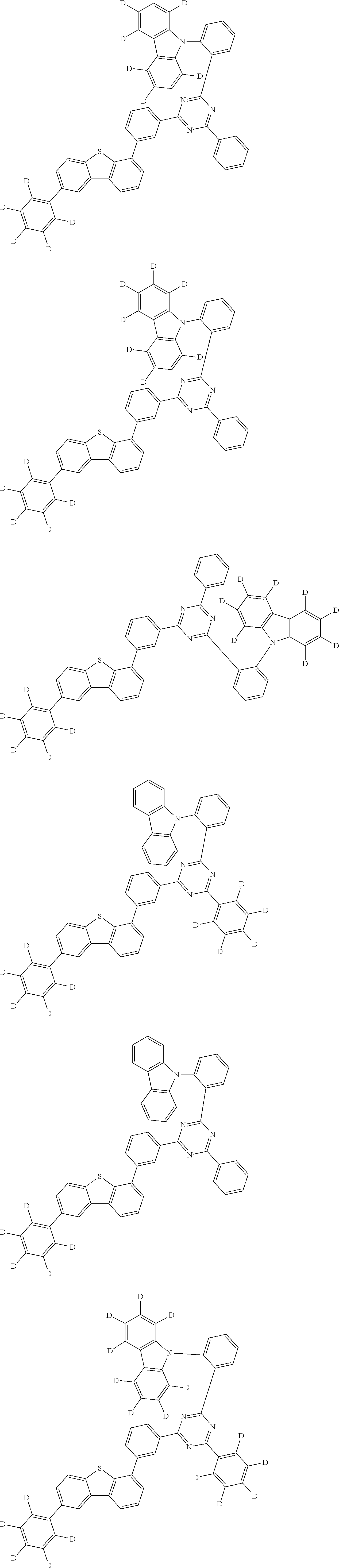

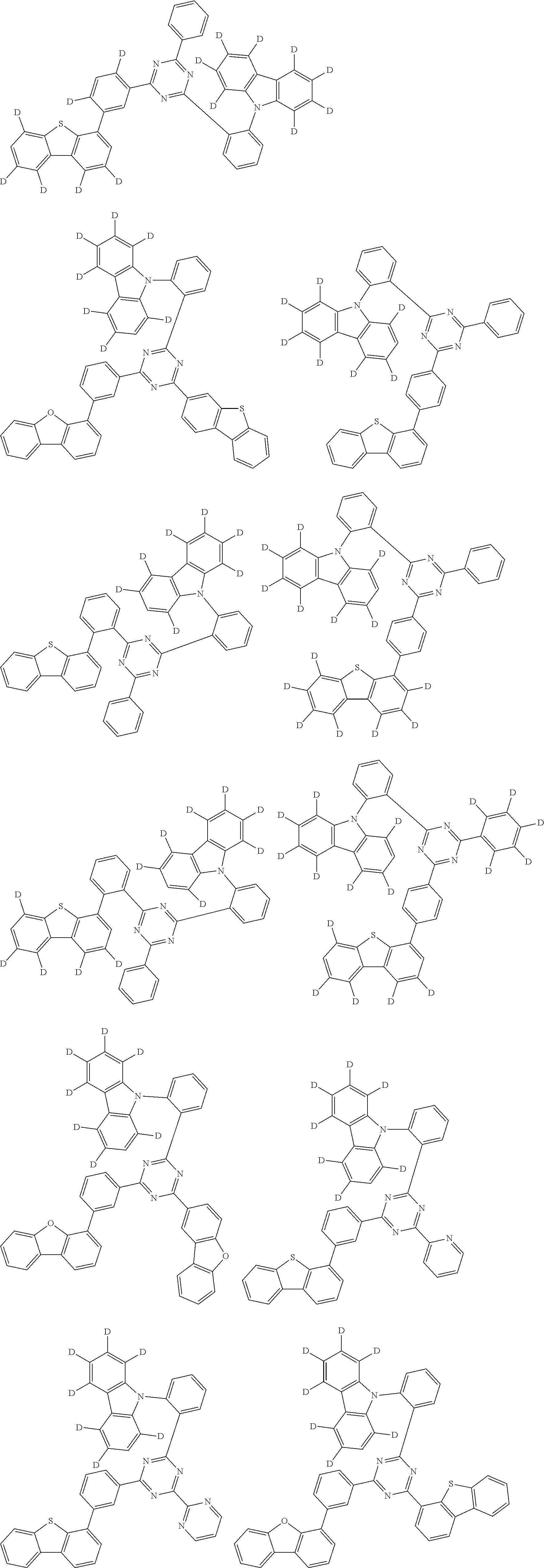

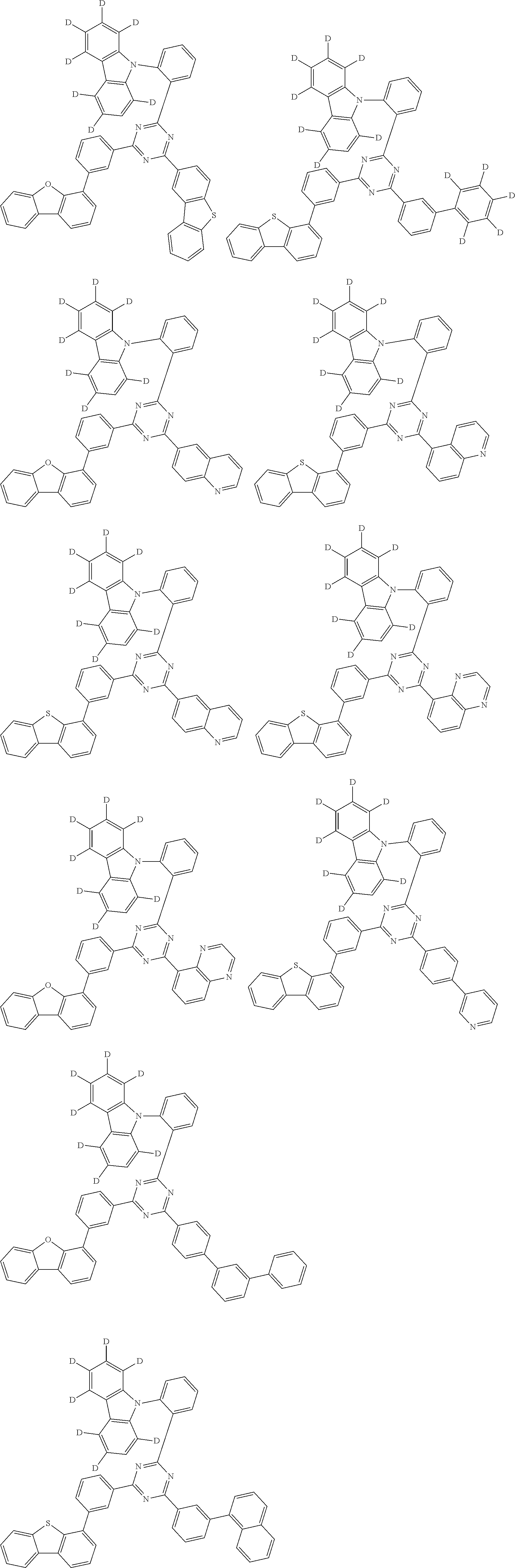

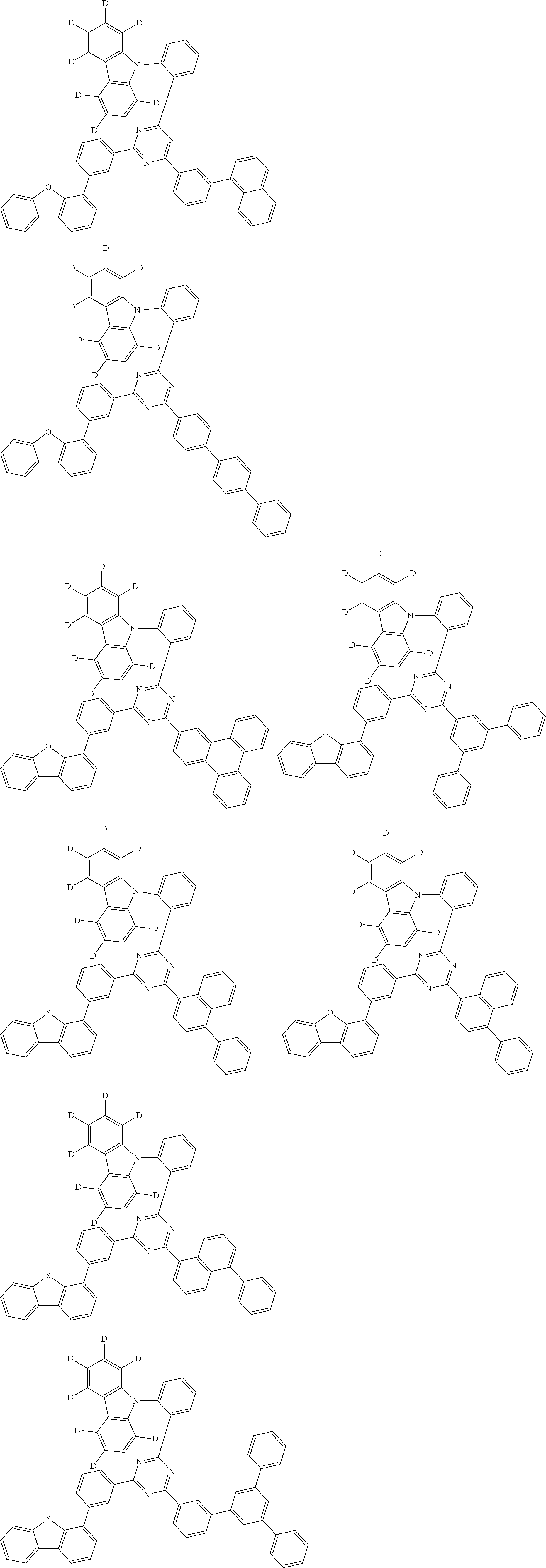

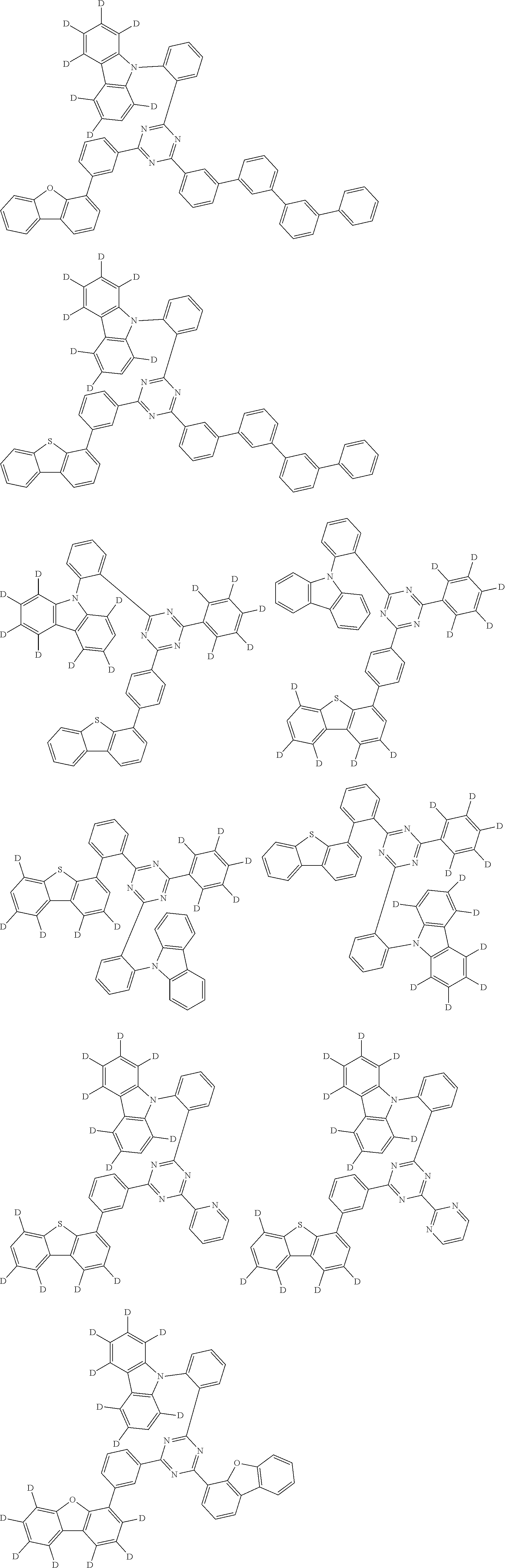

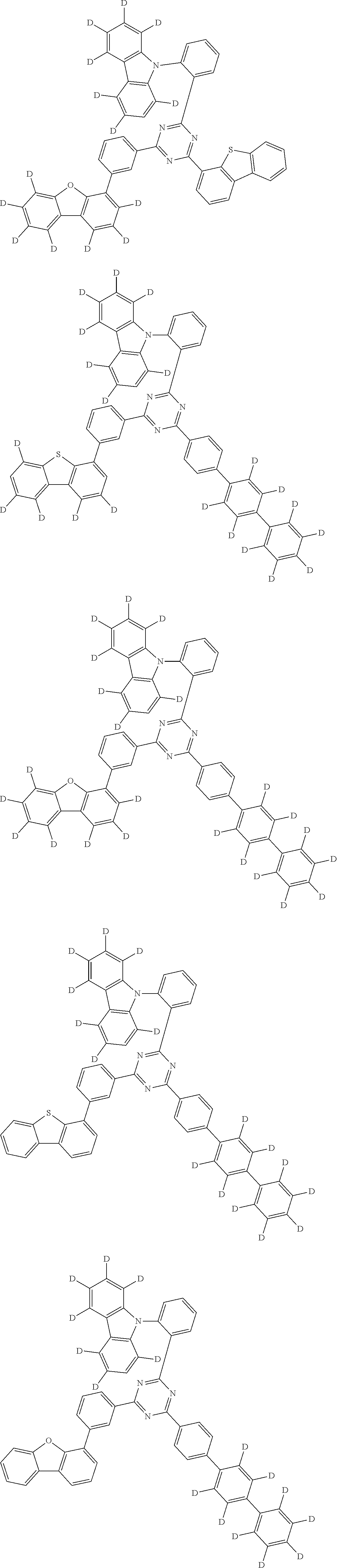

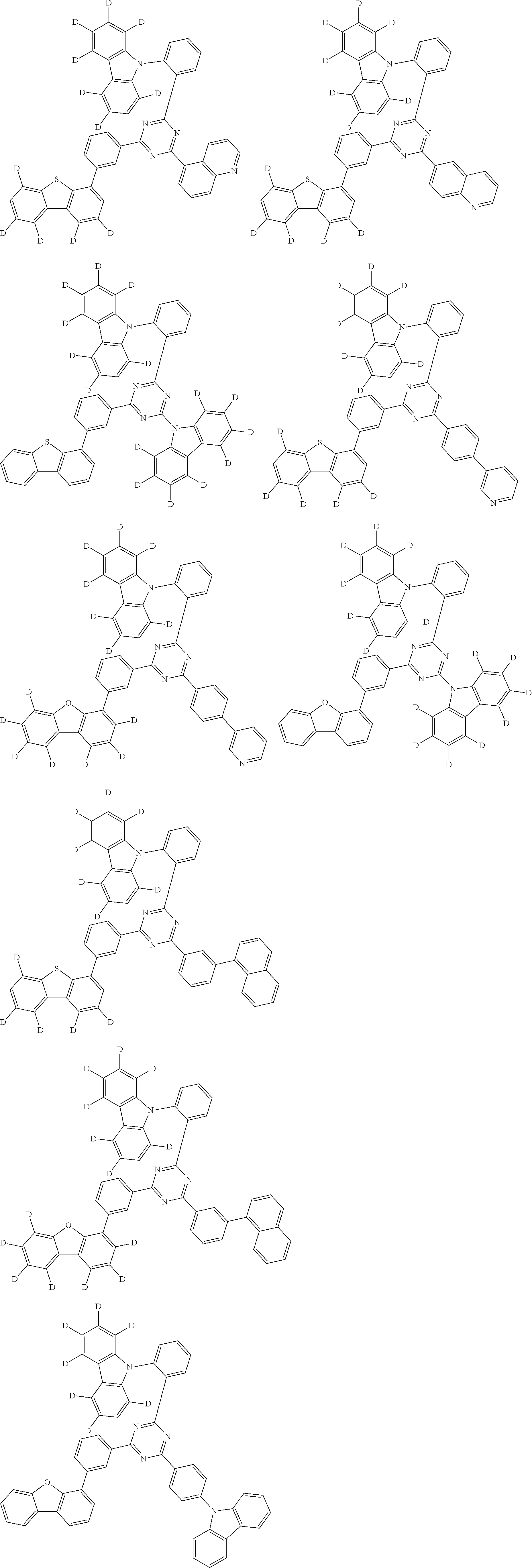

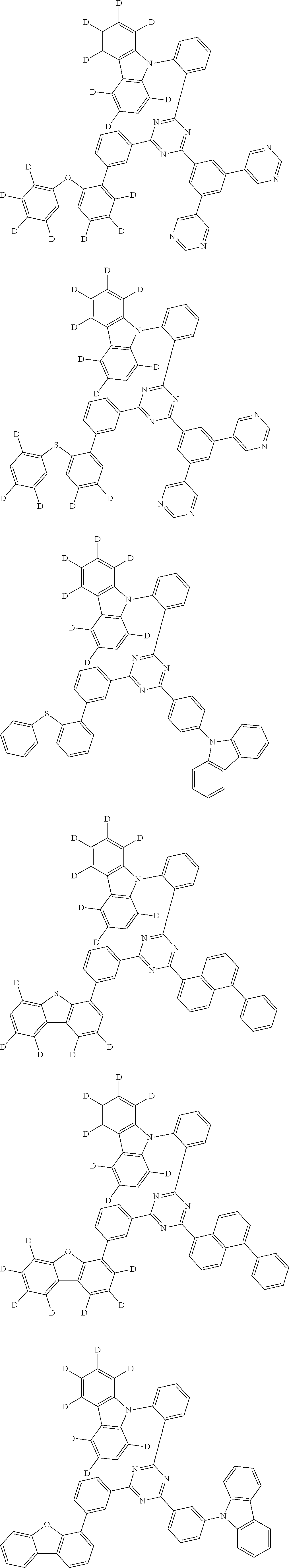

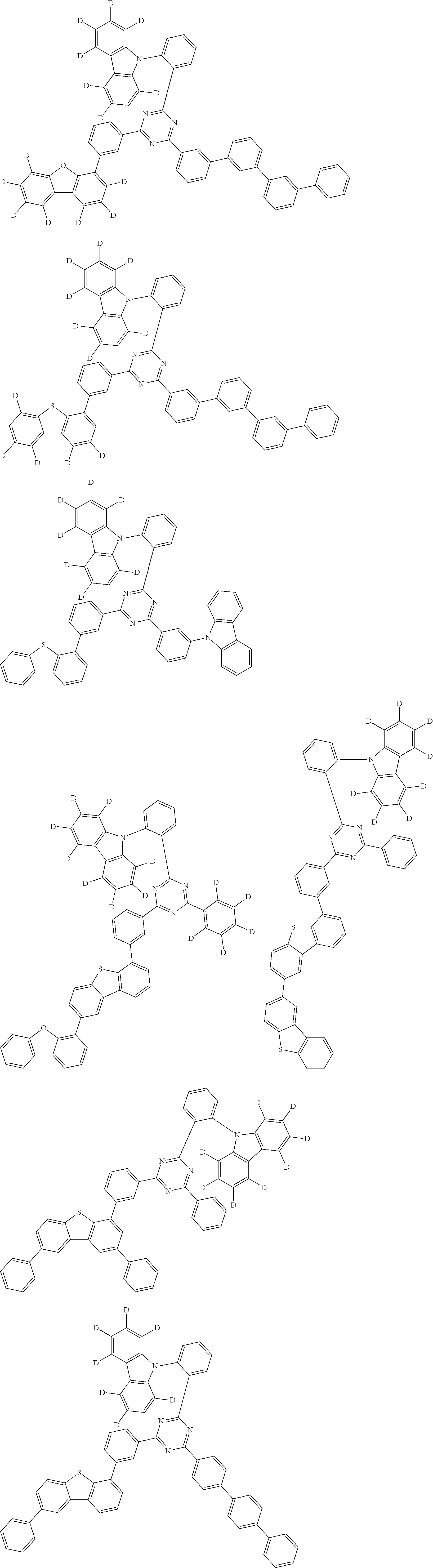

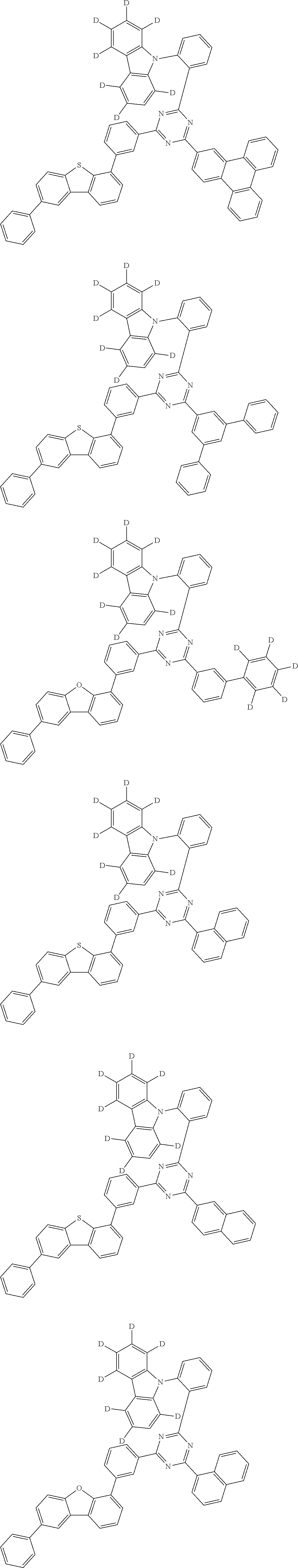

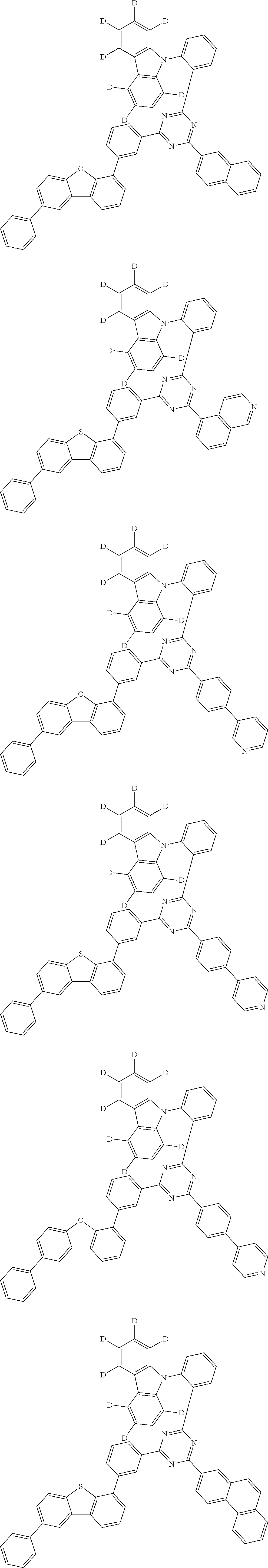

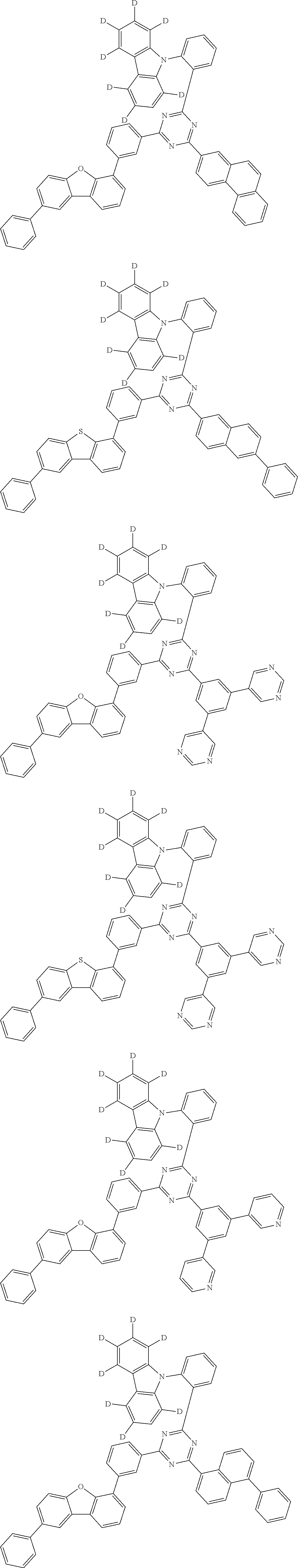

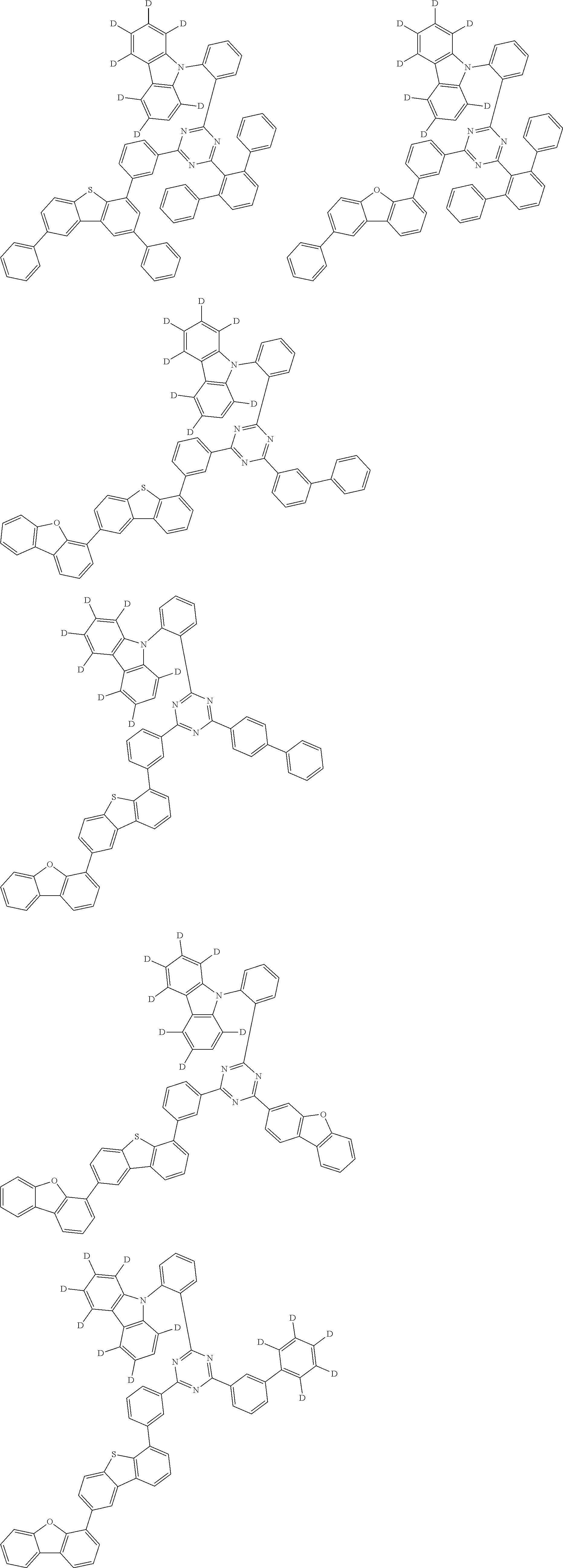

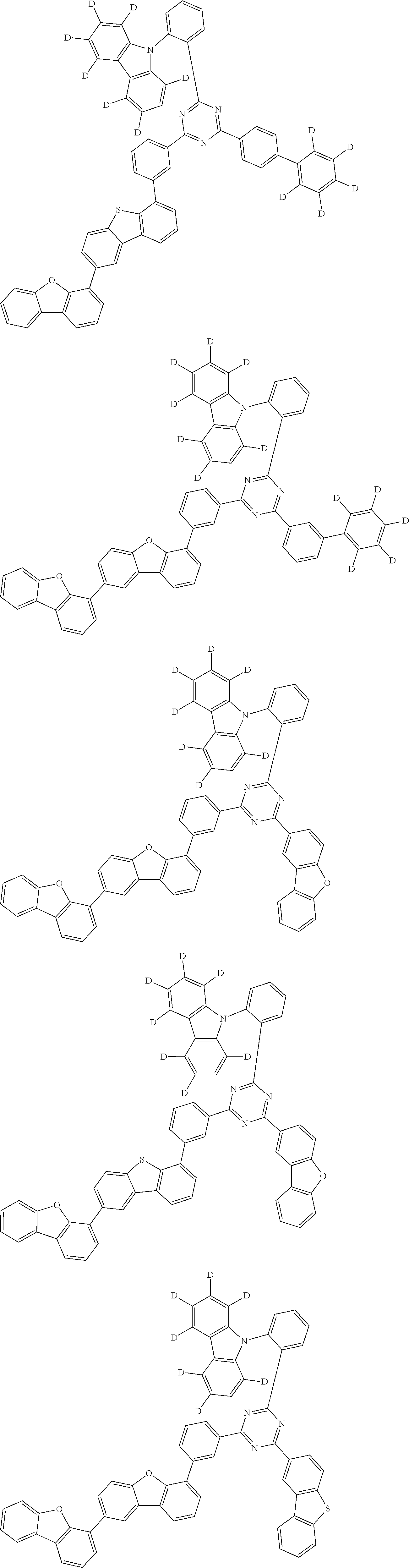

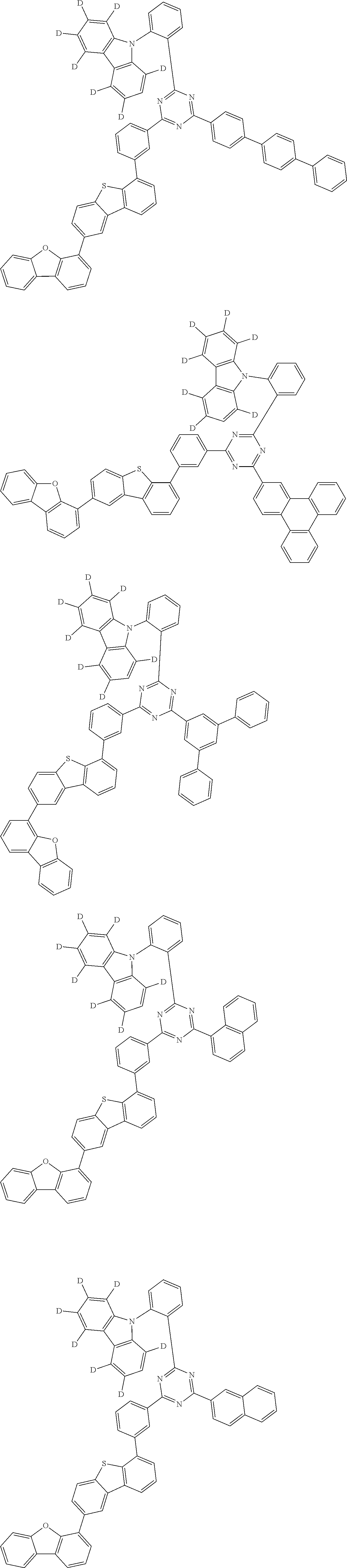

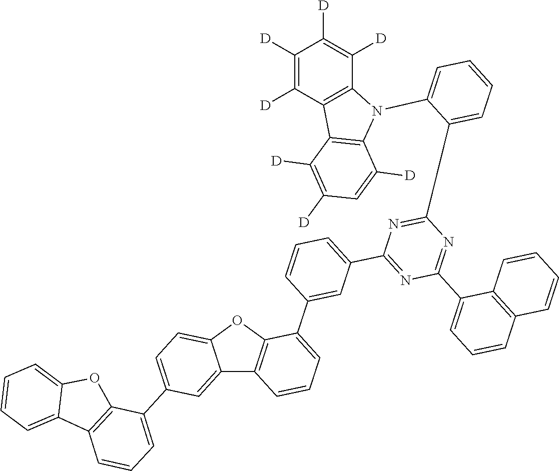 [SMILES: [2H]c1cc([2H])c2c(c1[2H])c1c([2H])c([2H])c([2H])c([2H])c1n2-c1ccccc1-c1nc(-c2cccc(-c3cccc4c3oc3ccc(-c5cccc6c5oc5ccccc56)cc34)c2)nc(-c2cccc3ccccc23)n1]

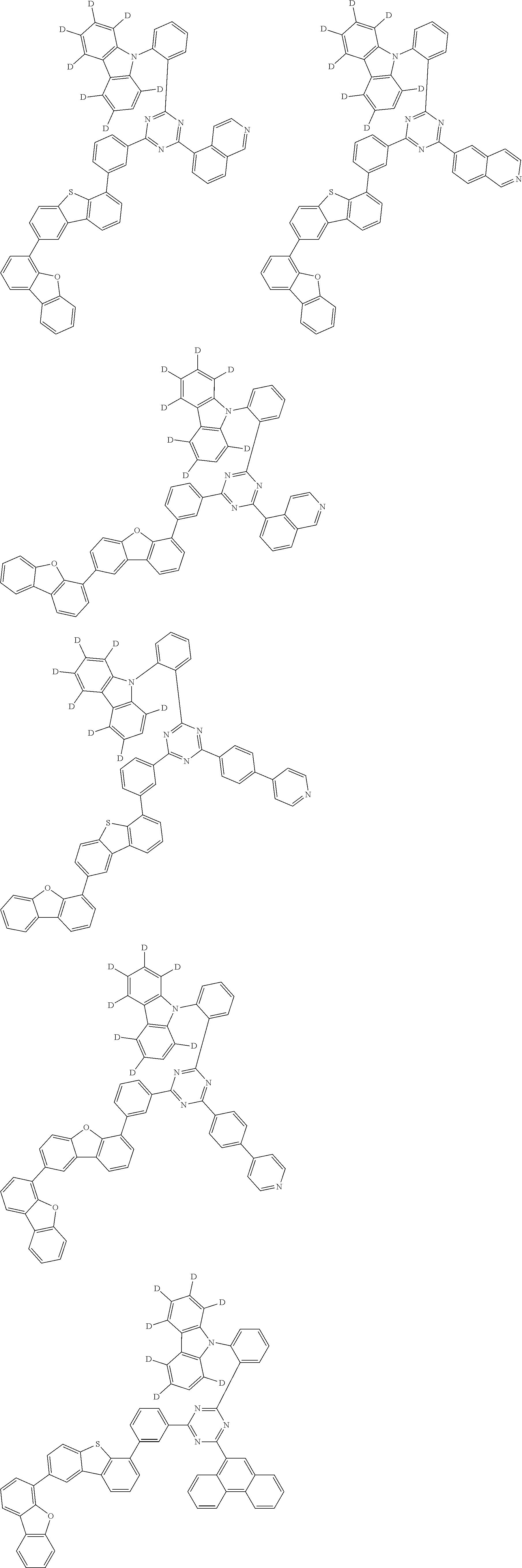

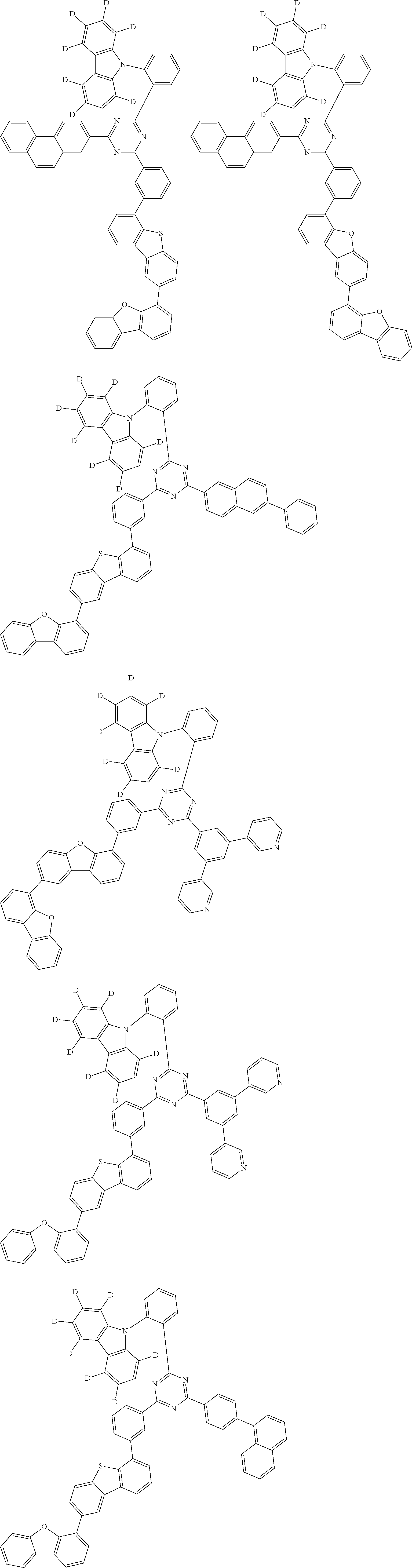

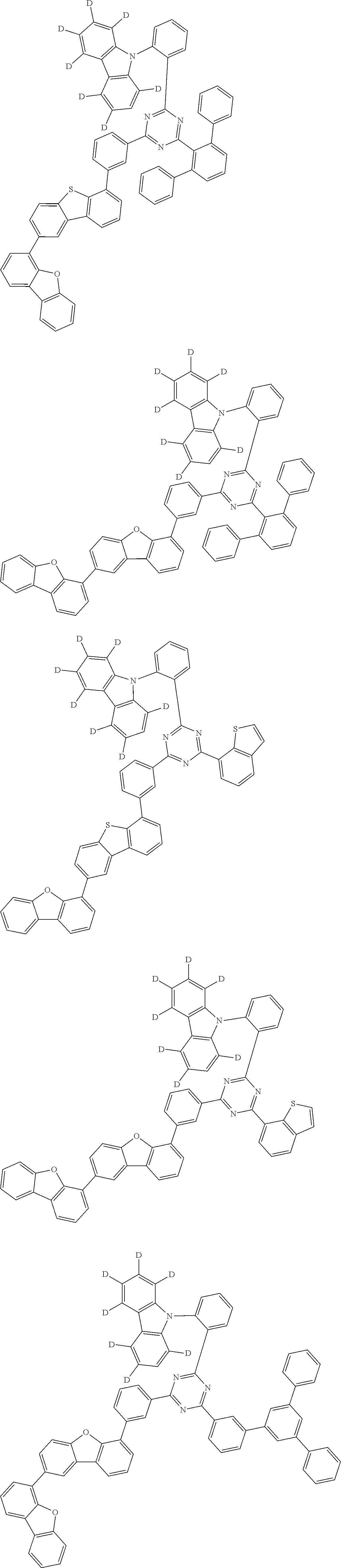

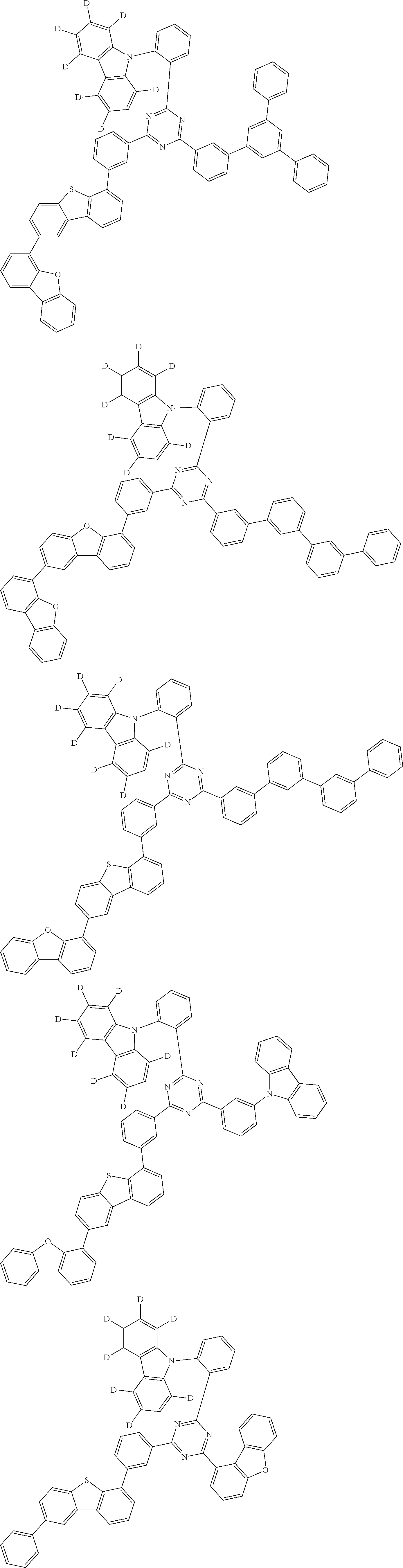

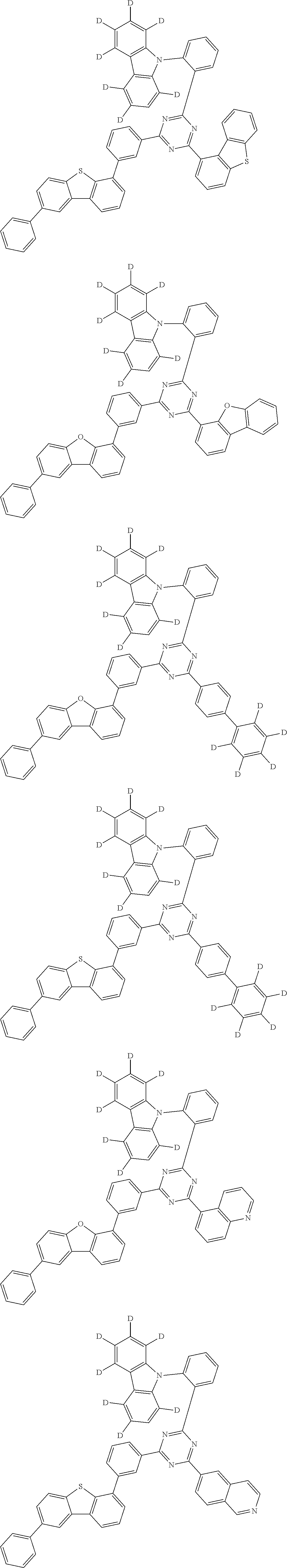

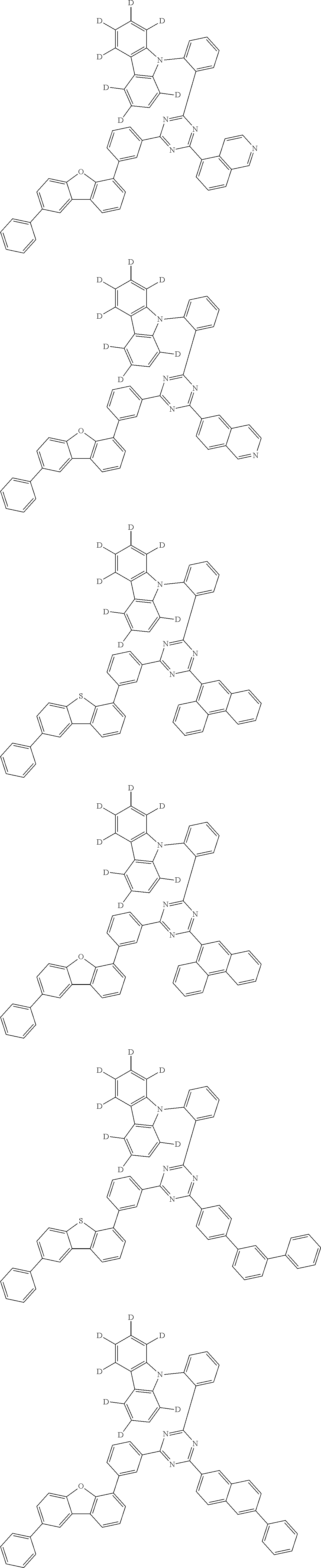

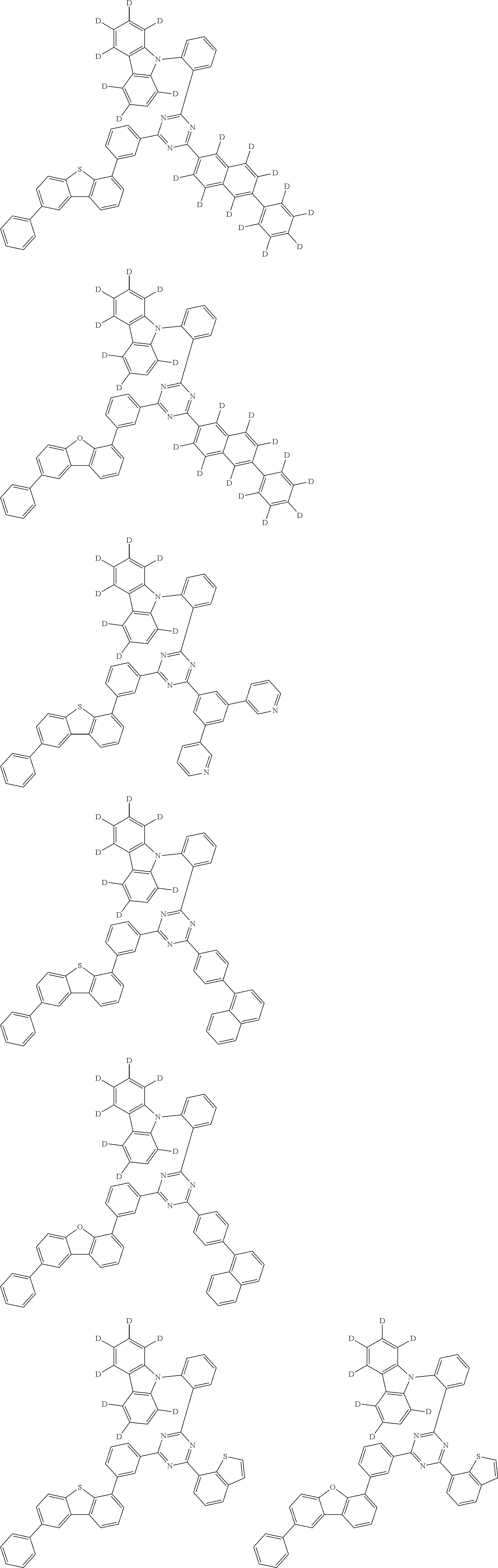

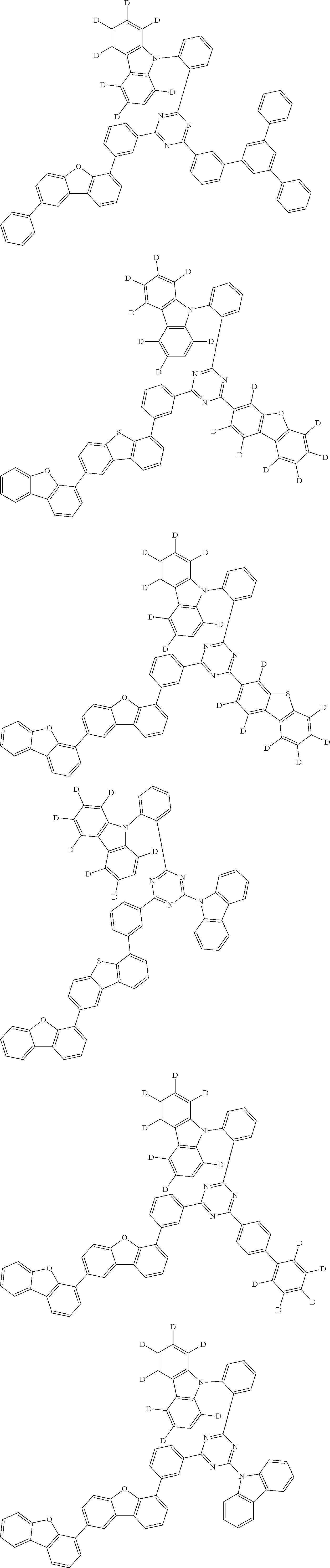

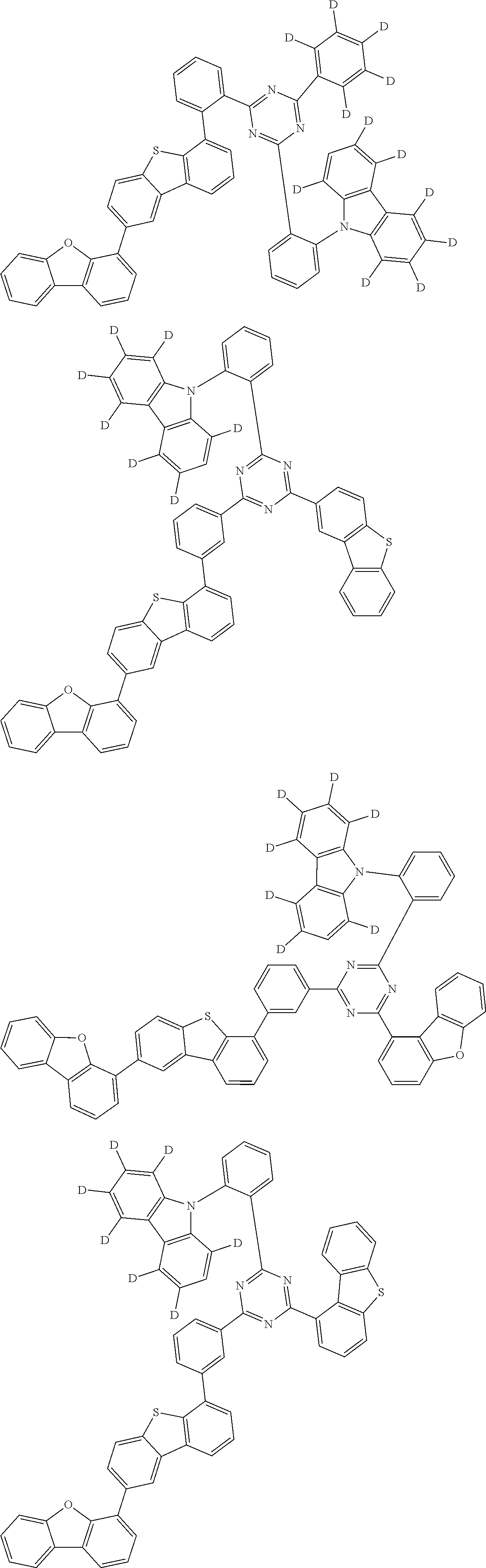

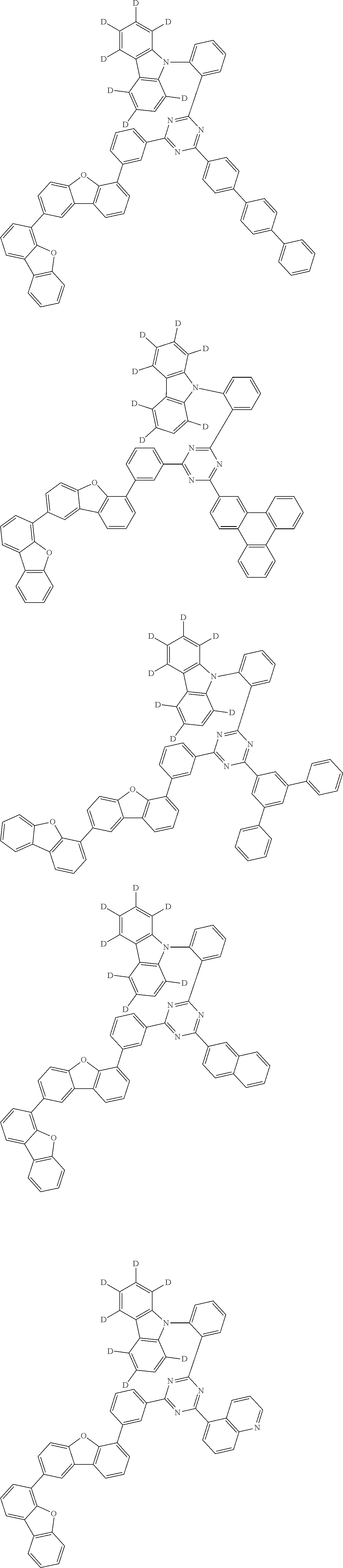

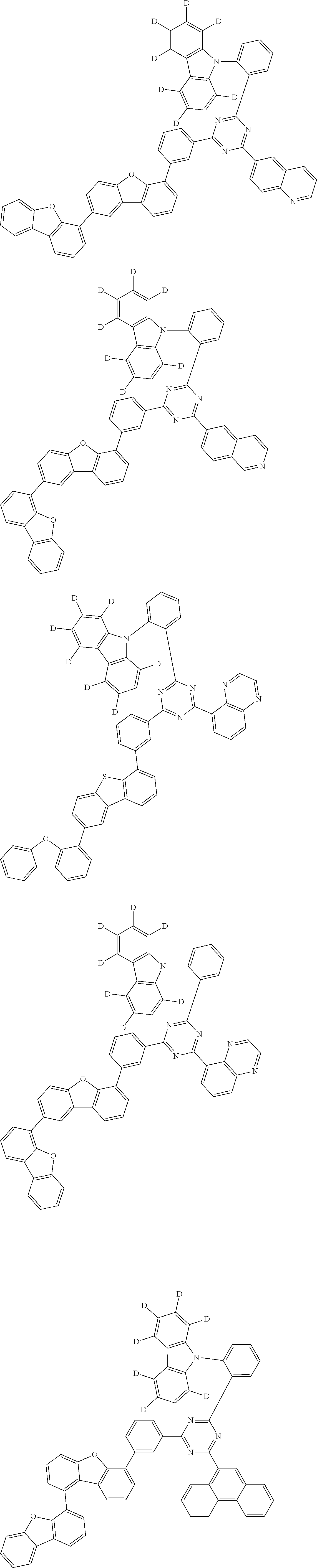

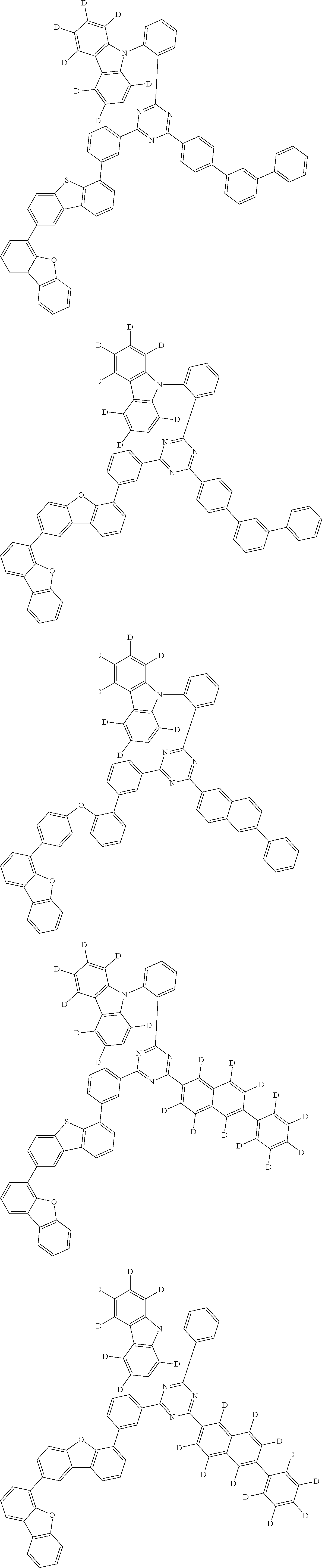

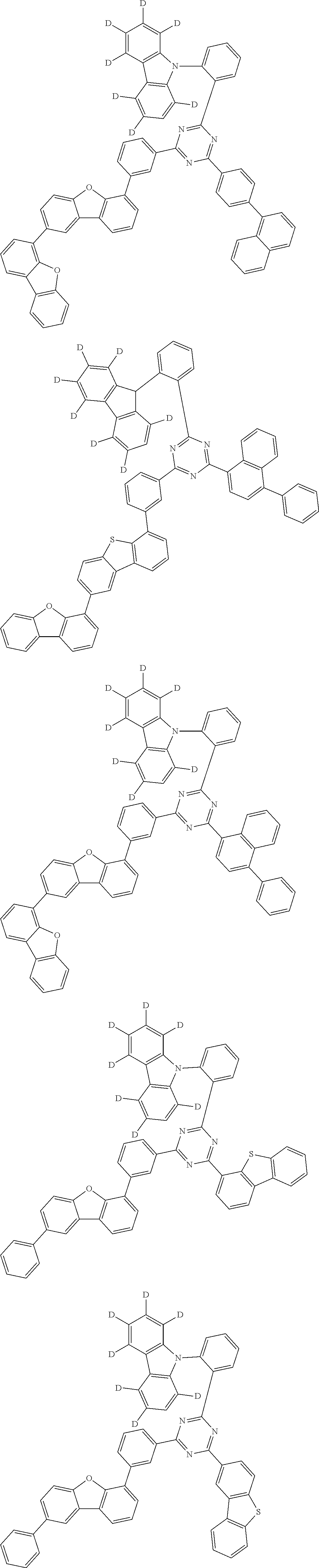

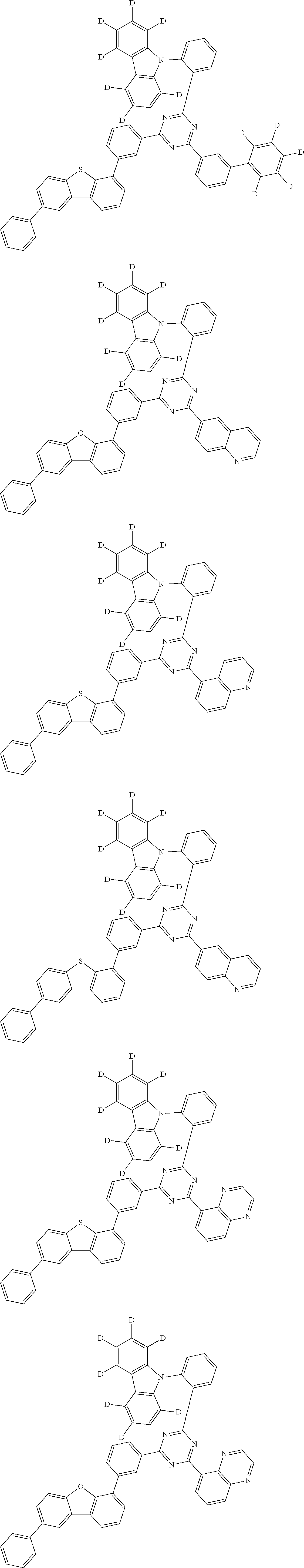

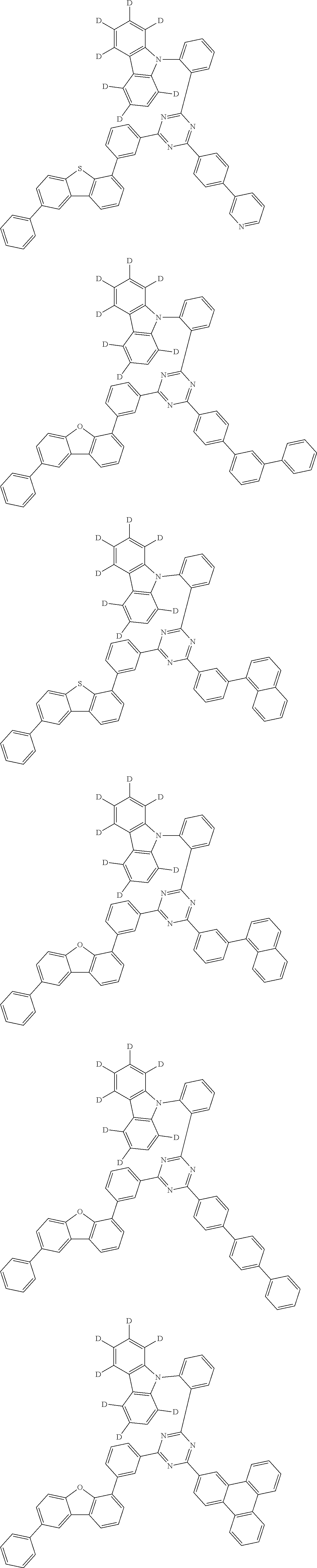

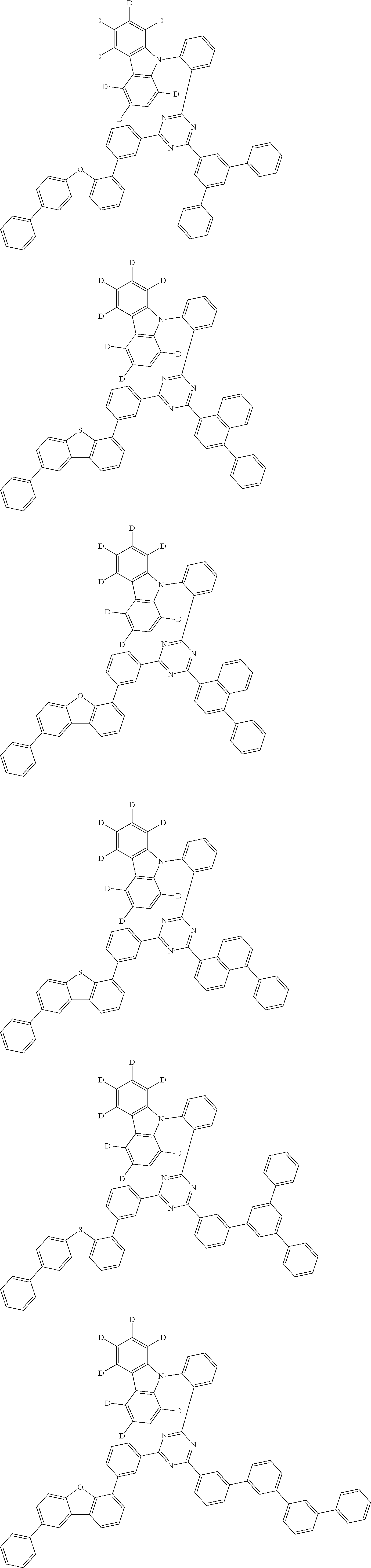

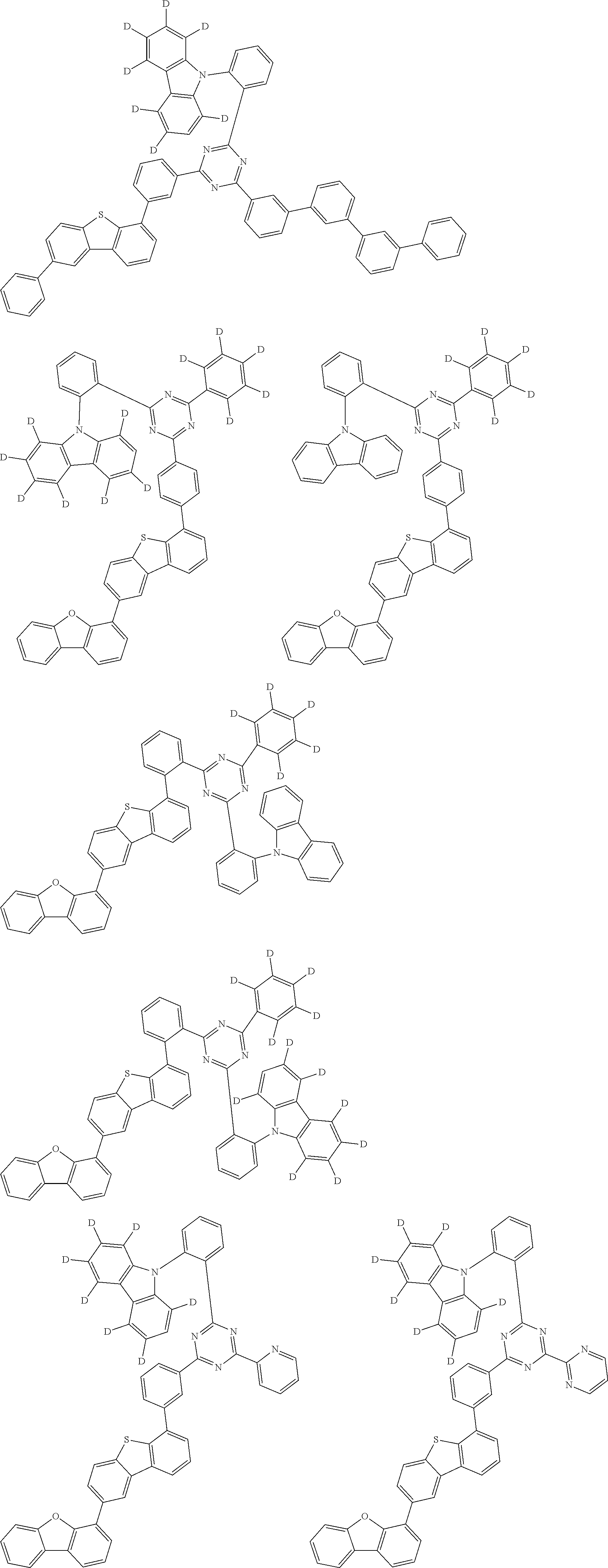

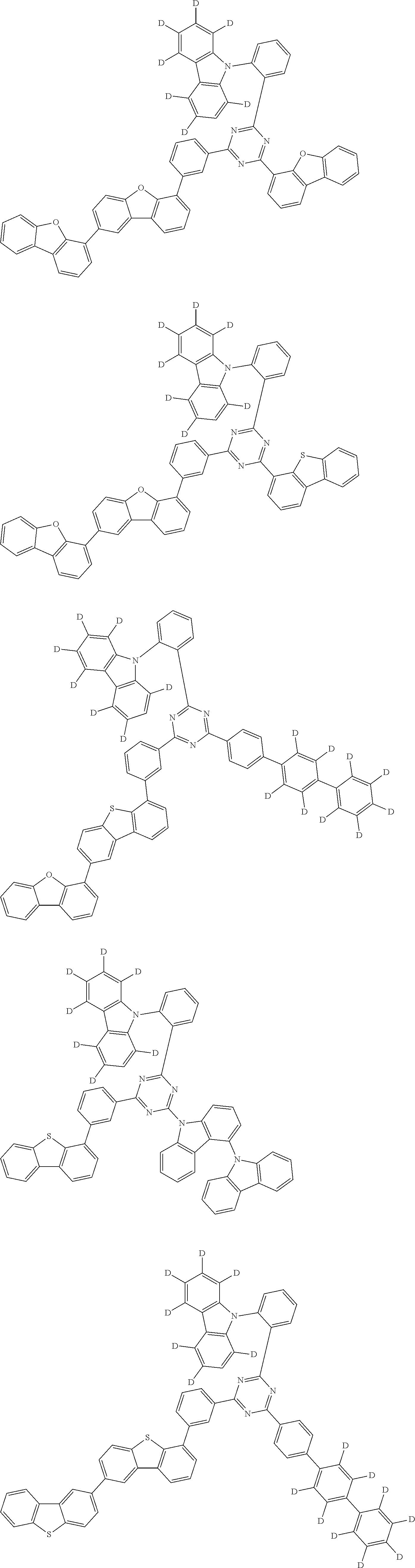

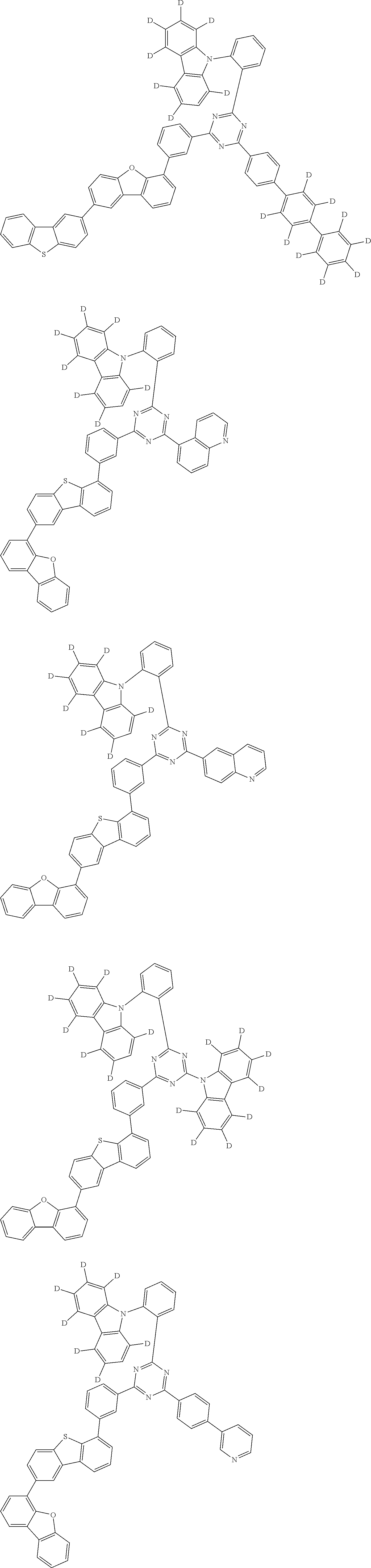

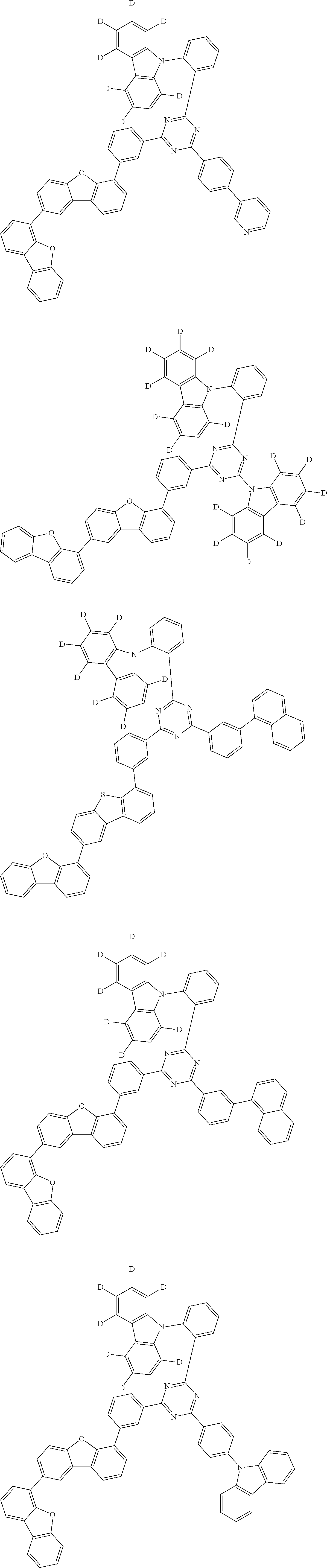

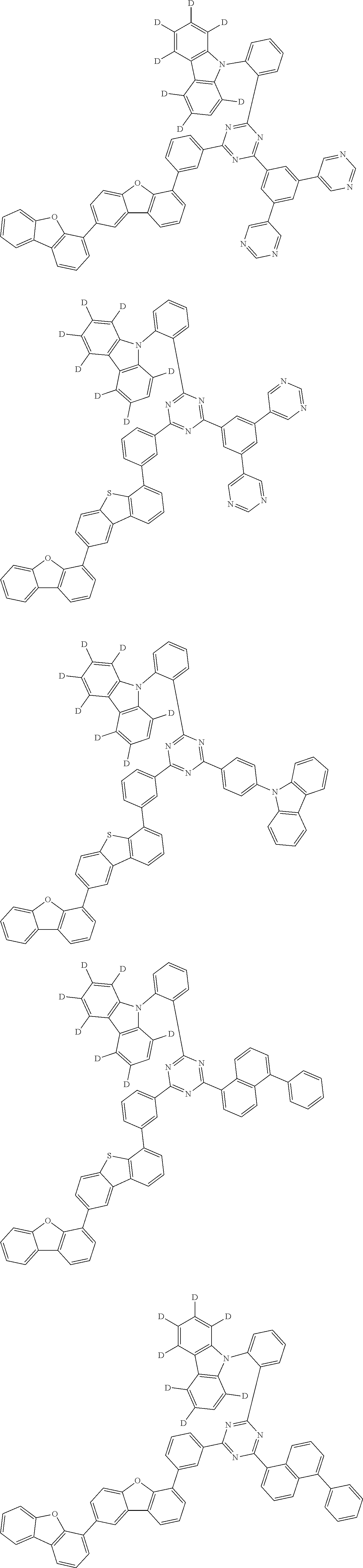

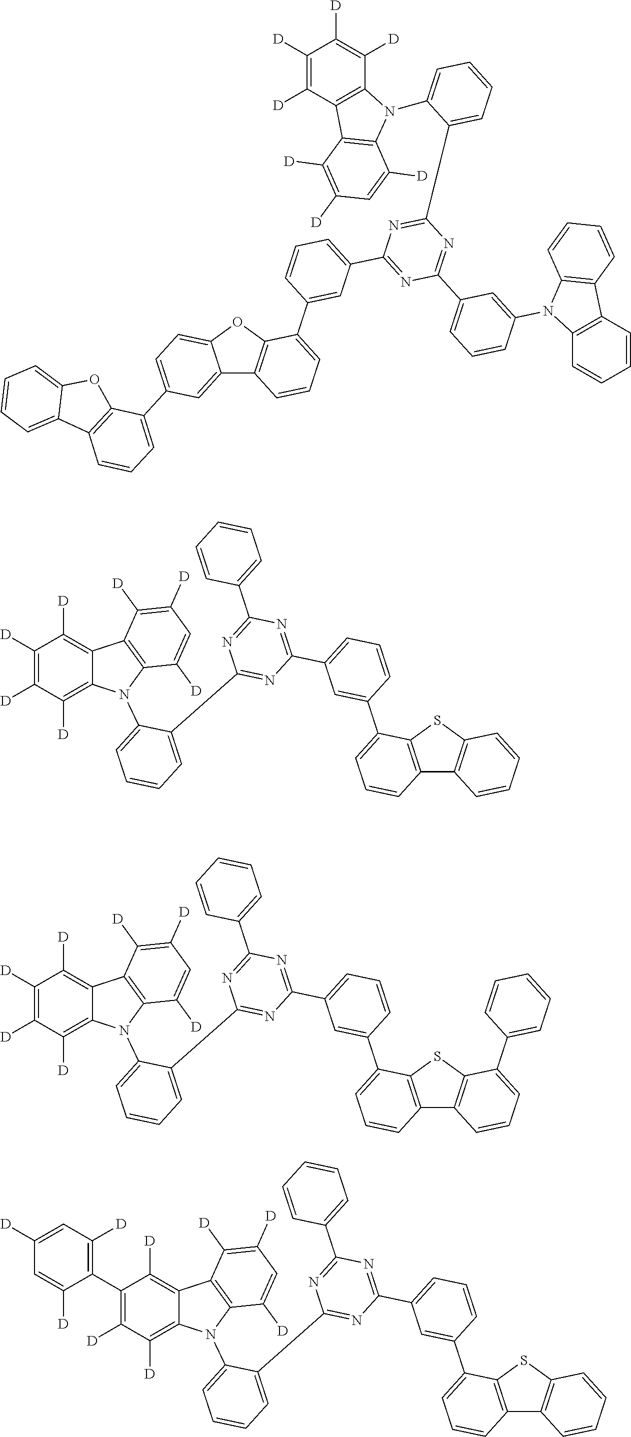

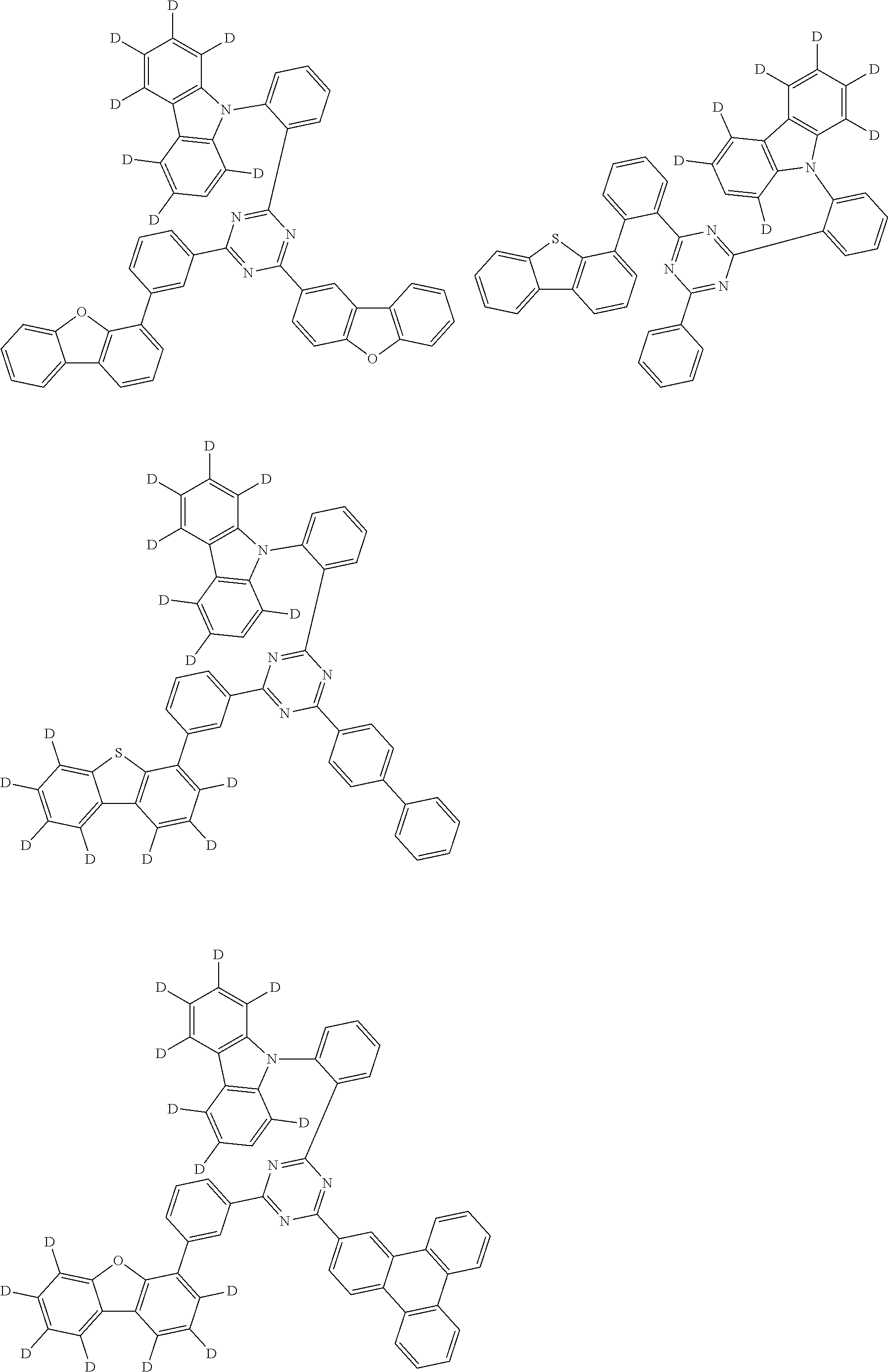

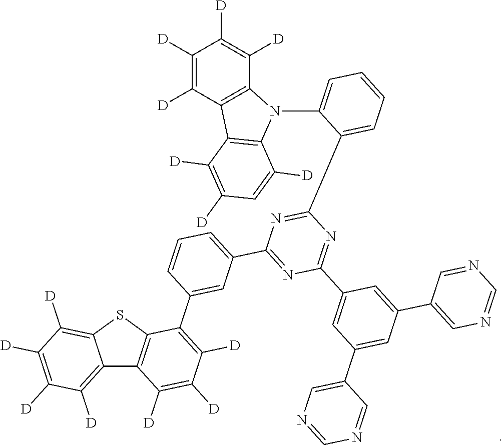 [SMILES: [2H]c1cc([2H])c2c(c1[2H])c1c([2H])c([2H])c([2H])c([2H])c1n2-c1ccccc1-c1nc(-c2cc(-c3cncnc3)cc(-c3cncnc3)c2)nc(-c2cccc(-c3c([2H])c([2H])c([2H])c4c3sc3c([2H])c([2H])c([2H])c([2H])c34)c2)n1]

9. An organic light emitting device comprising:
a first electrode;
a second electrode that is provided opposite to the first electrode; and
one or more organic material layers that are provided between the first electrode and the second electrode, wherein one or more layers of the organic material layers comprises the compound of Chemical Formula 1 of claim 1.

10. The organic light emitting device of claim 9, wherein:
the organic material layer is a light emitting layer.
